(19) United States
(12) Patent Application Publication
(10) Pub. No.: US 2008/0132412 A1
(43) Pub. Date: Jun. 5, 2008

(54) 7-Amino-6-Heteroaryl-1,2,4-Triazolo[1,5-A]Pyrimidines and Their Use for Controlling Harmful Fungi

(75) Inventors: Oliver Wagner, Neustadt (DE); Udo Hunger, Mannheim (DE)

Correspondence Address: HUTCHISON LAW GROUP PLLC, PO BOX 31686, RALEIGH, NC, 27612, US

(73) Assignee: BASF Aktiengesellschaft, Ludwigshafen (DE)

(21) Appl. No.: 11/793,197

(22) PCT Filed: Dec. 15, 2005
(86) PCT No.: PCT/EP2005/013523

§ 371 Date: Jun. 14, 2007

(30) Foreign Application Priority Data
Dec. 17, 2004 (DE) 102004060958.6
Dec. 23, 2004 (DE) 102004062199.3
Sep. 1, 2005 (DE) 2005041766.3

(51) Int. Cl. A01N 25/34; C07D 239/70; A01P 3/00; A01N 43/90

(52) U.S. Cl. 504/100; 544/263; 514/259.31

(57) ABSTRACT

The present invention relates to 7-amino-6-heteroaryl-1,2,4-triazolo[1,5-a]pyrimidine compounds of the formula (I)

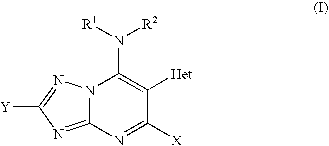

in which the meanings of the substituents R¹, R², Het, X and Y are as defined in the description.

The present invention relates to 7-amino-6-heteroaryl-1,2,4-triazolo[1,5-a]pyrimidine compounds of the formula (I)

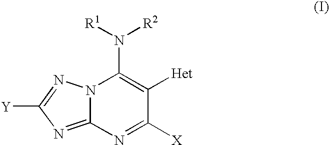

in which the substituents R¹, R², Het, X and Y are as defined below:
Het is a 6-membered heteroaromatic radical which contains one, two or three nitrogen atoms, where Het is unsubstituted or substituted by one, two, three or four identical or different substituents L,
R¹, R² independently of one another are hydrogen, (C₁-C₈)-alkyl, (C₁-C₈)-haloalkyl, (C₁-C₈)-alkoxy, (C₃-C₈)-cycloalkyl, (C₃-C₈)-cycloalkoxy, (C₅-C₁₀)-bicycloalkyl, (C₃-C₈)-halocycloalkyl, (C₂-C₈)-alkenyl, (C₂-C₈)-alkenyloxy, (C₄-C₁₀)-alka-dienyl, (C₂-C₈)-haloalkenyl, (C₃-C₆)-cycloalkenyl, (C₃-C₆)-halocycloalkenyl, (C₂-C₈)-alkynyl, (C₂-C₈)-alkynyloxy, (C₂-C₈)-haloalkynyl, NH₂, (C₁-C₈)-alkylamino, di-(C₁-C₈)-alkylamino, phenyl, naphthyl or a five- or six-membered saturated, partially unsaturated or aromatic heterocycle which contains one, two, three or four heteroatoms from the group consisting of O, N and S,
   R¹ and R² together with the nitrogen atom to which they are attached may also form a five- or six-membered heterocyclyl or heteroaryl which is attached via N and may contain one, two or three further heteroatoms from the group consisting of O, N and S as ring members and/or may carry one or more substituents from the group consisting of halogen, (C₁-C₆)-alkyl, (C₁-C₆)-haloalkyl, (C₂-C₆)-alkenyl, (C₂-C₆)-haloalkenyl, (C₁-C₆)-alkoxy, (C₁-C₆)-alkoxycarbonyl, (C₁-C₆)-haloalkoxy, (C₃-C₆)-alkenyloxy, (C₃-C₆)-haloalkenyloxy and/or in which two substituents attached to adjacent ring atoms may be (C₁-C₆)-alkylene, oxy-(C₂-C₄)-alkylene or oxy-(C₁-C₃)-alkyleneoxy;
   R¹ and/or R² may carry one, two, three or four identical or different groups R^{a}:
   R^{a} is halogen, cyano, nitro, hydroxyl, carboxyl, (C₁-C₆)-alkyl, (C₁-C₆)-haloalkyl, (C₁-C₆)-alkylcarbonyl, (C₃-C₆)-cycloalkyl, (C₁-C₆)-alkoxy, (C₁-C₆)-haloalkoxy, (C₁-C₆)-alkoxycarbonyl, (C₁-C₆)-alkylthio, (C₁-C₆)-alkylamino, di-(C₁-C₆)-alkylamino, (C₁-C₆)-alkylaminocarbonyl, di-(C₁-C₆)-alkylaminocarbonyl, (C₂-C₈)-alkenyl, (C₄-C₁₀)-alkadienyl, (C₂-C₈)-haloalkenyl, (C₃-C₈)-cycloalkenyl, (C₂-C₆)-alkenyloxy, (C₃-C₆)-haloalkenyloxy, (C₂-C₆)-alkynyl, (C₂-C₆)-haloalkynyl, (C₃-C₆)-alkynyloxy, (C₃-C₆)-haloalkynyloxy, (C₃-C₆)-cycloalkoxy, (C₃-C₆)-cycloalkenyloxy, oxy-(C₁-C₃)-alkyleneoxy, phenyl, naphthyl, a five-, six-, seven-, eight-, nine- or ten-membered saturated, partially unsaturated or aromatic heterocycle which contains one, two, three or four heteroatoms from the group consisting of O, N and S,
   where the aliphatic, alicyclic or aromatic groups in R^{a} for their part may be partially or fully halogenated and/or may carry one, two or three groups R^{b}:
   R^{b} is halogen, cyano, nitro, hydroxyl, mercapto, amino, carboxyl, aminocarbonyl, aminothiocarbonyl, alkyl, haloalkyl, alkenyl, alkadienyl, alkenyloxy, alkynyloxy, alkoxy, haloalkoxy, alkylthio, alkylamino, dialkylamino, formyl, alkylcarbonyl, alkylsulfonyl, alkylsulfinyl, alkoxycarbonyl, alkylcarbonyloxy, alkylaminocarbonyl, dialkylaminocarbonyl, alkylaminothiocarbonyl, dialkylaminothiocarbonyl, where the alkyl groups in these radicals contain 1 to 6 carbon atoms and the alkenyl, alkadienyl or alkynyl groups mentioned in these radicals contain 2 to 8 carbon atoms;
   and/or one, two or three of the following radicals:
   cycloalkyl, bicycloalkyl, cycloalkoxy, heterocyclyl, heterocyclyloxy, where the cyclic systems contain 3 to 10 ring members; aryl, aryloxy, arylthio, aryl-(C₁-C₆)-alkoxy, aryl-(C₁-C₆)-alkyl, heteroaryl, heteroaryloxy, heteroarylthio, where the aryl radicals preferably contain 6, 7, 8, 9 or 10 ring members and the heteroaryl radicals 5 or 6 ring members; where the cyclic systems may be partially or fully halogenated or substituted by alkyl or haloalkyl groups;
X is (C₁-C₈)-alkyl, (C₂-C₈)-alkenyl, (C₂-C₈)-alkynyl; where these groups may be partially or fully halogenated and/or may carry one, two or three substituents selected from the group consisting of nitro, cyano, (C₁-C₂)-alkoxy, (C₁-C₄)-alkoxycarbonyl, amino, (C₁-C₄)-alkylamino and di-(C₁-C₄)-alkylamino;
Y is hydrogen, halogen, cyano, (C₁-C₈)-alkyl, (C₁-C₈)-haloalkyl, (C₂-C₈) alkenyl, (C₂-C₈)-haloalkenyl, (C₂-C₈)-alkynyl, (C₂-C₈)-haloalkynyl, (C₁-C₈)-alkoxy, (C₁-C₈)-haloalkoxy, (C₃-C₈)-cycloalkyl, (C₃-C₈)-halocycloalkyl, (C₁-C₈)-alkylthio, (C₁-C₈)-alkylsulfinyl, (C₁-C₈)-alkylsulfonyl, NH₂, (C₁-C₈)-alkylamino, di-(C₁-C₈)-alkylamino or C(═O)A²;
   where
   L is selected from the group consisting of halogen, cyano, hydroxyl, cyanato (OCN), nitro, (C₁-C₈)-alkyl, (C₂-C₁₀)-alkenyl, (C₂-C₁₀)-alkynyl, (C₁-C₆)-haloalkyl, (C₂-C₁₀)-haloalkenyl, (C₁-C₆)-alkoxy, (C₂-C₁₀)-alkenyloxy, (C₂-C₁₀)-alkynyloxy, (C₁-C₆)-haloalkoxy, (C₃-C₆)-cycloalkyl, (C₃-C₆)-cycloalkenyl, (C₃-C₆)-cycloalkoxy, (C₁-C₈)-alkoximinoalkyl, (C₂-C₁₀)-alkenyloximinoalkyl, (C₂-C₁₀)-alkynyloximinoalkyl, (C₂-C₁₀)-alkynylcarbonyl, (C₃-C₆)-cycloalkylcarbonyl, phenyl, a five-, six-, seven-, eight-, nine- or ten-membered saturated, partially unsaturated or aromatic heterocycle which contains one, two, three or four heteroatoms from the group consisting of O, N and S, where the phenyl and the heterocycle are unsubstituted or substituted by one, two, three or four substituents independently of one another selected from the group consisting of halogen, (C₁-C₆)-alkyl, (C₁-C₆)-haloalkyl, (C₂-C₆)-alkenyl, (C₂-C₆)-haloalkenyl, (C₁-C₆)-alkoxy, (C₁-C₆)-alkoxycarbonyl, (C₁-C₆)-haloalkoxy, (C₃-C₆)-alkenyloxy, (C₃-C₆)-haloalkenyloxy and/or in which two substituents attached to adjacent ring atoms may be (C₁-C₆)-alkylene, oxy-(C₂-C₄)-alkylene or oxy-(C₁-C₃)-alkyleneoxy; amino, NR⁵R⁶, NR⁵—(C═O)—R⁶, S(═O)ₙA¹, C(═O)A², C(═S)A², a group —C(═N—OR⁷)(NR³R⁹) or a group —C(═N—NR¹⁰R¹¹)(NR¹²R¹³),
   in which
   R⁵, R⁶ independently of one another are selected from the group consisting of hydrogen, (C₁-C₆)-alkyl, (C₂-C₁₀)-alkenyl, (C₂-C₁₀)-alkynyl, (C₃-C₆)-cycloalkyl or (C₃-C₆)-cycloalkenyl, where the 5 last mentioned radicals may be partially or fully halogenated and/or may carry one, two, three or four radicals selected from the group consisting of cyano, (C₁-C₄)-alkoximino, (C₂-C₄)-alkenyloximino, (C₂-C₄)-alkynyloximino or (C₁-C₄)-alkoxy;
   A¹ is hydrogen, hydroxyl, (C₁-C₈)-alkyl, amino, (C₁-C₈)-alkylamino or di-(C₁-C₈)-alkylamino;
   n is 0, 1 or 2;
   A² is (C₂-C₈)-alkenyl, (C₁-C₈)-alkoxy, (C₁-C₆)-haloalkoxy, (C₂-C₁₀)-alkenyloxy, (C₂-C₁₀)-alkynyloxy or one of the groups mentioned Under A¹;
   R⁷, R⁸, R⁹, R¹⁰, R¹¹, R¹² and R¹³ independently of one another are selected from the group consisting of hydrogen, (C₁-C₆)-alkyl, (C₃-C₆)-cycloalkyl, (C₂-C₆)-alkenyl or (C₂-C₆)-alkynyl, where the four last-mentioned radicals may have one, two, three, four, five or six radicals R^{a}; or
   R⁸ and R⁹, R¹⁰ and R¹¹ and/or R¹² and R¹³ together with the nitrogen atom to which they are attached form a four-, five- or six-membered saturated or partially unsaturated ring which may carry one, two, three or four substituents independently of one another selected from the group R^{a};
      and the agriculturally acceptable salts of the compounds (I).

The invention furthermore provides compounds of the formula (I) and salts thereof where R¹, Het, X and Y are as defined above, where Het is not 3-chloro-5-(trifluoromethyl)pyridin-2-yl, 5-fluoropyrimidin-4-yl, 3-(trifluoromethyl)pyridin-2-yl or 5-chloropyrimidin-4-yl, and R² is an organic radical which contains 3 to 13 carbon atoms and one or more, for example 1, 2 or 3 silicon atoms, and also, if appropriate, 1 to 3 identical or different heteroatoms from the group consisting of oxygen, nitrogen and sulfur, and which is unsubstituted or carries 1, 2, 3 or 4 identical or different substituents selected from the group consisting of halogen atoms and the substituents R^{a}. The invention furthermore provides compounds of the formula I where Het, X and Y are as defined above and in which R¹ and R² together with the nitrogen atom, to which they are attached, are a heterocyclic ring having preferably 3 to 12 ring members which has one or more, for example 1, 2 or 3, silicon atoms and which is unsubstituted or carries 1, 2, 3 or 4 identical or different substituents selected from the group consisting of halogen atoms and the substituents R^{a}. Here, in one embodiment Het is not pyridin-2-yl or pyrimidin-4-yl. In this subject matter of the invention, Het is preferably pyridazinyl, pyrazinyl, 1,2,4-triazinyl or 1,3,5-triazinyl.

Furthermore, the present invention relates to compositions comprising at least one of the compounds according to the invention, to processes for preparing these compounds, to intermediates for preparing these compounds and to the agriculturally acceptable salts thereof, to the preparation of the intermediates and to the use of the compounds according to the invention for controlling phytopathogenic fungi.

Depending on the substitution pattern, the compounds of the formula (I) may have one or more centers of chirality, in which case they are present as enantiomer or diastereomer mixtures. The invention provides both the pure enantiomers or diastereomers or rotamers and mixtures thereof. Suitable compounds of the formula (I) also include all possible stereoisomers (cis/trans isomers) and mixtures thereof. The compounds according to the invention can be present in different crystal modifications, which may differ in their biological activity. They also form, part of the subject matter of the present invention.

7-Amino-6-heteroaryl-1,2,4-triazolo[1,5-a]pyrimidines and their use in the field of the control of microorganisms such as harmful fungi are known per se.

EP-A 613 900 relates to 7-amino-1,2,4-triazolo[1,5-a]pyrimidines and their use as fungicides, where the compounds contain a hydrogen atom, a halogen atom or an amino group in the 5-position. In the 6-position there is an optionally substituted cycloalkyl ring or a heterocyclic group. According to EP-A 613 900, a heterocyclic group is a 3- to 6-, preferably 5- to 6-membered ring system.

Intermediates of the formula (II) which are used for preparing fungicidally active triazolopyrimid-7-ylideneamines are known from WO 01/96341. In the 5-position, the intermediates may have a halogen atom, an amino or an alkoxy group. In position 6, there is a phenyl, cycloalkyl or a five- or six-membered heteroaryl group.

Intermediates of the formula (II) which are used for preparing fungicidally active 2-(cyanoamino)pyrimidines are known from WO 01/96314. In position 5, these compounds carry a hydrogen atom, a halogen atom, an alkyl, alkoxy, alkylthio or alkylamine group, preferably chloride. In position 6 there is a phenyl, cycloalkyl or a 5- or 6-membered heteroaryl group.

WO 04/011467 relates to 1,2,4-triazolo[1,5-a]pyrimidines which, in position 5, have a halogen atom, a cyano, alkoxy, alkylthio, alkylsulfenyl, alkylsulfonyl or alkoxycarbonyl group. In the 6-position there is a 5- or 6-membered heterocyclyl group which may be optionally substituted pyrrolyl, thienyl, pyrazolyl, imidazolyl, oxazolyl, isoxazolyl, thiazolyl, isothiazolyl or pyrimidinyl.

WO 04/108727 discloses 1,2,4-triazolo[1,5-a]pyrimidines and their use for controlling unwanted microorganisms. In position 5, these compounds have exclusively halogen radicals, position 6 of the pyrimidine ring is substituted either by pyridyl or by pyrimidyl radicals.

WO 04/113342 relates to 1,2,4-triazolo[1,5-a]pyrimidines which are substituted in the 2-position of the 1,2,4-triazolo[1,5-a]pyrimidine skeleton and which, in position 5, may exclusively carry a halogen group. In position 6 there is a 5- or 6-membered heterocyclyl radical having 1 to 4 heteroatoms, such as nitrogen, oxygen and/or sulfur.

With respect to their fungicidal action, the 1,2,4-triazolo[1,5-a]pyrimidines known from the prior art are not entirely satisfactory, or they have unwanted properties, such as poor compatibility with useful plants.

Accordingly, it is an object of the present invention to provide novel compounds having improved fungicidal activity and/or better compatibility with crop plants.

Surprisingly, this object is achieved by the 7-amino-6-heteroaryl-1,2,4-triazolo[1,5-a]pyrimidine compounds of the formula (I) according to the invention and by the agriculturally acceptable salts of the compounds (I).

According to the present invention, agriculturally useful salts include in particular the salts of those cations and the acid addition salts of those acids whose cations and anions, respectively, have no adverse effect on the fungicidal action of the compounds (I). Thus, suitable cations are in particular the ions of the alkali metals, preferably sodium and potassium, of the alkaline earth metals, preferably calcium, magnesium and barium, and of the transition metals, preferably manganese, copper, zinc and iron, and also the ammonium ion which, if desired, may bear from one to four (C₁-C₄)-alkyl substituents and/or one phenyl or benzyl substituent, preferably diisopropylammonium, tetramethylammonium, tetrabutylammonium, trimethylbenzylammonium, and also phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogenphosphate, hydrogenphosphate, phosphate, nitrate, bicarbonate, carbonate, hexafluorosilicate, hexafluorophosphate, benzoate, and also the anions of (C₁-C₄)-alkanoic acids, preferably formate, acetate, propionate and butyrate. They can be formed by reacting (I) with an acid of the corresponding anion, preferably hydrochloric acid, hydrobromic acid, sulfuric acid, phosphoric acid or nitric acid.

The compounds of the formula (I) according to the invention can be obtained by various routes analogously to processes, known per se, of the prior art. The compounds according to the invention can be prepared, in particular, as follows:

Compounds of the formula (I), in particular compounds of the formula (I) in which X is (C₁-C₈)-alkyl, preferably (C₁-C₄)-alkyl, (C₂-C₈)-alkenyl or (C₂-C₈)-alkynyl or a corresponding halogenated radical, i.e. (C₁-C₈)-haloalkyl, preferably (C₁-C₄)-haloalkyl, (C₂-C₈)-haloalkenyl or (C₂-C₈)-haloalkynyl, can be prepared, for example, by reacting a 7-halotriazolopyrimidine of the formula (II)

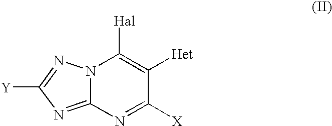

with an amine HNR¹R², where Hal is halogen and Het, X, Y, R¹ and R² are as defined for compounds of the formula (I). Furthermore, in this manner it is possible to prepare, preferably, compounds of the formula (I) in which X═(C₁-C₄)-alkoxy-(C₁-C₄)-alkyl, such as, in particular, methoxymethyl, or cyano-(C₁-C₄)-alkyl (see also Pharmazie 33, 1978, 42).

The reaction of the 7-halotriazolopyrimidine of the formula (II) with alkylamines is carried out analogously to the prior art cited at the outset or analogously to the methods described in WO 98/46608.

Advantageously, the process is carried out at temperatures in the range from 0° C. to 70° C., preferably from 10° C. to 35° C.

The reaction is preferably carried out in an inert solvent, for example an ether, such as, for example, dioxane, diethyl ether, diisopropyl ether, tert-butyl methyl ether or, in particular, tetrahydrofuran, a halogenated hydrocarbon, such as dichloromethane or dichloroethane, or an aromatic hydrocarbon, such as, for example, toluene or o-, m-, p-xylene, or in a mixture of the solvents mentioned above.

Preference is furthermore given to using a base, such as, for example, tertiary amines, in particular triethylamine, biscyclohexylmethylamine, pyridine, picoline or inorganic bases, such as potassium carbonate. It is also possible for excess amine HNR¹R² to serve as base.

The amines HNR¹R² used in this process are generally commercially available or can be prepared by processes generally known to the person skilled in the art.

The present invention furthermore provides compounds of the formula (II)

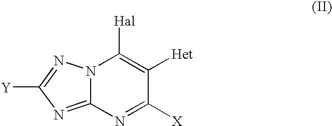

in which Hal is halogen and Het, X and Y are as defined for compounds of the formula (I). Hal is preferably chlorine or bromine. Particularly preferred compounds of the formula (I) according to the invention can be obtained from compounds of the formula (II) in which Het, X and/or Y are as defined in Tables 1 to 1387.

7-Halotriazolopyrimidines of the formula (II) can be obtained, for example, by reacting the corresponding 7-hydroxytriazolopyrimidine of the formula (III)

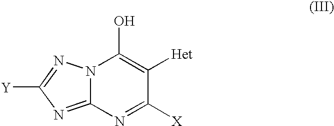

with a halogenating agent, where Het, X and Y are as defined for the compounds of the formula (I). See also Pharmazie 33, 1978, 42.

The halogenation is carried out analogously to the prior art cited at the outset or according to the methods described in WO-A 94/20501.

The halogenating agent used is advantageously a phosphorus oxyhalide or a phosphorus (V) halide, such as phosphorus pentachloride, phosphorus oxybromide or phosphorus oxychloride or a mixture of phosphorus oxychloride and phosphorus pentachloride.

The reaction of the compounds of the formula (III) with the halogenating agent is usually carried out at from 0° C. to 150° C., preferably from 80° C. to 125° C. [cf. also EP-A 770 615].

The reaction can be carried out in the absence of a solvent or in an inert solvent, for example a halogenated hydrocarbon, such as dichloromethane or dichloroethane, or an aromatic hydrocarbon, such as, for example, toluene or o-, m-, p-xylene or in a mixture of the solvents mentioned.

The present invention furthermore provides compounds of the formula (III)

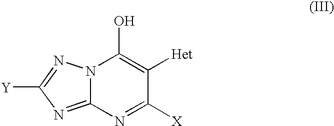

in which Het, X and Y are as defined for compounds of the formula (I). Particularly preferred compounds of the formula (I) or (II) can be obtained from compounds of the formula (III), win which Het, X and/or Y are as defined in Tables 1 to 1387.

7-Hydroxytriazolopyrimidines of the formula (III) can be prepared analogously to the methods described in Adv. Het. Chem. Vol. 57, p. 81ff. (1993). Compounds of the formula (III) can be obtained, for example, by reacting a compound of the formula (IV)

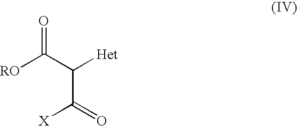

with a triazole of the formula (V)

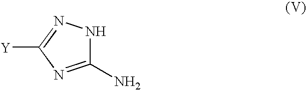

where Het, X and Y are as defined for compounds of the formula (I), where X is preferably (C₁-C₈)-alkyl, (C₂-C₈)-alkenyl, (C₂-C₈)-alkynyl, a corresponding halogenated radical or (C₁-C₄)-alkoxy-(C₁-C₄)-alkyl and R is alkyl, preferably (C₁-C₆)-alkyl, more preferably (C₁-C₄)-alkyl, in particular methyl or ethyl.

The reaction of a 3-amino-1,2,4-triazole (V) with a compound of the formula (IV) is usually carried out at temperatures of from 80° C. to 250° C., preferably from 120° C. to 180° C.

Preferably, the reaction is carried out without a solvent, or an inert organic solvent is used. The presence of a base may be preferred [cf. EP-A 770 615]. Furthermore, it may also be preferable to carry out the reaction in the presence of acetic acid under conditions generally known to the person skilled in the art.

Suitable solvents are, for example, aliphatic hydrocarbons, aromatic hydrocarbons, such as toluene, o-, m- and p-xylene, halogenated hydrocarbons, ethers, nitriles, ketones, alcohols, and also N-methylpyrrolidone, dimethyl sulfoxide, dimethylformamide and dimethylacetamide.

With particular preference, the reaction is carried out without solvent or in chlorobenzene, xylene, dimethyl sulfoxide or N-methylpyrrolidone. It is also possible to use mixtures of the solvents mentioned. If appropriate, catalytic amounts of acids, such as p-toluenesulfonic acid, acetic acid or propionic acid, may be added, too.

Suitable bases are, in general, inorganic compounds, such as alkali metal and alkaline earth metal hydroxides, alkali metal and alkaline earth metal oxides, alkali metal and alkaline earth metal hydrides, alkali metal amides, alkali metal and alkaline earth metal carbonates, and also alkali metal bicarbonates, such as, for example, potassium carbonate, organometallic compounds, in particular alkali metal alkyls, alkylmagnesium halides, and also alkali metal and alkaline earth metal alkoxides and dimethoxymagnesium, moreover organic bases, for example tertiary amines, such as trimethylamine, triethylamine, triisopropylethylamine, tributylamine and N-methylpiperidine, N-methylmorpholine, pyridine, substituted pyridines, such as collidine, lutidine and 4-dimethylaminopyridine, and also bicyclic amines. Particular preference is given to using tertiary amines, such as triethylamine, triisopropylethylamine, tributylamine, N-methylmorpholine or N-methylpiperidine.

The bases are generally used in catalytic amounts; however, they can also be used in equimolar amounts, in excess or, if appropriate, as solvent.

In general, the starting materials are reacted with one another in equimolar amounts. In terms of yield, it may be advantageous to employ an excess of base and the compound of the formula (IV), based on the 3-amino-1,2,4-triazole of the formula (V).

Some of the compounds of the formula (IV) are novel and also form part of the subject matter of the present invention, namely when Het has 1, 2 or 3 substituents which, independently of one another, are selected from the group consisting of cyano, hydroxyl, cyanato (OCN), (C₁-C₈)-alkyl, (C₂-C₁₀)-alkenyl, (C₂-C₁₀)-alkynyl, (C₁-C₆)-haloalkyl, (C₂-C₁₀)-haloalkenyl, (C₁-C₆)-alkoxy, (C₂-C₁₀)-alkenyloxy, (C₂-C₁₀)-alkynyloxy, (C₁-C₆)-haloalkoxy, (C₃-C₆)-cycloalkyl, (C₃-C₆)-cycloalkenyl, (C₃-C₆)-cycloalkoxy, (C₂-C₁₀)-alkenyloxycarbonyl, (C₂-C₁₀)-alkynyloxycarbonyl, (C₁-C₈)-alkoximinoalkyl, (C₂-C₁₀)-alkenyloximinoalkyl, (C₂-C₁₀)-alkynyloximinoalkyl, (C₁-C₈)-alkylcarbonyl, (C₂-C₁₀)-alkenylcarbonyl, (C₂-C₁₀)-alkynylcarbonyl, (C₃-C₆)-cycloalkylcarbonyl, S(═O)ₙA¹, C(═O)A², C(═S)A², a group —C(═N—OR⁷)(NR⁸R⁹) and a group —C(═N—NR¹⁰R¹¹)(NR¹²R¹³) in which A¹, A², R⁷, R⁸, R⁹, R¹⁰, R¹¹, R¹² and R¹³ are as defined above, where Het may additionally also have 1, 2 or 3 further halogen atoms as substituents L, except for compounds (IV) in which Het is 5-ethoxycarbonylpyridin-2-yl, 2-methylpyridin-3-yl, 4-chloro-6-methoxy-1,3,5-triazin-2-yl or 4,6-dimethoxy-1,3,5-triazin-2-yl.

The present invention furthermore relates in particular to compounds of the formula (IV) in which R and X are as defined above and Het is unsubstituted pyrimidyl or pyrimidyl which is substituted by one, two or three identical or different substituents L, in particular unsubstituted or substituted pyrimidyl-2-yl, pyrimidyl-4-yl or pyrimidyl-5-yl, except for compounds of the formula (IV) in which Het is 4,6-dimethoxy-5-nitropyrimidyl-2-yl or 2-(methylcarbonylamino)pyrimidyl-4-yl. Particular preference is given here to compounds of the formula (IV) in which Het represents the preferred pyrimidyl radicals of the examples of Tables 1 to 1387.

Compounds of the formula (IV) can be prepared analogously to standard processes in the sense of a mixed ester condensation from corresponding heteroarylacetic esters by reaction with the corresponding aliphatic alkyl (C₂-C₅)-carboxylates, such as ethyl acetate, ethyl propionate, ethyl butyrate or ethyl valerate or with a reactive derivative thereof, for example an acid chloride or an acid anhydride, in the presence of a strong base, for example an alkoxide, an alkali methylamide or an organolithium compound, for example analogously to the methods described in J. Chem. Soc. Perkin Trans 1967, 767 or in Eur. J. Org. Chem. 2002, p. 3986.

Alternatively, the compounds of the formula (I) according to the invention in which R¹ and R² are hydrogen can also be prepared by reacting a ketonitrile of the formula (IV-1)

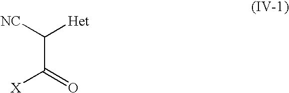

with a triazole of the formula (V), as stated above, where Het and X in formula (IV-1) have the meanings or preferred meanings mentioned for compounds of the formula (I) and X is preferably (C₁-C₈)-alkyl, (C₂-C₈)-alkenyl, (C₂-C₈)-alkynyl, a corresponding halogenated radical or (C₁-C₄)-alkoxy-(C₁-C₄)-alkyl.

The reaction can be carried out in the presence or absence of solvents. It is advantageous to employ solvents which are substantially inert to the starting materials and in which the starting materials are completely or partially soluble. Suitable solvents are in particular alcohols, such as ethanol, propanols, butanols, glycols or glycol monoethers, diethylene glycols or their monoethers, aromatic hydrocarbons, such as toluene, benzene or mesitylene, amides, such as dimethylformamide, diethylformamide, dibutylformamide, N,N-dimethylacetamide, lower alkanoic acids, such as formic acid, acetic acid, propionic acid or bases, as mentioned above, and mixtures of these solvents with water. The reaction temperatures are between 50 and 300° C., preferably from 50 to 150° C., when the reaction is carried out in solution.

The compounds of the formula (I) are, if appropriate after evaporation of the solvent or dilution with water, isolated as crystalline compounds.

Some of the substituted alkylcyanides of the formula (IV-1) required for this process are known, or they can be prepared analogously to known methods from alkyl cyanides and carboxylic esters with strong bases, for example alkali metal hydrides, alkali metal alkoxides, alkali metal amides or alkylmetal compounds [cf.: J. Amer. Chem. Soc. Vol. 73, (1951) p. 3766]. See also Bioorganic & Medicinal Chemistry Letters (2004), 14(15), 3943-3947.

The compounds of the formula (I) according to the invention, in particular the compounds of the formula (I) in which X is preferably (C₁-C₈)-alkyl, more preferably (C₁-C₄)-alkyl, (C₁-C₈)-haloalkyl, more preferably (C₁-C₄)-haloalkyl, (C₂-C₈)-alkenyl, (C₂-C₈)-haloalkenyl, (C₂-C₈)-alkynyl or (C₂-C₈)-haloalkynyl can also be prepared in an advantageous manner by reacting compounds (IIa)

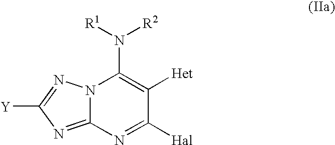

in which Hal is halogen, in particular chlorine or bromine, and Het, R¹, R² and Y are as defined for compounds of the formula (I), with an organometallic compound X^{a}—Mt, in which X^{a} is (C₁-C₈)-alkyl, preferably (C₁-C₄)-alkyl, (C₁-C₈)-haloalkyl, preferably (C₁-C₄)-haloalkyl, (C₂-C₈)-alkenyl, (C₂-C₈)-haloalkenyl, (C₂-C₈)-alkynyl, (C₂-C₈)-haloalkynyl and Mt is lithium, magnesium or zinc. Furthermore, it is also possible to prepare in this manner preferably compounds of the formula (I) in which X is (C₁-C₄)-alkoxy-(C₁-C₄)-alkyl or cyano-(C₁-C₄)-alkyl. In this manner, it is possible to prepare particularly advantageously, using the corresponding compounds X^{a}—Mt, compounds of the formula (I) in which X is (C₁-C₄)-alkyl, (C₂-C₆)-alkenyl or (C₂-C₆)-alkynyl.

The reaction is preferably carried out in the presence of catalytic or in particular at least equimolar amounts of transition metal salts and/or compounds, in particular in the presence of Cu salts, such as Cu(I) halides and especially Cu(I) iodide.

Preferably, the reaction is carried out in an inert organic solvent, for example one of the ethers mentioned above, in particular tetrahydrofuran, an aliphatic or cycloaliphatic hydrocarbon, such as hexane, cyclohexane and the like, an aromatic hydrocarbon, such as toluene, or a mixture of these solvents.

The temperatures which are preferred for the reaction are in the range from −100 to +100° C., in particular in the range from −80° C. to +40° C. Processes to achieve this are known, for example from the prior art cited at the outset (see, for example, WO 03/004465).

Compounds of the formula (IIa) can be prepared by reacting a 5,7-dihalotriazolopyrimidine of the formula (IIb)

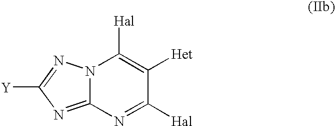

with the corresponding alkylamine HNR¹R². The conditions to be employed are analogous to those for reacting compounds of the formula (II) with amines, as stated above. Here, Het and Y are as defined for compounds of the formula (I).

5,7-Dihalotriazolopyrimidines of the formula (IIb) can be obtained, for example, by reacting the corresponding 5,7-dihydroxytriazolopyrimidine of the formula (IIc)

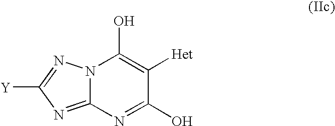

analogously to the reaction described above with a halogenating agent. Here, Het and Y are as defined for compounds of the formula (I).

5,7-Dihydroxytriazolopyrimidines of the formula (IIc) can be prepared by various routes, for example analogously to the methods described in Adv. Het. Chem. Vol. 57, p. 81ff. (1993) or analogously to the prior art cited at the outset. Thus, they can be obtained by reacting the corresponding aminotriazole of the formula (V) with a corresponding heteroarylmalonate of the formula (IVa).

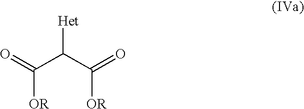

Here, R is alkyl, preferably (C₁-C₆)-alkyl, in particular methyl or ethyl. Het and Y (in formula (V)) are as defined above. The reaction conditions are analogous to those used when reacting compounds of the formula (IV) with compounds (V), as stated above.

Compounds of the formula (I) in which X is (C₁-C₈)-alkyl can also be prepared by reacting, in a first step, a compound of the formula (IIa), as described above, with a malonate of the formula (IVb)

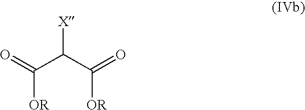

to give a compound of the formula (VI)

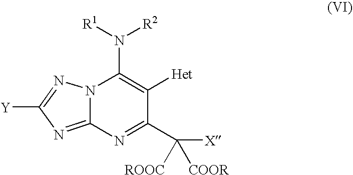

in which X″ is hydrogen or (C₁-C₇)-alkyl, preferably (C₁-C₃)-alkyl, and R is (C₁-C₄)-alkyl and Het, R¹, R² and Y are as defined for compounds of the formula (I). The resulting compound of the formula (VI) is hydrolyzed and the hydrolysis product is decarboxylated [cf. U.S. Pat. No. 5,994,360].

The malonates (IVb) are known from the literature, for example from J. Am. Chem. Soc., Vol. 64, 2714 (1942); J. Org. Chem., Vol. 39, 2172 (1974); Helv. Chim. Acta, Vol. 61, 1565 (1978), or they can be prepared in accordance with the literature cited.

The subsequent hydrolysis of the ester (VI) is carried out under conditions generally known to the person skilled in the art. Depending on the various structural elements, alkaline or acidic hydrolysis of the compounds (VI) may be advantageous. Under the conditions of the ester hydrolysis, there may already be complete or partial decarboxylation to the compounds of the formula (I).

The decarboxylation is usually carried out at temperatures of from 20° C. to 180° C., preferably from 50° C. to 120° C.

The decarboxylation is preferably carried out in an inert solvent, if appropriate in the presence of an acid. Suitable acids are hydrochloric acid, sulfuric acid, phosphoric acid, formic acid, acetic acid, p-toluenesulfonic acid.

Suitable solvents are water, aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide; with particular preference, the reaction is carried out in hydrochloric acid or acetic acid. It is also possible to use mixtures of the solvents mentioned.

The reaction mixtures obtained in the preparation of the compounds of the formula (I) or in the preparation of intermediates thereof can be worked up in the customary manner, for example by mixing with water, separating the phases and, if appropriate, chromatographic purification of the crude products. Some of the intermediates and end products are obtained in the form of colorless or slightly brownish viscous oils which can be purified or freed from volatile components under reduced pressure and at moderately elevated temperature. If the intermediates and end products are obtained as solids, purification can also be carried out by recrystallization or digestion.

If individual compounds of the formula (I) cannot be obtained directly by the routes described above, they can be prepared by derivatization of other compounds of the formula (I) according to the invention.

If the synthesis yields mixtures of isomers, a separation is generally not necessarily required since in some cases the individual isomers can be interconverted during work-up for use or during application (for example under the action of light, acids or bases). Such conversions may also take place after use, for example in the treatment of plants in the treated plant or in the harmful fungus to be controlled.

In the definitions of the variables given in the formulae above, collective terms are used which are generally representative of the particular substituents or the substituent moieties in composite groups. The term Cₙ-Cₘ indicates the number of carbon atoms possible in each case in the substituent or substituent moiety in question:

halogen: fluorine, chlorine, bromine and iodine;
alkyl and the alkyl moieties in composite groups such as alkyloxy, alkylthio, alkylsulfinyl and alkylsulfonyl: saturated straight-chain or branched hydrocarbon radicals having 1 to 4, 6 or 8 carbon atoms, where the alkyl radicals are preferably (C₁-C₈)-alkyl, in particular (C₁-C₆)-alkyl, radicals. According to the invention, it may furthermore be preferred to use short-chain alkyl groups such as (C₁-C₄)-alkyl, on the other hand, it may also be advantageous to employ alkyl groups having relatively long chains, such as (C₅-C₈)-alkyl. Examples are (C₁-C₆)-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl and the like;
haloalkyl: alkyl as defined above, in particular straight-chain or branched alkyl groups having 1 to 2, 4, 6 or 8 carbon atoms (as mentioned above), where some or all of the hydrogen atoms are replaced by halogen atoms as mentioned above. In one embodiment, the alkyl groups are substituted at least once or completely by a particular halogen atom, preferably fluorine, chlorine or bromine. In a further embodiment, the alkyl groups are partially or fully halogenated by different halogen atoms; in the case of mixed halogen substitutions, the combination of chlorine and fluorine is preferred.

Especially preferred are (C₁-C₃)-haloalkyl, more preferably (C₁-C₂)-haloalkyl, such as chloromethyl, bromomethyl, dichloromethyl, trichloromethyl, fluoromethyl, difluoro-methyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;

alkenyl and also the alkenyl moieties in composite groups, such as alkenyloxy: monounsaturated straight-chain or branched hydrocarbon radicals having 2 to 4, 2 to 6, 2 to 8 or 2 to 10 carbon atoms and one double bond in any position. Preference is given to (C₂-C₈)-alkenyl radicals, more preferably (C₄-C₆)-alkenyl radicals. According to the invention, it may furthermore be preferred to use small alkenyl groups, such as (C₂-C₄)-alkenyl, on the other hand, it may also be preferred to employ larger alkenyl groups, such as (C₅-C₈)-alkenyl.

Examples of preferred alkenyl radicals are (C₂-C₆)-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl and the like;

haloalkenyl: alkenyl as defined above, where in these groups at least one of the hydrogen atoms or all of the hydrogen atoms are replaced by halogen atoms as described above under haloalkyl, in particular fluorine, chlorine or bromine;
alkadienyl: doubly unsaturated straight-chain or branched hydrocarbon radicals having 4 to 10, preferably 6 to 8 carbon atoms [(C₄-C₁₀)-alkadienyl, preferably (C₆-C₈)-alkadienyl] and two double bonds in any position, for example 1,3-butadienyl, 1-methyl-1,3-butadienyl, 2-methyl-1,3-butadienyl, penta-1,3-dien-1-yl, hexa-1,4-dien-1-yl, hexa-1,4-dien-3-yl, hexa-1,4-dien-6-yl, hexa-1,5-dien-1-yl, hexa-1,5-dien-3-yl, hexa-1,5-dien-4-yl, hepta-1,4-dien-1-yl, hepta-1,4-dien-3-yl, hepta-1,4-dien-6-yl, hepta-1,4-dien-7-yl, hepta-1,5-dien-1-yl, hepta-1,5-dien-3-yl, hepta-1,5-dien-4-yl, hepta-1,5-dien-7-yl, hepta-1,6-dien-1-yl, hepta-1,6-dien-3-yl, hepta-1,6-dien-4-yl, hepta-1,6-dien-5-yl, hepta-1,6-dien-2-yl, octa-1,4-dien-1-yl, octa-1,4-dien-2-yl, octa-1,4-dien-3-yl, octa-1,4-dien-6-yl, octa-1,4-dien-7-yl, octa-1,5-dien-1-yl, octa-1,5-dien-3-yl, octa-1,5-dien-4-yl, octa-1,5-dien-7-yl, octa-1,6-dien-1-yl, octa-1,6-dien-3-yl, octa-1,6-dien-4-yl, octa-1,6-dien-5-yl, octa-1,6-dien-2-yl, deca-1,4-dienyl, deca-1,5-dienyl, deca-1,6-dienyl, deca-1,7-dienyl, deca-1,8-dienyl, deca-2,5-dienyl, deca-2,6-dienyl, deca-2,7-dienyl, deca-2,8-dienyl and the like;
haloalkadienyl: alkadienyl as defined above, where in these groups at least one of the hydrogen atoms or all of the hydrogen atoms are replaced by halogen atoms as described above under haloalkyl, in particular fluorine, chlorine or bromine;
alkynyl and the alkynyl moieties in composite groups such as alkynyloxy: straight-chain or branched hydrocarbon groups having 2 to 4, 2 to 6, 2 to 8 or 2 to 10 carbon atoms and one or two triple bonds in any position, but not adjacent to one another. Preference is given to (C₂-C₈)-alkynyl radicals, more preferably (C₄-C₆)-alkynyl radicals. Preferred examples are: (C₂-C₆)-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl and the like;
haloalkynyl: alkynyl as defined above, where in these groups at least one of the hydrogen atoms or all of the hydrogen atoms are replaced by halogen atoms as described above under haloalkyl, in particular fluorine, chlorine or bromine;
cycloalkyl and the cycloalkyl moieties in composite groups such as cycloalkoxy: monocyclic saturated hydrocarbon groups having 3 to 8, preferably 4 to 8, carbon ring members, such as cyclopropyl, cyclobutyl, cyclopentyl, cyclohexyl, cycloheptyl and cyclooctyl;
halocycloalkyl: cycloalkyl as defined above, where in these groups at least one of the hydrogen atoms or all of the hydrogen atoms are replaced by halogen atoms as described above under haloalkyl, in particular fluorine, chlorine or bromine;
cycloalkenyl: monocyclic monounsaturated hydrocarbon groups having 3 to 8, preferably 5 to 6 carbon ring members, such as cyclopenten-1-yl, cyclopenten-3-yl, cyclohexen-1-yl, cyclohexen-3-yl, cyclohexen-4-yl and the like;
halocycloalkenyl: cycloalkenyl as defined above, where in these groups at least one of the hydrogen atoms or all of the hydrogen atoms are replaced by halogen atoms as described above under haloalkyl, in particular fluorine, chlorine or bromine;
bicycloalkyl: a bicyclic hydrocarbon radical having 5 to 10, preferably 7 to 9, carbon atoms, such as bicyclo[2.2.1]hept-1-yl, bicyclo[2.2.1]hept-2-yl, bicyclo[2.2.1]hept-7-yl, bicyclo[2.2.2]oct-1-yl, bicyclo[2.2.2]oct-2-yl, bicyclo[3.3.0]octyl, bicyclo[4.4.0]decyl and the like;
halobicycloalkyl: bicycloalkyl as defined above, where in these groups at least one of the hydrogen atoms or all of the hydrogen atoms are replaced by halogen atoms as described above under haloalkyl, in particular fluorine, chlorine or bromine;
alkoxy: an alkyl group as defined above which is attached via an oxygen, preferably having 1 to 8, more preferably 2 to 6, carbon atoms. According to the invention it may be preferred to use small alkoxy groups, such as (C₁-C₄)-alkoxy, on the other hand, it may also be preferred to use larger alkoxy groups, such as (C₅-C₈)-alkoxy. Examples of preferred alkoxy groups are: methoxy, ethoxy, n-propoxy, 1-methylethoxy, butoxy, 1-methylpropoxy, 2-methylpropoxy or 1,1-dimethylethoxy, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy;

haloalkoxy: alkoxy as defined above, where in these groups at least one of the hydrogen atoms or all of the hydrogen atoms are replaced by halogen atoms as described above under haloalkyl, in particular fluorine, chlorine or bromine. Especially preferred are (C₁-C₄)-alkoxy radicals as mentioned above, which are partially or fully substituted by fluorine, chlorine, bromine and/or iodine, preferably by fluorine, i.e., for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy; and also (C₁-C₆)-haloalkoxy, such as, for example, 5-fluoropentoxy, 5-chloropentoxy, 5-bromopentoxy, 5-iodopentoxy, undecafluoropentoxy, 6-fluorohexoxy, 6-chlorohexoxy, 6-bromohexoxy, 6-iodohexoxy or dodecafluorohexoxy.

According to the invention, it may be preferred to use short-chain haloalkoxy groups, such as (C₁-C₄)-haloalkoxy, on the other hand, it may also be preferred to use relatively long-chain haloalkoxy groups, such as (C₅-C₈)-haloalkoxy.

Alkenyloxy: alkenyl as defined above which is attached via an oxygen atom. Preferred is (C₂-C₈)-alkenyloxy, more preferably (C₃-C₆)-alkenyloxy. According to the invention, it may be preferred to use short-chain alkenyloxy radicals, such as (C₂-C₄)-alkenyloxy, on the other hand, it may also be preferred to use relatively long-chain alkenyloxy groups, such as (C₅-C₈)-alkenyloxy.

Examples are in particular (C₃-C₆)-alkenyloxy, such as 1-propenyloxy, 2-propenyloxy, 1-methylethenyloxy, 1-butenyloxy, 2-butenyloxy, 3-butenyloxy, 1-methyl-1-propenyloxy, 2-methyl-1-propenyloxy, 1-methyl-2-propenyloxy, 2-methyl-2-propenyloxy, 1-pentenyl-oxy, 2-pentenyloxy, 3-pentenyloxy, 4-pentenyloxy, 1-methyl-1-butenyloxy, 2-methyl-1-butenyloxy, 3-methyl-1-butenyloxy, 1-methyl-2-butenyloxy, 2-methyl-2-butenyloxy, 3-methyl-2-butenyloxy, 1-methyl-3-butenyloxy, 2-methyl-3-butenyloxy, 3-methyl-3-butenyl, 1,1-dimethyl-2-propenyloxy, 1,2-dimethyl-1-propenyloxy, 1,2-dimethyl-2-propenyloxy, 1-ethyl-1-propenyloxy, 1-ethyl-2-propenyloxy, 1-hexenyloxy, 2-hexenyl-oxy, 3-hexenyloxy, 4-hexenyloxy, 5-hexenyloxy, 1-methyl-1-pentenyloxy, 2-methyl-1-pentenyloxy, 3-methyl-1-pentenyloxy, 4-methyl-1-pentenyloxy, 1-methyl-2-pentenyloxy, 2-methyl-2-pentenyloxy, 3-methyl-2-pentenyloxy, 4-methyl-2-pentenyloxy, 1-methyl-3-pentenyloxy, 2-methyl-3-pentenyloxy, 3-methyl-3-pentenyloxy, 4-methyl-3-pentenyloxy, 1-methyl-4-pentenyloxy, 2-methyl-4-pentenyloxy, 3-methyl-4-pentenyloxy, 4-methyl-4-pentenyloxy, 1,1-dimethyl-2-butenyloxy, 1,1-dimethyl-3-butenyloxy, 1,2-dimethyl-1-butenyloxy, 1,2-dimethyl-2-butenyloxy, 1,2-dimethyl-3-butenyloxy, 1,3-dimethyl-1-butenyloxy, 1,3-dimethyl-2-butenyloxy, 1,3-dimethyl-3-butenyloxy, 2,2-dimethyl-3-butenyloxy, 2,3-dimethyl-1-butenyloxy, 2,3-dimethyl-2-butenyloxy, 2,3-dimethyl-3-butenyloxy, 3,3-dimethyl-1-butenyloxy, 3,3-dimethyl-2-butenyloxy, 1-ethyl-1-butenyloxy, 1-ethyl-2-butenyloxy, 1-ethyl-3-butenyloxy, 2-ethyl-1-butenyloxy, 2-ethyl-2-butenyloxy, 2-ethyl-3-butenyloxy, 1,1,2-trimethyl-2-propenyloxy, 1-ethyl-1-methyl-2-propenyloxy, 1-ethyl-2-methyl-1-propenyloxy and 1-ethyl-2-methyl-2-propenyloxy;

haloalkenyloxy: alkenyloxy as defined above, where in these groups at least one of the hydrogen atoms or all of the hydrogen atoms are replaced by halogen atoms as described above under haloalkyl, in particular fluorine, chlorine or bromine;
alkynyloxy: alkynyl as mentioned above which is attached via an oxygen atom. Preferred is (C₂-C₈)-alkynyloxy, more preferably (C₃-C₆)-alkynyloxy. According to the invention, it may be preferred to use short-chain alkynyloxy radicals, such as (C₂-C₄)-alkynyloxy, on the other hand, it may also be preferred to use relatively long-chain alkynyloxy groups, such as (C₅-C₈)-alkynyloxy. Examples are: (C₃-C₆)-alkynyloxy, such as 2-propynyloxy, 2-butynyloxy, 3-butynyloxy, 1-methyl-2-propynyloxy, 2-pentynyloxy, 3-pentynyloxy, 4-pentynyloxy, 1-methyl-2-butynyloxy, 1-methyl-3-butynyloxy, 2-methyl-3-butynyloxy, 1-ethyl-2-propynyloxy, 2-hexynyloxy, 3-hexynyloxy, 4-hexynyloxy, 5-hexynyloxy, 1-methyl-2-pentynyloxy, 1-methyl-3-pentynyloxy and the like;
haloalkynyloxy: alkynyloxy as defined above, where in these groups at least one of the hydrogen atoms or all of the hydrogen atoms are replaced by halogen atoms as described above under haloalkyl, in particular fluorine, chlorine or bromine;
alkylene: divalent Unbranched chains of CH₂ groups. Preference is given to (C₁-C₆)-alkylene, more preference to (C₂-C₄)-alkylene; furthermore, it may be preferred to used (C₁-C₃)-alkylene groups. Examples of preferred alkylene radicals are CH₂, CH₂CH₂, CH₂CH₂CH₂, CH₂(CH₂)₂CH₂, CH₂(CH₂)₃CH₂ and CH₂(CH₂)₄CH₂;
oxyalkylene: alkylene as defined above, preferably with 2 to 4 CH₂ groups, where one valency is attached to the skeleton via an oxygen atom. Examples of preferred oxyalkylene radicals are OCH₂, OCH₂CH₂, OCH₂CH₂CH₂ and OCH₂(CH₂)₂CH₂;
oxyalkyleneoxy: alkylene as defined above, preferably with 1 to 3 CH₂ groups, where both valencies are attached to the skeleton via an oxygen atom. Examples of preferred oxyalkyleneoxy radicals are OCH₂O, OCH₂CH₂O and OCH₂CH₂CH₂O.

Alkylthio: alkyl as defined above which is attached via an S atom.

Alkylsulfinyl: alkyl as defined above which is attached via an SO group.

Alkylsulfonyl: alkyl as defined above which is attached via an S(O)₂ group.

Aryl: an aromatic hydrocarbon radical, (C₆-C₁₄)-aryl radicals being preferred and (C₆-C₁₀)-aryl radicals being particularly preferred. Examples of preferred aryl radicals are phenyl, naphthyl and anthryl.

The aryl radicals may be substituted by at least one halogen atom or fully by halogen atoms as defined above. According to the invention, it may be advantageous to employ haloaryl groups, where aryl is as defined above. Particularly preferred may be halophenyl and halonaphthyl.

Aryloxy: aryl as defined above, where the aryl radical is attached to the skeleton via an oxygen atom.

Arylthio: aryl as defined above, where the aryl radical is attached to the skeleton via a sulfur atom.

5-, 6-, 7-, 8-, 9- or 10-membered (five- to ten-membered) saturated, partially unsaturated or aromatic heterocycle which contains one, two, three or four heteroatoms from the group consisting of oxygen, nitrogen or sulfur:
a five- or six-membered saturated or partially unsaturated heterocycle (hereinbelow also referred to as heterocyclyl) which contains one, two, three or four heteroatoms from the group consisting of oxygen, nitrogen and sulfur as ring members: for example monocyclic saturated or partially unsaturated heterocycles containing, in addition to carbon ring members, one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-di-hydropyrazol-1-yl, 2,3-dihydropyrazol-2-yl, 2,3-dihydropyrazol-3-yl, 2,3-dihydro-pyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydro-oxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexa-hydropyrimidinyl, 5-hexahydropyrimidinyl, 2-piperazinyl, 1,3,5-hexahydrotriazin-2-yl and 1,2,4-hexahydrotriazin-3-yl and the corresponding -ylidene radicals;
a seven-membered saturated or partially unsaturated heterocycle containing one, two, three or four heteroatoms from the group consisting of oxygen, nitrogen and sulfur as ring members: for example mono- and bicyclic heterocycles having 7 ring members and containing, in addition to carbon ring members, one to three nitrogen atoms and/or one oxygen or sulfur atom or one or two oxygen and/or sulfur atoms, for example tetra- and hexahydroazepinyl, such as 2,3,4,5-tetrahydro[1H]azepin-1-, -2-, -3-, -4-, -5-, -6- or -7-yl, 3,4,5,6′-tetrahydro[2H]-azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, hexahydroazepin-1-, -2-, -3- or -4-yl, tetra- and hexahydrooxepinyl wie 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, -4-, -5-, -6- or -7-yl, hexahydroazepin-1-, -2-, -3- or -4-yl, tetra- and hexahydro-1,3-diazepinyl, tetra- and hexahydro-1,4-diazepinyl, tetra- and hexahydro-1,3-oxazepinyl, tetra- and hexahydro-1,4-oxazepinyl, tetra- and hexahydro-1,3-dioxepinyl, tetra- and hexahydro-1,4-dioxepinyl and the corresponding -ylidene radicals.
a five- or six-membered aromatic heterocycle which contains one, two, three or four heteroatoms from the group consisting of oxygen, nitrogen and sulfur: mono- or bicyclic heteroaryl, for example 5-membered heteroaryl which is attached via carbon and which contains one to three nitrogen atoms or one or two nitrogen atoms and/or one sulfur or oxygen atom as ring members, such as 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl; 5-membered heteroaryl which is attached via nitrogen and which contains one to three nitrogen atoms as ring members, such as pyrrol-1-yl, pyrazol-1-yl, imidazol-1-yl, 1,2,3-triazol-1-yl and 1,2,4-triazol-1-yl; 6-membered heteroaryl which contains one to three nitrogen atoms as ring members, such as pyridin-2-yl, pyridin-3-yl, pyridin-4-yl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl;

heteroaryloxy: heteroaryl as defined above where the heteroaryl radical is attached to the skeleton via an oxygen atom.

Heteroarylthio: heteroaryl as defined above where the heteroaryl radical is attached to the skeleton via an sulfur atom.

Examples of organic radicals which contain 3 to 13 carbon atoms and one or more silicon atoms and also, if appropriate, 1 to 3 identical or different heteroatoms from the group consisting of oxygen, nitrogen and sulfur and which may be unsubstituted or carry 1 to 4 identical or different halogen atoms, are SiMe₃, SiMe₂Et, SiMe₂CHMe₂, SiMe₂CH₂CHMe₂, SiMe₂CH₂CMe₃, SiMe₂OCHMe₂, SiMe₂OCH₂CHMe₂, CH₂SiMe₃, CH₂SiMe₂Et, CH₂SiMe₂CHMe₂, CH₂SiMe₂CH₂CHMe, CH₂SiMe₂OMe, CH₂SiMe₂OCHMe₂, CH₂SiMe₂OCH₂CHMe₂, CHMeSiMe₃, CHMeSiMe₂OMe, (CH₂)₂SiMe₃, (CH₂)₂SiMe₂Et, (CH₂)₂SiMe₂CHMe₂, (CH₂)₂SiMe₂CMe₃, (CH₂)₂SiMe₂CH₂CHMe₂, (CH₂)₂SiMe₂CH₂CH₂Me, (CH₂)₂SiMe₂CH₂CMe₃, (CH₂)₂SiMe₂OCHMe₂, (CH₂)₂SiMe₂OCH₂CHMe₂, CHMeCH₂SiMe₃, CHMeCH₂SiMe₂Et, CHMeCH₂SiMe₂CH₂CH₂Me, CHMeCH₂—SiMe₂CHMe₂, CHMeCH₂SiMe₂CMe₃, CHMeCH₂SiMe₂CH₂CHMe₂, CFMeCH₂SiMe₃, CHMeCH₂CH₂SiMe₂OMe, CHMeCH₂SiMe₂OCHMe₂, CHMeCH₂SiMe₂OCH₂CHMe₂, CH₂CHMeSiMe₃, CH₂CHMeSiMe₂Et, CH₂CHMeSiMe₂CHMe₂, CHMeCHMeSiMe₃, CMe₂CH₂SiMe₃, (CH₂)₃SiMe₃, (CH₂)₃SiMe₂Et, (CH₂)₃Si-Me₂CHMe₂, (CH₂)₃SiMe₂CH₂CHMe₂, (CH₂)₃SiMe₂OMe, (CH₂)₃SiMe₂OCHMe₂, (CH₂)₃SiMe₂OCH₂CHMe₂, CHMeCH₂CH₂SiMe₃, CHMeCH₂CH₂SiMe₂Et, CHMeCH₂CH₂SiMe₂CHMe₂, CHMeCH₂CH₂CH₂SiMe₂OMe, CHMeCH₂—CH₂SiMe₂OCHMe₂, CMe═CHSiMe₃, CH₂CH₂SiMe₂OMe, —C≡C—SiMe₃, —CH₂—C≡C—SiMe₃ and —CHMe—C≡C—SiMe₃, where Me is methyl and Et is ethyl.

The scope of the present invention embraces the (R) and (S) isomers or rotamers and the racemates of compounds of the formula (I) having chiral centers. The compounds according to the invention may be present in various crystal modifications which may differ in their biological activity. They are likewise provided by the present invention.

With a view to the intended use of the triazolopyrimidines of the formula (I), particular preference is given to the following meanings of the substituents, in each case on their own or in combination. The preferred substituents or preferred combinations of substituents apply correspondingly to the precursors of the compounds of the formula (I):

In particular for the fungicidal action of the compounds according to the invention, it may be preferred if R² is hydrogen. In one embodiment of the present invention, R² is hydrogen and R¹ is different from hydrogen. Furthermore, it may be preferred if at least one of the radicals R¹ and R² is different from hydrogen. Preference is likewise given to compounds of the formula (I) in which R¹ and R² are different from hydrogen. Among these, preference is given to compounds of the formula (I) in which R² is (C₁-C₄)-alkyl, especially methyl or ethyl.

In a further embodiment of the invention, R¹ and R² are both hydrogen.

Furthermore, for the fungicidal action of the compounds according to the invention, it is advantageous if the substituents R¹, X and Y independently of one another and preferably in combination particularly preferably have the meanings indicated below.

R¹ is in particular (C₁-C₈)-alkyl, preferably (C₁-C₆)-alkyl, (C₂-C₈)-alkenyl, preferably (C₂-C₆)-alkenyl, (C₂-C₈)-alkynyl, preferably (C₂-C₆)-alkynyl, (C₃-C₈)-cycloalkyl, preferably (C₃-C₆)-cycloalkyl, which may be substituted 1, 2, 3 or 4-times by halogen or (C₁-C₄)-alkyl, or (C₁-C₈)-haloalkyl. Here, it may be preferred for R² to be hydrogen or (C₁-C₄)-alkyl.

Among these, a particularly preferred embodiment relates to compounds of the formula (I) in which R¹ is a group B:

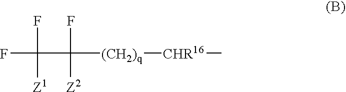

in which
Z¹ is hydrogen, fluorine or (C₁-C₄)-fluoroalkyl,
Z² is hydrogen or fluorine or
Z¹ and Z² together form a double bond;
q is 0 or 1; and
R¹⁶ is hydrogen or methyl.

Particular preference is moreover given to compounds of the formula (I) in which R¹ is (C₃-C₆)-cycloalkyl which may be substituted by (C₁-C₄)-alkyl.

Furthermore preferred compounds of the formula (I) are those in, which R¹ and R² together with the nitrogen atom to which they are attached form a five- or six-membered heterocyclyl or heteroaryl which is attached via nitrogen and which may contain one, two or three further heteroatoms from the group consisting of O, N and S as ring member, where the heterocyclyl or heteroaryl is unsubstituted or substituted by one or two identical or different substituents R^{a}. Here, R^{a} is preferably selected from the group consisting of halogen, (C₁-C₆)-alkyl and (C₁-C₆)-haloalkyl.

Especially preferred are compounds of the formula (I) in which R¹ and R² together with the nitrogen atom to which they are attached are saturated or monounsaturated, in particular 5- or 6-membered heterocyclyl as defined above. Among these, preference is given to those compounds in which R¹ and R² together with the nitrogen atom to Which they are attached form an optionally substituted piperidinyl, morpholinyl or thiomorpholinyl ring, especially a piperidinyl ring. Heterocyclyl is in particular preferred, which is unsubstituted or substituted by 1, 2 or 3 substituents R^{a}, preferred substituents R^{a} on heterocyclyl being selected from the group consisting of halogen, (C₁-C₄)-alkyl and (C₁-C₄)-haloalkyl. Among these, particular preference is given to compounds (I) in which R¹ and R² together with the nitrogen atom, to which they are attached, form a 4-methylpiperidine ring, a 4-trifluoromethylpiperidine ring, a morpholine ring or a 3,4-dimethylpiperidine ring and especially a 4-methylpiperidine ring or a 3,4-dimethylpiperidine ring.

The invention furthermore particularly preferably provides compounds (I) in which R¹ and R² together with the nitrogen atom to which they are attached are 5- or 6-membered heteroaryl as defined above which may be unsubstituted or substituted, preferably by 1, 2 or 3 groups R^{a}. In this case, group NR¹R² forms in particular a pyrazole ring which is optionally substituted in the manner described above and especially by 1 or 2 of the following radicals: halogen, (C₁-C₄)-alkyl or (C₁-C₄)-haloalkyl, in particular by 2 methyl groups or 2 trifluoromethyl groups in the 3,5-position.

Very particular preference is given to compounds of the formula (I) in which R¹ is selected from the group consisting of: CH(CH₃)CH₂CH₃, CH(CH₃)CH(CH₃)₂, CH(CH₃)C(CH₃)₃, CH(CH₃)CF₃, CH₂C(CH₃)═CH₂, CH₂CH═CH₂, cyclopentyl and cyclohexyl where R₂ in these cases is preferably hydrogen or methyl; and also to compounds (I) in which R¹ and R² together are —(CH₂)₂CH(CH₃)(CH₂)₂—, —(CH₂)₂CH(CF₃)(CH₂)₂— or —(CH₂)₂—O—(CH₂)₂—.

In the compounds of the formula (I) according to the invention and the corresponding precursors, X is as defined further above. X is in particular (C₁-C₄)-alkyl, (C₁-C₄)-haloalkyl, particularly preferred compounds of the formula (I) being those in which X is (C₁-C₂)-alkyl, in particular methyl. In one embodiment, X is (C₁-C₄)-alkyl, more preferably (C₁-C₂)-alkyl, i.e. methyl or ethyl, (C₁-C₄)-haloalkyl, such as, for example, fluoromethyl, chloromethyl, bromomethyl, difluoromethyl, dichloromethyl, trifluoromethyl, trichloromethyl, chlorofluoromethyl, dichlorofluoromethyl or chlorodifluoromethyl. Furthermore preferably, X is (C₂-C₆)-alkenyl, or (C₂-C₆)-haloalkenyl, more preferably (C₂-C₄)-alkenyl or (C₂-C₄)-haloalkenyl.

In a further embodiment, X is (C₁-C₄)-alkyl, in particular n-propyl, i-propyl, ethyl or methyl, which may be substituted by one or more cyano and/or alkoxy groups.

In one embodiment of the invention, X is cyano-(C₁-C₄)-alkyl, preferably cyano-(C₁-C₂)-alkyl, in particular —CH₂—CN. In a further embodiment of the invention, X is (C₁-C₄)-alkoxy-(C₁-C₄)-alkyl, in particular (C₁-C₂)-alkoxy-(C₁-C₂)-alkyl, such as methoxymethyl, or (C₁-C₄)-alkyl, in particular n-propyl, ethyl or methyl. In a preferred embodiment, R¹ and R² are furthermore hydrogen.

In the compounds of the formula (I) according to the invention and the corresponding precursors, Y is as defined above. Y is in particular hydrogen, halogen, cyano, (C₁-C₄)-alkyl, (C₁-C₄)-haloalkyl, (C₁-C₄)-alkoxy, (C₁-C₂)-haloalkoxy, (C₁-C₄)-alkylthio, (C₁-C₄)-alkylsulfinyl or (C₁-C₄)-alkylsulfonyl. Furthermore preferably, Y is hydrogen, halogen, preferably fluorine, chlorine or bromine, (C₁-C₄)-alkyl, (C₁-C₄)-haloalkyl, (C₃-C₆)-cycloalkyl or (C₃-C₆)-halocycloalkyl.

In a preferred embodiment, Y is hydrogen. According to a further preferred embodiment of the present invention, Y is halogen, preferably fluorine, chlorine or bromine.

According to a further preferred embodiment of the invention, Y is (C₁-C₄)-alkyl or (C₁-C₄)-haloalkyl, preferably (C₁-C₂)-alkyl or (C₁-C₂)-haloalkyl, in particular methyl or ethyl which may be substituted by one, two or three halogen atoms.

According to a further preferred embodiment of the present invention, Y is (C₃-C₆)-cycloalkyl or (C₃-C₆)-halocycloalkyl, particularly preferably cyclopropyl or halocyclopropyl which may carry one to three halogen atoms.

According to a further preferred embodiment, Y is NH₂. Among these, particular preference is given to compounds in which R¹=R²=hydrogen. Furthermore preferably, X in such compounds is (C₁-C₄)-alkyl, (C₁-C₂)-alkoxy-(C₁-C₄)-alkyl, in particular methyl, ethyl, n-propyl or methoxymethyl.

In the compounds according to the invention, Het, as defined above, is a 6-membered heteroaromatic radical which contains one, two or three nitrogen atoms, where Het is unsubstituted or substituted by one, two, three or four identical of different substituents L. In particular, Het is pyridinyl, pyridazinyl, pyrazinyl, 1,2,4-triazinyl, 1,3,5-triazinyl or pyrimidinyl. In a preferred embodiment, Het is selected from the group consisting of pyridinyl, pyridazinyl, pyrazinyl, 1,2,4-triazinyl and 1,3,5-triazinyl. In a further preferred embodiment, Het is pyrimidyl.

In a preferred embodiment of the invention, Het is unsubstituted. In a further preferred embodiment, Het has one, two, three or four, preferably one or two, identical or different substituents L.

Preferred substituents L on Het are halogen, cyano, nitro, NH₂, (C₁-C₆)-alkylamino, di-C₁-C₆-alkylamino, (C₁-C₆)-alkyl, (C₁-C₆)-haloalkyl, (C₁-C₆)-alkoxy, (C₁-C₆)-alkylamino, di-(C₁-C₆)-alkylamino, NH—C(O)—(C₁-C₆)-alkyl, a group C(S)A² and a group C(O)A². Here, A² is as defined above and is preferably (C₁-C₄)-alkoxy, NH₂, (C₁-C₄)-alkylamino or di-(C₁-C₄)-alkylamino. Especially preferred radicals L are, independently of one another, selected from the group consisting of fluorine, chlorine, bromine, cyano, nitro, (C₁-C₄)-alkyl, (C₁-C₄)-haloalkyl, (C₁-C₄)-alkoxy and (C₁-C₄)-alkoxycarbonyl, particularly preferably from the group consisting of fluorine, chlorine, (C₁-C₂)-alkyl, such as methyl or ethyl, (C₁-C₂)-fluoroalkyl, such as trifluoromethyl, (C₁-C₂)-alkoxy, such as Methoxy, or (C₁-C₂)-alkoxycarbonyl, such as methoxycarbonyl.

In compounds of the formula (I) which are furthermore preferred according to the invention, Het has 1, 2 or 3 substituents L which, independently of one another, are selected from the group consisting of halogen, cyano, nitro, NH₂, (C₁-C₆)-alkylamino, di-(C₁-C₆)-alkylamino, (C₁-C₆)-alkyl, (C₁-C₆)-haloalkyl, (C₁-C₆)-alkoxy, (C₁-C₆)-alkylamino, di-(C₁-C₆)-alkylamino, NH—C(O)—(C₁-C₆)-alkyl, a group C(S)A² and a group C(O)A².

According to a preferred embodiment of the invention, at least one of the heteroatoms of the 6-membered heteroaromatic radical Het and/or a substituent L is located in the position ortho to the point of attachment of Het to the triazolopyrimidine unit. Preferred substituents L in der ortho-position are fluorine, chlorine, bromine, iodine, (C₁-C₂)-alkyl, such as methyl or ethyl, (C₁-C₂)-fluoroalkyl, such as trifluoromethyl, and (C₁-C₂)-alkoxy, such as methoxy. Furthermore preferably, L is CN, methylthio, methylsulfinyl, methylsulfonyl, nitro or methoxymethyl. Also preferred are chlorine, bromine, iodine, in particular chlorine. Likewise preferably, L is CN, C₁-C₂-alkyl, such as methyl or ethyl, C₁-C₂-alkoxy, such as methoxy. Especially preferred are chlorine, methyl, CN, methoxy, methylthio.

According to a further preferred embodiment of the invention, Het has at least one substituent located in the meta- or para-position to the point of attachment of Het to the triazolopyrimidine unit.

A preferred embodiment of the invention relates to compounds of the formula (I) in which Het is pyridinyl which optionally has 1, 2, 3 or 4 substituents L.

Among these, preference is given to compounds of the formula I in which Het is 2-pyridinyl which has 1 or 2 substituents L. Among these, particular preference is given to those compounds in which one of the substituents L is located in the 5-position of the pyridinyl ring, i.e. para to the point of attachment. Among these, particular preference is moreover given to compounds I in which one of the substituents L is located in the 3-position, i.e. ortho to the point of attachment, of the pyridinyl ring. Among these, preference is moreover given to compounds of the formula I, in which the 2-pyridyl radical carries a substituent L in the 3-position and a further substituent in the 5-position. Here, L has in particular the meanings given as being preferred. The radical in the 3-position is in particular selected from the group consisting of chlorine, bromine, C₁-C₂-alkyl, such as methyl or ethyl, C₁-C₂-alkoxy, such as methoxy, methylthio, methylsulfinyl, methylsulfonyl, nitro and methoxymethyl, and is in particular chlorine or iodine. The radical in the 5-position is in particular selected from the group consisting of fluorine, chlorine, bromine, cyano, nitro, C₁-C₂-alkyl, such as methyl or ethyl, C₁-C₂-alkoxy, such as methoxy, C₁-C₂-alkoxycarbonyl, such as methoxycarbonyl or ethoxycarbonyl, CONH₂, C₁-C₂-alkylaminocarbonyl, such as methylaminocarbonyl or ethylaminocarbonyl, C₁-C₂-alkylcarbonyl, such as acetyl, and C(S)NH₂. Preference is given in particular to compounds of the formula I in which Het is one of the following radicals of the formulae Het-1, Het-2 or Het-3,

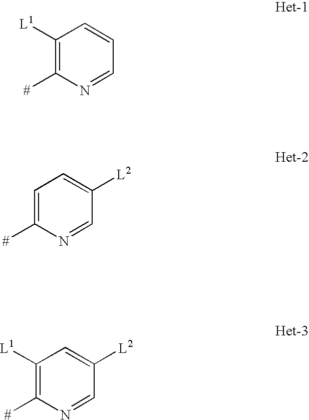

in which # is the point of attachment to the triazolopyrimidine unit and
L¹ is chlorine, bromine, C₁-C₂-alkyl, such as methyl or ethyl, C₁-C₂-alkoxy, such as methoxy, methylthio, methylsulfinyl, methylsulfonyl, nitro or methoxymethyl, especially chlorine, and
L² is fluorine, chlorine, bromine, iodine, cyano, nitro, C₁-C₂-alkyl; such as methyl or ethyl, C₁-C₂-alkoxy, such as methoxy, C₁-C₂-alkoxycarbonyl, such as methoxycarbonyl or ethoxycarbonyl, CONH₂, C₁-C₂-alkylaminocarbonyl, such as methylaminocarbonyl or ethylaminocarbonyl, C₁-C₂-alkylcarbonyl, such as acetyl or C(S)NH₂.

Preference is moreover given to compounds of the formula I in which Het is 3-pyridinyl which optionally has 1 or 2 substituents L. Preferred among these are those compounds which have a substituent L in the 2-position (ortho to the point of attachment and to the nitrogen of the pyridine ring) and/or a substituent L in the 4-position of the pyridine ring (ortho to the point of attachment and para to the nitrogen of the pyridine ring). Preferred are in particular compounds of the formula I in which Het is one of the following radicals of the formula Het-4, Het-5, Het-6, Het-7 or Het-8,

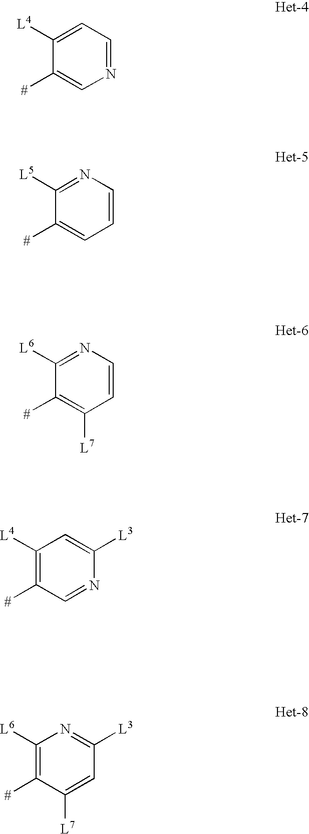

in which # is the point of attachment to the triazolopyrimidine unit and
L³ is fluorine, chlorine, bromine, iodine, cyano, nitro, C₁-C₂-alkyl, such as methyl or ethyl, C₁-C₂-alkoxy, such as methoxy, C₁-C₂-alkoxycarbonyl, such as methoxycarbonyl or ethoxycarbonyl, CONFH₂, C₁-C₂-alkylaminocarbonyl, such as methylaminocarbonyl or ethylaminocarbonyl, C₁-C₂-alkylcarbonyl, such as acetyl or C(S)NH₂,
L⁴ is fluorine, chlorine, bromine, iodine, C₁-C₂-alkyl, such as methyl or ethyl, C₁-C₂-alkoxy, such as methoxy, methylthio, methylsulfinyl, methylsulfonyl, nitro or methoxymethyl,
L⁵ is fluorine, chlorine, bromine, iodine, C₁-C₂-alkyl, such as methyl or ethyl, C₁-C₂-alkoxy, such as methoxy, methylthio, methylsulfinyl, methylsulfonyl, nitro or methoxymethyl, and
L⁶ and L⁷ independently of one another have one of the following meanings: fluorine, chlorine, bromine, iodine, C₁-C₂-alkyl, such as methyl or ethyl, C₁-C₂-alkoxy, such as methoxy, methylthio, methylsulfinyl, methylsulfonyl, nitro or methoxymethyl.

Moreover, preference is given to compounds of the formula I in which Het is 4-pyridinyl which optionally has 1 or 2 substituents L. Among these, preference is given to those compounds which have a substituent L in the 3-position and/or a substituent L in the 5-position of the pyridine ring.

Preference is also given to compounds of the formula I in which Het is one of the following radicals of the formula Het-9 or Het-10,

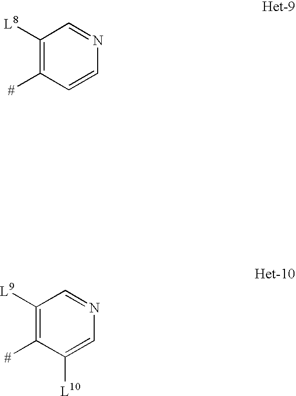

in which # is the point of attachment to the triazolopyrimidine unit and
L⁸ is fluorine, chlorine, bromine, iodine, C₁-C₂-alkyl, such as methyl or ethyl, C₁-C₂-alkoxy, such as methoxy, methylthio, methylsulfinyl, methylsulfonyl, nitro or methoxymethyl,
L⁹ is fluorine, chlorine, bromine, iodine, C₁-C₂-alkyl, such as methyl or ethyl, C₁-C₂-alkoxy, such as methoxy, methylthio, methylsulfinyl, methylsulfonyl, nitro or methoxymethyl, and
L¹⁰ is fluorine, chlorine, bromine, iodine, cyano, nitro, C₁-C₂-alkyl, such as methyl or ethyl, C₁-C₂-alkoxy, such as methoxy, C₁-C₂-alkoxycarbonyl, such as methoxycarbonyl or ethoxycarbonyl, CONH₂, C₁-C₂-alkylaminocarbonyl, such as methylaminocarbonyl or ethylaminocarbonyl, C₁-C₂-alkylcarbonyl, such as acetyl or C(S)NH₂.

A further preferred embodiment of the invention relates to compounds of the formula (I) in which Het is 2-pyrazinyl which optionally has 1, 2 or 3 substituents L.

A further preferred embodiment of the invention relates to compounds of the formula (I) in which Het is 3-pyridazinyl which optionally has 1, 2 or 3 substituents L.

A further preferred embodiment of the invention relates to compounds of the formula (I) in which Het is 1,3,5-triazinyl which optionally has 1 or 2 substituents L.

A further preferred embodiment of the invention relates to compounds of the formula (I) in which Het is unsubstituted pyrimidinyl or substituted pyrimidinyl which may have 1, 2 or 3 identical or different substituents L, in particular unsubstituted or substituted pyrimidin-2-yl, pyrimidin-4-yl or pyrimidin-5-yl.

Besides, R⁵ and R⁶ independently of one another are preferably hydrogen or (C₁-C₄)-alkyl.

R⁷ is preferably hydrogen or in particular (C₁-C₆)-alkyl.

R⁸ and R⁹ independently of one another are preferably hydrogen or (C₁-C₆)-alkyl.

R¹⁰, R¹¹, R¹² and R¹³ independently of one another are preferably selected from the group consisting of hydrogen and (C₁-C₆)-alkyl.

Furthermore, A¹ is preferably hydrogen, (C₁-C₆)-alkyl or amino. The index n is preferably 0, 1 or 2.

A² is preferably (C₁-C₄)-alkoxy, NH₂, (C₁-C₄)-alkylamino or di-(C₁-C₄)-alkylamino.

According to a preferred embodiment of the present invention, the substituents. R¹, R², X and Y in the compounds of the formula (I) or the precursors thereof are as defined below:
R¹, R² is hydrogen;
X is (C₁-C₄)-alkyl, (C₁-C₄)-alkoxy-(C₁-C₄)-alkyl, in particular methyl, ethyl, n-propyl or methoxymethyl;
Y is hydrogen, (C₁-C₄)-alkyl, NH₂, (C₁-C₄)-alkylamino or di-(C₁-C₄)-alkylamino, in particular hydrogen, methyl, ethyl or NH₂; and
   Het has the meanings or preferred meanings as given for compounds of the formula (I). Here, it may be preferred that Het is unsubstituted or substituted by one or more independently selected (C₁-C₈)-alkyl, (C₂-C₁₀)-alkenyl or (C₂-C₁₀)-alkynyl groups, such as, for example, ethyl, n-propyl, i-propyl, n-butyl, i-butyl, sec-butyl or tert-butyl.

Examples of preferred compounds of the formula (I) are the compounds (I) compiled in Tables 1 to 1387 below. The groups mentioned in Tables 1 to 1387 for a substituent Het are furthermore per se, independently of the combination in which they are mentioned, a particularly preferred embodiment of the substituent in question.

### Table 1

Compounds of the formula (I) in which X is methyl and Het is 2-pyridinyl and the combination of R¹, R² and Y for a compound corresponds in each case to one row of Table A.

### Table 2

Compounds of the formula (I) in which X is methyl and Het is 5-nitropyridin-2-yl and the combination of R¹, R² and Y for a compound corresponds in each case to one row of Table A.

### Table 3

Compounds of the formula (I) in which X is methyl and Het is 5-cyanopyridin-2-yl and the combination of R¹, R² and Y for a compound corresponds in each case to one row of Table A.

### Table 4

Compounds of the formula (I) in which X is methyl and Het is 5-methoxycarbonyl-pyridin-2-yl and the combination of R¹, R² and Y for a compound corresponds in each case to one row of Table A.

### Table 5

Compounds of the formula (I) in which X is methyl and Het is 5-methylpyridin-2-yl and the combination of R¹, R² and Y for a compound corresponds in each case to one row of Table A.

### Table 6

Compounds of the formula (I) in which X is methyl and Het is 4-methylpyridin-2-yl and the combination of R¹, R² and Y for a compound corresponds in each case to one row of Table A.

### Table 7

Compounds of the formula (I) in which X is methyl and Het is 3-methylpyridin-2-yl and the combination of R¹, R² and Y for a compound corresponds in each case to one row of Table A.

### Table 8

Compounds of the formula (I) in which X is methyl and Het is 3-ethylpyridin-2-yl and the combination of R¹, R² and Y for a compound corresponds in each case to one row of Table A.

### Table 9

Compounds of the formula (I) in which X is methyl and Het is 6-methylpyridin-2-yl and the combination of R¹, R² and Y for a compound corresponds in each case to one row of Table A.

### Table 10

Compounds of the formula (I) in which X is methyl and Het is 5-trifluoromethyl-pyridin-2-yl and the combination of R¹, R² and Y for a compound corresponds in each case to one row of Table A.

### Table 11

Compounds of the formula (I) in which X is methyl and Het is 3-trifluoromethyl-pyridin-2-yl and the combination of R¹, R² and Y for a compound corresponds in each case to one row of Table A.

### Table 12

Compounds of the formula (I) in which X is methyl and Het is 5-fluoropyridin-2-yl and the combination of R¹, R² and Y for a compound corresponds in each case to one row of Table A.

### Table 13

Compounds of the formula (I) in which X is methyl and Het is 3-fluoropyridin-2-yl and the combination of R¹, R² and Y for a compound corresponds in each case to one row of Table A.

### Table 14

Compounds of the formula (I) in which X is methyl and Het is 3,5-difluoropyridin-2-yl and the combination of R¹, R² and Y for a compound corresponds in each case to one row of Table A.

### Table 15

Compounds of the formula (I) in which X is methyl and Het is 3,5-dichloropyridin-2-yl and the combination of R¹, R² and Y for a compound corresponds in each case to one row of Table A.

### Table 16

Compounds of the formula (I) in which X is methyl and Het is pyridin-3-yl and the combination of R¹, R² and Y for a compound corresponds in each case to one row of Table A.

### Table 17

Compounds of the formula (I) in which X is methyl and Het is pyridin-4-yl and the combination of R¹, R² and Y for a compound corresponds in each case to one row of Table A.

### Table 18

Compounds of the formula (I) in which X is methyl and Het is pyrazin-2-yl and the combination of R¹, R² and Y for a compound corresponds in each case to one row of Table A.

### Table 19

Compounds of the formula (I) in which X is methyl and Het is pyridazin-3-yl and the combination of R¹, R² and Y for a compound corresponds in each case to one row of Table A.

### Table 20

Compounds of the formula (I) in which X is methyl and Het is 6-chloropyridazin-3-yl and the combination of R¹, R² and Y for a compound corresponds in each case to one row of Table A.

### Table 21

Compounds of the formula (I) in which X is methyl and Het is 6-methoxypyridazin-3-yl and the combination of R¹, R² and Y for a compound corresponds in each case to one row of Table A.

Table 22
Compounds of the formula (I) in which X is methyl and Het is 1,3,5-triazin-2-yl and the combination of R¹, R² and Y for a compound corresponds in each case to one row of Table A.

### Table 23

Compounds of the formula (I) in which X is methyl and Het is pyrimidin-2-yl and the combination of R¹, R² and Y for a compound corresponds in each case to one row of Table A.

### Table 24

Compounds of the formula (I) in which X is methyl and Het is pyrimidin-4-yl and the combination of R¹, R² and Y for a compound corresponds in each case to one row of Table A.

### Table 25

Compounds of the formula (I) in which X is methyl and Het is pyrimidin-5-yl and the combination of R¹, R² and Y for a compound corresponds in each case to one row of Table A.

### Table 26

Compounds of the formula (I) in which X is methyl and Het is 2-methyl-6-trifluoro-methylpyrimidin-4-yl and the combination of R¹, R² and Y for a compound corresponds in each case to one row of Table A.

### Table 27

Compounds of the formula (I) in which X is methyl and Het is 2,5-dimethyl-6-trifluoromethylpyrimidin-4-yl and the combination of R¹, R² and Y for a compound corresponds in each case to one row of Table A.

### Table 28

Compounds of the formula (I) in which X is methyl and Het is 6-trifluoromethylpyrimidin-4-yl and the combination of R¹, R² and Y for a compound corresponds in each case to one row of Table A.

### Table 29

Compounds of the formula (I) in which X is methyl and Het is 5-methyl-6-trifluoromethylpyrimidin-4-yl and the combination of R¹, R² and Y for a compound corresponds in each case to one row of Table A.

### Table 30

Compounds of the formula (I) in which X is methyl and Het is 5-chloro-6-methylpyrimidin-4-yl and the combination of R¹, R² and Y for a compound corresponds in each case to one row of Table A.

### Table 31

Compounds of the formula (I) in which X is methyl and Het is 5-bromo-6-methylpyrimidin-4-yl and the combination of R¹, R² and Y for a compound corresponds in each case to one row of Table A.

### Table 32

Compounds of the formula (I) in which X is methyl and Het is 5-fluoro-6-methylpyrimidin-4-yl and the combination of R¹, R² and Y for a compound corresponds in each case to one row of Table A.

### Table 33

Compounds of the formula (I) in which X is methyl and Het is 5-chloro-6-ethylpyrimidin-4-yl and the combination of R¹, R² and Y for a compound corresponds in each case to one row of Table A.

### Table 34

Compounds of the formula (I) in which X is methyl and Het is 5-bromo-6-ethylpyrimidin-4-yl and the combination of R¹, R² and Y for a compound corresponds in each case to one row of Table A.

### Table 35

Compounds of the formula (I) in which X is methyl and Het is 5-chloro-6-isopropyl-pyrimidin-4-yl and the combination of R¹, R² and Y for a compound corresponds in each case to one row of Table A.

### Table 36

Compounds of the formula (I) in which X is methyl and Het is 5-bromo-6-isopropyl-pyrimidin-4-yl and the combination of R¹, R² and Y for a compound corresponds in each case to one row of Table A.

### Table 37

Compounds of the formula (I) in which X is methyl and Het is 5-chloropyrimidin-4-yl and the combination of R¹, R² and Y for a compound corresponds in each case to one row of Table A.

### Table 38

Compounds of the formula (I) in which X is methyl and Het is 5-fluoropyrimidin-4-yl and the combination of R¹, R² and Y for a compound corresponds in each case to one row of Table A.

### Table 39

Compounds of the formula (I) in which X is methyl and Het is 5-bromopyrimidin-4-yl and the combination of R¹, R² and Y for a compound corresponds in each case to one row of Table A.

### Table 40

Compounds of the formula (I) in which X is methyl and Het is 2-methyl-5-fluoropyrimidin-4-yl and the combination of R¹, R² and Y for a compound corresponds in each case to one row of Table A.

### Table 41

Compounds of the formula (I) in which X is methyl and Het is 2-methyl-5-chloropyrimidin-4-yl and the combination of R¹, R² and Y for a compound corresponds in each case to one row of Table A.

### Table 42

Compounds of the formula (I) in which X is methyl and Het is 2-methyl-5-bromopyrimidin-4-yl and the combination of R¹, R² and Y for a compound corresponds in each case to one row of Table A.

### Table 43

Compounds of the formula (I) in which X is methyl and Het is 2,6-dimethyl-5-chloropyrimidin-4-yl and the combination of R¹, R² and Y for a compound corresponds in each case to one row of Table A.

### Table 44

Compounds of the formula (I) in which X is methyl and Het is 2,6-dimethyl-5-bromopyrimidin-4-yl and the combination of R¹, R² and Y for a compound corresponds in each case to one row of Table A.

### Table 45

Compounds of the formula (I) in which X is methyl and Het is 5-fluoro-6-fluoromethylpyrimidin-4-yl and the combination of R¹, R² and Y for a compound corresponds in each case to one row of Table A.

### Table 46

Compounds of the formula (I) in which X is methyl and Het is 5-chloro-6-methoxymethylpyrimidin-4-yl and the combination of R¹, R² and Y for a compound corresponds in each case to one row of Table A.

### Table 47

Compounds of the formula (I) in which X is methyl and Het is 5,6-dimethylpyrimidin-4-yl and the combination of R¹, R² and Y for a compound corresponds in each case to one row of Table A.

### Table 48

Compounds of the formula (I) in which X is methyl and Het is 2,5-dimethylpyrimidin-4-yl and the combination of R¹, R² and Y for a compound corresponds in each case to one row of Table A.

### Table 49

Compounds of the formula (I) in which X is methyl and Het is 2,5,6-trimethylpyrimidin-4-yl and the combination of R¹, R² and Y for a compound corresponds in each case to one row of Table A.

### Table 50

Compounds of the formula (I) in which X is methyl and Het is 5-methyl-6-methoxy-pyrimidin-4-yl and the combination of R¹, R² and Y for a compound corresponds in each case to one row of Table A.

### Table 51

Compounds of the formula (I) in which X is methyl and Het is 4,6-dimethylpyrimidin-2-yl and the combination of R¹, R² and Y for a compound corresponds in each case to one row of Table A.

### Table 52

Compounds of the formula (I) in which X is methyl and Het is 4,5,6-trimethylpyrimidin-2-yl and the combination of R¹, R² and Y for a compound corresponds in each case to one row of Table A.

### Table 53

Compounds of the formula (I) in which X is methyl and Het is 4,6-di(trifluoromethyl)-pyrimidin-2-yl and the combination of R¹, R² and Y for a compound corresponds in each case to one row of Table A.

### Table 54

Compounds of the formula (I) in which X is methyl and Het is 4,6-dimethyl-5-chloropyrimidin-2-yl and the combination of R¹, R² and Y for a compound corresponds in each case to one row of Table A.

### Table 55

Compounds of the formula (I) in which X is methyl and Het is 4,6-dimethyl-5-bromopyrimidin-2-yl and the combination of R¹, R² and Y for a compound corresponds in each case to one row of Table A.

### Table 56

Compounds of the formula (I) in which X is methyl and Het is 4-methylpyrimidin-5-yl and the combination of R¹, R² and Y for a compound corresponds in each case to one row of Table A.

### Table 57

Compounds of the formula (I) in which X is methyl and Het is 4,6-dimethylpyrimidin-5-yl and the combination of R¹, R² and Y for a compound corresponds in each case to one row of Table A.

### Table 58

Compounds of the formula (I) in which X is methyl and Het is 4-trifluoromethyl-6-methylpyrimidin-5-yl and the combination of R¹, R² and Y for a compound corresponds in each case to one row of Table A.

### Table 59

Compounds of the formula (I) in which X is methyl and Het is 2,4,6-trimethylpyrimidin-5-yl and the combination of R¹, R² and Y for a compound corresponds in each case to one row of Table A.

### Table 60

Compounds of the formula (I) in which X is methyl and Het is 3-chloropyridin-2-yl and the combination of R¹, R² and Y for a compound corresponds in each case to one row of Table A.

### Table 61

Compounds of the formula (I) in which X is methyl and Het is 3-bromopyridin-2-yl and the combination of R¹, R² and Y for a compound corresponds in each case to one row of Table A.

### Table 62

Compounds of the formula (I) in which X is methyl and Het is 3-iodopyridin-2-yl and the combination of R¹, R² and Y for a compound corresponds in each case to one row of Table A.

### Table 63

Compounds of the formula (I) in which X is methyl and Het is 3-methoxypyridin-2-yl and the combination of R¹, R² and Y for a compound corresponds in each case to one row of Table A.

### Table 64

Compounds of the formula (I) in which X is methyl and Het is 3-methylsulfanylpyridin-2-yl and the combination of R¹, R² and Y for a compound corresponds in each case to one row of Table A.

### Table 65

Compounds of the formula (I) in which X is methyl and Het is 3-methylsulfinylpyridin-2-yl and the combination of R¹, R² and Y for a compound corresponds in each case to one row of Table A.

### Table 66

Compounds of the formula (I) in which X is methyl and Het is 3-methylsulfonylpyridin-2-yl and the combination of R¹, R² and Y for a compound corresponds in each case to one row of Table A.

### Table 67

Compounds of the formula (I) in which X is methyl and Het is 3-nitropyridin-2-yl and the combination of R¹, R² and Y for a compound corresponds in each case to one row of Table A.

### Table 68

Compounds of the formula (I) in which X is methyl and Het is 3-methoxymethylpyridin-2-yl and the combination of R¹, R² and Y for a compound corresponds in each case to one row of Table A.

### Table 69

Compounds of the formula (I) in which X is methyl and Het is 5-chloropyridin-2-yl and the combination of R¹, R² and Y for a compound corresponds in each case to one row of Table A.

### Table 70

Compounds of the formula (I) in which X is methyl and Het is 5-bromopyridin-2-yl and the combination of R¹, R² and Y for a compound corresponds in each case to one row of Table A.

### Table 71

Compounds of the formula (I) in which X is methyl and Het is 5-methoxypyridin-2-yl and the combination of R¹, R² and Y for a compound corresponds in each case to one row of Table A.

### Table 72

Compounds of the formula (I) in which X is methyl and Het is 5-methylsulfanylpyridin-2-yl and the combination of R¹, R² and Y for a compound corresponds in each case to one row of Table A.

### Table 73

Compounds of the formula (I) in which X is methyl and Het is 5-methylsulfinylpyridin-2-yl and the combination of R¹, R² and Y for a compound corresponds in each case to one row of Table A.

### Table 74

Compounds of the formula (I) in which X is methyl and Het is 5-methylsulfonylpyridin-2-yl and the combination of R¹, R² and Y for a compound corresponds in each case to one row of Table A.

### Table 75

Compounds of the formula (I) in which X is methyl and Het is 5-methoxymethylpyridin-2-yl and the combination of R¹, R² and Y for a compound corresponds in each case to one row of Table A.

### Table 76

Compounds of the formula (I) in which X is methyl and Het is 5-ethylpyridin-2-yl and the combination of R¹, R² and Y for a compound corresponds in each case to one row of Table A.

### Table 77

Compounds of the formula (I) in which X is methyl and Het is 5-ethoxycarbonylpyridin-2-yl and the combination of R¹, R² and Y for a compound corresponds in each case to one row of Table A.

### Table 78

Compounds of the formula (I) in which X is methyl and Het is 5-carbamoylpyridin-2-yl and the combination of R¹, R² and Y for a compound corresponds in each case to one row of Table A.

### Table 79

Compounds of the formula (I) in which X is methyl and Het is 5-methylcarbamoylpyridin-2-yl and the combination of R¹, R² and Y for a compound corresponds in each case to one row of Table A.

### Table 80

Compounds of the formula (I) in which X is methyl and Het is 5-dimethylcarbamoylpyridin-2-yl and the combination of R¹, R² and Y for a compound corresponds in each case to one row of Table A.

### Table 81

Compounds of the formula (I) in which X is methyl and Het is 5-thiocarbamoylpyridin-2-yl and the combination of R¹, R² and Y for a compound corresponds in each case to one row of Table A.

### Table 82

Compounds of the formula (I) in which X is methyl and Het is 5-acetylpyridin-2-yl and the combination of R¹, R² and Y for a compound corresponds in each case to one row of Table A.

### Table 83

Compounds of the formula (I) in which X is methyl and Het is 5-methylpyridin-2-yl and the combination of R¹, R² and Y for a compound corresponds in each case to one row of Table A.

### Table 84

Compounds of the formula (I) in which X is methyl and Het is 5-nitropyridin-2-yl and the combination of R¹, R² and Y for a compound corresponds in each case to one row of Table A.

### Table 85

Compounds of the formula (I) in which X is methyl and Het is 5-cyanopyridin-2-yl and the combination of R¹, R² and Y for a compound corresponds in each case to one row of Table A.

### Table 86

Compounds of the formula (I) in which X is methyl and Het is 3-fluoro-5-chloropyridin-2-yl and the combination of R¹, R² and Y for a compound corresponds in each case to one row of Table A.

### Table 87

Compounds of the formula (I) in which X is methyl and Het is 3-fluoro-5-bromopyridin-2-yl and the combination of R¹, R² and Y for a compound corresponds in each case to one row of Table A.

### Table 88

Compounds of the formula (I) in which X is methyl and Het is 3-fluoro-5-methylpyridin-2-yl and the combination of R¹, R² and Y for a compound corresponds in each case to one row of Table A.

### Table 89

Compounds of the formula (I) in which X is methyl and Het is 3-fluoro-5-ethylpyridin-2-yl and the combination of R¹, R² and Y for a compound corresponds in each case to one row of Table A.

### Table 90

Compounds of the formula (I) in which X is methyl and Het is 3-fluoro-5-methoxypyridin-2-yl and the combination of R¹, R² and Y for a compound corresponds in each case to one row of Table A.

### Table 91

Compounds of the formula (I) in which X is methyl and Het is 3-fluoro-5-cyanopyridin-2-yl and the combination of R¹, R² and Y for a compound corresponds in each case to one row of Table A.

### Table 92

Compounds of the formula (I) in which X is methyl and Het is 3-fluoro-5-methoxycarbonylpyridin-2-yl and the combination of R¹, R² and Y for a compound corresponds in each case to one row of Table A.

### Table 93

Compounds of the formula (I) in which X is methyl and Het is 3-fluoro-5-ethoxycarbonylpyridin-2-yl and the combination of R¹, R² and Y for a compound corresponds in each case to one row of Table A.

### Table 94

Compounds of the formula (I) in which X is methyl and Het is 3-fluoro-5-carbamoylpyridin-2-yl and the combination of R¹, R² and Y for a compound corresponds in each case to one row of Table A.

### Table 95

Compounds of the formula (I) in which X is methyl and Het is 3-fluoro-5-methylcarbamoylpyridin-2-yl and the combination of R¹, R² and Y for a compound corresponds in each case to one row of Table A.

### Table 96

Compounds of the formula (I) in which X is methyl and Het is 3-fluoro-5-dimethylcarbamoylpyridin-2-yl and the combination of R¹, R² and Y for a compound corresponds in each case to one row of Table A.

### Table 97

Compounds of the formula (I) in which X is methyl and Het is 3-fluoro-5-thiocarbamoylpyridin-2-yl and the combination of R¹, R² and Y for a compound corresponds in each case to one row of Table A.

### Table 98

Compounds of the formula (I) in which X is methyl and Het is 3-fluoro-5-nitropyridin-2-yl and the combination of R¹, R² and Y for a compound corresponds in each case to one row of Table A.

### Table 99

Compounds of the formula (I) in which X is methyl and Het is 3-fluoro-5-acetylpyridin-2-yl and the combination of R¹, R² and Y for a compound corresponds in each case to one row of Table A.

### Table 100

Compounds of the formula (I) in which X is methyl and Het is 3-chloro-5-fluoropyridin-2-yl and the combination of R¹, R² and Y for a compound corresponds in each case to one row of Table A.

### Table 101

Compounds of the formula (I) in which X is methyl and Het is 3-chloro-5-bromopyridin-2-yl and the combination of R¹, R² and Y for a compound corresponds in each case to one row of Table A.

### Table 102

Compounds of the formula (I) in which X is methyl and Het is 3-chloro-5-methylpyridin-2-yl and the combination of R¹, R² and Y for a compound corresponds in each case to one row of Table A.

### Table 103

Compounds of the formula (I) in which X is methyl and Het is 3-chloro-5-ethylpyridin-2-yl and the combination of R¹, R² and Y for a compound corresponds in each case to one row of Table A.

### Table 104

Compounds of the formula (I) in which X is methyl and Het is 3-chloro-5-methoxypyridin-2-yl and the combination of R¹, R² and Y for a compound corresponds in each case to one row of Table A.

### Table 105

Compounds of the formula (I) in which X is methyl and Het is 3-chloro-5-cyanopyridin-2-yl and the combination of R¹, R² and Y for a compound corresponds in each case to one row of Table A.

### Table 106

Compounds of the formula (I) in which X is methyl and Het is 3-chloro-5-methoxycarbonylpyridin-2-yl and the combination of R¹, R² and Y for a compound corresponds in each case to one row of Table A.

### Table 107

Compounds of the formula (I) in which X is methyl and Het is 3-chloro-5-ethoxycarbonylpyridin-2-yl and the combination of R¹, R² and Y for a compound corresponds in each case to one row of Table A.

### Table 108

Compounds of the formula (I) in which X is methyl and Het is 3-chloro-5-carbamoylpyridin-2-yl and the combination of R¹, R² and Y for a compound corresponds in each case to one row of Table A.

### Table 109

Compounds of the formula (I) in which X is methyl and Het is 3-chloro-5-methylcarbamoylpyridin-2-yl and the combination of R¹, R² and Y for a compound corresponds in each case to one row of Table A.

### Table 110

Compounds of the formula (I) in which X is methyl and Het is 3-chloro-5-dimethylcarbamoylpyridin-2-yl and the combination of R¹, R² and Y for a compound corresponds in each case to one row of Table A.

### Table 111

Compounds of the formula (I) in which X is methyl and Het is 3-chloro-5-thiocarbamoylpyridin-2-yl and the combination of R¹, R² and Y for a compound corresponds in each case to one row of Table A.

### Table 112

Compounds of the formula (I) in which X is methyl and Het is 3-chloro-5-nitropyridin-2-yl and the combination of R¹, R² and Y for a compound corresponds in each case to one row of Table A.

### Table 113

Compounds of the formula (I) in which X is methyl and Het is 3-chloro-5-acetylpyridin-2-yl and the combination of R¹, R² and Y for a compound corresponds in each case to one row of Table A.

### Table 114

Compounds of the formula (I) in which X is methyl and Het is 3-methyl-5-fluoropyridin-2-yl and the combination of R¹, R² and Y for a compound corresponds in each case to one row of Table A.
   table 115

Compounds of the formula (I) in which X is methyl and Het is 3-methyl-5-chloropyridin-2-yl and the combination of R¹, R² and Y for a compound corresponds in each case to one row of Table A.

### Table 116

Compounds of the formula (I) in which X is methyl and Het is 3-methyl-5-bromopyridin-2-yl and the combination of R¹, R² and Y for a compound corresponds in each case to one row of Table A.

### Table 117

Compounds of the formula (I) in which X is methyl and Het is 3,5-dimethylpyridin-2-yl and the combination of R¹, R² and Y for a compound corresponds in each case to one row of Table A.

### Table 118

Compounds of the formula (I) in which X is methyl and Het is 3-methyl-5-ethylpyridin-2-yl and the combination of R¹, R² and Y for a compound corresponds in each case to one row of Table A.

### Table 119

Compounds of the formula (I) in which X is methyl and Het is 3-methyl-5-methoxypyridin-2-yl and the combination of R¹, R² and Y for a compound corresponds in each case to one row of Table A.

### Table 120

Compounds of the formula (I) in which X is methyl and Het is 3-methyl-5-cyanopyridin-2-yl and the combination of R¹, R² and Y for a compound corresponds in each case to one row of Table A.

### Table 121

Compounds of the formula (I) in which X is methyl and Het is 3-methyl-5-methoxycarbonylpyridin-2-yl and the combination of R¹, R² and Y for a compound corresponds in each case to one row of Table A.

### Table 122

Compounds of the formula (I) in which X is methyl and Het is 3-methyl-5-ethoxycarbonylpyridin-2-yl and the combination of R¹, R² and Y for a compound corresponds in each case to one row of Table A.

### Table 123

Compounds of the formula (I) in which X is methyl and Het is 3-methyl-5-carbamoylpyridin-2-yl and the combination of R¹, R² and Y for a compound corresponds in each case to one row of Table A.

### Table 124

Compounds of the formula (I) in which X is methyl and Het is 3-methyl-5-methylcarbamoylpyridin-2-yl and the combination of R¹, R² and Y for a compound corresponds in each case to one row of Table A.

### Table 125

Compounds of the formula (I) in which X is methyl and Het is 3-methyl-5-dimethylcarbamoylpyridin-2-yl and the combination of R¹, R² and Y for a compound corresponds in each case to one row of Table A.

### Table 126

Compounds of the formula (I) in which X is methyl and Het is 3-methyl-5-thiocarbamoylpyridin-2-yl and the combination of R¹, R² and Y for a compound corresponds in each case to one row of Table A.

### Table 127

Compounds of the formula (I) in which X is methyl and Het is 3-methyl-5-nitropyridin-2-yl and the combination of R¹, R² and Y for a compound corresponds in each case to one row of Table A.

### Table 128

Compounds of the formula (I) in which X is methyl and Het is 3-methyl-5-acetylpyridin-2-yl and the combination of R¹, R² and Y for a compound corresponds in each case to one row of Table A.

### Table 129

Compounds of the formula (I) in which X is methyl and Het is 3-bromo-5-fluoropyridin-2-yl and the combination of R¹, R² and Y for a compound corresponds in each case to one row of Table A.

### Table 130

Compounds of the formula (I) in which X is methyl and Het is 3-bromo-5-chloropyridin-2-yl and the combination of R¹, R² and Y for a compound corresponds in each case to one row of Table A.

### Table 131

Compounds of the formula (I) in which X is methyl and Het is 3-bromo-5-methylpyridin-2-yl and the combination of R¹, R² and Y for a compound corresponds in each case to one row of Table A.

### Table 132

Compounds of the formula (I) in which X is methyl and Het is 3-methylsulfonyl-5-fluoropyridin-2-yl and the combination of R¹, R² and Y for a compound corresponds in each case to one row of Table A.

### Table 133

Compounds of the formula (I) in which X is methyl and Het is 3-methylsulfonyl-5-chloropyridin-2-yl and the combination of R¹, R² and Y for a compound corresponds in each case to one row of Table A.

### Table 134

Compounds of the formula (I) in which X is methyl and Het is 3-methylsulfonyl-5-methylpyridin-2-yl and the combination of R¹, R² and Y for a compound corresponds in each case to one row of Table A.

### Table 135

Compounds of the formula (I) in which X is methyl and Het is 3-methoxy-5-fluoropyridin-2-yl and the combination of R¹, R² and Y for a compound corresponds in each case to one row of Table A.

### Table 136

Compounds of the formula (I) in which X is methyl and Het is 3-methoxy-5-chloropyridin-2-yl and the combination of R¹, R² and Y for a compound corresponds in each case to one row of Table A.

### Table 137

Compounds of the formula (I) in which X is methyl and Het is 3-methoxy-5-methylpyridin-2-yl and the combination of R¹, R² and Y for a compound corresponds in each case to one row of Table A.

### Table 138

Compounds of the formula (I) in which X is methyl and Het is 3-methylsulfanyl-5-fluoropyridin-2-yl and the combination of R¹, R² and Y for a compound corresponds in each case to one row of Table A.

### Table 139

Compounds of the formula (I) in which X is methyl and Het is 3-methylsulfanyl-5-chloropyridin-2-yl and the combination of R¹, R² and Y for a compound corresponds in each case to one row of Table A.

### Table 140

Compounds of the formula (I) in which X is methyl and Het is 3-methylsulfanyl-5-methylpyridin-2-yl and the combination of R¹, R² and Y for a compound corresponds in each case to one row of Table A.

### Table 141

Compounds of the formula (I) in which X is methyl and Het is 3-nitro-5-fluoropyridin-2-yl and the combination of R¹, R² and Y for a compound corresponds in each case to one row of Table A.

### Table 142

Compounds of the formula (I) in which X is methyl and Het is 3-nitro-5-chloropyridin-2-yl and the combination of R¹, R² and Y for a compound corresponds in each case to one row of Table A.

### Table 143

Compounds of the formula (I) in which X is methyl and Het is 3-nitro-5-methylpyridin-2-yl and the combination of R¹, R² and Y for a compound corresponds in each case to one row of Table A.

### Table 144

Compounds of the formula (I) in which X is methyl and Het is 3,5-dibromopyridin-2-yl and the combination of R¹, R² and Y for a compound corresponds in each case to one row of Table A.

### Table 145

Compounds of the formula (I) in which X is methyl and Het is 3-fluoropyridin-4-yl and the combination of R¹, R² and Y for a compound corresponds in each case to one row of Table A.

### Table 146

Compounds of the formula (I) in which X is methyl and Het is 3-chloropyridin-4-yl and the combination of R¹, R² and Y for a compound corresponds in each case to one row of Table A.

### Table 147

Compounds of the formula (I) in which X is methyl and Het is 3-bromopyridin-4-yl and the combination of R¹, R² and Y for a compound corresponds in each case to one row of Table A.

### Table 148

Compounds of the formula (I) in which X is methyl and Het is 3-methylpyridin-4-yl and the combination of R¹, R² and Y for a compound corresponds in each case to one row of Table A.

### Table 149

Compounds of the formula (I) in which X is methyl and Het is 3-methoxypyridin-4-yl and the combination of R¹, R² and Y for a compound corresponds in each case to one row of Table A.

### Table 150

Compounds of the formula (I) in which X is methyl and Het is 3-methylsulfanylpyridin-4-yl and the combination of R¹, R² and Y for a compound corresponds in each case to one row of Table A.

### Table 151

Compounds of the formula (I) in which X is methyl and Het is 3-methylsulfinylpyridin-4-yl and the combination of R¹, R² and Y for a compound corresponds in each case to one row of Table A.

### Table 152

Compounds of the formula (I) in which X is methyl and Het is 3-methylsulfonylpyridin-4-yl and the combination of R¹, R² and Y for a compound corresponds in each case to one row of Table A.

### Table 153

Compounds of the formula (I) in which X is methyl and Het is 3-nitropyridin-4-yl and the combination of R¹, R² and Y for a compound corresponds in each case to one row of Table A.

### Table 154

Compounds of the formula (I) in which X is methyl and Het is 3-methoxymethylpyridin-4-yl and the combination of R¹, R² and Y for a compound corresponds in each case to one row of Table A.

### Table 155

Compounds of the formula (I) in which X is methyl and Het is 3,5-difluoropyridin-4-yl and the combination of R¹, R² and Y for a compound corresponds in each case to one row of Table A.

### Table 156

Compounds of the formula (I) in which X is methyl and Het is 3-fluoro-5-chloropyridin-4-yl and the combination of R¹, R² and Y for a compound corresponds in each case to one row of Table A.

### Table 157

Compounds of the formula (I) in which X is methyl and Het is 3-fluoro-5-bromopyridin-4-yl and the combination of R¹, R² and Y for a compound corresponds in each case to one row of Table A.

### Table 158

Compounds of the formula (I) in which X is methyl and Het is 3-fluoro-5-methylpyridin-4-yl and the combination of R¹, R² and Y for a compound corresponds in each case to one row of Table A.

### Table 159

Compounds of the formula (I) in which X is methyl and Het is 3-fluoro-5-ethylpyridin-4-yl and the combination of R¹, R² and Y for a compound corresponds in each case to one row of Table A.

### Table 160

Compounds of the formula (I) in which X is methyl and Het is 3-fluoro-5-methoxypyridin-4-yl and the combination of R¹, R² and Y for a compound corresponds in each case to one row of Table A.

### Table 161

Compounds of the formula (I) in which X is methyl and Het is 3-fluoro-5-cyanopyridin-4-yl and the combination of R¹, R² and Y for a compound corresponds in each case to one row of Table A.

### Table 162

Compounds of the formula (I) in which X is methyl and Het is 3-fluoro-5-methoxycarbonylpyridin-4-yl and the combination of R¹, R² and Y for a compound corresponds in each case to one row of Table A.

### Table 163

Compounds of the formula (I) in which X is methyl and Het is 3-fluoro-5-ethoxycarbonylpyridin-4-yl and the combination of R¹, R² and Y for a compound corresponds in each case to one row of Table A.

### Table 164

Compounds of the formula (I) in which X is methyl and Het is 3-fluoro-5-carbamoylpyridin-4-yl and the combination of R¹, R² and Y for a compound corresponds in each case to one row of Table A.

### Table 165

Compounds of the formula (I) in which X is methyl and Het is 3-fluoro-5-methylcarbamoylpyridin-4-yl and the combination of R¹, R² and Y for a compound corresponds in each case to one row of Table A.

### Table 166

Compounds of the formula (I) in which X is methyl and Het is 3-fluoro-5-dimethylcarbamoylpyridin-4-yl and the combination of R¹, R² and Y for a compound corresponds in each case to one row of Table A.

### Table 167.

Compounds of the formula (I) in which X is methyl and Het is 3-fluoro-5-thiocarbamoylpyridin-4-yl and the combination of R¹, R² and Y for a compound corresponds in each case to one row of Table A.

### Table 168

Compounds of the formula (I) in which X is methyl and Het is 3-fluoro-5-nitropyridin-4-yl and the combination of R¹, R² and Y for a compound corresponds in each case to one row of Table A.

### Table 169

Compounds of the formula (I) in which X is methyl and Het is 3-fluoro-5-acetylpyridin-4-yl and the combination of R¹, R² and Y for a compound corresponds in each case to one row of Table A.

### Table 170

Compounds of the formula (I) in which X is methyl and Het is 3,5-dichloro-5-chloropyridin-4-yl and the combination of R¹, R² and Y for a compound corresponds in each case to one row of Table A.

### Table 171

Compounds of the formula (I) in which X is methyl and Het is 3-chloro-5-bromopyridin-4-yl and the combination of R¹, R² and Y for a compound corresponds in each case to one row of Table A.

### Table 172

Compounds of the formula (I) in which X is methyl and Het is 3-chloro-5-methylpyridin-4-yl and the combination of R¹, R² and Y for a compound corresponds in each case to one row of Table A.

### Table 173

Compounds of the formula (I) in which X is methyl and Het is 3-chloro-5-ethylpyridin-4-yl and the combination of R¹, R² and Y for a compound corresponds in each case to one row of Table A.

### Table 174

Compounds of the formula (I) in which X is methyl and Het is 3-chloro-5-methoxypyridin-4-yl and the combination of R¹, R² and Y for a compound corresponds in each case to one row of Table A.

### Table 175

Compounds of the formula (I) in which X is methyl and Het is 3-chloro-5-cyanopyridin-4-yl and the combination of R¹, R² and Y for a compound corresponds in each case to one row of Table A.

### Table 176

Compounds of the formula (I) in which X is methyl and Het is 3-chloro-5-methoxycarbonylpyridin-4-yl and the combination of R¹, R² and Y for a compound corresponds in each case to one row of Table A.

### Table 177

Compounds of the formula (I) in which X is methyl and Het is 3-chloro-5-ethoxycarbonylpyridin-4-yl and the combination of R¹, R² and Y for a compound corresponds in each case to one row of Table A.

### Table 178

Compounds of the formula (I) in which X is methyl and Het is 3-chloro-5-carbamoylpyridin-4-yl and the combination of R¹, R² and Y for a compound corresponds in each case to one row of Table A.

### Table 179

Compounds of the formula (I) in which X is methyl and Het is 3-chloro-5-methylcarbamoylpyridin-4-yl and the combination of R¹, R² and Y for a compound corresponds in each case to one row of Table A.

### Table 180

Compounds of the formula (I) in which X is methyl and Het is 3-chloro-5-dimethylcarbamoylpyridin-4-yl and the combination of R¹, R² and Y for a compound corresponds in each case to one row of Table A.

### Table 181

Compounds of the formula (I) in which X is methyl and Het is 3-chloro-5-thiocarbamoylpyridin-4-yl and the combination of R¹, R² and Y for a compound corresponds in each case to one row of Table A.

### Table 182

Compounds of the formula (I) in which X is methyl and Het is 3-chloro-5-nitropyridin-4-yl and the combination of R¹, R² and Y for a compound corresponds in each case to one row of Table A.

### Table 183

Compounds of the formula (I) in which X is methyl and Het is 3-chloro-5-acetylpyridin-4-yl and the combination of R¹, R² and Y for a compound corresponds in each case to one row of Table A.

### Table 184

Compounds of the formula (I) in which X is methyl and Het is 3,5-dimethylpyridin-4-yl and the combination of R¹, R² and Y for a compound corresponds in each case to one row of Table A.

### Table 185

Compounds of the formula (I) in which X is methyl and Het is 3-methyl-5-bromopyridin-4-yl and the combination of R¹, R² and Y for a compound corresponds in each case to one row of Table A.

### Table 186

Compounds of the formula (I) in which X is methyl and Het is 3-methyl-5-ethylpyridin-4-yl and the combination of R¹, R² and Y for a compound corresponds in each case to one row of Table A.

### Table 187

Compounds of the formula (I) in which X is methyl and Het is 3-methyl-5-methoxypyridin-4-yl and the combination of R¹, R² and Y for a compound corresponds in each case to one row of Table A.

### Table 188

Compounds of the formula (I) in which X is methyl and Het is 3-methyl-5-cyanopyridin-4-yl and the combination of R¹, R² and Y for a compound corresponds in each case to one row of Table A.

### Table 189

Compounds of the formula (I) in which X is methyl and Het is 3-methyl-5-methoxycarbonylpyridin-4-yl and the combination of R¹, R² and Y for a compound corresponds in each case to one row of Table A.

### Table 190

Compounds of the formula (I) in which X is methyl and Het is 3-methyl-5-ethoxycarbonylpyridin-4-yl and the combination of R¹, R² and Y for a compound corresponds in each case to one row of Table A.

### Table 191

Compounds of the formula (I) in which X is methyl and Het is 3-methyl-5-carbamoylpyridin-4-yl and the combination of R¹, R² and Y for a compound corresponds in each case to one row of Table A.

### Table 192

Compounds of the formula (I) in which X is methyl and Het is 3-methyl-5-methylcarbamoylpyridin-4-yl and the combination of R¹, R² and Y for a compound corresponds in each case to one row of Table A.

### Table 193

Compounds of the formula (I) in which X is methyl and Het is 3-methyl-5-dimethylcarbamoylpyridin-4-yl and the combination of R¹, R² and Y for a compound corresponds in each case to one row of Table A.

### Table 194

Compounds of the formula (I) in which X is methyl and Het is 3-methyl-5-thiocarbamoylpyridin-4-yl and the combination of R¹, R² and Y for a compound corresponds in each case to one row of Table A.

### Table 195

Compounds of the formula (I) in which X is methyl and Het is 3-methyl-5-nitropyridin-4-yl and the combination of R¹, R² and Y for a compound corresponds in each case to one row of Table A.

### Table 196

Compounds of the formula (I) in which X is methyl and Het is 3-methyl-5-acetylpyridin-4-yl and the combination of R¹, R² and Y for a compound corresponds in each case to one row of Table A.

### Table 197

Compounds of the formula (I) in which X is methyl and Het is 3-methylsulfanyl-5-fluoropyridin-4-yl and the combination of R¹, R² and Y for a compound corresponds in each case to one row of Table A.

### Table 198

Compounds of the formula (I) in which X is methyl and Het is 3-methylsulfanyl-5-chloropyridin-4-yl and the combination of R¹, R² and Y for a compound corresponds in each case to one row of Table A.

### Table 199

Compounds of the formula (I) in which X is methyl and Het is 3-methylsulfanyl-5-methylpyridin-4-yl and the combination of R¹, R² and Y for a compound corresponds in each case to one row of Table A.

### Table 200

Compounds of the formula (I) in which X is methyl and Het is 3-methylsulfinyl-5-fluoropyridin-4-yl and the combination of R¹, R² and Y for a compound corresponds in each case to one row of Table A.

### Table 201

Compounds of the formula (I) in which X is methyl and Het is 3-methylsulfinyl-5-chloropyridin-4-yl and the combination of R¹, R² and Y for a compound corresponds in each case to one row of Table A.

### Table 202

Compounds of the formula (I) in which X is methyl and Het is 3-methylsulfinyl-5-methylpyridin-4-yl and the combination of R¹, R² and Y for a compound corresponds in each case to one row of Table A.

### Table 203

Compounds of the formula (I) in which X is methyl and Het is 3-methylsulfonyl-5-fluoropyridin-4-yl and the combination of R¹, R² and Y for a compound corresponds in each case to one row of Table A.

### Table 204

Compounds of the formula (I) in which X is methyl and Het is 3-methylsulfonyl-5-chloropyridin-4-yl and the combination of R¹, R² and Y for a compound corresponds in each case to one row of Table A.

### Table 205

Compounds of the formula (I) in which X is methyl and Het is 3-methylsulfonyl-5-methylpyridin-4-yl and the combination of R¹, R² and Y for a compound corresponds in each case to one row of Table A.

### Table 206

Compounds of the formula (I) in which X is methyl and Het is 3-methoxymethyl-5-fluoropyridin-4-yl and the combination of R¹, R² and Y for a compound corresponds in each case to one row of Table A.

### Table 207

Compounds of the formula (I) in which X is methyl and Het is 3-methoxymethyl-5-chloropyridin-4-yl and the combination of R¹, R² and Y for a compound corresponds in each case to one row of Table A.

### Table 208

Compounds of the formula (I) in which X is methyl and Het is 3-methoxymethyl-5-methylpyridin-4-yl and the combination of R¹, R² and Y for a compound corresponds in each case to one row of Table A.

### Table 209

Compounds of the formula (I) in which X is methyl and Het is 2-fluoropyridin-3-yl and the combination of R¹, R² and Y for a compound corresponds in each case to one row of Table A.

### Table 210

Compounds of the formula (I) in which X is methyl and Het is 2-chloropyridin-3-yl and the combination of R¹, R² and Y for a compound corresponds in each case to one row of Table A.

### Table 211

Compounds of the formula (I) in which X is methyl and Het is 2-bromopyridin-3-yl and the combination of R¹, R² and Y for a compound corresponds in each case to one row of Table A.

### Table 212

Compounds of the formula (I) in which X is methyl and Het is 2-methylpyridin-3-yl and the combination of R¹, R² and Y for a compound corresponds in each case to one row of Table A.

### Table 213

Compounds of the formula (I) in which X is methyl and Het is 2-methoxypyridin-3-yl and the combination of R¹, R² and Y for a compound corresponds in each case to one row of Table A.

### Table 214

Compounds of the formula (I) in which X is methyl and Het is 2-methylsulfanylpyridin-3-yl and the combination of R¹, R² and Y for a compound corresponds in each case to one row of Table A.

### Table 215

Compounds of the formula (I) in which X is methyl and Het is 2-methylsulfinylpyridin-3-yl and the combination of R¹, R² and Y for a compound corresponds in each case to one row of Table A.

### Table 216

Compounds of the formula (I) in which X is methyl and Het is 2-methylsulfonylpyridin-3-yl and the combination of R¹, R² and Y for a compound corresponds in each case to one row of Table A.

### Table 217

Compounds of the formula (I) in which X is methyl and Het is 2-nitropyridin-3-yl and the combination of R¹, R² and Y for a compound corresponds in each case to one row of Table A.

### Table 218

Compounds of the formula (I) in which X is methyl and Het is 2-methoxymethylpyridin-3-yl and the combination of R¹, R² and Y for a compound corresponds in each case to one row of Table A.

### Table 219

Compounds of the formula (I) in which X is methyl and Het is 4-fluoropyridin-3-yl and the combination of R¹, R² and Y for a compound corresponds in each case to one row of Table A.

### Table 220

Compounds of the formula (I) in which X is methyl and Het is 4-chloropyridin-3-yl and the combination of R¹, R² and Y for a compound corresponds in each case to one row of Table A.

### Table 221

Compounds of the formula (I) in which X is methyl and Het is 4-bromopyridin-3-yl and the combination of R¹, R² and Y for a compound corresponds in each case to one row of Table A.

### Table 222

Compounds of the formula (I) in which X is methyl and Het is 4-methylpyridin-3-yl and the combination of R¹, R² and Y for a compound corresponds in each case to one row of Table A.

### Table 223

Compounds of the formula (I) in which X is methyl and Het is 4-methoxypyridin-3-yl and the combination of R¹, R² and Y for a compound corresponds in each case to one row of Table A.

### Table 224

Compounds of the formula (I) in which X is methyl and Het is 4-methylsulfanylpyridin-3-yl and the combination of R¹, R² and Y for a compound corresponds in each case to one row of Table A.

### Table 225

Compounds of the formula (I) in which X is methyl and Het is 4-methylsulfinylpyridin-3-yl and the combination of R¹, R² and Y for a compound corresponds in each case to one row of Table A.

### Table 226

Compounds of the formula (I) in which X is methyl and Het is 4-methylsulfonylpyridin-3-yl and the combination of R¹, R² and Y for a compound corresponds in each case to one row of Table A.

### Table 227

Compounds of the formula (I) in which X is methyl and Het is 4-nitropyridin-3-yl and the combination of R¹, R² and Y for a compound corresponds in each case to one row of Table A.

### Table 228

Compounds of the formula (I) in which. X is methyl and Het is 4-methoxymethylpyridin-3-yl and the combination of R¹, R² and Y for a compound corresponds in each case to one row of Table A.

### Table 229

Compounds of the formula (I) in which X is methyl and Het is 2,4-difluoropyridin-3-yl and the combination of R¹, R² and Y for a compound corresponds in each case to one row of Table A.

### Table 230

Compounds of the formula (I) in which X is methyl and Het is 2-fluoro-4-chloropyridin-3-yl and the combination of R¹, R² and Y for a compound corresponds in each case to one row of Table A.

### Table 231

Compounds of the formula (I) in which X is methyl and Het is 2-fluoro-4-bromopyridin-3-yl and the combination of R¹, R² and Y for a compound corresponds in each case to one row of Table A.

### Table 232.

Compounds of the formula (I) in which X is methyl and Het is 2-fluoro-4-methylpyridin-3-yl and the combination of R¹, R² and Y for a compound corresponds in each case to one row of Table A.

### Table 233

Compounds of the formula (I) in which X is methyl and Het is 2-fluoro-4-methoxypyridin-3-yl and the combination of R¹, R² and Y for a compound corresponds in each case to one row of Table A.

### Table 234

Compounds of the formula (I) in which X is methyl and Het is 2-fluoro-4-methylsulfanylpyridin-3-yl and the combination of R¹; R² and Y for a compound corresponds in each case to one row of Table A.

### Table 235

Compounds of the formula (I) in which X is methyl and Het is 2-fluoro-4-methylsulfinylpyridin-3-yl and the combination of R¹, R² and Y for a compound corresponds in each case to one row of Table A.

### Table 236

Compounds of the formula (I) in which X is methyl and Het is 2-fluoro-4-methylsulfonylpyridin-3-yl and the combination of R¹, R² and Y for a compound corresponds in each case to one row of Table A.

### Table 237

Compounds of the formula (I) in which X is methyl and Het is 2-fluoro-4-nitropyridin-3-yl and the combination of R¹, R² and Y for a compound corresponds in each case to one row of Table A.

### Table 238

Compounds of the formula (I) in which X is methyl and Het is 2-fluoro-4-methoxymethylpyridin-3-yl and the combination of R¹, R² and Y for a compound corresponds in each case to one row of Table A.

### Table 239

Compounds of the formula (I) in which X is methyl and Het is 2,4-dichloropyridin-3-yl and the combination of R¹, R² and Y for a compound corresponds in each case to one row of Table A.

### Table 240

Compounds of the formula (I) in which X is methyl and Het is 2-chloro-4-fluoropyridin-3-yl and the combination of R¹, R² and Y for a compound corresponds in each case to one row of Table A.

### Table 241

Compounds of the formula (I) in which X is methyl and Het is 2-chloro-4-bromopyridin-3-yl and the combination of R¹, R² and Y for a compound corresponds in each case to one row of Table A.

### Table 242

Compounds of the formula (I) in which X is methyl and Het is 2-chloro-4-methylpyridin-3-yl and the combination of R¹, R² and Y for a compound corresponds in each case to one row of Table A.

### Table 243

Compounds of the formula (I) in which X is methyl and Het is 2-chloro-4-methoxypyridin-3-yl and the combination of R¹, R² and Y for a compound corresponds in each case to one row of Table A.

### Table 244

Compounds of the formula (I) in which X is methyl and Het is 2-chloro-4-methylsulfanylpyridin-3-yl and the combination of R¹, R² and Y for a compound corresponds in each case to one row of Table A.

### Table 245

Compounds of the formula (I) in which X is methyl and Het is 2-chloro-4-methylsulfinylpyridin-3-yl and the combination of R¹, R² and Y for a compound corresponds in each case to one row of Table A.

### Table 246

Compounds of the formula (I) in which X is methyl and Het is 2-chloro-4-methylsulfonylpyridin-3-yl and the combination of R¹, R² and Y for a compound corresponds in each case to one row of Table A.

### Table 247

Compounds of the formula (I) in which X is methyl and Het is 2-chloro-4-nitropyridin-3-yl and the combination of R¹, R² and Y for a compound corresponds in each case to one row of Table A.

### Table 248

Compounds of the formula (I) in which X is methyl and Het is 2-chloro-4-methoxymethylpyridin-3-yl and the combination of R¹, R² and Y for a compound corresponds in each case to one row of Table A.

### Table 249

Compounds of the formula (I) in which X is methyl and Het is 2,4-dimethylpyridin-3-yl and the combination of R¹, R² and Y for a compound corresponds in each case to one row of Table A.

### Table 250

Compounds of the formula (I) in which X is methyl and Het is 2-methyl-4-chloropyridin-3-yl and the combination of R¹, R² and Y for a compound corresponds in each case to one row of Table A.

### Table 251

Compounds of the formula (I) in which X is methyl and Het is 2-methyl-4-fluoropyridin-3-yl and the combination of R¹, R² and Y for a compound corresponds in each case to one row of Table A.

### Table 252

Compounds of the formula (I) in which X is methyl and Het is 2-methyl-4-bromopyridin-3-yl and the combination of R¹, R² and Y for a compound corresponds in each case to one row of Table A.

### Table 253

Compounds of the formula (I) in which X is methyl and Het is 2-methyl-4-methoxypyridin-3-yl and the combination of R¹, R² and Y for a compound corresponds in each case to one row of Table A.

### Table 254

Compounds of the formula (I) in which X is methyl and Het is 2-methyl-4-methylsulfanylpyridin-3-yl and the combination of R¹, R² and Y for a compound corresponds in each case to one row of Table A.

### Table 255

Compounds of the formula (I) in which X is methyl and Het is 2-methyl-4-methylsulfinylpyridin-3-yl and the combination of R¹, R² and Y for a compound corresponds in each case to one row of Table A.

### Table 256

Compounds of the formula (I) in which X is methyl and Het is 2-methyl-4-methylsulfonylpyridin-3-yl and the combination of R¹, R² and Y for a compound corresponds in each case to one row of Table A.

### Table 257

Compounds of the formula (I) in which X is methyl and Het is 2-methyl-4-nitropyridin-3-yl and the combination of R¹, R² and Y for a compound corresponds in each case to one row of Table A.

### Table 258

Compounds of the formula (I) in which X is methyl and Het is 2-methyl-4-methoxymethylpyridin-3-yl and the combination of R¹, R² and Y for a compound corresponds in each case to one row of Table A.

### Table 259

Compounds of the formula (I) in which X is methyl and Het is 2-bromo-4-fluoropyridin-3-yl and the combination of R¹, R² and Y for a compound corresponds in each case to one row of Table A.

### Table 260

Compounds of the formula (I) in which X is methyl and Het is 2-bromo-4-chloropyridin-3-yl and the combination of R¹, R² and Y for a compound corresponds in each case to one row of Table A.

### Table 261

Compounds of the formula (I) in which X is methyl and Het is 2-bromo-4-methylpyridin-3-yl and the combination of R¹, R² and Y for a compound corresponds in each case to one row of Table A.

### Table 262

Compounds of the formula (I) in which X is methyl and Het is 2-methoxy-4-fluoropyridin-3-yl and the combination of R¹, R² and Y for a compound corresponds in each case to one row of Table A.

### Table 263

Compounds of the formula (I) in which X is methyl and Het is 2-methoxy-4-chloropyridin-3-yl and the combination of R¹, R² and Y for a compound corresponds in each case to one row of Table A.

### Table 264

Compounds of the formula (I) in which X is methyl and Het is 2-methoxy-4-methylpyridin-3-yl and the combination of R¹, R² and Y for a compound corresponds in each case to one row of Table A.

### Table 265

Compounds of the formula (I) in which X is methyl and Het is 2-methylsulfanyl-4-fluoropyridin-3-yl and the combination of R¹, R² and Y for a compound corresponds in each case to one row of Table A.

### Table 266

Compounds of the formula (I) in which X is methyl and Het is 2-methylsulfanyl-4-chloropyridin-3-yl and the combination of R¹, R² and Y for a compound corresponds in each case to one row of Table A.

### Table 267

Compounds of the formula (I) in which X is methyl and Het is 2-methylsulfanyl-4-methylpyridin-3-yl and the combination of R¹, R² and Y for a compound corresponds in each case to one row of Table A.

### Table 268

Compounds of the formula (I) in which X is methyl and Het is 2-methylsulfinyl-4-fluoropyridin-3-yl and the combination of R¹, R² and Y for a compound corresponds in each case to one row of Table A.

### Table 269

Compounds of the formula (I) in which X is methyl and Het is 2-methylsulfinyl-4-chloropyridin-3-yl and the combination of R¹, R² and Y for a compound corresponds in each case to one row of Table A.

### Table 270

Compounds of the formula (I) in which X is methyl and Het is 2-methylsulfinyl-4-methylpyridin-3-yl and the combination of R¹, R² and Y for a compound corresponds in each case to one row of Table A.

### Table 271

Compounds of the formula (I) in which X is methyl and Het is 2-methylsulfonyl-4-fluoropyridin-3-yl and the combination of R¹, R² and Y for a compound corresponds in each case to one row of Table A.

### Table 272

Compounds of the formula (I) in which X is methyl and Het is 2-methylsulfonyl-4-chloropyridin-3-yl and the combination of R¹, R² and Y for a compound corresponds in each case to one row of Table A.

### Table 273

Compounds of the formula (I) in which X is methyl and Het is 2-methylsulfonyl-4-methylpyridin-3-yl and the combination of R¹, R² and Y for a compound corresponds in each case to one row of Table A.

### Table 274

Compounds of the formula (I) in which X is methyl and Het is 2-nitro-4-fluoropyridin-3-yl and the combination of R¹, R² and Y for a compound corresponds in each case to one row of Table A.

### Table 275

Compounds of the formula (I) in which X is methyl and Het is 2-nitro-4-chloropyridin-3-yl and the combination of R¹, R² and Y for a compound corresponds in each case to one row of Table A.

### Table 276

Compounds of the formula (I) in which X is methyl and Het is 2-nitro-4-methylpyridin-3-yl and the combination of R¹, R² and Y for a compound corresponds in each case to one row of Table A.

### Table 277

Compounds of the formula (I) in which X is methyl and Het is 2-methoxymethyl-4-fluoropyridin-3-yl and the combination of R¹, R² and Y for a compound corresponds in each case to one row of Table A.

### Table 278

Compounds of the formula (I) in which X is methyl and Het is 2-methoxymethyl-4-chloropyridin-3-yl and the combination of R¹, R² and Y for a compound corresponds in each case to one row of Table A.

### Table 279

Compounds of the formula (I) in which X is methyl and Het is 2-methoxymethyl-4-methylpyridin-3-yl and the combination of R¹, R² and Y for a compound corresponds in each case to one row of Table A.

### Table 280

Compounds of the formula (I) in which X is methyl and Het is 4,6-difluoropyridin-3-yl and the combination of R¹, R² and Y for a compound corresponds in each case to one row of Table A.

### Table 281

Compounds of the formula (I) in which X is methyl and Het is 4-fluoro-6-chloropyridin-3-yl and the combination of R¹, R² and Y for a compound corresponds in each case to one row of Table A.

### Table 282

Compounds of the formula (I) in which X is methyl and Het is 4-fluoro-6-bromopyridin-3-yl and the combination of R¹, R² and Y for a compound corresponds in each case to one row of Table A.

### Table 283

Compounds of the formula (I) in which X is methyl and Het is 4-fluoro-6-methylpyridin-3-yl and the combination of R¹, R² and Y for a compound corresponds in each case to one row of Table A.

### Table 284

Compounds of the formula (I) in which X is methyl and Het is 4-fluoro-6-ethylpyridin-3-yl and the combination of R¹, R² and Y for a compound corresponds in each case to one row of Table A.

### Table 285

Compounds of the formula (I) in which X is methyl and Het is 4-fluoro-6-methoxypyridin-3-yl and the combination of R¹, R² and Y for a compound corresponds in each case to one row of Table A.

### Table 286

Compounds of the formula (I) in which X is methyl and Het is 4-fluoro-6-cyanopyridin-3-yl and the combination of R¹, R² and Y for a compound corresponds in each case to one row of Table A.

### Table 287

Compounds of the formula (I) in which X is methyl and Het is 4-fluoro-6-methoxycarbonylpyridin-3-yl and the combination of R¹, R² and Y for a compound corresponds in each case to one row of Table A.

### Table 288

Compounds of the formula (I) in which X is methyl and Het is 4-fluoro-6-ethoxycarbonylpyridin-3-yl and the combination of R¹, R² and Y for a compound corresponds in each case to one row of Table A.

### Table 289

Compounds of the formula (I) in which X is methyl and Het is 4-fluoro-6-carbamoylpyridin-3-yl and the combination of R¹, R² and Y for a compound corresponds in each case to one row of Table A.

### Table 290

Compounds of the formula (I) in which X is methyl and Het is 4-fluoro-6-methylcarbamoylpyridin-3-yl and the combination of R¹, R² and Y for a compound corresponds in each case to one row of Table A.

### Table 291

Compounds of the formula (I) in which X is methyl and Het is 4-fluoro-6-dimethylcarbamoylpyridin-3-yl and the combination of R¹, R² and Y for a compound corresponds in each case to one row of Table A.

### Table 292

Compounds of the formula (I) in which X is methyl and Het is 4-fluoro-6-thiocarbamoylpyridin-3-yl and the combination of R¹, R² and Y for a compound corresponds in each case to one row of Table A.

### Table 293

Compounds of the formula (I) in which X is methyl and Het is 4-fluoro-6-nitropyridin-3-yl and the combination of R¹, R² and Y for a compound corresponds in each case to one row of Table A.

### Table 294

Compounds of the formula (I) in which X is methyl and Het is 4-fluoro-6-acetylpyridin-3-yl and the combination of R¹, R² and Y for a compound corresponds in each case to one row of Table A.

### Table 295

Compounds of the formula (I) in which X is methyl and Het is 4,6-dichloropyridin-3-yl and the combination of R¹, R² and Y for a compound corresponds in each case to one row of Table A.

### Table 296

Compounds of the formula (I) in which X is methyl and Het is 4-chloro-6-fluoropyridin-3-yl and the combination of R¹, R² and Y for a compound corresponds in each case to one row of Table A.

### Table 297

Compounds of the formula (I) in which X is methyl and Het is 4-chloro-6-bromopyridin-3-yl and the combination of R¹, R² and Y for a compound corresponds in each case to one row of Table A.

### Table 298

Compounds of the formula (I) in which X is methyl and Het is 4-chloro-6-methylpyridin-3-yl and the combination of R¹, R² and Y for a compound corresponds in each case to one row of Table A.

### Table 299

Compounds of the formula (I) in which X is methyl and Het is 4-chloro-6-ethylpyridin-3-yl and the combination of R¹, R² and Y for a compound corresponds in each case to one row of Table A.

### Table 300

Compounds of the formula (I) in which X is methyl and Het is 4-chloro-6-methoxypyridin-3-yl and the combination of R¹, R² and Y for a compound corresponds in each case to one row of Table A.

### Table 301

Compounds of the formula (I) in which X is methyl and Het is 4-chloro-6-cyanopyridin-3-yl and the combination of R¹, R² and Y for a compound corresponds in each case to one row of Table A.

### Table 302

Compounds of the formula (I) in which X is methyl and Het is 4-chloro-6-methoxycarbonylpyridin-3-yl and the combination of R¹, R² and Y for a compound corresponds in each case to one row of Table A.

### Table 303

Compounds of the formula (I) in which X is methyl and Het is 4-chloro-6-ethoxycarbonylpyridin-3-yl and the combination of R¹, R² and Y for a compound corresponds in each case to one row of Table A.

### Table 304

Compounds of the formula (I) in which X is methyl and Het is 4-chloro-6-carbamoylpyridin-3-yl and the combination of R¹, R² and Y for a compound corresponds in each case to one row of Table A.

### Table 305

Compounds of the formula (I) in which X is methyl and Het is 4-chloro-6-methylcarbamoylpyridin-3-yl and the combination of R¹, R² and Y for a compound corresponds in each case to one row of Table A.

### Table 306

Compounds of the formula (I) in which X is methyl and Het is 4-chloro-6-dimethylcarbamoylpyridin-3-yl and the combination of R¹, R² and Y for a compound corresponds in each case to one row of Table A.

### Table 307

Compounds of the formula (I) in which X is methyl and Het is 4-chloro-6-thiocarbamoylpyridin-3-yl and the combination of R¹, R² and Y for a compound corresponds in each case to one row of Table A.

### Table 308

Compounds of the formula (I) in which X is methyl and Het is 4-chloro-6-nitropyridin-3-yl and the combination of R¹, R² and Y for a compound corresponds in each case to one row of Table A.

### Table 309

Compounds of the formula (I) in which X is methyl and Het is 4-chloro-6-acetylpyridin-3-yl and the combination of R¹, R² and Y for a compound corresponds in each case to one row of Table A.

### Table 310

Compounds of the formula (I) in which X is methyl and Het is 4-methyl-6-fluoropyridin-3-yl and the combination of R¹, R² and Y for a compound corresponds in each case to one row of Table A.

### Table 311

Compounds of the formula (I) in which X is methyl and Het is 4-methyl-6-chloropyridin-3-yl and the combination of R¹, R² and Y for a compound corresponds in each case to one row of Table A.

### Table 312

Compounds of the formula (I) in which X is methyl and Het is 4-methyl-6-bromopyridin-3-yl and the combination of R¹, R² and Y for a compound corresponds in each case to one row of Table A.

### Table 313

Compounds of the formula (I) in which X is methyl and Het is 4,6-dimethylpyridin-3-yl and the combination of R¹, R² and Y for a compound corresponds in each case to one row of Table A.

### Table 314

Compounds of the formula (I) in which X is methyl and Het is 4-methyl-6-ethylpyridin-3-yl and the combination of R¹, R² and Y for a compound corresponds in each case to one row of Table A.

### Table 315

Compounds of the formula (I) in which X is methyl and Het is 4-methyl-6-methoxypyridin-3-yl and the combination of R¹, R² and Y for a compound corresponds in each case to one row of Table A.

### Table 316

Compounds of the formula (I) in which X is methyl and Het is 4-methyl-6-cyanopyridin-3-yl and the combination of R¹, R² and Y for a compound corresponds in each case to one row of Table A.

### Table 317

Compounds of the formula (I) in which X is methyl and Het is 4-methyl-6-methoxycarbonylpyridin-3-yl and the combination of R¹, R² and Y for a compound corresponds in each case to one row of Table A.

### Table 318

Compounds of the formula (I) in which X is methyl and Het is 4-methyl-6-ethoxycarbonylpyridin-3-yl and the combination of R¹, R² and Y for a compound corresponds in each case to one row of Table A.

### Table 319

Compounds of the formula (I) in which X is methyl and Het is 4-methyl-6-carbamoylpyridin-3-yl and the combination of R¹, R² and Y for a compound corresponds in each case to one row of Table A.

### Table 320

Compounds of the formula (I) in which X is methyl and Het is 4-methyl-6-methylcarbamoylpyridin-3-yl and the combination of R¹, R² and Y for a compound corresponds in each case to one row of Table A.

### Table 321

Compounds of the formula (I) in which X is methyl and Het is 4-methyl-6-dimethylcarbamoylpyridin-3-yl and the combination of R¹, R² and Y for a compound corresponds in each case to one row of Table A.

### Table 322

Compounds of the formula (I) in which X is methyl and Het is 4-methyl-6-thiocarbamoylpyridin-3-yl and the combination of R¹, R² and Y for a compound corresponds in each case to one row of Table A.

### Table 323

Compounds of the formula (I) in which X is methyl and Het is 4-methyl-6-nitropyridin-3-yl and the combination of R¹, R² and Y for a compound corresponds in each case to one row of Table A.

### Table 324

Compounds of the formula (I) in which X is methyl and Het is 4-methyl-6-acetylpyridin-3-yl and the combination of R¹, R² and Y for a compound corresponds in each case to one row of Table A.

### Table 325

Compounds of the formula (I) in which X is methyl and Het is 4-bromo-6-fluoropyridin-3-yl and the combination of R¹, R² and Y for a compound corresponds in each case to one row of Table A.

### Table 326

Compounds of the formula (I) in which X is methyl and Het is 4-bromo-6-chloropyridin-3-yl and the combination of R¹, R² and Y for a compound corresponds in each case to one row of Table A.

### Table 327

Compounds of the formula (I) in which X is methyl and Het is 4-bromo-6-methylpyridin-3-yl and the combination of R¹, R² and Y for a compound corresponds in each case to one row of Table A.

### Table 328

Compounds of the formula (I) in which X is methyl and Het is 4-methoxy-6-fluoropyridin-3-yl and the combination of R¹, R² and Y for a compound corresponds in each case to one row of Table A.

### Table 329

Compounds of the formula (I) in which X is methyl and Het is 4-methoxy-6-chloropyridin-3-yl and the combination of R¹, R² and Y for a compound corresponds in each case to one row of Table A.

### Table 330

Compounds of the formula (I) in which X is methyl and Het is 4-methoxy-6-methylpyridin-3-yl and the combination of R¹, R² and Y for a compound corresponds in each case to one row of Table A.

### Table 331

Compounds of the formula (I) in which X is methyl and Het is 4-methylsulfanyl-6-fluoropyridin-3-yl and the combination of R¹, R² and Y for a compound corresponds in each case to one row of Table A.

### Table 332

Compounds of the formula (I) in which X is methyl and Het is 4-methylsulfanyl-6-chloropyridin-3-yl and the combination of R¹, R² and Y for a compound corresponds in each case to one row of Table A.

### Table 333

Compounds of the formula (I) in which X is methyl and Het is 4-methylsulfanyl-6-methylpyridin-3-yl and the combination of R¹, R² and Y for a compound corresponds in each case to one row of Table A.

### Table 334

Compounds of the formula (I) in which X is methyl and Het is 4-methylsulfinyl-6-fluoropyridin-3-yl and the combination of R¹, R² and Y for a compound corresponds in each case to one row of Table A.

### Table 335

Compounds of the formula (I) in which X is methyl and Het is 4-methylsulfinyl-6-chloropyridin-3-yl and the combination of R¹, R² and Y for a compound corresponds in each case to one row of Table A.

### Table 336

Compounds of the formula (I) in which X is methyl and Het is 4-methylsulfinyl-6-methylpyridin-3-yl and the combination of R¹, R² and Y for a compound corresponds in each case to one row of Table A.

### Table 337

Compounds of the formula (I) in which X is methyl and Het is 4-methylsulfonyl-6-fluoropyridin-3-yl and the combination of R¹, R² and Y for a compound corresponds in each case to one row of Table A.

### Table 338

Compounds of the formula (I) in which X is methyl and Het is 4-methylsulfonyl-6-chloropyridin-3-yl and the combination of R¹, R² and Y for a compound corresponds in each case to one row of Table A.

### Table 339

Compounds of the formula (I) in which X is methyl and Het is 4-methylsulfonyl-6-methylpyridin-3-yl and the combination of R¹, R² and Y for a compound corresponds in each case to one row of Table A.

### Table 340

Compounds of the formula (I) in which X is methyl and Het is 4-nitro-6-fluoropyridin-3-yl and the combination of R¹, R² and Y for a compound corresponds in each case to one row of Table A.

### Table 341

Compounds of the formula (I) in which X is methyl and Het is 4-nitro-6-chloropyridin-3-yl and the combination of R¹, R² and Y for a compound corresponds in each case to one row of Table A.

### Table 342

Compounds of the formula (I) in which X is methyl and Het is 4-nitro-6-methylpyridin-3-yl and the combination of R¹, R² and Y for a compound corresponds in each case to one row of Table A.

### Table 343

Compounds of the formula (I) in which X is methyl and Het is 4-methoxymethyl-6-fluoropyridin-3-yl and the combination of R¹, R² and Y for a compound corresponds in each case to one row of Table A.

### Table 344

Compounds of the formula (I) in which X is methyl and Het is 4-methoxymethyl-6-chloropyridin-3-yl and the combination of R¹, R² and Y for a compound corresponds in each case to one row of Table A.

### Table 345

Compounds of the formula (I) in which X is methyl and Het is 4-methoxymethyl-6-methylpyridin-3-yl and the combination of R¹, R² and Y for a compound corresponds in each case to one row of Table A.

### Table 346

Compounds of the formula (I) in which X is methyl and Het is 2,4-dichlor-6-methylpyridin-3-yl and the combination of R¹, R² and Y for a compound corresponds in each case to one row of Table A.

### Table 347

Compounds of the formula (I) in which X is ethyl and Het is 2-pyridinyl and the combination of R¹, R² and Y for a compound corresponds in each case to one row of Table A.

### Table 348

Compounds of the formula (I) in which X is ethyl and Het is 5-nitropyridin-2-yl and the combination of R¹, R² and Y for a compound corresponds in each case to one row of Table A.

### Table 349

Compounds of the formula (I) in which X is ethyl and Het is 5-cyanopyridin-2-yl and the combination of R¹, R² and Y for a compound corresponds in each case to one row of Table A.

### Table 350

Compounds of the formula (I) in which X is ethyl and Het is 5-methoxycarbonyl-pyridin-2-yl and the combination of R¹, R² and Y for a compound corresponds in each case to one row of Table A.

### Table 351

Compounds of the formula (I) in which X is ethyl and Het is 5-methylpyridin-2-yl and the combination of R¹, R² and Y for a compound corresponds in each case to one row of Table A.

### Table 352

Compounds of the formula (I) in which X is ethyl and Het is 4-methylpyridin-2-yl and the combination of R¹, R² and Y for a compound corresponds in each case to one row of Table A.

### Table 353

Compounds of the formula (I) in which X is ethyl and Het is 3-methylpyridin-2-yl and the combination of R¹, R² and Y for a compound corresponds in each case to one row of Table A.

### Table 354

Compounds of the formula (I) in which X is ethyl and Het is 3-ethylpyridin-2-yl and the combination of R¹, R² and Y for a compound corresponds in each case to one row of Table A.

### Table 355

Compounds of the formula (I) in which X is ethyl and Het is 6-methylpyridin-2-yl and the combination of R¹, R² and Y for a compound corresponds in each case to one row of Table A.

### Table 356

Compounds of the formula (I) in which X is ethyl and Het is 5-trifluoromethylpyridin-2-yl and the combination of R¹, R² and Y for a compound corresponds in each case to one row of Table A.

### Table 357

Compounds of the formula (I) in which X is ethyl and Het is 3-trifluoromethylpyridin-2-yl and the combination of R¹, R² and Y for a compound corresponds in each case to one row of Table A.

### Table 358

Compounds of the formula (I) in which X is ethyl and Het is 5-fluoropyridin-2-yl and the combination of R¹, R² and Y for a compound corresponds in each case to one row of Table A.

### Table 359

Compounds of the formula (I) in which X is ethyl and Het is 3-fluoropyridin-2-yl and the combination of R¹, R² and Y for a compound corresponds in each case to one row of Table A.

### Table 360

Compounds of the formula (I) in which X is ethyl and Het is 3,5-difluoropyridin-2-yl and the combination of R¹, R² and Y for a compound corresponds in each case to one row of Table A.

### Table 361

Compounds of the formula (I) in which X is ethyl and Het is 3,5-dichloropyridin-2-yl and the combination of R¹, R² and Y for a compound corresponds in each case to one row of Table A.

### Table 362

Compounds of the formula (I) in which X is ethyl and Het is pyridin-3-yl and the combination of R¹, R² and Y for a compound corresponds in each case to one row of Table A.

### Table 363

Compounds of the formula (I) in which X is ethyl and Het is pyridin-4-yl and the combination of R¹, R² and Y for a compound corresponds in each case to one row of Table A.

### Table 364

Compounds of the formula (I) in which X is ethyl and Het is pyrazin-2-yl and the combination of R¹, R² and Y for a compound corresponds in each case to one row of Table A.

### Table 365

Compounds of the formula (I) in which X is ethyl and Het is pyridazin-3-yl and the combination of R¹, R² and Y for a compound corresponds in each case to one row of Table A.

### Table 366

Compounds of the formula (I) in which X is ethyl and Het is 6-chloropyridazin-3-yl and the combination of R¹, R² and Y for a compound corresponds in each case to one row of Table A.

### Table 367

Compounds of the formula (I) in which X is ethyl and Het is 6-methoxypyridazin-3-yl and the combination of R¹, R² and Y for a compound corresponds in each case to one row of Table A.

### Table 368

Compounds of the formula (I) in which X is ethyl and Het is 1,3,5-triazin-2-yl and the combination of R¹, R² and Y for a compound corresponds in each case to one row of Table A.

### Table 369

Compounds of the formula (I) in which X is ethyl and Het is pyrimidin-2-yl and the combination of R¹, R² and Y for a compound corresponds in each case to one row of Table A.

### Table 370

Compounds of the formula (I) in which X is ethyl and Het is pyrimidin-4-yl and the combination of R¹, R² and Y for a compound corresponds in each case to one row of Table A.

### Table 371

Compounds of the formula (I) in which X is ethyl and Het is pyrimidin-5-yl and the combination of R¹, R² and Y for a compound corresponds in each case to one row of Table A.

### Table 372

Compounds of the formula (I) in which X is ethyl and Het is 2-methyl-6-trifluoromethylpyrimidin-4-yl and the combination of R¹, R² and Y for a compound corresponds in each case to one row of Table A.

### Table 373

Compounds of the formula (I) in which X is ethyl and Het is 2,5-dimethyl-6-trifluoromethylpyrimidin-4-yl and the combination of R¹, R² and Y for a compound corresponds in each case to one row of Table A.

### Table 374

Compounds of the formula (I) in which X is ethyl and Het is 6-trifluoromethylpyrimidin-4-yl and the combination of R¹, R² and Y for a compound corresponds in each case to one row of Table A.

### Table 375

Compounds of the formula (I) in which X is ethyl and Het is 5-methyl-6-trifluoromethylpyrimidin-4-yl and the combination of R¹, R² and Y for a compound corresponds in each case to one row of Table A.

### Table 376

Compounds of the formula (I) in which X is ethyl and Het is 5-chloro-6-methylpyrimidin-4-yl and the combination of R¹, R² and Y for a compound corresponds in each case to one row of Table A.

### Table 377

Compounds of the formula (I) in which X is ethyl and Het is 5-bromo-6-methylpyrimidin-4-yl and the combination of R¹, R² and Y for a compound corresponds in each case to one row of Table A.

### Table 378

Compounds of the formula (I) in which X is ethyl and Het is 5-fluoro-6-methylpyrimidin-4-yl and the combination of R¹, R² and Y for a compound corresponds in each case to one row of Table A.

### Table 379

Compounds of the formula (I) in which X is ethyl and Het is 5-chloro-6-ethylpyrimidin-4-yl and the combination of R¹, R² and Y for a compound corresponds in each case to one row of Table A.

### Table 380

Compounds of the formula (I) in which X is ethyl and Het is 5-bromo-6-ethylpyrimidin-4-yl and the combination of R¹, R² and Y for a compound corresponds in each case to one row of Table A.

### Table 381

Compounds of the formula (I) in which X is ethyl and Het is 5-chloro-6-isopropyl-pyrimidin-4-yl and the combination of R¹, R² and Y for a compound corresponds in each case to one row of Table A.

### Table 382

Compounds of the formula (I) in which X is ethyl and Het is 5-bromo-6-isopropyl-pyrimidin-4-yl and the combination of R¹, R² and Y for a compound corresponds in each case to one row of Table A.

### Table 383

Compounds of the formula (I) in which X is ethyl and Het is 5-chloropyrimidin-4-yl and the combination of R¹, R² and Y for a compound corresponds in each case to one row of Table A.

### Table 384

Compounds of the formula (I) in which X is ethyl and Het is 5-fluoropyrimidin-4-yl and the combination of R¹, R² and Y for a compound corresponds in each case to one row of Table A.

### Table 385

Compounds of the formula (I) in which X is ethyl and Het is 5-bromopyrimidin-4-yl and the combination of R¹, R² and Y for a compound corresponds in each case to one row of Table A.

### Table 386

Compounds of the formula (I) in which X is ethyl and Het is 2-methyl-5-fluoropyrimidin-4-yl and the combination of R¹, R² and Y for a compound corresponds in each case to one row of Table A.

### Table 387

Compounds of the formula (I) in which X is ethyl and Het is 2-methyl-5-chloropyrimidin-4-yl and the combination of R¹, R² and Y for a compound corresponds in each case to one row of Table A.

### Table 388

Compounds of the formula (I) in which X is ethyl and Het is 2-methyl-5-bromopyrimidin-4-yl and the combination of R¹, R² and Y for a compound corresponds in each case to one row of Table A.

### Table 389

Compounds of the formula (I) in which X is ethyl and Het is 2,6-dimethyl-5-chloropyrimidin-4-yl and the combination of R¹, R² and Y for a compound corresponds in each case to one row of Table A.

### Table 390

Compounds of the formula (I) in which X is ethyl and Het is 2,6-dimethyl-5-bromopyrimidin-4-yl and the combination of R¹, R² and Y for a compound corresponds in each case to one row of Table A.

### Table 391

Compounds of the formula (I) in which X is ethyl and Het is 5-fluoro-6-fluoromethyl-pyrimidin-4-yl and the combination of R¹, R² and Y for a compound corresponds in each case to one row of Table A.

### Table 392

Compounds of the formula (I) in which X is ethyl and Het is 5-chloro-6-methoxymethylpyrimidin-4-yl and the combination of R¹, R² and Y for a compound corresponds in each case to one row of Table A.

### Table 393

Compounds of the formula (I) in which X is ethyl and Het is 5,6-dimethylpyrimidin-4-yl and the combination of R¹, R² and Y for a compound corresponds in each case to one row of Table A.

### Table 394

Compounds of the formula (I) in which X is ethyl and Het is 2,5-dimethylpyrimidin-4-yl and the combination of R¹, R² and Y for a compound corresponds in each case to one row of Table A.

### Table 395

Compounds of the formula (I) in which X is ethyl and Het is 2,5,6-trimethylpyrimidin-4-yl and the combination of R¹, R² and Y for a compound corresponds in each case to one row of Table A.

### Table 396

Compounds of the formula (I) in which X is ethyl and Het is 5-methyl-6-methoxy-pyrimidin-4-yl and the combination of R¹, R² and Y for a compound corresponds in each case to one row of Table A.

### Table 397

Compounds of the formula (I) in which X is ethyl and Het is 4,6-dimethylpyrimidin-2-yl and the combination of R¹, R² and Y for a compound corresponds in each case to one row of Table A.

### Table 398

Compounds of the formula (I) in which X is ethyl and Het is 4,5,6-trimethylpyrimidin-2-yl and the combination of R¹, R² and Y for a compound corresponds in each case to one row of Table A.

### Table 399

Compounds of the formula (I) in which X is ethyl and Het is 4,6-di(trifluoromethyl)-pyrimidin-2-yl and the combination of R¹, R² and Y for a compound corresponds in each case to one row of Table A.

### Table 400

Compounds of the formula (I) in which X is ethyl and Het is 4,6-dimethyl-5-chloropyrimidin-2-yl and the combination of R¹, R² and Y for a compound corresponds in each case to one row of Table A.

### Table 401

Compounds of the formula (I) in which X is ethyl and Het is 4,6-dimethyl-5-bromopyrimidin-2-yl and the combination of R¹, R² and Y for a compound corresponds in each case to one row of Table A.

### Table 402

Compounds of the formula (I) in which X is ethyl and Het is 4-methylpyrimidin-5-yl and the combination of R¹, R² and Y for a compound corresponds in each case to one row of Table A.

### Table 403

Compounds of the formula (I) in which X is ethyl and Het is 4,6-dimethylpyrimidin-5-yl and the combination of R¹, R² and Y for a compound corresponds in each case to one row of Table A.

### Table 404

Compounds of the formula (I) in which X is ethyl and Het is 4-trifluoromethyl-6-methylpyrimidin-5-yl and the combination of R¹, R² and Y for a compound corresponds in each case to one row of Table A.

### Table 405

Compounds of the formula (I) in which X is ethyl and Het is 2,4,6-trimethylpyrimidin-5-yl and the combination of R¹, R² and Y for a compound corresponds in each case to one row of Table A.

### Table 406

Compounds of the formula (I) in which X is ethyl and Het is 3-chloropyridin-2-yl and the combination of R¹, R² and Y for a compound corresponds in each case to one row of Table A.

### Table 407

Compounds of the formula (I) in which X is ethyl and Het is 3-bromopyridin-2-yl and the combination of R¹, R² and Y for a compound corresponds in each case to one row of Table A.

### Table 408

Compounds of the formula (I) in which X is ethyl and Het is 3-iodopyridin-2-yl and the combination of R¹, R² and Y for a compound corresponds in each case to one row of Table A.

### Table 409

Compounds of the formula (I) in which X is ethyl and Het is 3-methoxypyridin-2-yl and the combination of R¹, R² and Y for a compound corresponds in each case to one row of Table A.

### Table 410

Compounds of the formula (I) in which X is ethyl and Het is 3-methylsulfanylpyridin-2-yl and the combination of R¹, R² and Y for a compound corresponds in each case to one row of Table A.

### Table 411

Compounds of the formula (I) in which X is ethyl and Het is 3-methylsulfinylpyridin-2-yl and the combination of R¹, R² and Y for a compound corresponds in each case to one row of Table A.

### Table 412

Compounds of the formula (I) in which X is ethyl and Het is 3-methylsulfonylpyridin-2-yl and the combination of R¹, R² and Y for a compound corresponds in each case to one row of Table A.

### Table 413

Compounds of the formula (I) in which X is ethyl and Het is 3-nitropyridin-2-yl and the combination of R¹, R² and Y for a compound corresponds in each case to one row of Table A.

### Table 414

Compounds of the formula (I) in which X is ethyl and Het is 3-methoxymethylpyridin-2-yl and the combination of R¹, R² and Y for a compound corresponds in each case to one row of Table A.

### Table 415

Compounds of the formula (I) in which X is ethyl and Het is 5-chloropyridin-2-yl and the combination of R¹, R² and Y for a compound corresponds in each case to one row of Table A.

### Table 416

Compounds of the formula (I) in which X is ethyl and Het is 5-bromopyridin-2-yl and the combination of R¹, R² and Y for a compound corresponds in each case to one row of Table A.

### Table 417

Compounds of the formula (I) in which X is ethyl and Het is 5-methoxypyridin-2-yl and the combination of R¹, R² and Y for a compound corresponds in each case to one row of Table A.

### Table 418

Compounds of the formula (I) in which X is ethyl and Het is 5-methylsulfanylpyridin-2-yl and the combination of R¹, R² and Y for a compound corresponds in each case to one row of Table A.

### Table 419

Compounds of the formula (I) in which X is ethyl and Het is 5-methylsulfinylpyridin-2-yl and the combination of R¹, R² and Y for a compound corresponds in each case to one row of Table A.

### Table 420

Compounds of the formula (I) in which X is ethyl and Het is 5-methylsulfonylpyridin-2-yl and the combination of R¹, R² and Y for a compound corresponds in each case to one row of Table A.

### Table 421

Compounds of the formula (I) in which X is ethyl and Het is 5-methoxymethylpyridin-2-yl and the combination of R¹, R² and Y for a compound corresponds in each case to one row of Table A.

### Table 422

Compounds of the formula (I) in which X is ethyl and Het is 5-ethylpyridin-2-yl and the combination of R¹, R² and Y for a compound corresponds in each case to one row of Table A.

### Table 423

Compounds of the formula (I) in which X is ethyl and Het is 5-ethoxycarbonylpyridin-2-yl and the combination of R¹, R² and Y for a compound corresponds in each case to one row of Table A.

### Table 424

Compounds of the formula (I) in which X is ethyl and Het is 5-carbamoylpyridin-2-yl and the combination of R¹, R² and Y for a compound corresponds in each case to one row of Table A.

### Table 425

Compounds of the formula (I) in which X is ethyl and Het is 5-methylcarbamoylpyridin-2-yl and the combination of R¹, R² and Y for a compound corresponds in each case to one row of Table A.

### Table 426

Compounds of the formula (I) in which X is ethyl and Het is 5-dimethylcarbamoylpyridin-2-yl and the combination of R¹, R² and Y for a compound corresponds in each case to one row of Table A.

### Table 427

Compounds of the formula (I) in which X is ethyl and Het is 5-thiocarbamoylpyridin-2-yl and the combination of R¹, R² and Y for a compound corresponds in each case to one row of Table A.

### Table 428

Compounds of the formula (I) in which X is ethyl and Het is 5-acetylpyridin-2-yl and the combination of R¹, R² and Y for a compound corresponds in each case to one row of Table A.

### Table 429

Compounds of the formula (I) in which X is ethyl and Het is 5-methylpyridin-2-yl and the combination of R¹, R² and Y for a compound corresponds in each case to one row of Table A.

### Table 430

Compounds of the formula (I) in which X is ethyl and Het is 5-nitropyridin-2-yl and the combination of R¹, R² and Y for a compound corresponds in each case to one row of Table A.

### Table 431

Compounds of the formula (I) in which X is ethyl and Het is 5-cyanopyridin-2-yl and the combination of R¹, R² and Y for a compound corresponds in each case to one row of Table A.

### Table 432

Compounds of the formula (I) in which X is ethyl and Het is 3-fluoro-5-chloropyridin-2-yl and the combination of R¹, R² and Y for a compound corresponds in each case to one row of Table A.

### Table 433

Compounds of the formula (I) in which X is ethyl and Het is 3-fluoro-5-bromopyridin-2-yl and the combination of R¹, R² and Y for a compound corresponds in each case to one row of Table A.

### Table 434

Compounds of the formula (I) in which X is ethyl and Het is 3-fluoro-5-methylpyridin-2-yl and the combination of R¹, R² and Y for a compound corresponds in each case to one row of Table A.

### Table 435

Compounds of the formula (I) in which X is ethyl and Het is 3-fluoro-5-ethylpyridin-2-yl and the combination of R¹, R² and Y for a compound corresponds in each case to one row of Table A.

### Table 436

Compounds of the formula (I) in which X is ethyl and Het is 3-fluoro-5-methoxypyridin-2-yl and the combination of R¹, R² and Y for a compound corresponds in each case to one row of Table A.

### Table 437

Compounds of the formula (I) in which X is ethyl and Het is 3-fluoro-5-cyanopyridin-2-yl and the combination of R¹, R² and Y for a compound corresponds in each case to one row of Table A.

### Table 438

Compounds of the formula (I) in which X is ethyl and Het is 3-fluoro-5-methoxycarbonylpyridin-2-yl and the combination of R¹, R² and Y for a compound corresponds in each case to one row of Table A.

### Table 439

Compounds of the formula (I) in which X is ethyl and Het is 3-fluoro-5-ethoxycarbonylpyridin-2-yl and the combination of R¹, R² and Y for a compound corresponds in each case to one row of Table A.

### Table 440

Compounds of the formula (I) in which X is ethyl and Het is 3-fluoro-5-carbamoylpyridin-2-yl and the combination of R¹, R² and Y for a compound corresponds in each case to one row of Table A.

### Table 441

Compounds of the formula (I) in which X is ethyl and Het is 3-fluoro-5-methylcarbamoylpyridin-2-yl and the combination of R¹, R² and Y for a compound corresponds in each case to one row of Table A.

### Table 442.

Compounds of the formula (I) in which X is ethyl and Het is 3-fluoro-5-dimethylcarbamoylpyridin-2-yl and the combination of R¹, R² and Y for a compound corresponds in each case to one row of Table A.

### Table 443

Compounds of the formula (I) in which X is ethyl and Het is 3-fluoro-5-thiocarbamoylpyridin-2-yl and the combination of R¹, R² and Y for a compound corresponds in each case to one row of Table A.

### Table 444

Compounds of the formula (I) in which X is ethyl and Het is 3-fluoro-5-nitropyridin-2-yl and the combination of R¹, R² and Y for a compound corresponds in each case to one row of Table A.

### Table 445

Compounds of the formula (I) in which X is ethyl and Het is 3-fluoro-5-acetylpyridin-2-yl and the combination of R¹, R² and Y for a compound corresponds in each case to one row of Table A.

### Table 446

Compounds of the formula (I) in which X is ethyl and Het is 3-chloro-5-fluoropyridin-2-yl and the combination of R¹, R² and Y for a compound corresponds in each case to one row of Table A.

### Table 447

Compounds of the formula (I) in which X is ethyl and Het is 3-chloro-5-bromopyridin-2-yl and the combination of R¹, R² and Y for a compound corresponds in each case to one row of Table A.

### Table 448

Compounds of the formula (I) in which X is ethyl and Het is 3-chloro-5-methylpyridin-2-yl and the combination of R¹, R² and Y for a compound corresponds in each case to one row of Table A.

### Table 449

Compounds of the formula (I) in which X is ethyl and Het is 3-chloro-5-ethylpyridin-2-yl and the combination of R¹, R² and Y for a compound corresponds in each case to one row of Table A.

### Table 450

Compounds of the formula (I) in which X is ethyl and Het is 3-chloro-5-methoxypyridin-2-yl and the combination of R¹, R² and Y for a compound corresponds in each case to one row of Table A.

### Table 451

Compounds of the formula (I) in which X is ethyl and Het is 3-chloro-5-cyanopyridin-2-yl and the combination of R¹, R² and Y for a compound corresponds in each case to one row of Table A.

### Table 452

Compounds of the formula (I) in which X is ethyl and Het is 3-chloro-5-methoxycarbonylpyridin-2-yl and the combination of R¹, R² and Y for a compound corresponds in each case to one row of Table A.

### Table 453

Compounds of the formula (I) in which X is ethyl and Het is 3-chloro-5-ethoxycarbonylpyridin-2-yl and the combination of R¹, R² and Y for a compound corresponds in each case to one row of Table A.

### Table 454

Compounds of the formula (I) in which X is ethyl and Het is 3-chloro-5-carbamoylpyridin-2-yl and the combination of R¹, R² and Y for a compound corresponds in each case to one row of Table A.

### Table 455

Compounds of the formula (I) in which X is ethyl and Het is 3-chloro-5-methylcarbamoylpyridin-2-yl and the combination of R¹, R² and Y for a compound corresponds in each case to one row of Table A.

### Table 456

Compounds of the formula (I) in which X is ethyl and Het is 3-chloro-5-dimethylcarbamoylpyridin-2-yl and the combination of R¹, R² and Y for a compound corresponds in each case to one row of Table A.

### Table 457

Compounds of the formula (I) in which X is ethyl and Het is 3-chloro-5-thiocarbamoylpyridin-2-yl and the combination of R¹, R² and Y for a compound corresponds in each case to one row of Table A.

### Table 458

Compounds of the formula (I) in which X is ethyl and Het is 3-chloro-5-nitropyridin-2-yl and the combination of R¹, R² and Y for a compound corresponds in each case to one row of Table A.

### Table 459

Compounds of the formula (I) in which X is ethyl and Het is 3-chloro-5-acetylpyridin-2-yl and the combination of R¹, R² and Y for a compound corresponds in each case to one row of Table A.

### Table 460

Compounds of the formula (I) in which X is ethyl and Het is 3-methyl-5-fluoropyridin-2-yl and the combination of R¹, R² and Y for a compound corresponds in each case to one row of Table A.

### Table 461

Compounds of the formula (I) in which X is ethyl and Het is 3-methyl-5-chloropyridin-2-yl and the combination of R¹, R² and Y for a compound corresponds in each case to one row of Table A.

### Table 462

Compounds of the formula (I) in which X is ethyl and Het is 3-methyl-5-bromopyridin-2-yl and the combination of R¹, R² and Y for a compound corresponds in each case to one row of Table A.

### Table 463

Compounds of the formula (I) in which X is ethyl and Het is 3,5-dimethylpyridin-2-yl and the combination of R¹, R² and Y for a compound corresponds in each case to one row of Table A.

### Table 464

Compounds of the formula (I) in which X is ethyl and Het is 3-methyl-5-ethylpyridin-2-yl and the combination of R¹, R² and Y for a compound corresponds in each case to one row of Table A.

### Table 465

Compounds of the formula (I) in which X is ethyl and Het is 3-methyl-5-methoxypyridin-2-yl and the combination of R¹, R² and Y for a compound corresponds in each case to one row of Table A.

### Table 466

Compounds of the formula (I) in which X is ethyl and Het is 3-methyl-5-cyanopyridin-2-yl and the combination of R¹, R² and Y for a compound corresponds in each case to one row of Table A.

### Table 467

Compounds of the formula (I) in which X is ethyl and Het is 3-methyl-5-methoxycarbonylpyridin-2-yl and the combination of R¹, R² and Y for a compound corresponds in each case to one row of Table A.

### Table 468

Compounds of the formula (I) in which X is ethyl and Het is 3-methyl-5-ethoxycarbonylpyridin-2-yl and the combination of R¹, R² and Y for a compound corresponds in each case to one row of Table A.

### Table 469

Compounds of the formula (I) in which X is ethyl and Het is 3-methyl-5-carbamoylpyridin-2-yl and the combination of R¹, R² and Y for a compound corresponds in each case to one row of Table A.

### Table 470

Compounds of the formula (I) in which X is ethyl and Het is 3-methyl-5-methylcarbamoylpyridin-2-yl and the combination of R¹, R² and Y for a compound corresponds in each case to one row of Table A.

### Table 471

Compounds of the formula (I) in which X is ethyl and Het is 3-methyl-5-dimethylcarbamoylpyridin-2-yl and the combination of R¹, R² and Y for a compound corresponds in each case to one row of Table A.

### Table 472

Compounds of the formula (I) in which X is ethyl and Het is 3-methyl-5-thiocarbamoylpyridin-2-yl and the combination of R¹, R² and Y for a compound corresponds in each case to one row of Table A.

### Table 473

Compounds of the formula (I) in which X is ethyl and Het is 3-methyl-5-nitropyridin-2-yl and the combination of R¹, R² and Y for a compound corresponds in each case to one row of Table A.

### Table 474

Compounds of the formula (I) in which X is ethyl and Het is 3-methyl-5-acetylpyridin-2-yl and the combination of R¹, R² and Y for a compound corresponds in each case to one row of Table A.

### Table 475

Compounds of the formula (I) in which X is ethyl and Het is 3-bromo-5-fluoropyridin-2-yl and the combination of R¹, R² and Y for a compound corresponds in each case to one row of Table A.

### Table 476

Compounds of the formula (I) in which X is ethyl and Het is 3-bromo-5-chloropyridin-2-yl and the combination of R¹, R² and Y for a compound corresponds in each case to one row of Table A.

### Table 477

Compounds of the formula (I) in which X is ethyl and Het is 3-bromo-5-methylpyridin-2-yl and the combination of R¹, R² and Y for a compound corresponds in each case to one row of Table A.

### Table 478

Compounds of the formula (I) in which X is ethyl and Het is 3-methylsulfonyl-5-fluoropyridin-2-yl and the combination of R¹, R² and Y for a compound corresponds in each case to one row of Table A.

### Table 479

Compounds of the formula (I) in which X is ethyl and Het is 3-methylsulfonyl-5-chloropyridin-2-yl and the combination of R¹, R² and Y for a compound corresponds in each case to one row of Table A.

### Table 480

Compounds of the formula (I) in which X is ethyl and Het is 3-methylsulfonyl-5-methylpyridin-2-yl and the combination of R¹, R² and Y for a compound corresponds in each case to one row of Table A.

### Table 481

Compounds of the formula (I) in which X is ethyl and Het is 3-methoxy-5-fluoropyridin-2-yl and the combination of R¹, R² and Y for a compound corresponds in each case to one row of Table A.

### Table 482

Compounds of the formula (I) in which X is ethyl and Het is 3-methoxy-5-chloropyridin-2-yl and the combination of R¹, R² and Y for a compound corresponds in each case to one row of Table A.

### Table 483

Compounds of the formula (I) in which X is ethyl and Het is 3-methoxy-5-methylpyridin-2-yl and the combination of R¹, R² and Y for a compound corresponds in each case to one row of Table A.

### Table 484

Compounds of the formula (I) in which X is ethyl and Het is 3-methylsulfanyl-5-fluoropyridin-2-yl and the combination of R¹, R² and Y for a compound corresponds in each case to one row of Table A.

### Table 485

Compounds of the formula (I) in which X is ethyl and Het is 3-methylsulfanyl-5-chloropyridin-2-yl and the combination of R¹, R² and Y for a compound corresponds in each case to one row of Table A.

### Table 486

Compounds of the formula (I) in which X is ethyl and Het is 3-methylsulfanyl-5-methylpyridin-2-yl and the combination of R¹, R² and Y for a compound corresponds in each case to one row of Table A.

### Table 487

Compounds of the formula (I) in which X is ethyl and Het is 3-nitro-5-fluoropyridin-2-yl and the combination of R¹, R² and Y for a compound corresponds in each case to one row of Table A.

### Table 488

Compounds of the formula (I) in which X is ethyl and Het is 3-nitro-5-chloropyridin-2-yl and the combination of R¹, R² and Y for a compound corresponds in each case to one row of Table A.

### Table 489

Compounds of the formula (I) in which X is ethyl and Het is 3-nitro-5-methylpyridin-2-yl and the combination of R¹, R² and Y for a compound corresponds in each case to one row of Table A.

### Table 490

Compounds of the formula (I) in which X is ethyl and Het is 3,5-dibromopyridin-2-yl and the combination of R¹, R² and Y for a compound corresponds in each case to one row of Table A.

### Table 491

Compounds of the formula (I) in which X is ethyl and Het is 3-fluoropyridin-4-yl and the combination of R¹, R² and Y for a compound corresponds in each case to one row of Table A.

### Table 492

Compounds of the formula (I) in which X is ethyl and Het is 3-chloropyridin-4-yl and the combination of R¹, R² and Y for a compound corresponds in each case to one row of Table A.

### Table 493

Compounds of the formula (I) in which X is ethyl and Het is 3-bromopyridin-4-yl and the combination of R¹, R² and Y for a compound corresponds in each case to one row of Table A.

### Table 494

Compounds of the formula (I) in which X is ethyl and Het is 3-methylpyridin-4-yl and the combination of R¹, R² and Y for a compound corresponds in each case to one row of Table A.

### Table 495

Compounds of the formula (I) in which X is ethyl and Het is 3-methoxypyridin-4-yl and the combination of R¹, R² and Y for a compound corresponds in each case to one row of Table A.

### Table 496

Compounds of the formula (I) in which X is ethyl and Het is 3-methylsulfanylpyridin-4-yl and the combination of R¹, R² and Y for a compound corresponds in each case to one row of Table A.

### Table 497

Compounds of the formula (I) in which X is ethyl and Het is 3-methylsulfinylpyridin-4-yl and the combination of R¹, R² and Y for a compound corresponds in each case to one row of Table A.

### Table 498

Compounds of the formula (I) in which X is ethyl and Het is 3-methylsulfonylpyridin-4-yl and the combination of R¹, R² and Y for a compound corresponds in each case to one row of Table A.

### Table 499

Compounds of the formula (I) in which X is ethyl and Het is 3-nitropyridin-4-yl and the combination of R¹, R² and Y for a compound corresponds in each case to one row of Table A.

### Table 500

Compounds of the formula (I) in which X is ethyl and Het is 3-methoxymethylpyridin-4-yl and the combination of R¹, R² and Y for a compound corresponds in each case to one row of Table A.

### Table 501

Compounds of the formula (I) in which X is ethyl and Het is 3,5-difluoropyridin-4-yl and the combination of R¹, R² and Y for a compound corresponds in each case to one row of Table A.

### Table 502

Compounds of the formula (I) in which X is ethyl and Het is 3-fluoro-5-chloropyridin-4-yl and the combination of R¹, R² and Y for a compound corresponds in each case to one row of Table A.

### Table 503

Compounds of the formula (I) in which X is ethyl and Het is 3-fluoro-5-bromopyridin-4-yl and the combination of R¹, R² and Y for a compound corresponds in each case to one row of Table A.

### Table 504

Compounds of the formula (I) in which X is ethyl and Het is 3-fluoro-5-methylpyridin-4-yl and the combination of R¹, R² and Y for a compound corresponds in each case to one row of Table A.

### Table 505

Compounds of the formula (I) in which X is ethyl and Het is 3-fluoro-5-ethylpyridin-4-yl and the combination of R¹, R² and Y for a compound corresponds in each case to one row of Table A.

### Table 506

Compounds of the formula (I) in which X is ethyl and Het is 3-fluoro-5-methoxypyridin-4-yl and the combination of R¹, R² and Y for a compound corresponds in each case to one row of Table A.

### Table 507

Compounds of the formula (I) in which X is ethyl and Het is 3-fluoro-5-cyanopyridin-4-yl and the combination of R¹, R² and Y for a compound corresponds in each case to one row of Table A.

### Table 508

Compounds of the formula (I) in which X is ethyl and Het is 3-fluoro-5-methoxycarbonylpyridin-4-yl and the combination of R¹, R² and Y for a compound corresponds in each case to one row of Table A.

### Table 509

Compounds of the formula (I) in which X is ethyl and Het is 3-fluoro-5-ethoxycarbonylpyridin-4-yl and the combination of R¹, R² and Y for a compound corresponds in each case to one row of Table A.

### Table 510

Compounds of the formula (I) in which X is ethyl and Het is 3-fluoro-5-carbamoylpyridin-4-yl and the combination of R¹, R² and Y for a compound corresponds in each case to one row of Table A.

### Table 511

Compounds of the formula (I) in which X is ethyl and Het is 3-fluoro-5-methylcarbamoylpyridin-4-yl and the combination of R¹, R² and Y for a compound corresponds in each case to one row of Table A.

### Table 512

Compounds of the formula (I) in which X is ethyl and Het is 3-fluoro-5-dimethylcarbamoylpyridin-4-yl and the combination of R¹, R² and Y for a compound corresponds in each case to one row of Table A.

### Table 513

Compounds of the formula (I) in which X is ethyl and Het is 3-fluoro-5-thiocarbamoylpyridin-4-yl and the combination of R¹, R² and Y for a compound corresponds in each case to one row of Table A.

### Table 514

Compounds of the formula (I) in which X is ethyl and Het is 3-fluoro-5-nitropyridin-4-yl and the combination of R¹, R² and Y for a compound corresponds in each case to one row of Table A.

### Table 515

Compounds of the formula (I) in which X is ethyl and Het is 3-fluoro-5-acetylpyridin-4-yl and the combination of R¹, R² and Y for a compound corresponds in each case to one row of Table A.

### Table 516

Compounds of the formula (I) in which X is ethyl and Het is 3,5-dichloro-5-chloropyridin-4-yl and the combination of R¹, R² and Y for a compound corresponds in each case to one row of Table A.

### Table 517

Compounds of the formula (I) in which X is ethyl and Het is 3-chloro-5-bromopyridin-4-yl and the combination of R¹, R² and Y for a compound corresponds in each case to one row of Table A.

### Table 518

Compounds of the formula (I) in which X is ethyl and Het is 3-chloro-5-methylpyridin-4-yl and the combination of R¹, R² and Y for a compound corresponds in each case to one row of Table A.

### Table 519

Compounds of the formula (I) in which X is ethyl and Het is 3-chloro-5-ethylpyridin-4-yl and the combination of R¹, R² and Y for a compound corresponds in each case to one row of Table A.

### Table 520

Compounds of the formula (I) in which X is ethyl and Het is 3-chloro-5-methoxypyridin-4-yl and the combination of R¹, R² and Y for a compound corresponds in each case to one row of Table A.

### Table 521

Compounds of the formula (I) in which X is ethyl and Het is 3-chloro-5-cyanopyridin-4-yl and the combination of R¹, R² and Y for a compound corresponds in each case to one row of Table A.

### Table 522

Compounds of the formula (I) in which X is ethyl and Het is 3-chloro-5-methoxycarbonylpyridin-4-yl and the combination of R¹, R² and Y for a compound corresponds in each case to one row of Table A.

### Table 523

Compounds of the formula (I) in which X is ethyl and Het is 3-chloro-5-ethoxycarbonylpyridin-4-yl and the combination of R¹, R² and Y for a compound corresponds in each case to one row of Table A.

### Table 524

Compounds of the formula (I) in which X is ethyl and Het is 3-chloro-5-carbamoylpyridin-4-yl and the combination of R¹, R² and Y for a compound corresponds in each case to one row of Table A.

### Table 525

Compounds of the formula (I) in which X is ethyl and Het is 3-chloro-5-methylcarbamoylpyridin-4-yl and the combination of R¹, R² and Y for a compound corresponds in each case to one row of Table A.

### Table 526

Compounds of the formula (I) in which X is ethyl and Het is 3-chloro-5-dimethylcarbamoylpyridin-4-yl and the combination of R¹, R² and Y for a compound corresponds in each case to one row of Table A.

### Table 527

Compounds of the formula (I) in which X is ethyl and Het is 3-chloro-5-thiocarbamoylpyridin-4-yl and the combination of R¹, R² and Y for a compound corresponds in each case to one row of Table A.

### Table 528

Compounds of the formula (I) in which X is ethyl and Het is 3-chloro-5-nitropyridin-4-yl and the combination of R¹, R² and Y for a compound corresponds in each case to one row of Table A.

### Table 529

Compounds of the formula (I) in which X is ethyl and Het is 3-chloro-5-acetylpyridin-4-yl and the combination of R¹, R² and Y for a compound corresponds in each case to one row of Table A.

### Table 530

Compounds of the formula (I) in which X is ethyl and Het is 3,5-dimethylpyridin-4-yl and the combination of R¹, R² and Y for a compound corresponds in each case to one row of Table A.

### Table 531

Compounds of the formula (I) in which X is ethyl and Het is 3-methyl-5-bromopyridin-4-yl and the combination of R¹, R² and Y for a compound corresponds in each case to one row of Table A.

### Table 532

Compounds of the formula (I) in which X is ethyl and Het is 3-methyl-5-ethylpyridin-4-yl and the combination of R¹, R² and Y for a compound corresponds in each case to one row of Table A.

### Table 533

Compounds of the formula (I) in which X is ethyl and Het is 3-methyl-5-methoxypyridin-4-yl and the combination of R¹, R² and Y for a compound corresponds in each case to one row of Table A.

### Table 534

Compounds of the formula (I) in which X is ethyl and Het is 3-methyl-5-cyanopyridin-4-yl and the combination of R¹, R² and Y for a compound corresponds in each case to one row of Table A.

### Table 535

Compounds of the formula (I) in which X is ethyl and Het is 3-methyl-5-methoxycarbonylpyridin-4-yl and the combination of R¹, R² and Y for a compound corresponds in each case to one row of Table A.

### Table 536

Compounds of the formula (I) in which X is ethyl and Het is 3-methyl-5-ethoxycarbonylpyridin-4-yl and the combination of R¹, R² and Y for a compound corresponds in each case to one row of Table A.

### Table 537

Compounds of the formula (I) in which X is ethyl and Het is 3-methyl-5-carbamoylpyridin-4-yl and the combination of R¹, R² and Y for a compound corresponds in each case to one row of Table A.

### Table 538

Compounds of the formula (I) in which X is ethyl and Het is 3-methyl-5-methylcarbamoylpyridin-4-yl and the combination of R¹, R² and Y for a compound corresponds in each case to one row of Table A.

### Table 539

Compounds of the formula (I) in which X is ethyl and Het is 3-methyl-5-dimethylcarbamoylpyridin-4-yl and the combination of R¹, R² and Y for a compound corresponds in each case to one row of Table A.

### Table 540

Compounds of the formula (I) in which X is ethyl and Het is 3-methyl-5-thiocarbamoylpyridin-4-yl and the combination of R¹, R² and Y for a compound corresponds in each case to one row of Table A.

### Table 541

Compounds of the formula (I) in which X is ethyl and Het is 3-methyl-5-nitropyridin-4-yl and the combination of R¹, R² and Y for a compound corresponds in each case to one row of Table A.

### Table 542

Compounds of the formula (I) in which X is ethyl and Het is 3-methyl-5-acetylpyridin-4-yl and the combination of R¹, R² and Y for a compound corresponds in each case to one row of Table A.

### Table 543

Compounds of the formula (I) in which X is ethyl and Het is 3-methylsulfanyl-5-fluoropyridin-4-yl and the combination of R¹, R² and Y for a compound corresponds in each case to one row of Table A.

### Table 544

Compounds of the formula (I) in which X is ethyl and Het is 3-methylsulfanyl-5-chloropyridin-4-yl and the combination of R¹, R² and Y for a compound corresponds in each case to one row of Table A.

### Table 545

Compounds of the formula (I) in which X is ethyl and Het is 3-methylsulfanyl-5-methylpyridin-4-yl and the combination of R¹, R² and Y for a compound corresponds in each case to one row of Table A.

### Table 546

Compounds of the formula (I) in which X is ethyl and Het is 3-methylsulfinyl-5-fluoropyridin-4-yl and the combination of R¹, R² and Y for a compound corresponds in each case to one row of Table A.

### Table 547

Compounds of the formula (I) in which X is ethyl and Het is 3-methylsulfinyl-5-chloropyridin-4-yl and the combination of R¹, R² and Y for a compound corresponds in each case to one row of Table A.

### Table 548

Compounds of the formula (I) in which X is ethyl and Het is 3-methylsulfinyl-5-methylpyridin-4-yl and the combination of R¹, R² and Y for a compound corresponds in each case to one row of Table A.

### Table 549

Compounds of the formula (I) in which X is ethyl and Het is 3-methylsulfonyl-5-fluoropyridin-4-yl and the combination of R¹, R² and Y for a compound corresponds in each case to one row of Table A.

### Table 550

Compounds of the formula (I) in which X is ethyl and Het is 3-methylsulfonyl-5-chloropyridin-4-yl and the combination of R¹, R² and Y for a compound corresponds in each case to one row of Table A.

### Table 551

Compounds of the formula (I) in which X is ethyl and Het is 3-methylsulfonyl-5-methylpyridin-4-yl and the combination of R¹, R² and Y for a compound corresponds in each case to one row of Table A.

### Table 552

Compounds of the formula (I) in which X is ethyl and Het is 3-methoxymethyl-5-fluoropyridin-4-yl and the combination of R¹, R² and Y for a compound corresponds in each case to one row of Table A.

### Table 553

Compounds of the formula (I) in which X is ethyl and Het is 3-methoxymethyl-5-chloropyridin-4-yl and the combination of R¹, R² and Y for a compound corresponds in each case to one row of Table A.

### Table 554

Compounds of the formula (I) in which X is ethyl and Het is 3-methoxymethyl-5-methylpyridin-4-yl and the combination of R¹, R² and Y for a compound corresponds in each case to one row of Table A.

### Table 555

Compounds of the formula (I) in which X is ethyl and Het is 2-fluoropyridin-3-yl and the combination of R¹, R² and Y for a compound corresponds in each case to one row of Table A.

### Table 556

Compounds of the formula (I) in which X is ethyl and Het is 2-chloropyridin-3-yl and the combination of R¹, R² and Y for a compound corresponds in each case to one row of Table A.

### Table 557

Compounds of the formula (I) in which X is ethyl and Het is 2-bromopyridin-3-yl and the combination of R¹, R² and Y for a compound corresponds in each case to one row of Table A.

### Table 558

Compounds of the formula (I) in which X is ethyl and Het is 2-methylpyridin-3-yl and the combination of R¹, R² and Y for a compound corresponds in each case to one row of Table A.

### Table 559

Compounds of the formula (I) in which X is ethyl and Het is 2-methoxypyridin-3-yl and the combination of R¹, R² and Y for a compound corresponds in each case to one row of Table A.

### Table 560

Compounds of the formula (I) in which X is ethyl and Het is 2-methylsulfanylpyridin-3-yl and the combination of R¹, R² and Y for a compound corresponds in each case to one row of Table A.

### Table 561

Compounds of the formula (I) in which X is ethyl and Het is 2-methylsulfinylpyridin-3-yl and the combination of R¹, R² and Y for a compound corresponds in each case to one row of Table A.

### Table 562

Compounds of the formula (I) in which X is ethyl and Het is 2-methylsulfonylpyridin-3-yl and the combination of R¹, R² and Y for a compound corresponds in each case to one row of Table A.

### Table 563

Compounds of the formula (I) in which X is ethyl and Het is 2-nitropyridin-3-yl and the combination of R¹, R² and Y for a compound corresponds in each case to one row of Table A.

### Table 564

Compounds of the formula (I) in which X is ethyl and Het is 2-methoxymethylpyridin-3-yl and the combination of R¹, R² and Y for a compound corresponds in each case to one row of Table A.

### Table 565

Compounds of the formula (I) in which X is ethyl and Het is 4-fluoropyridin-3-yl and the combination of R¹, R² and Y for a compound corresponds in each case to one row of Table A.

### Table 566

Compounds of the formula (I) in which X is ethyl and Het is 4-chloropyridin-3-yl and the combination of R¹, R² and Y for a compound corresponds in each case to one row of Table A.

### Table 567

Compounds of the formula (I) in which X is ethyl and Het is 4-bromopyridin-3-yl and the combination of R¹, R² and Y for a compound corresponds in each case to one row of Table A.

### Table 568

Compounds of the formula (I) in which X is ethyl and Het is 4-methylpyridin-3-yl and the combination of R¹, R² and Y for a compound corresponds in each case to one row of Table A.

### Table 569

Compounds of the formula (I) in which X is ethyl and Het is 4-methoxypyridin-3-yl and the combination of R¹, R² and Y for a compound corresponds in each case to one row of Table A.

### Table 570

Compounds of the formula (I) in which X is ethyl and Het is 4-methylsulfanylpyridin-3-yl and the combination of R¹, R² and Y for a compound corresponds in each case to one row of Table A.

### Table 571

Compounds of the formula (I) in which X is ethyl and Het is 4-methylsulfinylpyridin-3-yl and the combination of R¹, R² and Y for a compound corresponds in each case to one row of Table A.

### Table 572

Compounds of the formula (I) in which X is ethyl and Het is 4-methylsulfonylpyridin-3-yl and the combination of R¹, R² and Y for a compound corresponds in each case to one row of Table A.

### Table 573

Compounds of the formula (I) in which X is ethyl and Het is 4-nitropyridin-3-yl and the combination of R¹, R² and Y for a compound corresponds in each case to one row of Table A.

### Table 574

Compounds of the formula (I) in which X is ethyl and Het is 4-methoxymethylpyridin-3-yl and the combination of R¹, R² and Y for a compound corresponds in each case to one row of Table A.

### Table 575

Compounds of the formula (I) in which X is ethyl and Het is 2,4-difluoropyridin-3-yl and the combination of R¹, R² and Y for a compound corresponds in each case to one row of Table A.

### Table 576

Compounds of the formula (I) in which X is ethyl and Het is 2-fluoro-4-chloropyridin-3-yl and the combination of R¹, R² and Y for a compound corresponds in each case to one row of Table A.

### Table 577

Compounds of the formula (I) in which X is ethyl and Het is 2-fluoro-4-bromopyridin-3-yl and the combination of R¹, R² and Y for a compound corresponds in each case to one row of Table A.

### Table 578

Compounds of the formula (I) in which X is ethyl and Het is 2-fluoro-4-methylpyridin-3-yl and the combination of R¹, R² and Y for a compound corresponds in each case to one row of Table A.

### Table 579

Compounds of the formula (I) in which X is ethyl and Het is 2-fluoro-4-methoxypyridin-3-yl and the combination of R¹, R² and Y for a compound corresponds in each case to one row of Table A.

### Table 580

Compounds of the formula (I) in which X is ethyl and Het is 2-fluoro-4-methylsulfanylpyridin-3-yl and the combination of R¹, R² and Y for a compound corresponds in each case to one row of Table A.

### Table 581

Compounds of the formula (I) in which X is ethyl and Het is 2-fluoro-4-methylsulfinylpyridin-3-yl and the combination of R¹, R² and Y for a compound corresponds in each case to one row of Table A.

### Table 582

Compounds of the formula (I) in which X is ethyl and Het is 2-fluoro-4-methylsulfonylpyridin-3-yl and the combination of R¹, R² and Y for a compound corresponds in each case to one row of Table A.

### Table 583

Compounds of the formula (I) in which X is ethyl and Het is 2-fluoro-4-nitropyridin-3-yl and the combination of R¹, R² and Y for a compound corresponds in each case to one row of Table A.

### Table 584

Compounds of the formula (I) in which X is ethyl and Het is 2-fluoro-4-methoxymethylpyridin-3-yl and the combination of R¹, R² and Y for a compound corresponds in each case to one row of Table A.

### Table 585

Compounds of the formula (I) in which X is ethyl and Het is 2,4-dichloropyridin-3-yl and the combination of R¹, R² and Y for a compound corresponds in each case to one row of Table A.

### Table 586

Compounds of the formula (I) in which X is ethyl and Het is 2-chloro-4-fluoropyridin-3-yl and the combination of R¹, R² and Y for a compound corresponds in each case to one row of Table A.

### Table 587

Compounds of the formula (I) in which X is ethyl and Het is 2-chloro-4-bromopyridin-3-yl and the combination of R¹, R² and Y for a compound corresponds in each case to one row of Table A.

### Table 588

Compounds of the formula (I) in which X is ethyl and Het is 2-chloro-4-methylpyridin-3-yl and the combination of R¹, R² and Y for a compound corresponds in each case to one row of Table A.

### Table 589

Compounds of the formula (I) in which X is ethyl and Het is 2-chloro-4-methoxypyridin-3-yl and the combination of R¹, R¹² and Y for a compound corresponds in each case to one row of Table A.

### Table 590

Compounds of the formula (I) in which X is ethyl and Het is 2-chloro-4-methylsulfanylpyridin-3-yl and the combination of R¹, R² and Y for a compound corresponds in each case to one row of Table A.

### Table 591

Compounds of the formula (I) in which X is ethyl and Het is 2-chloro-4-methylsulfinylpyridin-3-yl and the combination of R¹, R² and Y for a compound corresponds in each case to one row of Table A.

### Table 592

Compounds of the formula (I) in which X is ethyl and Het is 2-chloro-4-methylsulfonylpyridin-3-yl and the combination of R¹, R² and Y for a compound corresponds in each case to one row of Table A.

### Table 593

Compounds of the formula (I) in which X is ethyl and Het is 2-chloro-4-nitropyridin-3-yl and the combination of R¹, R² and Y for a compound corresponds in each case to one row of Table A.

### Table 594

Compounds of the formula (I) in which X is ethyl and Het is 2-chloro-4-methoxymethylpyridin-3-yl and the combination of R¹, R² and Y for a compound corresponds in each case to one row of Table A.

### Table 595

Compounds of the formula (I) in which X is ethyl and Het is 2,4-dimethylpyridin-3-yl and the combination of R¹, R² and Y for a compound corresponds in each case to one row of Table A.

### Table 596

Compounds of the formula (I) in which X is ethyl and Het is 2-methyl-4-chloropyridin-3-yl and the combination of R¹, R² and Y for a compound corresponds in each case to one row of Table A.

### Table 597

Compounds of the formula (I) in which X is ethyl and Het is 2-methyl-4-fluoropyridin-3-yl and the combination of R¹, R² and Y for a compound corresponds in each case to one row of Table A.

### Table 598

Compounds of the formula (I) in which X is ethyl and Het is 2-methyl-4-bromopyridin-3-yl and the combination of R¹, R² and Y for a compound corresponds in each case to one row of Table A.

### Table 599

Compounds of the formula (I) in which X is ethyl and Het is 2-methyl-4-methoxypyridin-3-yl and the combination of R¹, R² and Y for a compound corresponds in each case to one row of Table A.

### Table 600

Compounds of the formula (I) in which X is ethyl and Het is 2-methyl-4-methylsulfanylpyridin-3-yl and the combination of R¹, R² and Y for a compound corresponds in each case to one row of Table A.

### Table 601

Compounds of the formula (I) in which X is ethyl and Het is 2-methyl-4-methylsulfinylpyridin-3-yl and the combination of R¹, R² and Y for a compound corresponds in each case to one row of Table A.

### Table 602

Compounds of the formula (I) in which X is ethyl and Het is 2-methyl-4-methylsulfonylpyridin-3-yl and the combination of R¹, R² and Y for a compound corresponds in each case to one row of Table A.

### Table 603

Compounds of the formula (I) in which X is ethyl and Het is 2-methyl-4-nitropyridin-3-yl and the combination of R¹, R² and Y for a compound corresponds in each case to one row of Table A.

### Table 604

Compounds of the formula (I) in which X is ethyl and Het is 2-methyl-4-methoxymethylpyridin-3-yl and the combination of R¹, R² and Y for a compound corresponds in each case to one row of Table A.

### Table 605

Compounds of the formula (I) in which X is ethyl and Het is 2-bromo-4-fluoropyridin-3-yl and the combination of R¹, R² and Y for a compound corresponds in each case to one row of Table A.

### Table 606

Compounds of the formula (I) in which X is ethyl and Het is 2-bromo-4-chloropyridin-3-yl and the combination of R¹, R² and Y for a compound corresponds in each case to one row of Table A.

### Table 607

Compounds of the formula (I) in which X is ethyl and Het is 2-bromo-4-methylpyridin-3-yl and the combination of R¹, R² and Y for a compound corresponds in each case to one row of Table A.

### Table 608

Compounds of the formula (I) in which X is ethyl and Het is 2-methoxy-4-fluoropyridin-3-yl and the combination of R¹, R² and Y for a compound corresponds in each case to one row of Table A.

### Table 609

Compounds of the formula (I) in which X is ethyl and Het is 2-methoxy-4-chloropyridin-3-yl and the combination of R¹, R² and Y for a compound corresponds in each case to one row of Table A.

### Table 610

Compounds of the formula (I) in which X is ethyl and Het is 2-methoxy-4-methylpyridin-3-yl and the combination of R¹, R² and Y for a compound corresponds in each case to one row of Table A.

### Table 611

Compounds of the formula (I) in which X is ethyl and Het is 2-methylsulfanyl-4-fluoropyridin-3-yl and the combination of R¹, R² and Y for a compound corresponds in each case to one row of Table A.

### Table 612

Compounds of the formula (I) in which X is ethyl and Het is 2-methylsulfanyl-4-chloropyridin-3-yl and the combination of R¹, R² and Y for a compound corresponds in each case to one row of Table A.

### Table 613

Compounds of the formula (I) in which X is ethyl and Het is 2-methylsulfanyl-4-methylpyridin-3-yl and the combination of R¹, R² and Y for a compound corresponds in each case to one row of Table A.

### Table 614

Compounds of the formula (I) in which X is ethyl and Het is 2-methylsulfinyl-4-fluoropyridin-3-yl and the combination of R¹, R² and Y for a compound corresponds in each case to one row of Table A.

### Table 615

Compounds of the formula (I) in which X is ethyl and Het is 2-methylsulfinyl-4-chloropyridin-3-yl and the combination of R¹, R² and Y for a compound corresponds in each case to one row of Table A.

### Table 616

Compounds of the formula (I) in which X is ethyl and Het is 2-methylsulfinyl-4-methylpyridin-3-yl and the combination of R¹, R² and Y for a compound corresponds in each case to one row of Table A.

### Table 617

Compounds of the formula (I) in which X is ethyl and Het is 2-methylsulfonyl-4-fluoropyridin-3-yl and the combination of R¹, R² and Y for a compound corresponds in each case to one row of Table A.

### Table 618

Compounds of the formula (I) in which X is ethyl and Het is 2-methylsulfonyl-4-chloropyridin-3-yl and the combination of R¹, R² and Y for a compound corresponds in each case to one row of Table A.

### Table 619

Compounds of the formula (I) in which X is ethyl and Het is 2-methylsulfonyl-4-methylpyridin-3-yl and the combination of R¹, R² and Y for a compound corresponds in each case to one row of Table A.

### Table 620

Compounds of the formula (I) in which X is ethyl and Het is 2-nitro-4-fluoropyridin-3-yl and the combination of R¹, R² and Y for a compound corresponds in each case to one row of Table A.

### Table 621

Compounds of the formula (I) in which X is ethyl and Het is 2-nitro-4-chloropyridin-3-yl and the combination of R¹, R² and Y for a compound corresponds in each case to one row of Table A.

### Table 622

Compounds of the formula (I) in which X is ethyl and Het is 2-nitro-4-methylpyridin-3-yl and the combination of R¹, R² and Y for a compound corresponds in each case to one row of Table A.

### Table 623

Compounds of the formula (I) in which X is ethyl and Het is 2-methoxymethyl-4-fluoropyridin-3-yl and the combination of R¹, R² and Y for a compound corresponds in each case to one row of Table A.

### Table 624

Compounds of the formula (I) in which X is ethyl and Het is 2-methoxymethyl-4-chloropyridin-3-yl and the combination of R¹, R² and Y for a compound corresponds in each case to one row of Table A.

### Table 625

Compounds of the formula (I) in which X is ethyl and Het is 2-methoxymethyl-4-methylpyridin-3-yl and the combination of R¹, R² and Y for a compound corresponds in each case to one row of Table A.

### Table 626

Compounds of the formula (I) in which X is ethyl and Het is 4,6-difluoropyridin-3-yl and the combination of R¹, R² and Y for a compound corresponds in each case to one row of Table A.

### Table 627

Compounds of the formula (I) in which X is ethyl and Het is 4-fluoro-6-chloropyridin-3-yl and the combination of R¹, R² and Y for a compound corresponds in each case to one row of Table A.

### Table 628

Compounds of the formula (I) in which X is ethyl and Het is 4-fluoro-6-bromopyridin-3-yl and the combination of R¹, R² and Y for a compound corresponds in each case to one row of Table A.

### Table 629

Compounds of the formula (I) in which X is ethyl and Het is 4-fluoro-6-methylpyridin-3-yl and the combination of R¹, R² and Y for a compound corresponds in each case to one row of Table A.

### Table 630

Compounds of the formula (I) in which X is ethyl and Het is 4-fluoro-6-ethylpyridin-3-yl and the combination of R¹, R² and Y for a compound corresponds in each case to one row of Table A.

### Table 631

Compounds of the formula (I) in which X is ethyl and Het is 4-fluoro-6-methoxypyridin-3-yl and the combination of R¹, R² and Y for a compound corresponds in each case to one row of Table A.

### Table 632

Compounds of the formula (I) in which X is ethyl and Het is 4-fluoro-6-cyanopyridin-3-yl and the combination of R¹, R² and Y for a compound corresponds in each case to one row of Table A.

### Table 633

Compounds of the formula (I) in which X is ethyl and Het is 4-fluoro-6-methoxycarbonylpyridin-3-yl and the combination of R¹, R² and Y for a compound corresponds in each case to one row of Table A.

### Table 634

Compounds of the formula (I) in which X is ethyl and Het is 4-fluoro-6-ethoxycarbonylpyridin-3-yl and the combination of R¹, R² and Y for a compound corresponds in each case to one row of Table A.

### Table 635

Compounds of the formula (I) in which X is ethyl and Het is 4-fluoro-6-carbamoylpyridin-3-yl and the combination of R¹, R² and Y for a compound corresponds in each case to one row of Table A.

### Table 636

Compounds of the formula (I) in which X is ethyl and Het is 4-fluoro-6-methylcarbamoylpyridin-3-yl and the combination of R¹, R² and Y for a compound corresponds in each case to one row of Table A.

### Table 637

Compounds of the formula (I) in which X is ethyl and Het is 4-fluoro-6-dimethylcarbamoylpyridin-3-yl and the combination of R¹, R² and Y for a compound corresponds in each case to one row of Table A.

### Table 638

Compounds of the formula (I) in which X is ethyl and Het is 4-fluoro-6-thiocarbamoylpyridin-3-yl and the combination of R¹, R² and Y for a compound corresponds in each case to one row of Table A.

### Table 639

Compounds of the formula (I) in which X is ethyl and Het is 4-fluoro-6-nitropyridin-3-yl and the combination of R¹, R² and Y for a compound corresponds in each case to one row of Table A.

### Table 640

Compounds of the formula (I) in which X is ethyl and Het is 4-fluoro-6-acetylpyridin-3-yl and the combination of R¹, R² and Y for a compound corresponds in each case to one row of Table A.

### Table 641

Compounds of the formula (I) in which X is ethyl and Het is 4,6-dichloropyridin-3-yl and the combination of R¹, R² and Y for a compound corresponds in each case to one row of Table A.

### Table 642

Compounds of the formula (I) in which X is ethyl and Het is 4-chloro-6-fluoropyridin-3-yl and the combination of R¹, R² and Y for a compound corresponds in each case to one row of Table A.

### Table 643

Compounds of the formula (I) in which X is ethyl and Het is 4-chloro-6-bromopyridin-3-yl and the combination of R¹, R² and Y for a compound corresponds in each case to one row of Table A.

### Table 644

Compounds of the formula (I) in which X is ethyl and Het is 4-chloro-6-methylpyridin-3-yl and the combination of R¹, R² and Y for a compound corresponds in each case to one row of Table A.

### Table 645

Compounds of the formula (I) in which X is ethyl and Het is 4-chloro-6-ethylpyridin-3-yl and the combination of R¹, R² and Y for a compound corresponds in each case to one row of Table A.

### Table 646

Compounds of the formula (I) in which X is ethyl and Het is 4-chloro-6-methoxypyridin-3-yl and the combination of R¹, R² and Y for a compound corresponds in each case to one row of Table A.

### Table 647

Compounds of the formula (I) in which X is ethyl and Het is 4-chloro-6-cyanopyridin-3-yl and the combination of R¹, R² and Y for a compound corresponds in each case to one row of Table A.

### Table 648

Compounds of the formula (I) in which X is ethyl and Het is 4-chloro-6-methoxycarbonylpyridin-3-yl and the combination of R¹, R² and Y for a compound corresponds in each case to one row of Table A.

### Table 649

Compounds of the formula (I) in which X is ethyl and Het is 4-chloro-6-ethoxycarbonylpyridin-3-yl and the combination of R¹, R² and Y for a compound corresponds in each case to one row of Table A.

### Table 650

Compounds of the formula (I) in which X is ethyl and Het is 4-chloro-6-carbamoylpyridin-3-yl and the combination of R¹, R² and Y for a compound corresponds in each case to one row of Table A.

### Table 651

Compounds of the formula (I) in which X is ethyl and Het is 4-chloro-6-methylcarbamoylpyridin-3-yl and the combination of R¹, R² and Y for a compound corresponds in each case to one row of Table A.

### Table 652

Compounds of the formula (I) in which X is ethyl and Het is 4-chloro-6-dimethylcarbamoylpyridin-3-yl and the combination of R¹, R² and Y for a compound corresponds in each case to one row of Table A.

### Table 653

Compounds of the formula (I) in which X is ethyl and Het is 4-chloro-6-thiocarbamoylpyridin-3-yl and the combination of R¹, R² and Y for a compound corresponds in each case to one row of Table A.

### Table 654

Compounds of the formula (I) in which X is ethyl and Het is 4-chloro-6-nitropyridin-3-yl and the combination of R¹, R² and Y for a compound corresponds in each case to one row of Table A.

### Table 655

Compounds of the formula (I) in which X is ethyl and Het is 4-chloro-6-acetylpyridin-3-yl and the combination of R¹, R² and Y for a compound corresponds in each case to one row of Table A.

### Table 656

Compounds of the formula (I) in which X is ethyl and Het is 4-methyl-6-fluoropyridin-3-yl and the combination of R¹, R² and Y for a compound corresponds in each case to one row of Table A.

### Table 657

Compounds of the formula (I) in which X is ethyl and Het is 4-methyl-6-chloropyridin-3-yl and the combination of R¹, R² and Y for a compound corresponds in each case to one row of Table A.

### Table 658

Compounds of the formula (I) in which X is ethyl and Het is 4-methyl-6-bromopyridin-3-yl and the combination of R¹, R² and Y for a compound corresponds in each case to one row of Table A.

### Table 659

Compounds of the formula (I) in which X is ethyl and Het is 4,6-dimethylpyridin-3-yl and the combination of R¹, R² and Y for a compound corresponds in each case to one row of Table A.

### Table 660

Compounds of the formula (I) in which X is ethyl and Het is 4-methyl-6-ethylpyridin-3-yl and the combination of R¹, R² and Y for a compound corresponds in each case to one row of Table A.

### Table 661

Compounds of the formula (I) in which X is ethyl and Het is 4-methyl-6-methoxypyridin-3-yl and the combination of R¹, R² and Y for a compound corresponds in each case to one row of Table A.

### Table 662

Compounds of the formula (I) in which X is ethyl and Het is 4-methyl-6-cyanopyridin-3-yl and the combination of R¹, R² and Y for a compound corresponds in each case to one row of Table A.

### Table 663

Compounds of the formula (I) in which X is ethyl and Het is 4-methyl-6-methoxycarbonylpyridin-3-yl and the combination of R¹, R² and Y for a compound corresponds in each case to one row of Table A.

### Table 664

Compounds of the formula (I) in which X is ethyl and Het is 4-methyl-6-ethoxycarbonylpyridin-3-yl and the combination of R¹, R² and Y for a compound corresponds in each case to one row of Table A.

### Table 665

Compounds of the formula (I) in which X is ethyl and Het is 4-methyl-6-carbamoylpyridin-3-yl and the combination of R¹, R² and Y for a compound corresponds in each case to one row of Table A.

### Table 666

Compounds of the formula (I) in which X is ethyl and Het is 4-methyl-6-methylcarbamoylpyridin-3-yl and the combination of R¹, R² and Y for a compound corresponds in each case to one row of Table A.

### Table 667

Compounds of the formula (I) in which X is ethyl and Het is 4-methyl-6-dimethylcarbamoylpyridin-3-yl and the combination of R¹, R² and Y for a compound corresponds in each case to one row of Table A.

### Table 668

Compounds of the formula (I) in which X is ethyl and Het is 4-methyl-6-thiocarbamoylpyridin-3-yl and the combination of R¹, R² and Y for a compound corresponds in each case to one row of Table A.

### Table 669

Compounds of the formula (I) in which X is ethyl and Het is 4-methyl-6-nitropyridin-3-yl and the combination of R¹, R² and Y for a compound corresponds in each case to one row of Table A.

### Table 670

Compounds of the formula (I) in which X is ethyl and Het is 4-methyl-6-acetylpyridin-3-yl and the combination of R¹, R² and Y for a compound corresponds in each case to one row of Table A.

### Table 671

Compounds of the formula (I) in which X is ethyl and Het is 4-bromo-6-fluoropyridin-3-yl and the combination of R¹, R² and Y for a compound corresponds in each case to one row of Table A.

### Table 672

Compounds of the formula (I) in which X is ethyl and Het is 4-bromo-6-chloropyridin-3-yl and the combination of R¹, R² and Y for a compound corresponds in each case to one row of Table A.

### Table 673

Compounds of the formula (I) in which X is ethyl and Het is 4-bromo-6-methylpyridin-3-yl and the combination of R¹, R² and Y for a compound corresponds in each case to one row of Table A.

### Table 674

Compounds of the formula (I) in which X is ethyl and Het is 4-methoxy-6-fluoropyridin-3-yl and the combination of R¹, R² and Y for a compound corresponds in each case to one row of Table A.

### Table 675

Compounds of the formula (I) in which X is ethyl and Het is 4-methoxy-6-chloropyridin-3-yl and the combination of R¹, R² and Y for a compound corresponds in each case to one row of Table A:

### Table 676

Compounds of the formula (I) in which X is ethyl and Het is 4-methoxy-6-methylpyridin-3-yl and the combination of R¹, R² and Y for a compound corresponds in each case to one row of Table A.

### Table 677

Compounds of the formula (I) in which X is ethyl and Het is 4-methylsulfanyl-6-fluoropyridin-3-yl and the combination of R¹, R² and Y for a compound corresponds in each case to one row of Table A.

### Table 678

Compounds of the formula (I) in which X is ethyl and Het is 4-methylsulfanyl-6-chloropyridin-3-yl and the combination of R¹, R² and Y for a compound corresponds in each case to one row of Table A.

### Table 679

Compounds of the formula (I) in which X is ethyl and Het is 4-methylsulfanyl-6-methylpyridin-3-yl and the combination of R¹, R² and Y for a compound corresponds in each case to one row of Table A.

### Table 680

Compounds of the formula (I) in which X is ethyl and Het is 4-methylsulfinyl-6-fluoropyridin-3-yl and the combination of R¹, R² and Y for a compound corresponds in each case to one row of Table A.

### Table 681

Compounds of the formula (I) in which X is ethyl and Het is 4-methylsulfinyl-6-chloropyridin-3-yl and the combination of R¹, R² and Y for a compound corresponds in each case to one row of Table A.

### Table 682

Compounds of the formula (I) in which X is ethyl and Het is 4-methylsulfinyl-6-methylpyridin-3-yl and the combination of R¹, R² and Y for a compound corresponds in each case to one row of Table A.

### Table 683

Compounds of the formula (I) in which X is ethyl and Het is 4-methylsulfonyl-6-fluoropyridin-3-yl and the combination of R¹, R² and Y for a compound corresponds in each case to one row of Table A.

### Table 684

Compounds of the formula (I) in which X is ethyl and Het is 4-methylsulfonyl-6-chloropyridin-3-yl and the combination of R¹, R² and Y for a compound corresponds in each case to one row of Table A.

### Table 685

Compounds of the formula (I) in which X is ethyl and Het is 4-methylsulfonyl-6-methylpyridin-3-yl and the combination of R¹, R² and Y for a compound corresponds in each case to one row of Table A.

### Table 686

Compounds of the formula (I) in which X is ethyl and Het is 4-nitro-6-fluoropyridin-3-yl and the combination of R¹, R² and Y for a compound corresponds in each case to one row of Table A.

### Table 687

Compounds of the formula (I) in which X is ethyl and Het is 4-nitro-6-chloropyridin-3-yl and the combination of R¹, R² and Y for a compound corresponds in each case to one row of Table A.

### Table 688

Compounds of the formula (I) in which X is ethyl and Het is 4-nitro-6-methylpyridin-3-yl and the combination of R¹, R² and Y for a compound corresponds in each case to one row of Table A.

### Table 689

Compounds of the formula (I) in which X is ethyl and Het is 4-methoxymethyl-6-fluoropyridin-3-yl and the combination of R¹, R² and Y for a compound corresponds in each case to one row of Table A.

### Table 690

Compounds of the formula (I) in which X is ethyl and Het is 4-methoxymethyl-6-chloropyridin-3-yl and the combination of R¹, R² and Y for a compound corresponds in each case to one row of Table A.

### Table 691

Compounds of the formula (I) in which X is ethyl and Het is 4-methoxymethyl-6-methylpyridin-3-yl and the combination of R¹, R² and Y for a compound corresponds in each case to one row of Table A.

### Table 692

Compounds of the formula (I) in which X is ethyl and Het is 2,4-dichloro-6-methylpyridin-3-yl and the combination of R¹, R² and Y for a compound corresponds in each case to one row of Table A.

### Table 693

Compounds of the formula (I) in which X is n-propyl and Het is 2-pyridinyl and the combination of R¹, R² and Y for a compound corresponds in each case to one row of Table A.

### Table 694

Compounds of the formula (I) in which X is n-propyl and Het is 5-nitropyridin-2-yl and the combination of R¹, R² and Y for a compound corresponds in each case to one row of Table A.

### Table 695

Compounds of the formula (I) in which X is n-propyl and Het is 5-cyanopyridin-2-yl and the combination of R¹, R² and Y for a compound corresponds in each case to one row of Table A.

### Table 696

Compounds of the formula (I) in which X is n-propyl and Het is 5-methoxycarbonyl-pyridin-2-yl and the combination of R¹, R² and Y for a compound corresponds in each case to one row of Table A.

### Table 697

Compounds of the formula (I) in which X is n-propyl and Het is 5-methylpyridin-2-yl and the combination of R¹, R² and Y for a compound corresponds in each case to one row of Table A.

### Table 698

Compounds of the formula (I) in which X is n-propyl and Het is 4-methylpyridin-2-yl and the combination of R¹, R² and Y for a compound corresponds in each case to one row of Table A.

### Table 699

Compounds of the formula (I) in which X is n-propyl and Het is 3-methylpyridin-2-yl and the combination of R¹, R² and Y for a compound corresponds in each case to one row of Table A.

### Table 700

Compounds of the formula (I) in which X is n-propyl and Het is 3-ethylpyridin-2-yl and the combination of R¹, R² and Y for a compound corresponds in each case to one row of Table A.

### Table 701

Compounds of the formula (I) in which X is n-propyl and Het is 6-methylpyridin-2-yl and the combination of R¹, R² and Y for a compound corresponds in each case to one row of Table A.

### Table 702

Compounds of the formula (I) in which X is n-propyl and Het is 5-trifluoromethyl-pyridin-2-yl and the combination of R¹, R² and Y for a compound corresponds in each case to one row of Table A.

### Table 703

Compounds of the formula (I) in which X is n-propyl and Het is 3-trifluoromethyl-pyridin-2-yl and the combination of R¹, R² and Y for a compound corresponds in each case to one row of Table A.

### Table 704

Compounds of the formula (I) in which X is n-propyl and Het is 5-fluoropyridin-2-yl and the combination of R¹, R² and Y for a compound corresponds in each case to one row of Table A.

### Table 705

Compounds of the formula (I) in which X is n-propyl and Het is 3-fluoropyridin-2-yl and the combination of R¹, R² and Y for a compound corresponds in each case to one row of Table A.

### Table 706

Compounds of the formula (I) in which X is n-propyl and Het is 3,5-difluoropyridin-2-yl and the combination of R¹, R² and Y for a compound corresponds in each case to one row of Table A.

### Table 707

Compounds of the formula (I) in which X is n-propyl and Het is 3,5-dichloropyridin-2-yl and the combination of R¹, R² and Y for a compound corresponds in each case to one row of Table A.

### Table 708

Compounds of the formula (I) in which X is n-propyl and Het is pyridin-3-yl and the combination of R¹, R² and Y for a compound corresponds in each case to one row of Table A.

### Table 709

Compounds of the formula (I) in which X is n-propyl and Het is pyridin-4-yl and the combination of R¹, R² and Y for a compound corresponds in each case to one row of Table A.

### Table 710

Compounds of the formula (I) in which X is n-propyl and Het is pyrazin-2-yl and the combination of R¹, R² and Y for a compound corresponds in each case to one row of Table A.

### Table 711

Compounds of the formula (I) in which X is n-propyl and Het is pyridazin-3-yl and the combination of R¹, R² and Y for a compound corresponds in each case to one row of Table A.

### Table 712

Compounds of the formula (I) in which X is n-propyl and Het is 6-chloropyridazin-3-yl and the combination of R¹, R² and Y for a compound corresponds in each case to one row of Table A.

### Table 713

Compounds of the formula (I) in which X is n-propyl and Het is 6-methoxy-pyridazin-3-yl and the combination of R¹, R² and Y for a compound corresponds in each case to one row of Table A.

### Table 714

Compounds of the formula (I) in which X is n-propyl and Het is 1,3,5-triazin-2-yl and the combination of R¹, R² and Y for a compound corresponds in each case to one row of Table A.

### Table 715

Compounds of the formula (I) in which X is n-propyl and Het is pyrimidin-2-yl and the combination of R¹, R² and Y for a compound corresponds in each case to one row of Table A.

### Table 716

Compounds of the formula (I) in which X is n-propyl and Het is pyrimidin-4-yl and the combination of R¹, R² and Y for a compound corresponds in each case to one row of Table A.

### Table 717

Compounds of the formula (I) in which X is n-propyl and Het is pyrimidin-5-yl and the combination of R¹, R² and Y for a compound corresponds in each case to one row of Table A.

### Table 718

Compounds of the formula (I) in which X is n-propyl and Het is 2-methyl-6-trifluoromethylpyrimidin-4-yl and the combination of R¹, R² and Y for a compound corresponds in each case to one row of Table A.

### Table 719

Compounds of the formula (I) in which X is n-propyl and Het is 2,5-dimethyl-6-trifluoromethylpyrimidin-4-yl and the combination of R¹, R² and Y for a compound corresponds in each case to one row of Table A.

### Table 720

Compounds of the formula (I) in which X is n-propyl and Het is 6-trifluoromethylpyrimidin-4-yl and the combination of R¹, R² and Y for a compound corresponds in each case to one row of Table A.

### Table 721

Compounds of the formula (I) in which X is n-propyl and Het is 5-methyl-6-trifluoromethylpyrimidin-4-yl and the combination of R¹, R² and Y for a compound corresponds in each case to one row of Table A.

### Table 722

Compounds of the formula (I) in which X is n-propyl and Het is 5-chloro-6-methylpyrimidin-4-yl and the combination of R¹, R² and Y for a compound corresponds in each case to one row of Table A.

### Table 723

Compounds of the formula (I) in which X is n-propyl and Het is 5-bromo-6-methylpyrimidin-4-yl and the combination of R¹, R² and Y for a compound corresponds in each case to one row of Table A.

### Table 724

Compounds of the formula (I) in which X is n-propyl and Het is 5-fluoro-6-methylpyrimidin-4-yl and the combination of R¹, R² and Y for a compound corresponds in each case to one row of Table A.

### Table 725

Compounds of the formula (I) in which X is n-propyl and Het is 5-chloro-6-ethylpyrimidin-4-yl and the combination of R¹, R² and Y for a compound corresponds in each case to one row of Table A.

### Table 726

Compounds of the formula (I) in which X is n-propyl and Het is 5-bromo-6-ethylpyrimidin-4-yl and the combination of R¹, R² and Y for a compound corresponds in each case to one row of Table A.

### Table 727

Compounds of the formula (I) in which X is n-propyl and Het is 5-chloro-6-isopropyl-pyrimidin-4-yl and the combination of R¹, R² and Y for a compound corresponds in each case to one row of Table A.

### Table 728

Compounds of the formula (I) in which X is n-propyl and Het is 5-bromo-6-isopropylpyrimidin-4-yl and the combination of R¹, R² and Y for a compound corresponds in each case to one row of Table A.

### Table 729

Compounds of the formula (I) in which X is n-propyl and Het is 5-chloropyrimidin-4-yl and the combination of R¹, R² and Y for a compound corresponds in each case to one row of Table A.

### Table 730

Compounds of the formula (I) in which X is n-propyl and Het is 5-fluoropyrimidin-4-yl and the combination of R¹, R² and Y for a compound corresponds in each case to one row of Table A.

### Table 731

Compounds of the formula (I) in which X is n-propyl and Het is 5-bromopyrimidin-4-yl and the combination of R¹, R² and Y for a compound corresponds in each case to one row of Table A.

### Table 732

Compounds of the formula (I) in which X is n-propyl and Het is 2-methyl-5-fluoropyrimidin-4-yl and the combination of R¹, R² and Y for a compound corresponds in each case to one row of Table A.

### Table 733

Compounds of the formula (I) in which X is n-propyl and Het is 2-methyl-5-chloropyrimidin-4-yl and the combination of R¹, R² and Y for a compound corresponds in each case to one row of Table A.

### Table 734

Compounds of the formula (I) in which X is n-propyl and Het is 2-methyl-5-bromopyrimidin-4-yl and the combination of R¹, R² and Y for a compound corresponds in each case to one row of Table A.

### Table 735

Compounds of the formula (I) in which X is n-propyl and Het is 2,6-dimethyl-5-chloropyrimidin-4-yl and the combination of R¹, R² and Y for a compound corresponds in each case to one row of Table A.

### Table 736

Compounds of the formula (I) in which X is n-propyl and Het is 2,6-dimethyl-5-bromopyrimidin-4-yl and the combination of R¹, R² and Y for a compound corresponds in each case to one row of Table A.

### Table 737

Compounds of the formula (I) in which X is n-propyl and Het is 5-fluoro-6-fluoromethylpyrimidin-4-yl and the combination of R¹, R² and Y for a compound corresponds in each case to one row of Table A.

### Table 738

Compounds of the formula (I) in which X is n-propyl and Het is 5-chloro-6-methoxymethylpyrimidin-4-yl and the combination of R¹, R² and Y for a compound corresponds in each case to one row of Table A.

### Table 739

Compounds of the formula (I) in which X is n-propyl and Het is 5,6-dimethylpyrimidin-4-yl and the combination of R¹, R² and Y for a compound corresponds in each case to one row of Table A.

### Table 740

Compounds of the formula (I) in which X is n-propyl and Het is 2,5-dimethylpyrimidin-4-yl and the combination of R¹, R² and Y for a compound corresponds in each case to one row of Table A.

### Table 741

Compounds of the formula (I) in which X is n-propyl and Het is 2,5,6-trimethylpyrimidin-4-yl and the combination of R¹, R² and Y for a compound corresponds in each case to one row of Table A.

### Table 742

Compounds of the formula (I) in which X is n-propyl and Het is 5-methyl-6-methoxy-pyrimidin-4-yl and the combination of R¹, R² and Y for a compound corresponds in each case to one row of Table A.

### Table 743

Compounds of the formula (I) in which X is n-propyl and Het is 4,6-dimethylpyrimidin-2-yl and the combination of R¹, R² and Y for a compound corresponds in each case to one row of Table A.

### Table 744

Compounds of the formula (I) in which X is n-propyl and Het is 4,5,6-trimethylpyrimidin-2-yl and the combination of R¹, R² and Y for a compound corresponds in each case to one row of Table A.

### Table 745

Compounds of the formula (I) in which X is n-propyl and Het is 4,6-di(trifluoromethyl)pyrimidin-2-yl and the combination of R¹, R² and Y for a compound corresponds in each case to one row of Table A.

### Table 746

Compounds of the formula (I) in which X is n-propyl and Het is 4,6-dimethyl-5-chloropyrimidin-2-yl and the combination of R¹, R² and Y for a compound corresponds in each case to one row of Table A.

### Table 747

Compounds of the formula (I) in which X is n-propyl and Het is 4,6-dimethyl-5-bromopyrimidin-2-yl and the combination of R¹, R² and Y for a compound corresponds in each case to one row of Table A.

### Table 748

Compounds of the formula (I) in which X is n-propyl and Het is 4-methylpyrimidin-5-yl and the combination of R¹, R² and Y for a compound corresponds in each case to one row of Table A.

### Table 749

Compounds of the formula (I) in which X is n-propyl and Het is 4,6-dimethylpyrimidin-5-yl and the combination of R¹, R² and Y for a compound corresponds in each case to one row of Table A.

### Table 750

Compounds of the formula (I) in which X is n-propyl and Het is 4-trifluoromethyl-6-methylpyrimidin-5-yl and the combination of R¹, R² and Y for a compound corresponds in each case to one row of Table A.

### Table 751

Compounds of the formula (I) in which X is n-propyl and Het is 2,4,6-trimethylpyrimidin-5-yl and the combination of R¹, R² and Y for a compound corresponds in each case to one row of Table A.

### Table 752

Compounds of the formula (I) in which X is n-propyl and Het is 3-chloropyridin-2-yl and the combination of R¹, R² and Y for a compound corresponds in each case to one row of Table A.

### Table 753

Compounds of the formula (I) in which X is n-propyl and Het is 3-bromopyridin-2-yl and the combination of R¹, R² and Y for a compound corresponds in each case to one row of Table A.

### Table 754

Compounds of the formula (I) in which X is n-propyl and Het is 3-iodopyridin-2-yl and the combination of R¹, R² and Y for a compound corresponds in each case to one row of Table A.

### Table 755

Compounds of the formula (I) in which X is n-propyl and Het is 3-methoxypyridin-2-yl and the combination of R¹, R² and Y for a compound corresponds in each case to one row of Table A.

### Table 756

Compounds of the formula (I) in which X is n-propyl and Het is 3-methylsulfanylpyridin-2-yl and the combination of R¹, R² and Y for a compound corresponds in each case to one row of Table A.

### Table 757

Compounds of the formula (I) in which X is n-propyl and Het is 3-methylsulfinylpyridin-2-yl and the combination of R¹, R² and Y for a compound corresponds in each case to one row of Table A.

### Table 758

Compounds of the formula (I) in which X is n-propyl and Het is 3-methylsulfonylpyridin-2-yl and the combination of R¹, R² and Y for a compound corresponds in each case to one row of Table A.

### Table 759

Compounds of the formula (I) in which X is n-propyl and Het is 3-nitropyridin-2-yl and the combination of R¹, R² and Y for a compound corresponds in each case to one row of Table A.

### Table 760

Compounds of the formula (I) in which X is n-propyl and Het is 3-methoxymethylpyridin-2-yl and the combination of R¹, R² and Y for a compound corresponds in each case to one row of Table A.

### Table 761

Compounds of the formula (I) in which X is n-propyl and Het is 5-chloropyridin-2-yl and the combination of R¹, R² and Y for a compound corresponds in each case to one row of Table A.

### Table 762

Compounds of the formula (I) in which X is n-propyl and Het is 5-bromopyridin-2-yl and the combination of R¹, R² and Y for a compound corresponds in each case to one row of Table A.

### Table 763

Compounds of the formula (I) in which X is n-propyl and Het is 5-methoxypyridin-2-yl and the combination of R¹, R² and Y for a compound corresponds in each case to one row of Table A.

### Table 764

Compounds of the formula (I) in which X is n-propyl and Het is 5-methylsulfanylpyridin-2-yl and the combination of R¹, R² and Y for a compound corresponds in each case to one row of Table A.

### Table 765

Compounds of the formula (I) in which X is n-propyl and Het is 5-methylsulfinylpyridin-2-yl and the combination of R¹, R² and Y for a compound corresponds in each case to one row of Table A.

### Table 766

Compounds of the formula (I) in which X is n-propyl and Het is 5-methylsulfonylpyridin-2-yl and the combination of R¹, R² aid Y for a compound corresponds in each case to one row of Table A.

### Table 767

Compounds of the formula (I) in which X is n-propyl and Het is 5-methoxymethylpyridin-2-yl and the combination of R¹, R² and Y for a compound corresponds in each case to one row of Table A.

### Table 768

Compounds of the formula (I) in which X is n-propyl and Het is 5-ethylpyridin-2-yl and the combination of R¹, R² and Y for a compound corresponds in each case to one row of Table A.

### Table 769

Compounds of the formula (I) in which X is n-propyl and Het is 5-ethoxycarbonylpyridin-2-yl and the combination of R¹, R² and Y for a compound corresponds in each case to one row of Table A.

### Table 770

Compounds of the formula (I) in which X is n-propyl and Het is 5-carbamoylpyridin-2-yl and the combination of R¹, R² and Y for a compound corresponds in each case to one row of Table A.

### Table 771

Compounds of the formula (I) in which X is n-propyl and Het is 5-methylcarbamoylpyridin-2-yl and the combination of R¹, R² and Y for a compound corresponds in each case to one row of Table A.

### Table 772

Compounds of the formula (I) in which X is n-propyl and Het is 5-dimethylcarbamoylpyridin-2-yl and the combination of R¹, R² and Y for a compound corresponds in each case to one row of Table A.

### Table 773

Compounds of the formula (I) in which X is n-propyl and Het is 5-Thiocarbamoylpyridin-2-yl and the combination of R¹, R² and Y for a compound corresponds in each case to one row of Table A.

### Table 774

Compounds of the formula (I) in which X is n-propyl and Het is 5-acetylpyridin-2-yl and the combination of R¹, R² and Y for a compound corresponds in each case to one row of Table A.

### Table 775

Compounds of the formula (I) in which X is n-propyl and Het is 5-methylpyridin-2-yl and the combination of R¹, R² and Y for a compound corresponds in each case to one row of Table A.

### Table 776

Compounds of the formula (I) in which X is n-propyl and Het is 5-nitropyridin-2-yl and the combination of R¹, R² and Y for a compound corresponds in each case to one row of Table A.

### Table 777

Compounds of the formula (I) in which X is n-propyl and Het is 5-cyanopyridin-2-yl and the combination of R¹, R² and Y for a compound corresponds in each case to one row of Table A.

### Table 778

Compounds of the formula (I) in which X is n-propyl and Het is 3-fluoro-5-chloropyridin-2-yl and the combination of R¹, R² and Y for a compound corresponds in each case to one row of Table A.

### Table 779

Compounds of the formula (I) in which X is n-propyl and Het is 3-fluoro-5-bromopyridin-2-yl and the combination of R¹, R² and Y for a compound corresponds in each case to one row of Table A.

### Table 780

Compounds of the formula (I) in which X is n-propyl and Het is 3-fluoro-5-methylpyridin-2-yl and the combination of R¹, R² and Y for a compound corresponds in each case to one row of Table A.

### Table 781

Compounds of the formula (I) in which X is n-propyl and Het is 3-fluoro-5-ethylpyridin-2-yl and the combination of R¹, R² and Y for a compound corresponds in each case to one row of Table A.

### Table 782

Compounds of the formula (I) in which X is n-propyl and Het is 3-fluoro-5-methoxypyridin-2-yl and the combination of R¹, R² and Y for a compound corresponds in each case to one row of Table A.

### Table 783

Compounds of the formula (I) in which X is n-propyl and Het is 3-fluoro-5-cyanopyridin-2-yl and the combination of R¹, R² and Y for a compound corresponds in each case to one row of Table A.

### Table 784

Compounds of the formula (I) in which X is n-propyl and Het is 3-fluoro-5-methoxycarbonylpyridin-2-yl and the combination of R¹, R² and Y for a compound corresponds in each case to one row of Table A.

### Table 785

Compounds of the formula (I) in which X is n-propyl and Het is 3-fluoro-5-ethoxycarbonylpyridin-2-yl and the combination of R¹, R² and Y for a compound corresponds in each case to one row of Table A.

### Table 786

Compounds of the formula (I) in which X is n-propyl and Het is 3-fluoro-5-carbamoylpyridin-2-yl and the combination of R¹, R² and Y for a compound corresponds in each case to one row of Table A.

### Table 787

Compounds of the formula (I) in which X is n-propyl and Het is 3-fluoro-5-methylcarbamoylpyridin-2-yl and the combination of R¹, R² and Y for a compound corresponds in each case to one row of Table A.

### Table 788

Compounds of the formula (I) in which X is n-propyl and Het is 3-fluoro-5-dimethylcarbamoylpyridin-2-yl and the combination of R¹, R² and Y for a compound corresponds in each case to one row of Table A.

### Table 789

Compounds of the formula (I) in which X is n-propyl and Het is 3-fluoro-5-thiocarbamoylpyridin-2-yl and the combination of R¹, R² and Y for a compound corresponds in each case to one row of Table A.

### Table 790

Compounds of the formula (I) in which X is n-propyl and Het is 3-fluoro-5-nitropyridin-2-yl and the combination of R¹, R² and Y for a compound corresponds in each case to one row of Table A.

### Table 791

Compounds of the formula (I) in which X is n-propyl and Het is 3-fluoro-5-acetylpyridin-2-yl and the combination of R¹, R² and Y for a compound corresponds in each case to one row of Table A.

### Table 792

Compounds of the formula (I) in which X is n-propyl and Het is 3-chloro-5-fluoropyridin-2-yl and the combination of R¹, R² and Y for a compound corresponds in each case to one row of Table A:

### Table 793

Compounds of the formula (I) in which X is n-propyl and Het is 3-chloro-5-bromopyridin-2-yl and the combination of R¹, R² and Y for a compound corresponds in each case to one row of Table A.

### Table 794

Compounds of the formula (I) in which X is n-propyl and Het is 3-chloro-5-methylpyridin-2-yl and the combination of R¹, R² and Y for a compound corresponds in each case to one row of Table A.

### Table 795

Compounds of the formula (I) in which X is n-propyl and Het is 3-chloro-5-ethylpyridin-2-yl and the combination of R¹, R² and Y for a compound corresponds in each case to one row of Table A.

### Table 796

Compounds of the formula (I) in which X is n-propyl and Het is 3-chloro-5-methoxypyridin-2-yl and the combination of R¹, R² and Y for a compound corresponds in each case to one row of Table A.

### Table 797

Compounds of the formula (I) in which X is n-propyl and Het is 3-chloro-5-cyanopyridin-2-yl and the combination of R¹, R² and Y for a compound corresponds in each case to one row of Table A.

### Table 798

Compounds of the formula (I) in which X is n-propyl and Het is 3-chloro-5-methoxycarbonylpyridin-2-yl and the combination of R¹, R² and Y for a compound corresponds in each case to one row of Table A.

### Table 799

Compounds of the formula (I) in which X is n-propyl and Het is 3-chloro-5-ethoxycarbonylpyridin-2-yl and the combination of R¹, R² and Y for a compound corresponds in each case to one row of Table A.

### Table 800

Compounds of the formula (I) in which X is n-propyl and Het is 3-chloro-5-carbamoylpyridin-2-yl and the combination of R¹, R² and Y for a compound corresponds in each case to one row of Table A.

### Table 801

Compounds of the formula (I) in which X is n-propyl and Het is 3-chloro-5-methylcarbamoylpyridin-2-yl and the combination of R¹, R² and Y for a compound corresponds in each case to one row of Table A.

### Table 802

Compounds of the formula (I) in which X is n-propyl and Het is 3-chloro-5-dimethylcarbamoylpyridin-2-yl and the combination of R¹, R² and Y for a compound corresponds in each case to one row of Table A.

### Table 803

Compounds of the formula (I) in which X is n-propyl and Het is 3-chloro-5-thiocarbamoylpyridin-2-yl and the combination of R¹, R² and Y for a compound corresponds in each case to one row of Table A.

### Table 804

Compounds of the formula (I) in which X is n-propyl and Het is 3-chloro-5-nitropyridin-2-yl and the combination of R¹, R² and Y for a compound corresponds in each case to one row of Table A.

### Table 805

Compounds of the formula (I) in which X is n-propyl and Het is 3-chloro-5-acetylpyridin-2-yl and the combination of R¹, R² and Y for a compound corresponds in each case to one, row of Table A.

### Table 806

Compounds of the formula (I) in which X is n-propyl and Het is 3-methyl-5-fluoropyridin-2-yl and the combination of R¹, R² and Y for a compound corresponds in each case to one row of Table A.

### Table 807

Compounds of the formula (I) in which X is n-propyl and Het is 3-methyl-5-chloropyridin-2-yl and the combination of R¹, R² and Y for a compound corresponds in each case to one row of Table A.

### Table 808

Compounds of the formula (I) in which X is n-propyl and Het is 3-methyl-5-bromopyridin-2-yl and the combination of R¹, R² and Y for a compound corresponds in each case to one row of Table A.

### Table 809

Compounds of the formula (I) in which X is n-propyl and Het is 3,5-dimethylpyridin-2-yl and the combination of R¹, R² and Y for a compound corresponds in each case to one row of Table A.

### Table 810

Compounds of the formula (I) in which X is n-propyl and Het is 3-methyl-5-ethylpyridin-2-yl and the combination of R¹, R² and Y for a compound corresponds in each case to one row of Table A.

### Table 811

Compounds of the formula (I) in which X is n-propyl and Het is 3-methyl-5-methoxypyridin-2-yl and the combination of R¹, R² and Y for a compound corresponds in each case to one row of Table A.

### Table 812

Compounds of the formula (I) in which X is n-propyl and Het is 3-methyl-5-cyanopyridin-2-yl and the combination of R¹, R² and Y for a compound corresponds in each case to one row of Table A.

### Table 813

Compounds of the formula (I) in which X is n-propyl and Het is 3-methyl-5-methoxycarbonylpyridin-2-yl and the combination of R¹, R² and Y for a compound corresponds in each case to one row of Table A.

### Table 814

Compounds of the formula (I) in which X is n-propyl and Het is 3-methyl-5-ethoxycarbonylpyridin-2-yl and the combination of R¹, R² and Y for a compound corresponds in each case to one row of Table A.

### Table 815

Compounds of the formula (I) in which X is n-propyl and Het is 3-methyl-5-carbamoylpyridin-2-yl and the combination of R¹, R² and Y for a compound corresponds in each case to one row of Table A.

### Table 816

Compounds of the formula (I) in which X is n-propyl and Het is 3-methyl-5-methylcarbamoylpyridin-2-yl and the combination of R¹, R² and Y for a compound corresponds in each case to one row of Table A.

### Table 817

Compounds of the formula (I) in which X is n-propyl and Het is 3-methyl-5-dimethylcarbamoylpyridin-2-yl and the combination of R¹, R² and Y for a compound corresponds in each case to one row of Table A.

### Table 818

Compounds of the formula (I) in which X is n-propyl and Het is 3-methyl-5-thiocarbamoylpyridin-2-yl and the combination of R¹, R² and Y for a compound corresponds in each case to one row of Table A.

### Table 819

Compounds of the formula (I) in which X is n-propyl and Het is 3-methyl-5-nitropyridin-2-yl and the combination of R¹, R² and Y for a compound corresponds in each case to one row of Table A.

### Table 820

Compounds of the formula (I) in which X is n-propyl and Het is 3-methyl-5-acetylpyridin-2-yl and the combination of R¹, R² and Y for a compound corresponds in each case to one row of Table A.

### Table 821

Compounds of the formula (I) in which X is n-propyl and Het is 3-bromo-5-fluoropyridin-2-yl and the combination of R¹, R² and Y for a compound corresponds in each case to one row of Table A.

### Table 822

Compounds of the formula (I) in which X is n-propyl and Het is 3-bromo-5-chloropyridin-2-yl and the combination of R¹, R² and Y for a compound corresponds in each case to one row of Table A.

### Table 823

Compounds of the formula (I) in which X is n-propyl and Het is 3-bromo-5-methylpyridin-2-yl and the combination of R¹, R² and Y for a compound corresponds in each case to one row of Table A.

### Table 824

Compounds of the formula (I) in which X is n-propyl and Het is 3-methylsulfonyl-5-fluoropyridin-2-yl and the combination of R¹, R² and Y for a compound corresponds in each case to one row of Table A.

### Table 825

Compounds of the formula (I) in which X is n-propyl and Het is 3-methylsulfonyl-5-chloropyridin-2-yl and the combination of R¹, R² and Y for a compound corresponds in each case to one row of Table A.

### Table 826

Compounds of the formula (I) in which X is n-propyl and Het is 3-methylsulfonyl-5-methylpyridin-2-yl and the combination of R¹, R² and Y for a compound corresponds in each case to one row of Table A.

### Table 827

Compounds of the formula (I) in which X is n-propyl and Het is 3-methoxy-5-fluoropyridin-2-yl and the combination of R¹, R² and Y for a compound corresponds in each case to one row of Table A.

### Table 828

Compounds of the formula (I) in which X is n-propyl and Het is 3-methoxy-5-chloropyridin-2-yl and the combination of R¹, R² and Y for a compound corresponds in each case to one row of Table A.

### Table 829

Compounds of the formula (I) in which X is n-propyl and Het is 3-methoxy-5-methylpyridin-2-yl and the combination of R¹, R² and Y for a compound corresponds in each case to one row of Table A.

### Table 830

Compounds of the formula (I) in which X is n-propyl and Het is 3-methylsulfanyl-5-fluoropyridin-2-yl and the combination of R¹, R² and Y for a compound corresponds in each case to one row of Table A.

### Table 831

Compounds of the formula (I) in which X is n-propyl and Het is 3-methylsulfanyl-5-chloropyridin-2-yl and the combination of R¹, R² and Y for a compound corresponds in each case to one row of Table A.

### Table 832

Compounds of the formula (I) in which X is n-propyl and Het is 3-methylsulfanyl-5-methylpyridin-2-yl and the combination of R¹, R² and Y for a compound corresponds in each case to one row of Table A.

### Table 833

Compounds of the formula (I) in which X is n-propyl and Het is 3-nitro-5-fluoropyridin-2-yl and the combination of R¹, R² and Y for a compound corresponds in each case to one row of Table A.

### Table 834

Compounds of the formula (I) in which X is n-propyl and Het is 3-nitro-5-chloropyridin-2-yl and the combination of R¹, R² and Y for a compound corresponds in each case to one row of Table A.

### Table 835

Compounds of the formula (I) in which X is n-propyl and Het is 3-nitro-5-methylpyridin-2-yl and the combination of R¹, R² and Y for a compound corresponds in each case to one row of Table A.

### Table 836

Compounds of the formula (I) in which X is n-propyl and Het is 3,5-dibromopyridin-2-yl and the combination of R¹, R² and Y for a compound corresponds in each case to one row of Table A.

### Table 837

Compounds of the formula (I) in which X is n-propyl and Het is 3-fluoropyridin-4-yl and the combination of R¹, R² and Y for a compound corresponds in each case to one row of Table A.

### Table 838

Compounds of the formula (I) in which X is n-propyl and Het is 3-chloropyridin-4-yl and the combination of R¹, R² and Y for a compound corresponds in each case to one row of Table A.

### Table 839

Compounds of the formula (I) in which X is n-propyl and Het is 3-bromopyridin-4-yl and the combination of R¹, R² and Y for a compound corresponds in each case to one row of Table A.

### Table 840

Compounds of the formula (I) in which X is n-propyl and Het is 3-methylpyridin-4-yl and the combination of R¹, R² and Y for a compound corresponds in each case to one row of Table A.

### Table 841

Compounds of the formula (I) in which X is n-propyl and Het is 3-methoxypyridin-4-yl and the combination of R¹, R² and Y for a compound corresponds in each case to one row of Table A.

### Table 842

Compounds of the formula (I) in which X is n-propyl and Het is 3-methylsulfanylpyridin-4-yl and the combination of R¹, R² and Y for a compound corresponds in each case to one row of Table A.

### Table 843

Compounds of the formula (I) in which X is n-propyl and Het is 3-methylsulfinylpyridin-4-yl and the combination of R¹, R² and Y for a compound corresponds in each case to one row of Table A.

### Table 844

Compounds of the formula (I) in which X is n-propyl and Het is 3-methylsulfonylpyridin-4-yl and the combination of R¹, R² and Y for a compound corresponds in each case to one row of Table A.

### Table 845

Compounds of the formula (I) in which X is n-propyl and Het is 3-nitropyridin-4-yl and the combination of R¹, R² and Y for a compound corresponds in each case to one row of Table A.

### Table 846

Compounds of the formula (I) in which X is n-propyl and Het is 3-methoxymethylpyridin-4-yl and the combination of R¹, R² and Y for a compound corresponds in each case to one row of Table A.

### Table 847

Compounds of the formula (I) in which X is n-propyl and Het is 3,5-difluoropyridin-4-yl and the combination of R¹, R² and Y for a compound corresponds in each case to one row of Table A.

### Table 848

Compounds of the formula (I) in which X is n-propyl and Het is 3-fluoro-5-chloropyridin-4-yl and the combination of R¹, R² and Y for a compound corresponds in each case to one row of Table A.

### Table 849

Compounds of the formula (I) in which X is n-propyl and Het is 3-fluoro-5-bromopyridin-4-yl and the combination of R¹, R² and Y for a compound corresponds in each case to one row of Table A.

### Table 850

Compounds of the formula (I) in which X is n-propyl and Het is 3-fluoro-5-methylpyridin-4-yl and the combination of R¹, R² and Y for a compound corresponds in each case to one row of Table A.

### Table 851

Compounds of the formula (I) in which X is n-propyl and Het is 3-fluoro-5-ethylpyridin-4-yl and the combination of R¹, R² and Y for a compound corresponds in each case to one row of Table A.

### Table 852

Compounds of the formula (I) in which X is n-propyl and Het is 3-fluoro-5-methoxypyridin-4-yl and the combination of R¹, R² and Y for a compound corresponds in each case to one row of Table A.

### Table 853

Compounds of the formula (I) in which X is n-propyl and Het is 3-fluoro-5-cyanopyridin-4-yl and the combination of R¹, R² and Y for a compound corresponds in each case to one row of Table A.

### Table 854

Compounds of the formula (I) in which X is n-propyl and Het is 3-fluoro-5-methoxycarbonylpyridin-4-yl and the combination of R¹, R² and Y for a compound corresponds in each case to one row of Table A.

### Table 855

Compounds of the formula (I) in which X is n-propyl and Het is 3-fluoro-5-ethoxycarbonylpyridin-4-yl and the combination of R¹, R² and Y for a compound corresponds in each case to one row of Table A.

### Table 856

Compounds of the formula (I) in which X is n-propyl and Het is 3-fluoro-5-carbamoylpyridin-4-yl and the combination of R¹, R² and Y for a compound corresponds in each case to one row of Table A.

### Table 857

Compounds of the formula (I) in which X is n-propyl and Het is 3-fluoro-5-methylcarbamoylpyridin-4-yl and the combination of R¹, R² and Y for a compound corresponds in each case to one row of Table A.

### Table 858

Compounds of the formula (I) in which X is n-propyl and Het is 3-fluoro-5-dimethylcarbamoylpyridin-4-yl and the combination of R¹, R² and Y for a compound corresponds in each case to one row of Table A.

### Table 859

Compounds of the formula (I) in which X is n-propyl and Het is 3-fluoro-5-thiocarbamoylpyridin-4-yl and the combination of R¹, R² and Y for a compound corresponds in each case to one row of Table A.

### Table 860

Compounds of the formula (I) in which X is n-propyl and Het is 3-fluoro-5-nitropyridin-4-yl and the combination of R¹, R² and Y for a compound corresponds in each case to one row of Table A.

### Table 861

Compounds of the formula (I) in which X is n-propyl and Het is 3-fluoro-5-acetylpyridin-4-yl and the combination of R¹, R² and Y for a compound corresponds in each case to one row of Table A.

### Table 862

Compounds of the formula (I) in which X is n-propyl and Het is 3,5-dichloro-5-chloropyridin-4-yl and the combination of R¹, R² and Y for a compound corresponds in each case to one row of Table A.

### Table 863

Compounds of the formula (I) in which X is n-propyl and Het is 3-chloro-5-bromopyridin-4-yl and the combination of R¹, R² and Y for a compound corresponds in each case to one row of Table A.

### Table 864

Compounds of the formula (I) in which X is n-propyl and Het is 3-chloro-5-methylpyridin-4-yl and the combination of R¹, R² and Y for a compound corresponds in each case to one row of Table A.

### Table 865

Compounds of the formula (I) in which X is n-propyl and Het is 3-chloro-5-ethylpyridin-4-yl and the combination of R¹, R² and Y for a compound corresponds in each case to one row of Table A.

### Table 866

Compounds of the formula (I) in which X is n-propyl and Het is 3-chloro-5-methoxypyridin-4-yl and the combination of R¹, R² and Y for a compound corresponds in each case to one row of Table A.

### Table 867

Compounds of the formula (I) in which X is n-propyl and Het is 3-chloro-5-cyanopyridin-4-yl and the combination of R¹, R² and Y for a compound corresponds in each case to one row of Table A.

### Table 868

Compounds of the formula (I) in which X is n-propyl and Het is 3-chloro-5-methoxycarbonylpyridin-4-yl and the combination of R¹, R² and Y for a compound corresponds in each case to one row of Table A.

### Table 869

Compounds of the formula (I) in which X is n-propyl and Het is 3-chloro-5-ethoxycarbonylpyridin-4-yl and the combination of R¹, R² and Y for a compound corresponds in each case to one row of Table A.

### Table 870

Compounds of the formula (I) in which X is n-propyl and Het is 3-chloro-5-carbamoylpyridin-4-yl and the combination of R¹, R² and Y for a compound corresponds in each case to one row of Table A.

### Table 871

Compounds of the formula (I) in which X is n-propyl and Het is 3-chloro-5-methylcarbamoylpyridin-4-yl and the combination of R¹, R² and Y for a compound corresponds in each case to one row of Table A.

### Table 872

Compounds of the formula (I) in which X is n-propyl and Het is 3-chloro-5-dimethylcarbamoylpyridin-4-yl and the combination of R¹, R² and Y for a compound corresponds in each case to one row of Table A.

### Table 873

Compounds of the formula (I) in which X is n-propyl and Het is 3-chloro-5-thiocarbamoylpyridin-4-yl and the combination of R¹, R² and Y for a compound corresponds in each case to one row of Table A.

### Table 874

Compounds of the formula (I) in which X is n-propyl and Het is 3-chloro-5-nitropyridin-4-yl and the combination of R¹, R² and Y for a compound corresponds in each case to one row of Table A.

### Table 875

Compounds of the formula (I) in which X is n-propyl and Het is 3-chloro-5-acetylpyridin-4-yl and the combination of R¹, R² and Y for a compound corresponds in each case to one row of Table A.

### Table 876

Compounds of the formula (I) in which X is n-propyl and Het is 3,5-dimethylpyridin-4-yl and the combination of R¹, R² and Y for a compound corresponds in each case to one row of Table A.

### Table 877

Compounds of the formula (I) in which X is n-propyl and Het is 3-methyl-5-bromopyridin-4-yl and the combination of R¹, R² and Y for a compound corresponds in each case to one row of Table A.

### Table 878

Compounds of the formula (I) in which X is n-propyl and Het is 3-methyl-5-ethylpyridin-4-yl and the combination of R¹, R² and Y for a compound corresponds in each case to one row of Table A.

### Table 879

Compounds of the formula (I) in which X is n-propyl and Het is 3-methyl-5-methoxypyridin-4-yl and the combination of R¹, R² and Y for a compound corresponds in each case to one row of Table A.

### Table 880

Compounds of the formula (I) in which X is n-propyl and Het is 3-methyl-5-cyanopyridin-4-yl and the combination of R¹, R² and Y for a compound corresponds in each case to one row of Table A.

### Table 881

Compounds of the formula (I) in which X is n-propyl and Het is 3-methyl-5-methoxycarbonylpyridin-4-yl and the combination of R¹, R² and Y for a compound corresponds in each case to one row of Table A.

### Table 882

Compounds of the formula (I) in which X is n-propyl and Het is 3-methyl-5-ethoxycarbonylpyridin-4-yl and the combination of R¹, R² and Y for a compound corresponds in each case to one row of Table A.

### Table 883

Compounds of the formula (I) in which X is n-propyl and Het is 3-methyl-5-carbamoylpyridin-4-yl and the combination of R¹, R² and Y for a compound corresponds in each case to one row of Table A.

### Table 884

Compounds of the formula (I) in which X is h-propyl and Het is 3-methyl-5-methylcarbamoylpyridin-4-yl and the combination of R¹, R² and Y for a compound corresponds in each case to one row of Table A.

### Table 885

Compounds of the formula (I) in which X is n-propyl and Het is 3-methyl-5-dimethylcarbamoylpyridin-4-yl and the combination of R¹, R² and Y for a compound corresponds in each case to one row of Table A.

### Table 886

Compounds of the formula (I) in which X is n-propyl and Het is 3-methyl-5-thiocarbamoylpyridin-4-yl and the combination of R¹, R² and Y for a compound corresponds in each case to one row of Table A.

### Table 887

Compounds of the formula (I) in which X is n-propyl and Het is 3-methyl-5-nitropyridin-4-yl and the combination of R¹, R² and Y for a compound corresponds in each case to one row of Table A.

### Table 888

Compounds of the formula (I) in which X is n-propyl and Het is 3-methyl-5-acetylpyridin-4-yl and the combination of R¹, R² and Y for a compound corresponds in each case to one row of Table A.

### Table 889

Compounds of the formula (I) in which X is n-propyl and Het is 3-methylsulfanyl-5-fluoropyridin-4-yl and the combination of R¹, R² and Y for a compound corresponds in each case to one row of Table A.

### Table 890

Compounds of the formula (I) in which X is n-propyl and Het is 3-methylsulfanyl-5-chloropyridin-4-yl and the combination of R¹, R² and Y for a compound corresponds in each case to one row of Table A.

### Table 891

Compounds of the formula (I) in which X is n-propyl and Het is 3-methylsulfanyl-5-methylpyridin-4-yl and the combination of R¹, R² and Y for a compound corresponds in each case to one row of Table A.

### Table 892

Compounds of the formula (I) in which X is n-propyl and Het is 3-methylsulfinyl-5-fluoropyridin-4-yl and the combination of R¹, R² and Y for a compound corresponds in each case to one row of Table A.

### Table 893

Compounds of the formula (I) in which X is n-propyl and Het is 3-methylsulfinyl-5-chloropyridin-4-yl and the combination of R¹, R² and Y for a compound corresponds in each case to one row of Table A.

### Table 894

Compounds of the formula (I) in which X is n-propyl and Het is 3-methylsulfinyl-5-methylpyridin-4-yl and the combination of R¹, R² and Y for a compound corresponds in each case to one row of Table A.

### Table 895

Compounds of the formula (I) in which X is n-propyl and Het is 3-methylsulfonyl-5-fluoropyridin-4-yl and the combination of R¹, R² and Y for a compound corresponds in each case to one row of Table A.

### Table 896

Compounds of the formula (I) in which X is n-propyl and Het is 3-methylsulfonyl-5-chloropyridin-4-yl and the combination of R¹, R² and Y for a compound corresponds in each case to one row of Table A.

### Table 897

Compounds of the formula (I) in which X is n-propyl and Het is 3-methylsulfonyl-5-methylpyridin-4-yl and the combination of R¹, R² and Y for a compound corresponds in each case to one row of Table A.

### Table 898

Compounds of the formula (I) in which X is n-propyl and Het is 3-methoxymethyl-5-fluoropyridin-4-yl and the combination of R¹, R² and Y for a compound corresponds in each case to one row of Table A.

### Table 899

Compounds of the formula (I) in which X is n-propyl and Het is 3-methoxymethyl-5-chloropyridin-4-yl and the combination of R¹, R² and Y for a compound corresponds in each case to one row of Table A.

### Table 900

Compounds of the formula (I) in which X is n-propyl and Het is 3-methoxymethyl-5-methylpyridin-4-yl and the combination of R¹, R² and Y for a compound corresponds in each case to one row of Table A.

### Table 901

Compounds of the formula (I) in which X is n-propyl and Het is 2-fluoropyridin-3-yl and the combination of R¹, R² and Y for a compound corresponds in each case to one row of Table A.

### Table 902

Compounds of the formula (I) in which X is n-propyl and Het is 2-chloropyridin-3-yl and the combination of R¹, R² and Y for a compound corresponds in each case to one row of Table A.

### Table 903

Compounds of the formula (I) in which X is n-propyl and Het is 2-bromopyridin-3-yl and the combination of R¹, R² and Y for a compound corresponds in each case to one row of Table A.

### Table 904

Compounds of the formula (I) in which X is n-propyl and Het is 2-methylpyridin-3-yl and the combination of R¹, R² and Y for a compound corresponds in each case to one row of Table A.

### Table 905

Compounds of the formula (I) in which X is n-propyl and Het is 2-methoxypyridin-3-yl and the combination of R¹, R² and Y for a compound corresponds in each case to one row of Table A.

### Table 906

Compounds of the formula (I) in which X is n-propyl and Het is 2-methylsulfanylpyridin-3-yl and the combination of R¹, R² and Y for a compound corresponds in each case to one row of Table A.

### Table 907

Compounds of the formula (I) in which X is n-propyl and Het is 2-methylsulfinylpyridin-3-yl and the combination of R¹, R² and Y for a compound corresponds in each case to one row of Table A.

### Table 908

Compounds of the formula (I) in which X is n-propyl and Het is 2-methylsulfonylpyridin-3-yl and the combination of R¹, R² and Y for a compound corresponds in each case to one row of Table A.

### Table 909

Compounds of the formula (I) in which X is n-propyl and Het is 2-nitropyridin-3-yl and the combination of R¹, R² and Y for a compound corresponds in each case to one row of Table A.

### Table 910

Compounds of the formula (I) in which X is n-propyl and Het is 2-methoxymethylpyridin-3-yl and the combination of R¹, R² and Y for a compound corresponds in each case to one row of Table A.

### Table 911

Compounds of the formula (I) in which X is n-propyl and Het is 4-fluoropyridin-3-yl and the combination of R¹, R² and Y for a compound corresponds in each case to one row of Table A.

### Table 912

Compounds of the formula (I) in which X is n-propyl and Het is 4-chloropyridin-3-yl and the combination of R¹, R² and Y for a compound corresponds in each case to one row of Table A.

### Table 913

Compounds of the formula (I) in which X is n-propyl and Het is 4-bromopyridin-3-yl and the combination of R¹, R² and Y for a compound corresponds in each case to one row of Table A.

### Table 914

Compounds of the formula (I) in which X is n-propyl and Het is 4-methylpyridin-3-yl and the combination of R¹, R² and Y for a compound corresponds in each case to one row of Table A.

### Table 915

Compounds of the formula (I) in which X is n-propyl and Het is 4-methoxypyridin-3-yl and the combination of R¹, R² and Y for a compound corresponds in each case to one row of Table A.

### Table 916

Compounds of the formula (I) in which X is n-propyl and Het is 4-methylsulfanylpyridin-3-yl and the combination of R¹, R² and Y for a compound corresponds in each case to one row of Table A.

### Table 917

Compounds of the formula (I) in which X is n-propyl and Het is 4-methylsulfinylpyridin-3-yl and the combination of R¹, R² and Y for a compound corresponds in each case to one row of Table A.

### Table 918

Compounds of the formula (I) in which X is n-propyl and Het is 4-methylsulfonylpyridin-3-yl and the combination of R¹, R² and Y for a compound corresponds in each case to one row of Table A.

### Table 919

Compounds of the formula (I) in which X is n-propyl and Het is 4-nitropyridin-3-yl and the combination of R¹, R² and Y for a compound corresponds in each case to one row of Table A.

### Table 920

Compounds of the formula (I) in which X is n-propyl and Het is 4-methoxymethylpyridin-3-yl and the combination of R¹, R² and Y for a compound corresponds in each case to one row of Table A.

### Table 921

Compounds of the formula (I) in which X is n-propyl and Het is 2,4-difluoropyridin-3-yl and the combination of R¹, R² and Y for a compound corresponds in each case to one row of Table A.

### Table 922

Compounds of the formula (I) in which X is n-propyl and Het is 2-fluoro-4-chloropyridin-3-yl and the combination of R¹, R² and Y for a compound corresponds in each case to one row of Table A.

### Table 923

Compounds of the formula (I) in which X is n-propyl and Het is 2-fluoro-4-bromopyridin-3-yl and the combination of R¹, R² and Y for a compound corresponds in each case to one row of Table A.

### Table 924

Compounds of the formula (I) in which X is h-propyl and Het is 2-fluoro-4-methylpyridin-3-yl and the combination of R¹, R² and Y for a compound corresponds in each case to one row of Table A.

### Table 925

Compounds of the formula (I) in which X is n-propyl and Het is 2-fluoro-4-methoxypyridin-3-yl and the combination of R¹, R² and Y for a compound corresponds in each case to one row of Table A.

### Table 926

Compounds of the formula (I) in which X is n-propyl and Het is 2-fluoro-4-methylsulfanylpyridin-3-yl and the combination of R¹, R² and Y for a compound corresponds in each case to one row of Table A.

### Table 927

Compounds of the formula (I) in which X is n-propyl and Het is 2-fluoro-4-methylsulfinylpyridin-3-yl and the combination of R¹, R² and Y for a compound corresponds in each case to one row of Table A.

### Table 928

Compounds of the formula (I) in which X is n-propyl and Het is 2-fluoro-4-methylsulfonylpyridin-3-yl and the combination of R¹, R² and Y for a compound corresponds in each case to one row of Table A.

### Table 929

Compounds of the formula (I) in which X is n-propyl and Het is 2-fluoro-4-nitropyridin-3-yl and the combination of R¹, R² and Y for a compound corresponds in each case to one row of Table A.

### Table 930

Compounds of the formula (I) in which X is n-propyl and Het is 2-fluoro-4-methoxymethylpyridin-3-yl and the combination of R¹, R² and Y for a compound corresponds in each case to one row of Table A.

### Table 931

Compounds of the formula (I) in which X is n-propyl and Het is 2,4-dichloropyridin-3-yl and the combination of R¹, R² and Y for a compound corresponds in each case to one row of Table A.

### Table 932

Compounds of the formula (I) in which X is n-propyl and Het is 2-chloro-4-fluoropyridin-3-yl and the combination of R¹, R² and Y for a compound corresponds in each case to one row of Table A.

### Table 933

Compounds of the formula (I) in which X is n-propyl and Het is 2-chloro-4-bromopyridin-3-yl and the combination of R¹, R² and Y for a compound corresponds in each case to one row of Table A.

### Table 934

Compounds of the formula (I) in which X is n-propyl and Het is 2-chloro-4-methylpyridin-3-yl and the combination of R¹, R² and Y for a compound corresponds in each case to one row of Table A.

### Table 935

Compounds of the formula (I) in which X is n-propyl and Het is 2-chloro-4-methoxypyridin-3-yl and the combination of R¹, R² and Y for a compound corresponds in each case to one row of Table A.

### Table 936

Compounds of the formula (I) in which X is n-propyl and Het is 2-chloro-4-methylsulfanylpyridin-3-yl and the combination of R¹, R² and Y for a compound corresponds in each case to one row of Table A.

### Table 937

Compounds of the formula (I) in which X is n-propyl and Het is 2-chloro-4-methylsulfinylpyridin-3-yl and the combination of R¹, R² and Y for a compound corresponds in each case to one row of Table A.

### Table 938

Compounds of the formula (I) in which X is n-propyl and Het is 2-chloro-4-methylsulfonylpyridin-3-yl and the combination of R¹, R² and Y for a compound corresponds in each case to one row of Table A.

### Table 939

Compounds of the formula (I) in which X is n-propyl and Het is 2-chloro-4-nitropyridin-3-yl and the combination of R¹, R² and Y for a compound corresponds in each case to one row of Table A.

### Table 940

Compounds of the formula (I) in which X is n-propyl and Het is 2-chloro-4-methoxymethylpyridin-3-yl and the combination of R¹, R² and Y for a compound corresponds in each case to one row of Table A.

### Table 941

Compounds of the formula (I) in which X is n-propyl and Het is 2,4-dimethylpyridin-3-yl and the combination of R¹, R² and Y for a compound corresponds in each case to one row of Table A.

### Table 942

Compounds of the formula (I) in which X is n-propyl and Het is 2-methyl-4-chloropyridin-3-yl and the combination of R¹, R² and Y for a compound corresponds in each case to one row of Table A.

### Table 943

Compounds of the formula (I) in which X is n-propyl and Het is 2-methyl-4-fluoropyridin-3-yl and the combination of R¹, R² and Y for a compound corresponds in each case to one row of Table A.

### Table 944

Compounds of the formula (I) in which X is n-propyl and Het is 2-methyl-4-bromopyridin-3-yl and the combination of R¹, R² and Y for a compound corresponds in each case to one row of Table A.

### Table 945

Compounds of the formula (I) in which X is n-propyl and Het is 2-methyl-4-methoxypyridin-3-yl and the combination of R¹, R² and Y for a compound corresponds in each case to one row of Table A.

### Table 946

Compounds of the formula (I) in which X is n-propyl and Het is 2-methyl-4-methylsulfanylpyridin-3-yl and the combination of R¹, R² and Y for a compound corresponds in each case to one row of Table A.

### Table 947

Compounds of the formula (I) in which X is n-propyl and Het is 2-methyl-4-methylsulfinylpyridin-3-yl and the combination of R¹, R² and Y for a compound corresponds in each case to one row of Table A.

### Table 948

Compounds of the formula (I) in which X is n-propyl and Het is 2-methyl-4-methylsulfonylpyridin-3-yl and the combination of R¹, R² and Y for a compound corresponds in each case to one row of Table A.

### Table 949

Compounds of the formula (I) in which X is n-propyl and Het is 2-methyl-4-nitropyridin-3-yl and the combination of R¹, R² and Y for a compound corresponds in each case to one row of Table A.

### Table 950

Compounds of the formula (I) in which X is n-propyl and Het is 2-methyl-4-methoxymethylpyridin-3-yl and the combination of R¹, R² and Y for a compound corresponds in each case to one row of Table A.

### Table 951

Compounds of the formula (I) in which X is n-propyl and Het is 2-bromo-4-fluoropyridin-3-yl and the combination of R¹, R² and Y for a compound corresponds in each case to one row of Table A.

### Table 952

Compounds of the formula (I) in which X is n-propyl and Het is 2-bromo-4-chloropyridin-3-yl and the combination of R¹, R² and Y for a compound corresponds in each case to one row of Table A.

### Table 953

Compounds of the formula (I) in which X is n-propyl and Het is 2-bromo-4-methylpyridin-3-yl and the combination of R¹, R² and Y for a compound corresponds in each case to one row of Table A.

### Table 954

Compounds of the formula (I) in which X is n-propyl and Het is 2-methoxy-4-fluoropyridin-3-yl and the combination of R¹, R² and Y for a compound corresponds in each case to one row of Table A.

### Table 955

Compounds of the formula (I) in which X is n-propyl and Het is 2-methoxy-4-chloropyridin-3-yl and the combination of R¹, R² and Y for a compound corresponds in each case to one row of Table A.

### Table 956

Compounds of the formula (I) in which X is n-propyl and Het is 2-methoxy-4-methylpyridin-3-yl and the combination of R¹, R² and Y for a compound corresponds in each case to one row of Table A.

### Table 957

Compounds of the formula (I) in which X is n-propyl and Het is 2-methylsulfanyl-4-fluoropyridin-3-yl and the combination of R¹, R² and Y for a compound corresponds in each case to one row of Table A.

### Table 958

Compounds of the formula (I) in which X is n-propyl and Het is 2-methylsulfanyl-4-chloropyridin-3-yl and the combination of R¹, R² and Y for a compound corresponds in each case to one row of Table A.

### Table 959

Compounds of the formula (I) in which X is n-propyl and Het is 2-methylsulfanyl-4-methylpyridin-3-yl and the combination of R¹, R² and Y for a compound corresponds in each case to one row of Table A.

### Table 960

Compounds of the formula (I) in which X is n-propyl and Het is 2-methylsulfinyl-4-fluoropyridin-3-yl and the combination of R¹, R² and Y for a compound corresponds in each case to one row of Table A.

### Table 961

Compounds of the formula (I) in which X is n-propyl and Het is 2-methylsulfinyl-4-chloropyridin-3-yl and the combination of R¹, R² and Y for a compound corresponds in each case to one row of Table A.

### Table 962

Compounds of the formula (I) in which X is n-propyl and Het is 2-methylsulfinyl-4-methylpyridin-3-yl and the combination of R¹, R² and Y for a compound corresponds in each case to one row of Table A.

### Table 963

Compounds of the formula (I) in which X is n-propyl and Het is 2-methylsulfonyl-4-fluoropyridin-3-yl and the combination of R¹, R² and Y for a compound corresponds in each case to one row of Table A.

### Table 964

Compounds of the formula (I) in which X is n-propyl and Het is 2-methylsulfonyl-4-chloropyridin-3-yl and the combination of R¹, R² and Y for a compound corresponds in each case to one row of Table A.

### Table 965

Compounds of the formula (I) in which X is h-propyl and Het is 2-methylsulfonyl-4-methylpyridin-3-yl and the combination of R¹, R² and Y for a compound corresponds in each case to one row of Table A.

### Table 966

Compounds of the formula (I) in which X is n-propyl and Het is 2-nitro-4-fluoropyridin-3-yl and the combination of R¹, R² and Y for a compound corresponds in each case to one row of Table A.

### Table 967

Compounds of the formula (I) in which X is n-propyl and Het is 2-nitro-4-chloropyridin-3-yl and the combination of R¹, R² and Y for a compound corresponds in each case to one row of Table A.

### Table 968

Compounds of the formula (I) in which X is n-propyl and Het is 2-nitro-4-methylpyridin-3-yl and the combination of R¹, R² and Y for a compound corresponds in each case to one row of Table A.

### Table 969

Compounds of the formula (I) in which X is n-propyl and Het is 2-methoxymethyl-4-fluoropyridin-3-yl and the combination of R¹, R² and Y for a compound corresponds in each case to one row of Table A.

### Table 970

Compounds of the formula (I) in which X is n-propyl and Het is 2-methoxymethyl-4-chloropyridin-3-yl and the combination of R¹, R² and Y for a compound corresponds in each case to one row of Table A.

### Table 971

Compounds of the formula (I) in which X is n-propyl and Het is 2-methoxymethyl-4-methylpyridin-3-yl and the combination of R¹, R² and Y for a compound corresponds in each case to one row of Table A.

### Table 972

Compounds of the formula (I) in which X is n-propyl and Het is 4,6-difluoropyridin-3-yl and the combination of R¹, R² and Y for a compound corresponds in each case to one row of Table A.

### Table 973

Compounds of the formula (I) in which X is n-propyl and Het is 4-fluoro-6-chloropyridin-3-yl and the combination of R¹, R² and Y for a compound corresponds in each case to one row of Table A.

### Table 974

Compounds of the formula (I) in which X is n-propyl and Het is 4-fluoro-6-bromopyridin-3-yl and the combination of R¹, R² and Y for a compound corresponds in each case to one row of Table A.

### Table 975

Compounds of the formula (I) in which X is n-propyl and Het is 4-fluoro-6-methylpyridin-3-yl and the combination of R¹, R² and Y for a compound corresponds in each case to one row of Table A.

### Table 976

Compounds of the formula (I) in which X is n-propyl and Het is 4-fluoro-6-ethylpyridin-3-yl and the combination of R¹, R² and Y for a compound corresponds in each case to one row of Table A.

### Table 977

Compounds of the formula (I) in which X is n-propyl and Het is 4-fluoro-6-methoxypyridin-3-yl and the combination of R¹, R² and Y for a compound corresponds in each case to one row of Table A.

### Table 978

Compounds of the formula (I) in which X is n-propyl and Het is 4-fluoro-6-cyanopyridin-3-yl and the combination of R¹, R² and Y for a compound corresponds in each case to one row of Table A.

### Table 979

Compounds of the formula (I) in which X is n-propyl and Het is 4-fluoro-6-methoxycarbonylpyridin-3-yl and the combination of R¹, R² and Y for a compound corresponds in each case to one row of Table A.

### Table 980

Compounds of the formula (I) in which X is n-propyl and Het is 4-fluoro-6-ethoxycarbonylpyridin-3-yl and the combination of R¹, R² and Y for a compound corresponds in each case to one row of Table A.

### Table 981

Compounds of the formula (I) in which X is n-propyl and Het is 4-fluoro-6-carbamoylpyridin-3-yl and the combination of R¹, R² and Y for a compound corresponds in each case to one row of Table A.

### Table 982

Compounds of the formula (I) in which X is n-propyl and Het is 4-fluoro-6-methylcarbamoylpyridin-3-yl and the combination of R¹, R² and Y for a compound corresponds in each case to one row of Table A.

### Table 983

Compounds of the formula (I) in which X is n-propyl and Het is 4-fluoro-6-dimethylcarbamoylpyridin-3-yl and the combination of R¹, R² and Y for a compound corresponds in each case to one row of Table A.

### Table 984

Compounds of the formula (I) in which X is n-propyl and Het is 4-fluoro-6-thiocarbamoylpyridin-3-yl and the combination of R¹, R² and Y for a compound corresponds in each case to one row of Table A.

### Table 985

Compounds of the formula (I) in which X is n-propyl and Het is 4-fluoro-6-nitropyridin-3-yl and the combination of R¹, R² and Y for a compound corresponds in each case to one row of Table A.

### Table 986

Compounds of the formula (I) in which X is n-propyl and Het is 4-fluoro-6-acetylpyridin-3-yl and the combination of R¹, R² and Y for a compound corresponds in each case to one row of Table A.

### Table 987

Compounds of the formula (I) in which X is n-propyl and Het is 4,6-dichloropyridin-3-yl and the combination of R¹, R² and Y for a compound corresponds in each case to one row of Table A.

### Table 988

Compounds of the formula (I) in which X is n-propyl and Het is 4-chloro-6-fluoropyridin-3-yl and the combination of R¹, R² and Y for a compound corresponds in each case to one row of Table A.

### Table 989

Compounds of the formula (I) in which X is h-propyl and Het is 4-chloro-6-bromopyridin-3-yl and the combination of R¹, R² and Y for a compound corresponds in each case to one row of Table A.

### Table 990

Compounds of the formula (I) in which X is n-propyl and Het is 4-chloro-6-methylpyridin-3-yl and the combination of R¹, R² and Y for a compound corresponds in each case to one row of Table A.

### Table 991

Compounds of the formula (I) in which X is n-propyl and Het is 4-chloro-6-ethylpyridin-3-yl and the combination of R¹, R² and Y for a compound corresponds in each case to one row of Table A.

### Table 992

Compounds of the formula (I) in which X is n-propyl and Het is 4-chloro-6-methoxypyridin-3-yl and the combination of R¹, R² and Y for a compound corresponds in each case to one row of Table A.

### Table 993

Compounds of the formula (I) in which X is n-propyl and Het is 4-chloro-6-cyanopyridin-3-yl and the combination of R¹, R² and Y for a compound corresponds in each case to one row of Table A.

### Table 994

Compounds of the formula (I) in which X is n-propyl and Het is 4-chloro-6-methoxycarbonylpyridin-3-yl and the combination of R¹, R² and Y for a compound corresponds in each case to one row of Table A.

### Table 995

Compounds of the formula (I) in which X is n-propyl and Het is 4-chloro-6-ethoxycarbonylpyridin-3-yl and the combination of R¹, R² and Y for a compound corresponds in each case to one row of Table A.

### Table 996

Compounds of the formula (I) in which X is n-propyl and Het is 4-chloro-6-carbamoylpyridin-3-yl and the combination of R¹, R² and Y for a compound corresponds in each case to one row of Table A.

### Table 997

Compounds of the formula (I) in which X is n-propyl and Het is 4-chloro-6-methylcarbamoylpyridin-3-yl and the combination of R¹, R² and Y for a compound corresponds in each case to one row of Table A.

### Table 998

Compounds of the formula (I) in which X is n-propyl and Het is 4-chloro-6-dimethylcarbamoylpyridin-3-yl and the combination of R¹, R² and Y for a compound corresponds in each case to one row of Table A.

### Table 999

Compounds of the formula (I) in which X is n-propyl and Het is 4-chloro-6-thiocarbamoylpyridin-3-yl and the combination of R¹, R² and Y for a compound corresponds in each case to one row of Table A.

### Table 1000

Compounds of the formula (I) in which X is n-propyl and Het is 4-chloro-6-nitropyridin-3-yl and the combination of R¹, R² and Y for a compound corresponds in each case to one row of Table A.

### Table 1001

Compounds of the formula (I) in which X is n-propyl and Het is 4-chloro-6-acetylpyridin-3-yl and the combination of R¹, R² and Y for a; compound corresponds in each case to one row of Table A.

### Table 1002

Compounds of the formula (I) in which X is n-propyl and Het is 4-methyl-6-fluoropyridin-3-yl and the combination of R¹, R² and Y for a compound corresponds in each case to one row of Table A.

### Table 1003

Compounds of the formula (I) in which X is n-propyl and Het is 4-methyl-6-chloropyridin-3-yl and the combination of R¹, R² and Y for a compound corresponds in each case to one row of Table A.

### Table 1004

Compounds of the formula (I) in which X is n-propyl and Het is 4-methyl-6-bromopyridin-3-yl and the combination of R¹, R² and Y for a compound corresponds in each case to one row of Table A.

### Table 1005

Compounds of the formula (I) in which X is n-propyl and Het is 4,6-dimethylpyridin-3-yl and the combination of R¹, R² and Y for a compound corresponds in each case to one row of Table A.

### Table 1006

Compounds of the formula (I) in which X is n-propyl and Het is 4-methyl-6-ethylpyridin-3-yl and the combination of R¹, R² and Y for a compound corresponds in each case to one row of Table A.

### Table 1007

Compounds of the formula (I) in which X is n-propyl and Het is 4-methyl-6-methoxypyridin-3-yl and the combination of R¹, R² and Y for a compound corresponds in each case to one row of Table A.

### Table 1008

Compounds of the formula (I) in which X is n-propyl and Het is 4-methyl-6-cyanopyridin-3-yl and the combination of R¹, R² and Y for a compound corresponds in each case to one row of Table A.

### Table 1009

Compounds of the formula (I) in which X is n-propyl and Het is 4-methyl-6-methoxycarbonylpyridin-3-yl and the combination of R¹, R² and Y for a compound corresponds in each case to one row of Table A.

### Table 1010

Compounds of the formula (I) in which X is n-propyl and Het is 4-methyl-6-ethoxycarbonylpyridin-3-yl and the combination of R¹, R² and Y for a compound corresponds in each case to one row of Table A.

### Table 1011

Compounds of the formula (I) in which X is n-propyl and Het is 4-methyl-6-carbamoylpyridin-3-yl and the combination of R¹, R² and Y for a compound corresponds in each case to one row of Table A.

### Table 1012

Compounds of the formula (I) in which X is n-propyl and Het is 4-methyl-6-methylcarbamoylpyridin-3-yl and the combination of R¹, R² and Y for a compound corresponds in each case to one row of Table A.

### Table 1013

Compounds of the formula (I) in which X is n-propyl and Het is 4-methyl-6-dimethylcarbamoylpyridin-3-yl and the combination of R¹, R² and Y for a compound corresponds in each case to one row of Table A.

### Table 1014

Compounds of the formula (I) in which X is n-propyl and Het is 4-methyl-6-thiocarbamoylpyridin-3-yl and the combination of R¹, R² and Y for a compound corresponds in each case to one row of Table A.

### Table 1015

Compounds of the formula (I) in which X is n-propyl and Het is 4-methyl-6-nitropyridin-3-yl and the combination of R¹, R² and Y for a compound corresponds in each case to one row of Table A.

### Table 1016

Compounds of the formula (I) in which X is n-propyl and Het is 4-methyl-6-acetylpyridin-3-yl and the combination of R¹, R² and Y for a compound corresponds in each case to one row of Table A.

### Table 1017

Compounds of the formula (I) in which X is n-propyl and Het is 4-bromo-6-fluoropyridin-3-yl and the combination of R¹, R² and Y for a compound corresponds in each case to one row of Table A.

### Table 1018

Compounds of the formula (I) in which X is n-propyl and Het is 4-bromo-6-chloropyridin-3-yl and the combination of R¹, R² and Y for a compound corresponds in each case to one row of Table A.

### Table 1019

Compounds of the formula (I) in which X is n-propyl and Het is 4-bromo-6-methylpyridin-3-yl and the combination of R¹, R² and Y for a compound corresponds in each case to one row of Table A.

### Table 1020

Compounds of the formula (I) in which X is n-propyl and Het is 4-methoxy-6-fluoropyridin-3-yl and the combination of R¹, R² and Y for a compound corresponds in each case to one row of Table A.

### Table 1021

Compounds of the formula (I) in which X is n-propyl and Het is 4-methoxy-6-chloropyridin-3-yl and the combination of R¹, R² and Y for a compound corresponds in each case to one row of Table A.

### Table 1022

Compounds of the formula (I) in which X is n-propyl and Het is 4-methoxy-6-methylpyridin-3-yl and the combination of R¹, R² and Y for a compound corresponds in each case to one row of Table A.

### Table 1023

Compounds of the formula (I) in which X is n-propyl and Het is 4-methylsulfanyl-6-fluoropyridin-3-yl and the combination of R¹, R² and Y for a compound corresponds in each case to one row of Table A.

### Table 1024

Compounds of the formula (I) in which X is n-propyl and Het is 4-methylsulfanyl-6-chloropyridin-3-yl and the combination of R¹, R² and Y for a compound corresponds in each case to one row of Table A.

### Table 1025

Compounds of the formula (I) in which X is n-propyl and Het is 4-methylsulfanyl-6-methylpyridin-3-yl and the combination of R¹, R² and Y for a compound corresponds in each case to one row of Table A.

### Table 1026

Compounds of the formula (I) in which X is n-propyl and Het is 4-methylsulfinyl-6-fluoropyridin-3-yl and the combination of R¹, R² and Y for a compound corresponds in each case to one row of Table A.

### Table 1027

Compounds of the formula (I) in which X is n-propyl and Het is 4-methylsulfinyl-6-chloropyridin-3-yl and the combination of R¹, R² and Y for a compound corresponds in each case to one row of Table A.

### Table 1028

Compounds of the formula (I) in which X is n-propyl and Het is 4-methylsulfinyl-6-methylpyridin-3-yl and the combination of R¹, R² and Y for a compound corresponds in each case to one row of Table A.

### Table 1029

Compounds of the formula (I) in which X is n-propyl and Het is 4-methylsulfonyl-6-fluoropyridin-3-yl and the combination of R¹, R² and Y for a compound corresponds in each case to one row of Table A.

### Table 1030

Compounds of the formula (I) in which X is n-propyl and Het is 4-methylsulfonyl-6-chloropyridin-3-yl and the combination of R¹, R² and Y for a compound corresponds in each case to one row of Table A.

### Table 1031

Compounds of the formula (I) in which X is n-propyl and Het is 4-methylsulfonyl-6-methylpyridin-3-yl and the combination of R¹, R² and Y for a compound corresponds in each case to one row of Table A.

### Table 1032

Compounds of the formula (I) in which X is n-propyl and Het is 4-nitro-6-fluoropyridin-3-yl and the combination of R¹, R² and Y for a compound corresponds in each case to one row of Table A.

### Table 1033

Compounds of the formula (i) in which X is n-propyl and Het is 4-nitro-6-chloropyridin-3-yl and the combination of R¹, R² and Y for a compound corresponds in each case to one row of Table A.

### Table 1034

Compounds of the formula (I) in which X is n-propyl and Het is 4-nitro-6-methylpyridin-3-yl and the combination of R¹, R² and Y for a compound corresponds in each case to one row of Table A.

### Table 1035

Compounds of the formula (I) in which X is n-propyl and Het is 4-methoxymethyl-6-fluoropyridin-3-yl and the combination of R¹, R² and Y for a compound corresponds in each case to one row of Table A.

### Table 1036

Compounds of the formula (I) in which X is n-propyl and Het is 4-methoxymethyl-6-chloropyridin-3-yl and the combination of R¹, R² and Y for a compound corresponds in each case to one row of Table A.

### Table 1037

Compounds of the formula (I) in which X is n-propyl and Het is 4-methoxymethyl-6-methylpyridin-3-yl and the combination of R¹, R² and Y for a compound corresponds in each case to one row of Table A.

### Table 1038

Compounds of the formula (I) in which X is n-propyl and Het is 2,4-dichlor-6-methylpyridin-3-yl and the combination of R¹, R² and Y for a compound corresponds in each case to one row of Table A.

### Table 1039

Compounds of the formula (I) in which X is methoxymethyl and Het is 2-pyridinyl and the combination of R¹, R² and Y for a compound corresponds in each case to one row of Table A.

### Table 1040

Compounds of the formula (I) in which X is methoxymethyl and Het is 5-nitropyridin-2-yl and the combination of R¹, R² and Y for a compound corresponds in each case to one row of table A.

### Table 1041

Compounds of the formula (I) in which X is methoxymethyl and Het is 5-cyano-pyridin-2-yl and the combination of R¹, R² and Y for a compound corresponds in each case to one row of Table A.

### Table 1042

Compounds of the formula (I) in which X is methoxymethyl and Het is 5-methoxy-carbonylpyridin-2-yl and the combination of R¹, R² and Y for a compound corresponds in each case to one row of Table A.

### Table 1043

Compounds of the formula (I) in which X is methoxymethyl and Het is 5-methyl-pyridin-2-yl and the combination of R¹, R² and Y for a compound corresponds in each case to one row of Table A.

### Table 1044

Compounds of the formula (I) in which X is methoxymethyl and Het is 4-methyl-pyridin-2-yl and the combination of R¹, R² and Y for a compound corresponds in each case to one row of Table A.

### Table 1045

Compounds of the formula (I) in which X is methoxymethyl and Het is 3-methyl-pyridin-2-yl and the combination of R¹, R² and Y for a compound corresponds in each case to one row of Table A.

### Table 1046

Compounds of the formula (I) in which X is methoxymethyl and Het is 3-ethyl-pyridin-2-yl and the combination of R¹, R² and Y for a compound corresponds in each case to one row of Table A.

### Table 1047

Compounds of the formula (I) in which X is methoxymethyl and Het is 6-methyl-pyridin-2-yl and the combination of R¹, R² and Y for a compound corresponds in each case to one row of Table A.

### Table 1048

Compounds of the formula (I) in which X is methoxymethyl and Het is 5-trifluoromethylpyridin-2-yl and the combination of R¹, R² and Y for a compound corresponds in each case to one row of Table A.

### Table 1049

Compounds of the formula (I) in which X is methoxymethyl and Het is 3-trifluoromethylpyridin-2-yl and the combination of R¹, R² and Y for a compound corresponds in each case to one row of Table A.

### Table 1050

Compounds of the formula (I) in which X is methoxymethyl and Het is 5-fluoropyridin-2-yl and the combination of R¹, R² and Y for a compound corresponds in each case to one row of Table A.

### Table 1051

Compounds of the formula (I) in which X is methoxymethyl and Het is 3-fluoropyridin-2-yl and the combination of R¹, R² and Y for a compound corresponds in each case to one row of Table A.

### Table 1052

Compounds of the formula (I) in which X is methoxymethyl and Het is 3,5-difluoropyridin-2-yl and the combination of R¹, R² and Y for a compound corresponds in each case to one row of Table A.

### Table 1053

Compounds of the formula (I) in which X is methoxymethyl and Het is 3,5-dichloropyridin-2-yl and the combination of R¹, R² and Y for a compound corresponds in each case to one row of Table A.

### Table 1054

Compounds of the formula (I) in which X is methoxymethyl and Het is pyridin-3-yl and the combination of R¹, R² and Y for a compound corresponds in each case to one row of Table A.

Table 1055

Compounds of the formula (I) in which X is methoxymethyl and Het is pyridin-4-yl and the combination of R¹, R² and Y for a compound corresponds in each case to one row of Table A.

### Table 1056

Compounds of the formula (I) in which X is methoxymethyl and Het is pyrazin-2-yl and the combination of R¹, R² and Y for a compound corresponds in each case to one row of Table A.

### Table 1057

Compounds of the formula (I) in which X is methoxymethyl and Het is pyridazin-3-yl and the combination of R¹, R² and Y for a compound corresponds in each case to one row of Table A.

### Table 1058

Compounds of the formula (I) in which X is methoxymethyl and Het is 6-chloropyridazin-3-yl and the combination of R¹, R² and Y for a compound corresponds in each case to one row of Table A.

### Table 1059

Compounds of the formula (I) in which X is methoxymethyl and Het is 6-methoxy-pyridazin-3-yl and the combination of R¹, R² and Y for a compound corresponds in each case to one row of Table A.

### Table 1060

Compounds of the formula (I) in which X is methoxymethyl and Het is 1,3,5-triazin-2-yl and the combination of R¹, R² and Y for a compound corresponds in each case to one row of Table A.

### Table 1061

Compounds of the formula (I) in which X is methoxymethyl and Het is pyrimidin-2-yl and the combination of R¹, R² and Y for a compound corresponds in each case to one row of Table A.

### Table 1062

Compounds of the formula (I) in which X is methoxymethyl and Het is pyrimidin-4-yl and the combination of R¹, R² and Y for a compound corresponds in each case to one row of Table A.

### Table 1063

Compounds of the formula (I) in which X is methoxymethyl and Het is pyrimidin-5-yl and the combination of R¹, R² and Y for a compound corresponds in each case to one row of Table A.

### Table 1064

Compounds of the formula (I) in which X is methoxymethyl and Het is 2-methyl-6-trifluoromethylpyrimidin-4-yl and the combination of R¹, R² and Y for a compound corresponds in each case to one row of Table A.

### Table 1065

Compounds of the formula (I) in which X is methoxymethyl and Het is 2,5-dimethyl-6-trifluoromethylpyrimidin-4-yl and the combination of R¹, R² and Y for a compound corresponds in each case to one row of Table A.

### Table 1066

Compounds of the formula (I) in which X is methoxymethyl and Het is 6-trifluoromethylpyrimidin-4-yl and the combination of R¹, R² and Y for a compound corresponds in each case to one row of Table A.

### Table 1067

Compounds of the formula (I) in which X is methoxymethyl and Het is 5-methyl-6-trifluoromethylpyrimidin-4-yl and the combination of R¹, R² and Y for a compound corresponds in each case to one row of Table A.

### Table 1068

Compounds of the formula (I) in which X is methoxymethyl and Het is 5-chloro-6-methylpyrimidin-4-yl and the combination of R¹, R² and Y for a compound corresponds in each case to one row of Table A.

### Table 1069

Compounds of the formula (I) in which X is methoxymethyl and Het is 5-bromo-6-methylpyrimidin-4-yl and the combination of R¹, R² and Y for a compound corresponds in each case to one row of Table A.

### Table 1070

Compounds of the formula (I) in which X is methoxymethyl and Het is 5-fluoro-6-methylpyrimidin-4-yl and the combination of R¹, R² and Y for a compound corresponds in each case to one row of Table A.

### Table 1071

Compounds of the formula (I) in which X is methoxymethyl and Het is 5-chloro-6-ethylpyrimidin-4-yl and the combination of R¹, R² and Y for a compound corresponds in each case to one row of Table A.

### Table 1072

Compounds of the formula (I) in which X is methoxymethyl and Het is 5-bromo-6-ethylpyrimidin-4-yl and the combination of R¹, R² and Y for a compound corresponds in each case to one row of Table A.

### Table 1073

Compounds of the formula (I) in which X is methoxymethyl and Het is 5-chloro-6-isopropylpyrimidin-4-yl and the combination of R¹, R² and Y for a compound corresponds in each case to one row of Table A.

### Table 1074

Compounds of the formula (I) in which X is methoxymethyl and Het is 5-bromo-6-isopropylpyrimidin-4-yl and the combination of R¹, R² and Y for a compound corresponds in each case to one row of Table A.

### Table 1075

Compounds of the formula (I) in which X is methoxymethyl and Het is 5-chloropyrimidin-4-yl and the combination of R¹, R² and Y for a compound corresponds in each case to one row of Table A.

### Table 1076

Compounds of the formula (I) in which X is methoxymethyl and Het is 5-fluoropyrimidin-4-yl and the combination of R¹, R² and Y for a compound corresponds in each case to one row of Table A.

### Table 1077

Compounds of the formula (I) in which X is methoxymethyl and Het is 5-bromopyrimidin-4-yl and the combination of R¹, R² and Y for a compound corresponds in each case to one row of Table A.

### Table 1078

Compounds of the formula (I) in which X is methoxymethyl and Het is 2-methyl-5-fluoropyrimidin-4-yl and the combination of R¹, R² and Y for a compound corresponds in each case to one row of Table A.

### Table 1079

Compounds of the formula (I) in which X is methoxymethyl and Het is 2-methyl-5-chloropyrimidin-4-yl and the combination of R¹, R² and Y for a compound corresponds in each case to one row of Table A.

### Table 1080

Compounds of the formula (I) in which X is methoxymethyl and Het is 2-methyl-5-bromopyrimidin-4-yl and the combination of R¹, R² and Y for a compound corresponds in each case to one row of Table A.

### Table 1081

Compounds of the formula (I) in which X is methoxymethyl and Het is 2,6-dimethyl-5-chloropyrimidin-4-yl and the combination of R¹, R² and Y for a compound corresponds in each case to one row of Table A.

### Table 1082

Compounds of the formula (I) in which X is methoxymethyl and Het is 2,6-dimethyl-5-bromopyrimidin-4-yl and the combination of R¹, R² and Y for a compound corresponds in each case to one row of Table A.

### Table 1083

Compounds of the formula (I) in which X is methoxymethyl and Het is 5-fluoro-6-fluoromethylpyrimidin-4-yl and the combination of R¹, R² and Y for a compound corresponds in each case to one row of Table A.

### Table 1084

Compounds of the formula (I) in which X is methoxymethyl and Het is 5-chloro-6-methoxymethylpyrimidin-4-yl and the combination of R¹, R² and Y for a compound corresponds in each case to one row of Table A.

### Table 1085

Compounds of the formula (I) in which X is methoxymethyl and Het is 5,6-dimethylpyrimidin-4-yl and the combination of R¹, R² and Y for a compound corresponds in each case to one row of Table A.

### Table 1086

Compounds of the formula (I) in which X is methoxymethyl and Het is 2,5-dimethylpyrimidin-4-yl and the combination of R¹, R² and Y for a compound corresponds in each case to one row of Table A.

### Table 1087

Compounds of the formula (I) in which X is methoxymethyl and Het is 2,5,6-trimethylpyrimidin-4-yl and the combination of R¹, R² and Y for a compound corresponds in each case to one row of Table A.

### Table 1088

Compounds of the formula (I) in which X is methoxymethyl and Het is 5-methyl-6-methoxypyrimidin-4-yl and the combination of R¹, R² and Y for a compound corresponds in each case to one row of Table A.

### Table 1089

Compounds of the formula (I) in which X is methoxymethyl and Het is 4,6-dimethylpyrimidin-2-yl and the combination of R¹, R² and Y for a compound corresponds in each case to one row of Table A.

### Table 1090

Compounds of the formula (I) in which X is methoxymethyl and Het is 4,5,6-trimethylpyrimidin-2-yl and the combination of R¹, R² and Y for a compound corresponds in each case to one row of Table A.

### Table 1091

Compounds of the formula (I) in which X is methoxymethyl and Het is 4,6-di(trifluoromethyl)pyrimidin-2-yl and the combination of R¹, R² and Y for a compound corresponds in each case to one row of Table A.

### Table 1092

Compounds of the formula (I) in which X is methoxymethyl and Het is 4,6-dimethyl-5-chloropyrimidin-2-yl and the combination of R¹, R² and Y for a compound corresponds in each case to one row of Table A.

### Table 1093

Compounds of the formula (I) in which X is methoxymethyl and Het is 4,6-dimethyl-5-bromopyrimidin-2-yl and the combination of R¹, R² and Y for a compound corresponds in each case to one row of Table A.

### Table 1094

Compounds of the formula (I) in which X is methoxymethyl and Het is 4-methylpyrimidin-5-yl and the combination of R¹, R² and Y for a compound corresponds in each case to one row of Table A.

### Table 1095

Compounds of the formula (I) in which X is methoxymethyl and Het is 4,6-dimethylpyrimidin-5-yl and the combination of R¹, R² and Y for a compound corresponds in each case to one row of Table A.

### Table 1096

Compounds of the formula (I) in which X is methoxymethyl and Het is 4-trifluoromethyl-6-methylpyrimidin-5-yl and the combination of R¹, R² and Y for a compound corresponds in each case to one row of Table A.

### Table 1097

Compounds of the formula (I) in which X is methoxymethyl and Het is 2,4,6-trimethylpyrimidin-5-yl and the combination of R¹, R² and Y for a compound corresponds in each case to one row of Table A.

### Table 1098

Compounds of the formula (I) in which X is methoxymethyl and Het is 3-chloropyridin-2-yl and the combination of R¹, R² and Y for a compound corresponds in each case to one row of Table A.

### Table 1099

Compounds of the formula (I) in which X is methoxymethyl and Het is 3-bromopyridin-2-yl and the combination of R¹, R² and Y for a compound corresponds in each case to one row of Table A.

### Table 1100

Compounds of the formula (I) in which X is methoxymethyl and Het is 3-iodopyridin-2-yl and the combination of R¹, R² and Y for a compound corresponds in each case to one row of Table A.

### Table 1101

Compounds of the formula (I) in which X is methoxymethyl and Het is 3-methoxypyridin-2-yl and the combination of R¹, R² and Y for a compound corresponds in each case to one row of Table A.

### Table 1102

Compounds of the formula (I) in which X is methoxymethyl and Het is 3-methylsulfanylpyridin-2-yl and the combination of R¹, R² and Y for a compound corresponds in each case to one row of Table A.

### Table 1103

Compounds of the formula (I) in which X is methoxymethyl and Het is 3-methylsulfinylpyridin-2-yl and the combination of R¹, R² and Y for a compound corresponds in each case to one row of Table A.

### Table 1104

Compounds of the formula (I) in which X is methoxymethyl and Het is 3-methylsulfonylpyridin-2-yl and the combination of R¹, R² and Y for a compound corresponds in each case to one row of Table A.

### Table 1105

Compounds of the formula (I) in which X is methoxymethyl and Het is 3-nitropyridin-2-yl and the combination of R¹, R² and Y for a compound corresponds in each case to one row of Table A.

### Table 1106

Compounds of the formula (I) in which X is methoxymethyl and Het is 3-methoxymethylpyridin-2-yl and the combination of R¹, R² and Y for a compound corresponds in each case to one row of Table A.

### Table 1107

Compounds of the formula (I) in which X is methoxymethyl and Het is 5-chloropyridin-2-yl and the combination of R¹, R² and Y for a compound corresponds in each case to one row of Table A.

### Table 1108

Compounds of the formula (I) in which X is methoxymethyl and Het is 5-bromopyridin-2-yl and the combination of R¹, R² and Y for a compound corresponds in each case to one row of Table A.

### Table 1109

Compounds of the formula (I) in which X is methoxymethyl and Het is 5-methoxypyridin-2-yl and the combination of R¹, R² and Y for a compound corresponds in each case to one row of Table A.

### Table 1110

Compounds of the formula (I) in which X is methoxymethyl and Het is 5-methylsulfanylpyridin-2-yl and the combination of R¹, R² and Y for a compound corresponds in each case to one row of Table A.

### Table 1111

Compounds of the formula (I) in which X is methoxymethyl and Het is 5-methylsulfinylpyridin-2-yl and the combination of R¹, R² and Y for a compound corresponds in each case to one row of Table A.

### Table 1112

Compounds of the formula (I) in which X is methoxymethyl and Het is 5-methylsulfonylpyridin-2-yl and the combination of R¹, R² and Y for a compound corresponds in each case to one row of Table A.

### Table 1113

Compounds of the formula (I) in which X is methoxymethyl and Het is 5-methoxymethylpyridin-2-yl and the combination of R¹, R² and Y for a compound corresponds in each case to one row of Table A.

### Table 1114

Compounds of the formula (I) in which X is methoxymethyl and Het is 5-ethylpyridin-2-yl and the combination of R¹, R² and Y for a compound corresponds in each case to one row of Table A.

### Table 1115

Compounds of the formula (I) in which X is methoxymethyl and Het is 5-ethoxycarbonylpyridin-2-yl and the combination of R¹, R² and Y for a compound corresponds in each case to one row of Table A.

### Table 1116

Compounds of the formula (I) in which X is methoxymethyl and Het is 5-carbamoylpyridin-2-yl and the combination of R¹, R² and Y for a compound corresponds in each case to one row of Table A.

### Table 1117

Compounds of the formula (I) in which X is methoxymethyl and Het is 5-methylcarbamoylpyridin-2-yl and the combination of R¹, R² and Y for a compound corresponds in each case to one row of Table A.

### Table 1118

Compounds of the formula (I) in which X is methoxymethyl and Het is 5-dimethylcarbamoylpyridin-2-yl and the combination of R¹, R² and Y for a compound corresponds in each case to one row of Table A.

### Table 1119

Compounds of the formula (I) in which X is methoxymethyl and Het is 5-thiocarbamoylpyridin-2-yl and the combination of R¹, R² and Y for a compound corresponds in each case to one row of Table A.

### Table 1120

Compounds of the formula (I) in which X is methoxymethyl and Het is 5-acetylpyridin-2-yl and the combination of R¹, R² and Y for a compound corresponds in each case to one row of Table A.

### Table 1121

Compounds of the formula (I) in which X is methoxymethyl and Het is 5-methylpyridin-2-yl and the combination of R¹, R² and Y for a compound corresponds in each case to one row of Table A.

### Table 1122

Compounds of the formula (I) in which X is methoxymethyl and Het is 5-nitropyridin-2-yl and the combination of R¹, R² and Y for a compound corresponds in each case to one row of Table A.

### Table 1123

Compounds of the formula (I) in which X is methoxymethyl and Het is 5-cyanopyridin-2-yl and the combination of R¹, R² and Y for a compound corresponds in each case to one row of Table A.

### Table 1124

Compounds of the formula (I) in which X is methoxymethyl and Het is 3-fluoro-5-chloropyridin-2-yl and the combination of R¹, R² and Y for a compound corresponds in each case to one row of Table A.

### Table 1125

Compounds of the formula (I) in which X is methoxymethyl and Het is 3-fluoro-5-bromopyridin-2-yl and the combination of R¹, R² and Y for a compound corresponds in each case to one row of Table A.

### Table 1126

Compounds of the formula (I) in which X is methoxymethyl and Het is 3-fluoro-5-methylpyridin-2-yl and the combination of R¹, R² and Y for a compound corresponds in each case to one row of Table A.

### Table 1127

Compounds of the formula (I) in which X is methoxymethyl and Het is 3-fluoro-5-ethylpyridin-2-yl and the combination of R¹, R² and Y for a compound corresponds in each case to one row of Table A.

### Table 1128

Compounds of the formula (I) in which X is methoxymethyl and Het is 3-fluoro-5-methoxypyridin-2-yl and the combination of R¹, R² and Y for a compound corresponds in each case to one row of Table A.

### Table 1129

Compounds of the formula (I) in which X is methoxymethyl and Het is 3-fluoro-5-cyanopyridin-2-yl and the combination of R¹, R² and Y for a compound corresponds in each case to one row of Table A.

### Table 1130

Compounds of the formula (I) in which X is methoxymethyl and Het is 3-fluoro-5-methoxycarbonylpyridin-2-yl and the combination of R¹, R² and Y for a compound corresponds in each case to one row of Table A.

### Table 1131

Compounds of the formula (I) in which X is methoxymethyl and Het is 3-fluoro-5-ethoxycarbonylpyridin-2-yl and the combination of R¹, R² and Y for a compound corresponds in each case to one row of Table A.

### Table 1132

Compounds of the formula (I) in which X is methoxymethyl and Het is 3-fluoro-5-carbamoylpyridin-2-yl and the combination of R¹, R² and Y for a compound corresponds in each case to one row of Table A.

### Table 1133

Compounds of the formula (I) in which X is methoxymethyl and Het is 3-fluoro-5-methylcarbamoylpyridin-2-yl and the combination of R¹, R² and Y for a compound corresponds in each case to one row of Table A.

### Table 1134

Compounds of the formula (I) in which X is methoxymethyl and Het is 3-fluoro-5-dimethylcarbamoylpyridin-2-yl and the combination of R¹, R² and Y for a compound corresponds in each case to one row of Table A.

### Table 1135

Compounds of the formula (I) in which X is methoxymethyl and Het is 3-fluoro-5-thiocarbamoylpyridin-2-yl and the combination of R¹, R² and Y for a compound corresponds in each case to one row of Table A.

### Table 1136

Compounds of the formula (I) in which X is methoxymethyl and Het is 3-fluoro-5-nitropyridin-2-yl and the combination of R¹, R² and Y for a compound corresponds in each case to one row of Table A.

### Table 1137

Compounds of the formula (I) in which X is methoxymethyl and Het is 3-fluoro-5-acetylpyridin-2-yl and the combination of R¹, R² and Y for a compound corresponds in each case to one row of Table A.

### Table 1138

Compounds of the formula (I) in which X is methoxymethyl and Het is 3-chloro-5-fluoropyridin-2-yl and the combination of R¹, R² and Y for a compound corresponds in each case to one row of Table A.

### Table 1139

Compounds of the formula (I) in which X is methoxymethyl and Het is 3-chloro-5-bromopyridin-2-yl and the combination of R¹, R² and Y for a compound corresponds in each case to one row of Table A.

### Table 1140

Compounds of the formula (I) in which X is methoxymethyl and Het is 3-chloro-5-methylpyridin-2-yl and the combination of R¹, R² and Y for a compound corresponds in each case to one row of Table A.

### Table 1141

Compounds of the formula (I) in which X is methoxymethyl and Het is 3-chloro-5-ethylpyridin-2-yl and the combination of R¹, R² and Y for a compound corresponds in each case to one row of Table A.

### Table 1142

Compounds of the formula (I) in which X is methoxymethyl and Het is 3-chloro-5-methoxypyridin-2-yl and the combination of R¹, R² and Y for a compound corresponds in each case to one row of Table A.

### Table 1143

Compounds of the formula (I) in which X is methoxymethyl and Het is 3-chloro-5-cyanopyridin-2-yl and the combination of R¹, R² and Y for a compound corresponds in each case to one row of Table A.

### Table 1144

Compounds of the formula (I) in which X is methoxymethyl and Het is 3-chloro-5-methoxycarbonylpyridin-2-yl and the combination of R¹, R² and Y for a compound corresponds in each case to one row of Table A.

### Table 1145

Compounds of the formula (I) in which X is methoxymethyl and Het is 3-chloro-5-ethoxycarbonylpyridin-2-yl and the combination of R¹, R² and Y for a compound corresponds in each case to one row of Table A.

### Table 1146

Compounds of the formula (I) in which X is methoxymethyl and Het is 3-chloro-5-carbamoylpyridin-2-yl and the combination of R¹, R² and Y for a compound corresponds in each case to one row of Table A.

### Table 1147

Compounds of the formula (I) in which X is methoxymethyl and Het is 3-chloro-5-methylcarbamoylpyridin-2-yl and the combination of R¹, R² and Y for a compound corresponds in each case to one row of Table A.

### Table 1148

Compounds of the formula (I) in which X is methoxymethyl and Het is 3-chloro-5-dimethylcarbamoylpyridin-2-yl and the combination of R¹, R² and Y for a compound corresponds in each case to one row of Table A.

### Table 1149

Compounds of the formula (I) in which X is methoxymethyl and Het is 3-chloro-5-thiocarbamoylpyridin-2-yl and the combination of R¹, R² and Y for a compound corresponds in each case to one row of Table A.

### Table 1150

Compounds of the formula (I) in which X is methoxymethyl and Het is 3-chloro-5-nitropyridin-2-yl and the combination of R¹, R² and Y for a compound corresponds in each case to one row of Table A.

### Table 1151

Compounds of the formula (I) in which X is methoxymethyl and Het is 3-chloro-5-acetylpyridin-2-yl and the combination of R¹, R² and Y for a compound corresponds in each case to one row of Table A.

### Table 1152

Compounds of the formula (I) in which X is methoxymethyl and Het is 3-methyl-5-fluoropyridin-2-yl and the combination of R¹, R² and Y for a compound corresponds in each case to one row of Table A.

### Table 1153

Compounds of the formula (I) in which X is methoxymethyl and Het is 3-methyl-5-chloropyridin-2-yl and the combination of R¹, R² and Y for a compound corresponds in each case to one row of Table A.

### Table 1154

Compounds of the formula (I) in which X is methoxymethyl and Het is 3-methyl-5-bromopyridin-2-yl and the combination of R¹, R² and Y for a compound corresponds in each case to one row of Table A.

### Table 1155

Compounds of the formula (I) in which X is methoxymethyl and Het is 3,5-dimethylpyridin-2-yl and the combination of R¹, R² and Y for a compound corresponds in each case to one row of Table A.

### Table 1156

Compounds of the formula (I) in which X is methoxymethyl and Het is 3-methyl-5-ethylpyridin-2-yl and the combination of R¹, R² and Y for a compound corresponds in each case to one row of Table A.

### Table 1157

Compounds of the formula (I) in which X is methoxymethyl and Het is 3-methyl-5-methoxypyridin-2-yl and the combination of R¹, R² and Y for a compound corresponds in each case to one row of Table A.

### Table 1158

Compounds of the formula (I) in which X is methoxymethyl and Het is 3-methyl-5-cyanopyridin-2-yl and the combination of R¹, R² and Y for a compound corresponds in each case to one row of Table A.

### Table 1159

Compounds of the formula (I) in which X is methoxymethyl and Het is 3-methyl-5-methoxycarbonylpyridin-2-yl and the combination of R¹, R² and Y for a compound corresponds in each case to one row of Table A.

### Table 1160

Compounds of the formula (I) in which X is methoxymethyl and Het is 3-methyl-5-ethoxycarbonylpyridin-2-yl and the combination of R¹, R² and Y for a compound corresponds in each case to one row of Table A.

### Table 1161

Compounds of the formula (I) in which X is methoxymethyl and Het is 3-methyl-5-carbamoylpyridin-2-yl and the combination of R¹, R² and Y for a compound corresponds in each case to one row of Table A.

### Table 1162

Compounds of the formula (I) in which X is methoxymethyl and Het is 3-methyl-5-methylcarbamoylpyridin-2-yl and the combination of R¹, R² and Y for a compound corresponds in each case to one row of Table A.

### Table 1163

Compounds of the formula (I) in which X is methoxymethyl and Het is 3-methyl-5-dimethylcarbamoylpyridin-2-yl and the combination of R¹, R² and Y for a compound corresponds in each case to one row of Table A.

### Table 1164

Compounds of the formula (I) in which X is methoxymethyl and Het is 3-methyl-5-thiocarbamoylpyridin-2-yl and the combination of R¹, R² and Y for a compound corresponds in each case to one row of Table A.

### Table 1165

Compounds of the formula (I) in which X is methoxymethyl and Het is 3-methyl-5-nitropyridin-2-yl and the combination of R¹, R² and Y for a compound corresponds in each case to one row of Table A.

### Table 1166

Compounds of the formula (I) in which X is methoxymethyl and Het is 3-methyl-5-acetylpyridin-2-yl and the combination of R¹, R² and Y for a compound corresponds in each case to one row of Table A.

### Table 1167

Compounds of the formula (I) in which X is methoxymethyl and Het is 3-bromo-5-fluoropyridin-2-yl and the combination of R¹, R² and Y for a compound corresponds in each case to one row of Table A.

### Table 1168

Compounds of the formula (I) in which X is methoxymethyl and Het is 3-bromo-5-chloropyridin-2-yl and the combination of R¹, R² and Y for a compound corresponds in each case to one row of Table A.

### Table 1169

Compounds of the formula (I) in which X is methoxymethyl and Het is 3-bromo-5-methylpyridin-2-yl and the combination of R¹, R² and Y for a compound corresponds in each case to one row of Table A.

### Table 1170

Compounds of the formula (I) in which X is methoxymethyl and Het is 3-methylsulfonyl-5-fluoropyridin-2-yl and the combination of R¹, R² and Y for a compound corresponds in each case to one row of Table A.

### Table 1171

Compounds of the formula (I) in which X is methoxymethyl and Het is 3-methylsulfonyl-5-chloropyridin-2-yl and the combination of R¹, R² and Y for a compound corresponds in each case to one row of Table A.

### Table 1172

Compounds of the formula (I) in which X is methoxymethyl and Het is 3-methylsulfonyl-5-methylpyridin-2-yl and the combination of R¹, R² and Y for a compound corresponds in each case to one row of Table A.

### Table 1173

Compounds of the formula (I) in which X is methoxymethyl and Het is 3-methoxy-5-fluoropyridin-2-yl and the combination of R¹, R² and Y for a compound corresponds in each case to one row of Table A.

### Table 1174

Compounds of the formula (I) in which X is methoxymethyl and Het is 3-methoxy-5-chloropyridin-2-yl and the combination of R¹, R² and Y for a compound corresponds in each case to one row of Table A.

### Table 1175

Compounds of the formula (I) in which X is methoxymethyl and Het is 3-methoxy-5-methylpyridin-2-yl and the combination of R¹, R² and Y for a compound corresponds in each case to one row of Table A.

### Table 1176

Compounds of the formula (I) in which X is methoxymethyl and Het is 3-methylsulfanyl-5-fluoropyridin-2-yl and the combination of R¹, R² and Y for a compound corresponds in each case to one row of Table A.

### Table 1177

Compounds of the formula (I) in which X is methoxymethyl and Het is 3-methylsulfanyl-5-chloropyridin-2-yl and the combination of R¹, R² and Y for a compound corresponds in each case to one row of Table A.

### Table 1178

Compounds of the formula (I) in which X is methoxymethyl and Het is 3-methylsulfanyl-5-methylpyridin-2-yl and the combination of R¹, R² and Y for a compound corresponds in each case to one row of Table A.

### Table 1179

Compounds of the formula (I) in which X is methoxymethyl and Het is 3-nitro-5-fluoropyridin-2-yl and the combination of R¹, R² and Y for a compound corresponds in each case to one row of Table A.

### Table 1180

Compounds of the formula (I) in which X is methoxymethyl and Het is 3-nitro-5-chloropyridin-2-yl and the combination of R¹, R² and Y for a compound corresponds in each case to one row of Table A.

### Table 1181

Compounds of the formula (I) in which X is methoxymethyl and Het is 3-nitro-5-methylpyridin-2-yl and the combination of R¹, R² and Y for a compound corresponds in each case to one row of Table A.

### Table 1182

Compounds of the formula (I) in which X is methoxymethyl and Het is 3,5-dibromopyridin-2-yl and the combination of R¹, R² and Y for a compound corresponds in each case to one row of Table A.

### Table 1183

Compounds of the formula (I) in which X is methoxymethyl and Het is 3-fluoropyridin-4-yl and the combination of R¹, R² and Y for a compound corresponds in each case to one row of Table A.

### Table 1184

Compounds of the formula (I) in which X is methoxymethyl and Het is 3-chloropyridin-4-yl and the combination of R¹, R² and Y for a compound corresponds in each case to one row of Table A.

### Table 1185

Compounds of the formula (I) in which X is methoxymethyl and Het is 3-bromopyridin-4-yl and the combination of R¹, R² and Y for a compound corresponds in each case to one row of Table A.

### Table 1186

Compounds of the formula (I) in which X is methoxymethyl and Het is 3-methylpyridin-4-yl and the combination of R¹, R² and Y for a compound corresponds in each case to one row of Table A.

### Table 1187

Compounds of the formula (I) in which X is methoxymethyl and Het is 3-methoxypyridin-4-yl and the combination of R¹, R² and Y for a compound corresponds in each case to one row of Table A.

### Table 1188.

Compounds of the formula (I) in which X is methoxymethyl and Het is 3-methylsulfanylpyridin-4-yl and the combination of R¹, R² and Y for a compound corresponds in each case to one row of Table A.

### Table 1189

Compounds of the formula (I) in which X is methoxymethyl and Het is 3-methylsulfinylpyridin-4-yl and the combination of R¹, R² and Y for a compound corresponds in each case to one row of Table A.

### Table 1190

Compounds of the formula (I) in which X is methoxymethyl and Het is 3-methylsulfonylpyridin-4-yl and the combination of R¹, R² and Y for a compound corresponds in each case to one row of Table A.

### Table 1191

Compounds of the formula (I) in which X is methoxymethyl and Het is 3-nitropyridin-4-yl and the combination of R¹, R² and Y for a compound corresponds in each case to one row of Table A.

### Table 1192

Compounds of the formula (I) in which X is methoxymethyl and Het is 3-methoxymethylpyridin-4-yl and the combination of R¹, R² and Y for a compound corresponds in each case to one row of Table A.

### Table 1193

Compounds of the formula (I) in which X is methoxymethyl and Het is 3,5-difluoropyridin-4-yl and the combination of R¹, R² and Y for a compound corresponds in each case to one row of Table A.

### Table 1194

Compounds of the formula (I) in which X is methoxymethyl and Het is 3-fluoro-5-chloropyridin-4-yl and the combination of R¹, R² and Y for a compound corresponds in each case to one row of Table A.

### Table 1195

Compounds of the formula (I) in which X is methoxymethyl and Het is 3-fluoro-5-bromopyridin-4-yl and the combination of R¹, R² and Y for a compound corresponds in each case to one row of Table A.

### Table 1196

Compounds of the formula (I) in which X is methoxymethyl and Het is 3-fluoro-5-methylpyridin-4-yl and the combination of R¹, R² and Y for a compound corresponds in each case to one row of Table A.

### Table 1197

Compounds of the formula (I) in which X is methoxymethyl and Het is 3-fluoro-5-ethylpyridin-4-yl and the combination of R¹, R² and Y for a compound corresponds in each case to one row of Table A.

### Table 1198

Compounds of the formula (I) in which X is methoxymethyl and Het is 3-fluoro-5-methoxypyridin-4-yl and the combination of R¹, R² and Y for a compound corresponds in each case to one row of Table A.

### Table 1199

Compounds of the formula (I) in which X is methoxymethyl and Het is 3-fluoro-5-cyanopyridin-4-yl and the combination of R¹, R² and Y for a compound corresponds in each case to one row of Table A.

### Table 1200

Compounds of the formula (I) in which X is methoxymethyl and Het is 3-fluoro-5-methoxycarbonylpyridin-4-yl and the combination of R¹, R² and Y for a compound corresponds in each case to one row of Table A.

### Table 1201

Compounds of the formula (I) in which X is methoxymethyl and Het is 3-fluoro-5-ethoxycarbonylpyridin-4-yl and the combination of R¹, R² and Y for a compound corresponds in each case to one row of Table A.

### Table 1202

Compounds of the formula (I) in which X is methoxymethyl and Het is 3-fluoro-5-carbamoylpyridin-4-yl and the combination of R¹, R² and Y for a compound corresponds in each case to one row of Table A.

### Table 1203

Compounds of the formula (I) in which X is methoxymethyl and Het is 3-fluoro-5-methylcarbamoylpyridin-4-yl and the combination of R¹, R² and Y for a compound corresponds in each case to one row of Table A.

### Table 1204

Compounds of the formula (I) in which X is methoxymethyl and Het is 3-fluoro-5-dimethylcarbamoylpyridin-4-yl and the combination of R¹, R² and Y for a compound corresponds in each case to one row of Table A.

### Table 1205

Compounds of the formula (I) in which X is methoxymethyl and Het is 3-fluoro-5-thiocarbamoylpyridin-4-yl and the combination of R¹, R² and Y for a compound corresponds in each case to one row of Table A.

### Table 1206

Compounds of the formula (I) in which X is methoxymethyl and Het is 3-fluoro-5-nitropyridin-4-yl and the combination of R¹, R² and Y for a compound corresponds in each case to one row of Table A.

### Table 1207

Compounds of the formula (I) in which X is methoxymethyl and Het is 3-fluoro-5-acetylpyridin-4-yl and the combination of R¹, R² and Y for a compound corresponds in each case to one row of Table A.

### Table 1208

Compounds of the formula (I) in which X is methoxymethyl and Het is 3,5-dichloro-5-chloropyridin-4-yl and the combination of R¹, R² and Y for a compound corresponds in each case to one row of Table A.

### Table 1209

Compounds of the formula (I) in which X is methoxymethyl and Het is 3-chloro-5-bromopyridin-4-yl and the combination of R¹, R² and Y for a compound corresponds in each case to one row of Table A.

### Table 1210

Compounds of the formula (I) in which X is methoxymethyl and Het is 3-chloro-5-methylpyridin-4-yl and the combination of R¹, R² and Y for a compound corresponds in each case to one row of Table A.

### Table 1211

Compounds of the formula (I) in which X is methoxymethyl and Het is 3-chloro-5-ethylpyridin-4-yl and the combination of R¹, R² and Y for a compound corresponds in each case to one row of Table A.

### Table 1212

Compounds of the formula (I) in which X is methoxymethyl and Het is 3-chloro-5-methoxypyridin-4-yl and the combination of R¹, R² and Y for a compound corresponds in each case to one row of Table A.

### Table 1213

Compounds of the formula (I) in which X is methoxymethyl and Het is 3-chloro-5-cyanopyridin-4-yl and the combination of R¹, R² and Y for a compound corresponds in each case to one row of Table A.

### Table 1214

Compounds of the formula (I) in which X is methoxymethyl and Het is 3-chloro-5-methoxycarbonylpyridin-4-yl and the combination of R¹, R² and Y for a compound corresponds in each case to one row of Table A.

### Table 1215

Compounds of the formula (I) in which X is methoxymethyl and Het is 3-chloro-5-ethoxycarbonylpyridin-4-yl and the combination of R¹, R² and Y for a compound corresponds in each case to one row of Table A.

### Table 1216

Compounds of the formula (I) in which X is methoxymethyl and Het is 3-chloro-5-carbamoylpyridin-4-yl and the combination of R¹, R² and Y for a compound corresponds in each case to one row of Table A.

### Table 1217

Compounds of the formula (I) in which X is methoxymethyl and Het is 3-chloro-5-methylcarbamoylpyridin-4-yl and the combination of R¹, R² and Y for a compound corresponds in each case to one row of Table A.

### Table 1218

Compounds of the formula (I) in which X is methoxymethyl and Het is 3-chloro-5-dimethylcarbamoylpyridin-4-yl and the combination of R¹, R² and Y for a compound corresponds in each case to one row of Table A.

### Table 1219

Compounds of the formula (I) in which X is methoxymethyl and Het is 3-chloro-5-thiocarbamoylpyridin-4-yl and the combination of R¹, R² and Y for a compound corresponds in each case to one row of Table A.

### Table 1220

Compounds of the formula (I) in which X is methoxymethyl and Het is 3-chloro-5-nitropyridin-4-yl and the combination of R¹, R² and Y for a compound corresponds in each case to one row of Table A.

### Table 1221

Compounds of the formula (I) in which X is methoxymethyl and Het is 3-chloro-5-acetylpyridin-4-yl and the combination of R¹, R² and Y for a compound corresponds in each case to one row of Table A.

### Table 1222

Compounds of the formula (I) in which X is methoxymethyl and Het is 3,5-dimethylpyridin-4-yl and the combination of R¹, R² and Y for a compound corresponds in each case to one row of Table A.

### Table 1223

Compounds of the formula (I) in which X is methoxymethyl and Het is 3-methyl-5-bromopyridin-4-yl and the combination of R¹, R² and Y for a compound corresponds in each case to one row of Table A.

### Table 1224

Compounds of the formula (I) in which X is methoxymethyl and Het is 3-methyl-5-ethylpyridin-4-yl and the combination of R¹, R² and Y for a compound corresponds in each case to one row of Table A.

### Table 1225

Compounds of the formula (I) in which X is methoxymethyl and Het is 3-methyl-5-methoxypyridin-4-yl and the combination of R¹, R² and Y for a compound corresponds in each case to one row of Table A.

### Table 1226

Compounds of the formula (I) in which X is methoxymethyl and Het is 3-methyl-5-cyanopyridin-4-yl and the combination of R¹, R² and Y for a compound corresponds in each case to one row of Table A.

### Table 1227

Compounds of the formula (I) in which X is methoxymethyl and Het is 3-methyl-5-methoxycarbonylpyridin-4-yl and the combination of R¹, R² and Y for a compound corresponds in each case to one row of Table A.

### Table 1228

Compounds of the formula (I) in which X is methoxymethyl and Het is 3-methyl-5-ethoxycarbonylpyridin-4-yl and the combination of R¹, R² and Y for a compound corresponds in each case to one row of Table A.

### Table 1229

Compounds of the formula (I) in which X is methoxymethyl and Het is 3-methyl-5-carbamoylpyridin-4-yl and the combination of R¹, R² and Y for a compound corresponds in each case to one row of Table A.

### Table 1230

Compounds of the formula (I) in which X is methoxymethyl and Het is 3-methyl-5-methylcarbamoylpyridin-4-yl and the combination of R¹, R² and Y for a compound corresponds in each case to one row of Table A.

### Table 1231

Compounds of the formula (I) in which X is methoxymethyl and Het is 3-methyl-5-dimethylcarbamoylpyridin-4-yl and the combination of R¹, R² and Y for a compound corresponds in each case to one row of Table A.

### Table 1232

Compounds of the formula (I) in which X is methoxymethyl and Het is 3-methyl-5-thiocarbamoylpyridin-4-yl and the combination of R¹, R² and Y for a compound corresponds in each case to one row of Table A.

### Table 1233

Compounds of the formula (I) in which X is methoxymethyl and Het is 3-methyl-5-nitropyridin-4-yl and the combination of R¹, R² and Y for a compound corresponds in each case to one row of Table A.

### Table 1234

Compounds of the formula (I) in which X is methoxymethyl and Het is 3-methyl-5-acetylpyridin-4-yl and the combination of R¹, R² and Y for a compound corresponds in each case to one row of Table A.

### Table 1235

Compounds of the formula (I) in which X is methoxymethyl and Het is 3-methylsulfanyl-5-fluoropyridin-4-yl and the combination of R¹, R² and Y for a compound corresponds in each case to one row of Table A.

### Table 1236

Compounds of the formula (I) in which X is methoxymethyl and Het is 3-methylsulfanyl-5-chloropyridin-4-yl and the combination of R¹, R² and Y for a compound corresponds in each case to one row of Table A.

### Table 1237

Compounds of the formula (I) in which X is methoxymethyl and Het is 3-methylsulfanyl-5-methylpyridin-4-yl and the combination of R¹, R² and Y for a compound corresponds in each case to one row of Table A.

### Table 1238

Compounds of the formula (I) in which X is methoxymethyl and Het is 3-methylsulfinyl-5-fluoropyridin-4-yl and the combination of R¹, R² and Y for a compound corresponds in each case to one row of Table A.

### Table 1239

Compounds of the formula (I) in which X is methoxymethyl and Het is 3-methylsulfinyl-5-chloropyridin-4-yl and the combination of R¹, R² and Y for a compound corresponds in each case to one row of Table A.

### Table 1240

Compounds of the formula (I) in which X is methoxymethyl and Het is 3-methylsulfinyl-5-methylpyridin-4-yl and the combination of R¹, R² and Y for a compound corresponds in each case to one row of Table A.

### Table 1241

Compounds of the formula (I) in which X is methoxymethyl and Het is 3-methylsulfonyl-5-fluoropyridin-4-yl and the combination of R¹, R² and Y for a compound corresponds in each case to one row of Table A.

### Table 1242

Compounds of the formula (I) in which X is methoxymethyl and Het is 3-methylsulfonyl-5-chloropyridin-4-yl and the combination of R¹, R² and Y for a compound corresponds in each case to one row of Table A.

### Table 1243

Compounds of the formula (I) in which X is methoxymethyl and Het is 3-methylsulfonyl-5-methylpyridin-4-yl and the combination of R¹, R² and Y for a compound corresponds in each case to one row of Table A.

### Table 1244

Compounds of the formula (I) in which X is methoxymethyl and Het is 3-methoxymethyl-5-fluoropyridin-4-yl and the combination of R¹, R² and Y for a compound corresponds in each case to one row of Table A.

### Table 1245

Compounds of the formula (I) in which X is methoxymethyl and Het is 3-methoxymethyl-5-chloropyridin-4-yl and the combination of R¹, R² and Y for a compound corresponds in each case to one row of Table A.

### Table 1246

Compounds of the formula (I) in which X is methoxymethyl and Het is 3-methoxymethyl-5-methylpyridin-4-yl and the combination of R¹, R² and Y for a compound corresponds in each case to one row of Table A.

### Table 1247

Compounds of the formula (I) in which X is methoxymethyl and Het is 2-fluoropyridin-3-yl and the combination of R¹, R² and Y for a compound corresponds in each case to one row of Table A.

### Table 1248

Compounds of the formula (I) in which X is methoxymethyl and Het is 2-chloropyridin-3-yl and the combination of R¹, R² and Y for a compound corresponds in each case to one row of Table A.

### Table 1249

Compounds of the formula (I) in which X is methoxymethyl and Het is 2-bromopyridin-3-yl and the combination of R¹, R² and Y for a compound corresponds in each case to one row of Table A.

### Table 1250

Compounds of the formula (I) in which X is methoxymethyl and Het is 2-methylpyridin-3-yl and the combination of R¹, R² and Y for a compound corresponds in each case to one row of Table A.

### Table 1251

Compounds of the formula (I) in which X is methoxymethyl and Het is 2-methoxypyridin-3-yl and the combination of R¹, R² and Y for a compound corresponds in each case to one row of Table A.

### Table 1252

Compounds of the formula (I) in which X is methoxymethyl and Het is 2-methylsulfanylpyridin-3-yl and the combination of R¹, R² and Y for a compound corresponds in each case to one row of Table A.

### Table 1253

Compounds of the formula (I) in which X is methoxymethyl and Het is 2-methylsulfinylpyridin-3-yl and the combination of R¹, R² and Y for a compound corresponds in each case to one row of Table A.

### Table 1254

Compounds of the formula (I) in which X is methoxymethyl and Het is 2-methylsulfonylpyridin-3-yl and the combination of R¹, R² and Y for a compound corresponds in each case to one row of Table A.

### Table 1255

Compounds of the formula (I) in which X is methoxymethyl and Het is 2-nitropyridin-3-yl and the combination of R¹, R² and Y for a compound corresponds in each case to one row of Table A.

### Table 1256

Compounds of the formula (I) in which X is methoxymethyl and Het is 2-methoxymethylpyridin-3-yl and the combination of R¹, R² and Y for a compound corresponds in each case to one row of Table A.

### Table 1257

Compounds of the formula (I) in which X is methoxymethyl and Het is 4-fluoropyridin-3-yl and the combination of R¹, R² and Y for a compound corresponds in each case to one row of Table A.

### Table 1258

Compounds of the formula (I) in which X is methoxymethyl and Het is 4-chloropyridin-3-yl and the combination of R¹, R² and Y for a compound corresponds in each case to one row of Table A.

### Table 1259

Compounds of the formula (I) in which X is methoxymethyl and Het is 4-bromopyridin-3-yl and the combination of R¹, R² and Y for a compound corresponds in each case to one row of Table A.

### Table 1260

Compounds of the formula (I) in which X is methoxymethyl and Het is 4-methylpyridin-3-yl and the combination of R¹, R² and Y for a compound corresponds in each case to one row of Table A.

### Table 1261

Compounds of the formula (I) in which X is methoxymethyl and Het is 4-methoxypyridin-3-yl and the combination of R¹, R² and Y for a compound corresponds in each case to one row of Table A.

### Table 1262

Compounds of the formula (I) in which X is methoxymethyl and Het is 4-methylsulfanylpyridin-3-yl and the combination of R¹, R² and Y for a compound corresponds in each case to one row of Table A.

### Table 1263

Compounds of the formula (I) in which X is methoxymethyl and Het is 4-methylsulfinylpyridin-3-yl and the combination of R¹, R² and Y for a compound corresponds in each case to one row of Table A.

### Table 1264

Compounds of the formula (I) in which X is methoxymethyl and Het is 4-methylsulfonylpyridin-3-yl and the combination of R¹, R² and Y for a compound corresponds in each case to one row of Table A.

### Table 1265

Compounds of the formula (I) in which X is methoxymethyl and Het is 4-nitropyridin-3-yl and the combination of R¹, R² and Y for a compound corresponds in each case to one row of Table A.

### Table 1266

Compounds of the formula (I) in which X is methoxymethyl and Het is 4-methoxymethylpyridin-3-yl and the combination of R¹, R² and Y for a compound corresponds in each case to one row of Table A.

### Table 1267

Compounds of the formula (I) in which X is methoxymethyl and Het is 2,4-difluoropyridin-3-yl and the combination of R¹, R² and Y for a compound corresponds in each case to one row of Table A.

### Table 1268

Compounds of the formula (I) in which X is methoxymethyl and Het is 2-fluoro-4-chloropyridin-3-yl and the combination of R¹, R² and Y for a compound corresponds in each case to one row of Table A.

### Table 1269

Compounds of the formula (I) in which X is methoxymethyl and Het is 2-fluoro-4-bromopyridin-3-yl and the combination of R¹, R² and Y for a compound corresponds in each case to one row of Table A.

### Table 1270

Compounds of the formula (I) in which X is methoxymethyl and Het is 2-fluoro-4-methylpyridin-3-yl and the combination of R¹, R² and Y for a compound corresponds in each case to one row of Table A.

### Table 1271

Compounds of the formula (I) in which X is methoxymethyl and Het is 2-fluoro-4-methoxypyridin-3-yl and the combination of R¹, R² and Y for a compound corresponds in each case to one row of Table A.

### Table 1272

Compounds of the formula (I) in which X is methoxymethyl and Het is 2-fluoro-4-methylsulfanylpyridin-3-yl and the combination of R¹, R² and Y for a compound corresponds in each case to one row of Table A.

### Table 1273

Compounds of the formula (I) in which X is methoxymethyl and Het is 2-fluoro-4-methylsulfinylpyridin-3-yl and the combination of R¹, R² and Y for a compound corresponds in each case to one row of Table A.

### Table 1274

Compounds of the formula (I) in which X is methoxymethyl and Het is 2-fluoro-4-methylsulfonylpyridin-3-yl and the combination of R¹, R² and Y for a compound corresponds in each case to one row of Table A.

### Table 1275

Compounds of the formula (I) in which X is methoxymethyl and Het is 2-fluoro-4-nitropyridin-3-yl and the combination of R¹, R² and Y for a compound corresponds in each case to one row of Table A.

### Table 1276

Compounds of the formula (I) in which X is methoxymethyl and Het is 2-fluoro-4-methoxymethylpyridin-3-yl and the combination of R¹, R² and Y for a compound corresponds in each case to one row of Table A.

### Table 1277

Compounds of the formula (I) in which X is methoxymethyl and Het is 2,4-dichloropyridin-3-yl and the combination of R¹, R² and Y for a compound corresponds in each case to one row of Table A.

### Table 1278

Compounds of the formula (I) in which X is methoxymethyl and Het is 2-chloro-4-fluoropyridin-3-yl and the combination of R¹, R² and Y for a compound corresponds in each case to one row of Table A.

### Table 1279

Compounds of the formula (I) in which X is methoxymethyl and Het is 2-chloro-4-bromopyridin-3-yl and the combination of R¹, R² and Y for a compound corresponds in each case to one row of Table A.

### Table 1280

Compounds of the formula (I) in which X is methoxymethyl and Het is 2-chloro-4-methylpyridin-3-yl and the combination of R¹, R² and Y for a compound corresponds in each case to one row of Table A.

### Table 1281

Compounds of the formula (I) in which X is methoxymethyl and Het is 2-chloro-4-methoxypyridin-3-yl and the combination of R¹, R² and Y for a compound corresponds in each case to one row of Table A.

### Table 1282

Compounds of the formula (I) in which X is methoxymethyl and Het is 2-chloro-4-methylsulfanylpyridin-3-yl and the combination of R¹, R² and Y for a compound corresponds in each case to one row of Table A.

### Table 1283

Compounds of the formula (I) in which X is methoxymethyl and Het is 2-chloro-4-methylsulfinylpyridin-3-yl and the combination of R¹, R² and Y for a compound corresponds in each case to one row of Table A.

### Table 1284

Compounds of the formula (I) in which X is methoxymethyl and Het is 2-chloro-4-methylsulfonylpyridin-3-yl and the combination of R¹, R² and Y for a compound corresponds in each case to one row of Table A.

### Table 1285

Compounds of the formula (I) in which X is methoxymethyl and Het is 2-chloro-4-nitropyridin-3-yl and the combination of R¹, R² and Y for a compound corresponds in each case to one row of Table A.

### Table 1286

Compounds of the formula (I) in which X is methoxymethyl and Het is 2-chloro-4-methoxymethylpyridin-3-yl and the combination of R¹, R² and Y for a compound corresponds in each case to one row of Table A.

### Table 1287

Compounds of the formula (I) in which X is methoxymethyl and Het is 2,4-dimethylpyridin-3-yl and the combination of R¹, R² and Y forma compound corresponds in each case to one row of Table A.

### Table 1288

Compounds of the formula (I) in which X is methoxymethyl and Het is 2-methyl-4-chloropyridin-3-yl and the combination of R¹, R² and Y for a compound corresponds in each case to one row of Table A.

### Table 1289

Compounds of the formula (I) in which X is methoxymethyl and Het is 2-methyl-4-fluoropyridin-3-yl and the combination of R¹, R² and Y for a compound corresponds in each case to one row of Table A.

### Table 1290

Compounds of the formula (I) in which X is methoxymethyl and Het is 2-methyl-4-bromopyridin-3-yl and the combination of R¹, R² and Y for a compound corresponds in each case to one row of Table A.

### Table 1291

Compounds of the formula (I) in which X is methoxymethyl and Het is 2-methyl-4-methoxypyridin-3-yl and the combination of R¹, R² and Y for a compound corresponds in each case to one row of Table A.

### Table 1292

Compounds of the formula (I) in which X is methoxymethyl and Het is 2-methyl-4-methylsulfanylpyridin-3-yl and the combination of R¹, R² and Y for a compound corresponds in each case to one row of Table A.

### Table 1293

Compounds of the formula (I) in which X is methoxymethyl and Het is 2-methyl-4-methylsulfinylpyridin-3-yl and the combination of R¹, R² and Y for a compound corresponds in each case to one row of Table A.

### Table 1294

Compounds of the formula (I) in which X is methoxymethyl and Het is 2-methyl-4-methylsulfonylpyridin-3-yl and the combination of R¹, R² and Y for a compound corresponds in each case to one row of Table A.

### Table 1295

Compounds of the formula (I) in which X is methoxymethyl and Het is 2-methyl-4-nitropyridin-3-yl and the combination of R¹, R² and Y for a compound corresponds in each case to one row of Table A.

### Table 1296

Compounds of the formula (I) in which X is methoxymethyl and Het is 2-methyl-4-methoxymethylpyridin-3-yl and the combination of R¹, R² and Y for a compound corresponds in each case to one row of Table A.

### Table 1297

Compounds of the formula (I) in which X is methoxymethyl and Het is 2-bromo-4-fluoropyridin-3-yl and the combination of R¹, R² and Y for a compound corresponds in each case to one row of Table A.

### Table 1298

Compounds of the formula (I) in which X is methoxymethyl and Het is 2-bromo-4-chloropyridin-3-yl and the combination of R¹, R² and Y for a compound corresponds in each case to one row of Table A.

### Table 1299

Compounds of the formula (I) in which X is methoxymethyl and Het is 2-bromo-4-methylpyridin-3-yl and the combination of R¹, R² and Y for a compound corresponds in each case to one row of Table A.

### Table 1300

Compounds of the formula (I) in which X is methoxymethyl and Het is 2-methoxy-4-fluoropyridin-3-yl and the combination of R¹, R² and Y for a compound corresponds in each case to one row of Table A.

### Table 1301

Compounds of the formula (I) in which X is methoxymethyl and Het is 2-methoxy-4-chloropyridin-3-yl and the combination of R¹, R² and Y for a compound corresponds in each case to one row of Table A.

### Table 1302

Compounds of the formula (I) in which X is methoxymethyl and Het is 2-methoxy-4-methylpyridin-3-yl and the combination of R¹, R² and Y for a compound corresponds in each case to one row of Table A.

### Table 1303

Compounds of the formula (I) in which X is methoxymethyl and Het is 2-methylsulfanyl-4-fluoropyridin-3-yl and the combination of R¹, R² and Y for a compound corresponds in each case to one row of Table A.

### Table 1304

Compounds of the formula (I) in which X is methoxymethyl and Het is 2-methylsulfanyl-4-chloropyridin-3-yl and the combination of R¹, R² and Y for a compound corresponds in each case to one row of Table A.

### Table 1305

Compounds of the formula (I) in which X is methoxymethyl and Het is 2-methylsulfanyl-4-methylpyridin-3-yl and the combination of R¹, R² and Y for a compound corresponds in each case to one row of Table A.

### Table 1306

Compounds of the formula (I) in which X is methoxymethyl and Het is 2-methylsulfinyl-4-fluoropyridin-3-yl and the combination of R¹, R² and Y for a compound corresponds in each case to one row of Table A.

### Table 1307

Compounds of the formula (I) in which X is methoxymethyl and Het is 2-methylsulfinyl-4-chloropyridin-3-yl and the combination of R¹, R² and Y for a compound corresponds in each case to one row of Table A.

### Table 1308

Compounds of the formula (I) in which X is methoxymethyl and Het is 2-methylsulfinyl-4-methylpyridin-3-yl and the combination of R¹, R² and Y for a compound corresponds in each case to one row of Table A.

### Table 1309

Compounds of the formula (I) in which X is methoxymethyl and Het is 2-methylsulfonyl-4-fluoropyridin-3-yl and the combination of R¹, R² and Y for a compound corresponds in each case to one row of Table A.

### Table 1310

Compounds of the formula (I) in which X is methoxymethyl and Het is 2-methylsulfonyl-4-chloropyridin-3-yl and the combination of R¹, R² and Y for a compound corresponds in each case to one row of Table A.

### Table 1311

Compounds of the formula (I) in which X is methoxymethyl and Het is 2-methylsulfonyl-4-methylpyridin-3-yl and the combination of R¹, R² and Y for a compound corresponds in each case to one row of Table A.

### Table 1312

Compounds of the formula (I) in which X is methoxymethyl and Het is 2-nitro-4-fluoropyridin-3-yl and the combination of R¹, R² and Y for a compound corresponds in each case to one row of Table A.

### Table 1313

Compounds of the formula (I) in which X is methoxymethyl and Het is 2-nitro-4-chloropyridin-3-yl and the combination of R¹, R² and Y for a compound corresponds in each case to one row of Table A.

### Table 1314

Compounds of the formula (I) in which X is methoxymethyl and Het is 2-nitro-4-methylpyridin-3-yl and the combination of R¹, R² and Y for a compound corresponds in each case to one row of Table A.

### Table 1315

Compounds of the formula (I) in which X is methoxymethyl and Het is 2-methoxymethyl-4-fluoropyridin-3-yl and the combination of R¹, R² and Y for a compound corresponds in each case to one row of Table A.

### Table 1316

Compounds of the formula (I) in which X is methoxymethyl and Het is 2-methoxymethyl-4-chloropyridin-3-yl and the combination of R¹, R² and Y for a compound corresponds in each case to one row of Table A.

### Table 1317

Compounds of the formula (I) in which X is methoxymethyl and Het is 2-methoxymethyl-4-methylpyridin-3-yl and the combination of R¹, R² and Y for a compound corresponds in each case to one row of Table A.

### Table 1318

Compounds of the formula (I) in which X is methoxymethyl and Het is 4,6-difluoropyridin-3-yl and the combination of R¹, R² and Y for a compound corresponds in each case to one row of Table A.

### Table 1319

Compounds of the formula (I) in which X is methoxymethyl and Het is 4-fluoro-6-chloropyridin-3-yl and the combination of R¹, R² and Y for a compound corresponds in each case to one row of Table A.

### Table 1320

Compounds of the formula (I) in which X is methoxymethyl and Het is 4-fluoro-6-bromopyridin-3-yl and the combination of R¹, R² and Y for a compound corresponds in each case to one row of Table A.

### Table 1321

Compounds of the formula (I) in which X is methoxymethyl and Het is 4-fluoro-6-methylpyridin-3-yl and the combination of R¹, R² and Y for a compound corresponds in each case to one row of Table A.

### Table 1322

Compounds of the formula (I) in which X is methoxymethyl and Het is 4-fluoro-6-ethylpyridin-3-yl and the combination of R¹, R² and Y for a compound corresponds in each case to one row of Table A.

### Table 323

Compounds of the formula (I) in which X is methoxymethyl and Het is 4-fluoro-6-methoxypyridin-3-yl and the combination of R¹, R² and Y for a compound corresponds in each case to one row of Table A.

### Table 1324

Compounds of the formula (I) in which X is methoxymethyl and Het is 4-fluoro-6-cyanopyridin-3-yl and the combination of R¹, R² and Y for a compound corresponds in each case to one row of Table A.

### Table 1325

Compounds of the formula (I) in which X is methoxymethyl and Het is 4-fluoro-6-methoxycarbonylpyridin-3-yl and the combination of R¹, R² and Y for a compound corresponds in each case to one row of Table A.

### Table 1326

Compounds of the formula (I) in which X is methoxymethyl and Het is 4-fluoro-6-ethoxycarbonylpyridin-3-yl and the combination of R¹, R² and Y for a compound corresponds in each case to one row of Table A.

### Table 1327

Compounds of the formula (I) in which X is methoxymethyl and Het is 4-fluoro-6-carbamoylpyridin-3-yl and the combination of R¹, R² and Y for a compound corresponds in each case to one row of Table A.

### Table 1328

Compounds of the formula (I) in which X is methoxymethyl and Het is 4-fluoro-6-methylcarbamoylpyridin-3-yl and the combination of R¹, R² and Y for a compound corresponds in each case to one row of Table A.

### Table 1329

Compounds of the formula (I) in which X is methoxymethyl and Het is 4-fluoro-6-dimethylcarbamoylpyridin-3-yl and the combination of R¹, R² and Y for a compound corresponds in each case to one row of Table A.

### Table 1330

Compounds of the formula (I) in which X is methoxymethyl and Het is 4-fluoro-6-thiocarbamoylpyridin-3-yl and the combination of R¹, R² and Y for a compound corresponds in each case to one row of Table A.

### Table 1331

Compounds of the formula (I) in which X is methoxymethyl and Het is 4-fluoro-6-nitropyridin-3-yl and the combination of R¹, R² and Y for a compound corresponds in each case to one row of Table A.

### Table 1332

Compounds of the formula (I) in which X is methoxymethyl and Het is 4-fluoro-6-acetylpyridin-3-yl and the combination of R¹, R² and Y for a compound corresponds in each case to one row of Table A.

### Table 1333

Compounds of the formula (I) in which X is methoxymethyl and Het is 4,6-dichloropyridin-3-yl and the combination of R¹, R² and Y for a compound corresponds in each case to one row of Table A.

### Table 1334

Compounds of the formula (I) in which X is methoxymethyl and Het is 4-chloro-6-fluoropyridin-3-yl and the combination of R¹, R² and Y for a compound corresponds in each case to one row of Table A.

### Table 1335

Compounds of the formula (I) in which X is methoxymethyl and Het is 4-chloro-6-bromopyridin-3-yl and the combination of R¹, R² and Y for a compound corresponds in each case to one row of Table A.

### Table 1336

Compounds of the formula (I) in which X is methoxymethyl and Het is 4-chloro-6-methylpyridin-3-yl and the combination of R¹, R² and Y for a compound corresponds in each case to one row of Table A.

### Table 1337

Compounds of the formula (I) in which X is methoxymethyl and Het is 4-chloro-6-ethylpyridin-3-yl and the combination of R¹, R² and Y for a compound corresponds in each case to one row of Table A.

### Table 1338

Compounds of the formula (I) in which X is methoxymethyl and Het is 4-chloro-6-methoxypyridin-3-yl and the combination of R¹, R² and Y for a compound corresponds in each case to one row of table A.

### Table 1339

Compounds of the formula (I) in which X is methoxymethyl and Het is 4-chloro-6-cyanopyridin-3-yl and the combination of R¹, R² and Y for a compound corresponds in each case to one row of Table A.

### Table 1340

Compounds of the formula (I) in which X is methoxymethyl and Het is 4-chloro-6-methoxycarbonylpyridin-3-yl and the combination of R¹, R² and Y for a compound corresponds in each case to one row of Table A.

### Table 1341

Compounds of the formula (I) in which X is methoxymethyl and Het is 4-chloro-6-ethoxycarbonylpyridin-3-yl and the combination of R¹, R² and Y for a compound corresponds in each case to one row of Table A.

### Table 1342

Compounds of the formula (I) in which X is methoxymethyl and Het is 4-chloro-6-carbamoylpyridin-3-yl and the combination of R¹, R² and Y for a compound corresponds in each case to one row of Table A.

### Table 1343

Compounds of the formula (I) in which X is methoxymethyl and Het is 4-chloro-6-methylcarbamoylpyridin-3-yl and the combination of R¹, R² and Y for a compound corresponds in each case to one row of Table A.

### Table 1344

Compounds of the formula (I) in which X is methoxymethyl and Het is 4-chloro-6-dimethylcarbamoylpyridin-3-yl and the combination of R¹, R² and Y for a compound corresponds in each case to one row of Table A.

### Table 1345

Compounds of the formula (I) in which X is methoxymethyl and Het is 4-chloro-6-thiocarbamoylpyridin-3-yl and the combination of R¹, R² and Y for a compound corresponds in each case to one row of Table A.

### Table 1346

Compounds of the formula (I) in which X is methoxymethyl and Het is 4-chloro-6-nitropyridin-3-yl and the combination of R¹, R² and Y for a compound corresponds in each case to one row of Table A.

### Table 1347

Compounds of the formula (I) in which X is methoxymethyl and Het is 4-chloro-6-acetylpyridin-3-yl and the combination of R¹, R² and Y for a compound corresponds in each case to one row of Table A.

### Table 1348

Compounds of the formula (I) in which X is methoxymethyl and Het is 4-methyl-6-fluoropyridin-3-yl and the combination of R¹, R² and Y for a compound corresponds in each case to one row of Table A.

### Table 1349

Compounds of the formula (I) in which X is methoxymethyl and Het is 4-methyl-6-chloropyridin-3-yl and the combination of R¹, R² and Y for a compound corresponds in each case to one row of Table A.

### Table 1350

Compounds of the formula (I) in which X is methoxymethyl and Het is 4-methyl-6-bromopyridin-3-yl and the combination of R¹, R² and Y for a compound corresponds in each case to one row of Table A.

### Table 1351

Compounds of the formula (I) in which X is methoxymethyl and Het is 4,6-dimethylpyridin-3-yl and the combination of R¹, R² and Y for a compound corresponds in each case to one row of Table A.

### Table 1352

Compounds of the formula (I) in which X is methoxymethyl and Het is 4-methyl-6-ethylpyridin-3-yl and the combination of R¹, R² and Y for a compound corresponds in each case to one row of Table A.

### Table 1353

Compounds of the formula (I) in which X is methoxymethyl and Het is 4-methyl-6-methoxypyridin-3-yl and the combination of R¹, R² and Y for a compound corresponds in each case to one row of Table A.

### Table 1354

Compounds of the formula (I) in which X is methoxymethyl and Het is 4-methyl-6-cyanopyridin-3-yl and the combination of R¹, R² and Y for a compound corresponds in each case to one row of Table A.

### Table 1355

Compounds of the formula (I) in which X is methoxymethyl and Het is 4-methyl-6-methoxycarbonylpyridin-3-yl and the combination of R¹, R² and Y for a compound corresponds in each case to one row of Table A.

### Table 1356

Compounds of the formula (I) in which X is methoxymethyl and Het is 4-methyl-6-ethoxycarbonylpyridin-3-yl and the combination of R¹, R² and Y for a compound corresponds in each case to one row of Table A.

### Table 1357

Compounds of the formula (I) in which X is methoxymethyl and Het is 4-methyl-6-carbamoylpyridin-3-yl and the combination of R¹, R² and Y for a compound corresponds in each case to one row of Table A.

### Table 1358

Compounds of the formula (I) in which X is methoxymethyl and Het is 4-methyl-6-methylcarbamoylpyridin-3-yl and the combination of R¹, R² and Y for a compound corresponds in each case to one row of Table A.

### Table 1359

Compounds of the formula (I) in which X is methoxymethyl and Het is 4-methyl-6-dimethylcarbamoylpyridin-3-yl and the combination of R¹, R² and Y for a compound corresponds in each case to one row of Table A.

### Table 1360

Compounds of the formula (I) in which X is methoxymethyl and Het is 4-methyl-6-thiocarbamoylpyridin-3-yl and the combination of R¹, R² and Y for a compound corresponds in each case to one row of Table A.

### Table 1361

Compounds of the formula (I) in which X is methoxymethyl and Het is 4-methyl-6-nitropyridin-3-yl and the combination of R¹, R² and Y for a compound corresponds in each case to one row of Table A.

### Table 1362

Compounds of the formula (I) in which X is methoxymethyl and Het is 4-methyl-6-acetylpyridin-3-yl and the combination of R¹, R² and Y for a compound corresponds in each case to one row of Table A.

### Table 1363

Compounds of the formula (I) in which X is methoxymethyl and Het is 4-bromo-6-fluoropyridin-3-yl and the combination of R¹, R² and Y for a compound corresponds in each case to one row of Table A.

### Table 1364

Compounds of the formula (I) in which X is methoxymethyl and Het is 4-bromo-6-chloropyridin-3-yl and the combination of R¹, R² and Y for a compound corresponds in each case to one row of Table A.

### Table 1365

Compounds of the formula (I) in which X is methoxymethyl and Het is 4-bromo-6-methylpyridin-3-yl and the combination of R¹, R² and Y for a compound corresponds in each case to one row of table A.

### Table 1366

Compounds of the formula (I) in which X is methoxymethyl and Het is 4-methoxy-6-fluoropyridin-3-yl and the combination of R¹, R² and Y for a compound corresponds in each case to one row of Table A.

### Table 1367

Compounds of the formula (I) in which X is methoxymethyl and Het is 4-methoxy-6-chloropyridin-3-yl and the combination of R¹, R² and Y for a compound corresponds in each case to one row of Table A.

### Table 1368

Compounds of the formula (I) in which X is methoxymethyl and Het is 4-methoxy-6-methylpyridin-3-yl and the combination of R¹, R² and Y for a compound corresponds in each case to one row of Table A.

### Table 1369

Compounds of the formula (I) in which X is methoxymethyl and Het is 4-methylsulfanyl-6-fluoropyridin-3-yl and the combination of R¹, R² and Y for a compound corresponds in each case to one row of Table A.

### Table 1370

Compounds of the formula (I) in which X is methoxymethyl and Het is 4-methylsulfanyl-6-chloropyridin-3-yl and the combination of R¹, R² and Y for a compound corresponds in each case to one row of Table A.

### Table 1371

Compounds of the formula (I) in which X is methoxymethyl and Het is 4-methylsulfanyl-6-methylpyridin-3-yl and the combination of R¹, R² and Y for a compound corresponds in each case to one row of Table A.

### Table 1372

Compounds of the formula (I) in which X is methoxymethyl and Het is 4-methylsulfinyl-6-fluoropyridin-3-yl and the combination of R¹, R² and Y for a compound corresponds in each case to one row of Table A.

### Table 1373

Compounds of the formula (I) in which X is methoxymethyl and Het is 4-methylsulfinyl-6-chloropyridin-3-yl and the combination of R¹, R² and Y for a compound corresponds in each case to one row of Table A.

### Table 1374

Compounds of the formula (I) in which X is methoxymethyl and Het is 4-methylsulfinyl-6-methylpyridin-3-yl and the combination of R¹, R² and Y for a compound corresponds in each case to one row of Table A.

### Table 1375

Compounds of the formula (I) in which X is methoxymethyl and Het is 4-methylsulfonyl-6-fluoropyridin-3-yl and the combination of R¹, R² and Y for a compound corresponds in each case to one row of Table A.

### Table 1376

Compounds of the formula (I) in which X is methoxymethyl and Het is 4-methylsulfonyl-6-chloropyridin-3-yl and the combination of R¹, R² and Y for a compound corresponds in each case to one row of Table A.

### Table 1377

Compounds of the formula (I) in which X is methoxymethyl and Het is 4-methylsulfonyl-6-methylpyridin-3-yl and the combination of R¹, R² and Y for a compound corresponds in each case to one row of Table A.

### Table 1378

Compounds of the formula (I) in which X is methoxymethyl and Het is 4-nitro-6-fluoropyridin-3-yl and the combination of R¹, R² and Y for a compound corresponds in each case to one row of Table A.

### Table 1379

Compounds of the formula (I) in which X is methoxymethyl and Het is 4-nitro-6-chloropyridin-3-yl and the combination of R¹, R² and Y for a compound corresponds in each case to one row of Table A.

### Table 1380

Compounds of the formula (I) in which X is methoxymethyl and Het is 4-nitro-6-methylpyridin-3-yl and the combination of R¹, R² and Y for a compound corresponds in each case to one row of Table A.

### Table 1381

Compounds of the formula (I) in which X is methoxymethyl and Het is 4-methoxymethyl-6-fluoropyridin-3-yl and the combination of R¹, R² and Y for a compound corresponds in each case to one row of Table A.

### Table 1382

Compounds of the formula (I) in which X is methoxymethyl and Het is 4-methoxymethyl-6-chloropyridin-3-yl and the combination of R¹, R² and Y for a compound corresponds in each case to one row of Table A.

### Table 1383

Compounds of the formula (I) in which X is methoxymethyl and Het is 4-methoxymethyl-6-methylpyridin-3-yl and the combination of R¹, R² and Y for a compound corresponds in each case to one row of Table A.

### Table 1384

Compounds of the formula (I) in which X is methoxymethyl and Het is 2,4-dichloro-6-methylpyridin-3-yl and the combination of R¹, R² and Y for a compound corresponds in each case to one row of Table A.

### Table 1385

Compounds of the formula (I) in which X is methyl and R¹ and R² are hydrogen and Het and Y for a compound correspond in each case to one row of Table B.

### Table 1386

Compounds of the formula (I) in which X is ethyl and R¹ and R² are hydrogen and Het and Y for a compound correspond in each case to one row of Table B.

### Table 1387

Compounds of the formula (I) in which X is methoxymethyl and R¹ and R² are hydrogen and Het and Y for a compound correspond in each case to one row of Table B.

TABLE A

| Row | R¹ | R² | Y |
|---|---|---|---|
| A-1 | H | H | H |
| A-2 | CH₃ | H | H |
| A-3 | CH₃ | CH₃ | H |
| A-4 | CH₂CH₃ | H | H |
| A-5 | CH₂CH₃ | CH₃ | H |
| A-6 | CH₂CH₃ | CH₂CH₃ | H |
| A-7 | CH₂CF₃ | H | H |
| A-8 | CH₂CF₃ | CH₃ | H |
| A-9 | CH₂CF₃ | CH₂CH₃ | H |
| A-10 | CH₂CCl₃ | H | H |
| A-11 | CH₂CCl₃ | CH₃ | H |
| A-12 | CH₂CCl₃ | CH₂CH₃ | H |
| A-13 | CH₂CH₂CH₃ | H | H |
| A-14 | CH₂CH₂CH₃ | CH₃ | H |
| A-15 | CH₂CH₂CH₃ | CH₂CH₃ | H |
| A-16 | CH₂CH₂CH₃ | CH₂CH₂CH₃ | H |
| A-17 | CH(CH₃)₂ | H | H |
| A-18 | CH(CH₃)₂ | CH₃ | H |
| A-19 | CH(CH₃)₂ | CH₂CH₃ | H |
| A-20 | CH₂CH(CH₃)₂ | H | H |
| A-21 | CH₂CH(CH₃)₂ | CH₃ | H |
| A-22 | CH₂CH(CH₃)₂ | CH₂CH₃ | H |
| A-23 | CH₂CH(CH₃)₂ | CH₂CH₂CH₃ | H |
| A-24 | CH₂CH₂CH₂CH₃ | H | H |
| A-25 | CH₂CH₂CH₂CH₃ | CH₃ | H |
| A-26 | CH₂CH₂CH₂CH₃ | CH₂CH₃ | H |
| A-27 | CH₂CH₂CH₂CH₃ | CH₂CH₂CH₃ | H |
| A-28 | CH₂CH₂CH₂CH₃ | CH₂CH₂CH₂CH₃ | H |
| A-29 | (±) CH(CH₃)—CH₂CH₃ | H | H |
| A-30 | (±) CH(CH₃)—CH₂CH₃ | CH₃ | H |
| A-31 | (±) CH(CH₃)—CH₂CH₃ | CH₂CH₃ | H |
| A-32 | (S) CH(CH₃)—CH₂CH₃ | H | H |
| A-33 | (S) CH(CH₃)—CH₂CH₃ | CH₃ | H |
| A-34 | (S) CH(CH₃)—CH₂CH₃ | CH₂CH₃ | H |
| A-35 | (R) CH(CH₃)—CH₂CH₃ | H | H |
| A-36 | (R) CH(CH₃)—CH₂CH₃ | CH₃ | H |
| A-37 | (R) CH(CH₃)—CH₂CH₃ | CH₂CH₃ | H |
| A-38 | (±) CH(CH₃)—CH(CH₃)₂ | H | H |
| A-39 | (±) CH(CH₃)—CH(CH₃)₂ | CH₃ | H |
| A-40 | (±) CH(CH₃)—CH(CH₃)₂ | CH₂CH₃ | H |
| A-41 | (S) CH(CH₃)—CH(CH₃)₂ | H | H |
| A-42 | (S) CH(CH₃)—CH(CH₃)₂ | CH₃ | H |
| A-43 | (S) CH(CH₃)—CH(CH₃)₂ | CH₂CH₃ | H |
| A-44 | (R) CH(CH₃)—CH(CH₃)₂ | H | H |
| A-45 | (R) CH(CH₃)—CH(CH₃)₂ | CH₃ | H |
| A-46 | (R) CH(CH₃)—CH(CH₃)₂ | CH₂CH₃ | H |
| A-47 | (±) CH(CH₃)—C(CH₃)₃ | H | H |
| A-48 | (±) CH(CH₃)—C(CH₃)₃ | CH₃ | H |
| A-49 | (±) CH(CH₃)—C(CH₃)₃ | CH₂CH₃ | H |
| A-50 | (S) CH(CH₃)—C(CH₃)₃ | H | H |
| A-51 | (S) CH(CH₃)—C(CH₃)₃ | CH₃ | H |
| A-52 | (S) CH(CH₃)—C(CH₃)₃ | CH₂CH₃ | H |
| A-53 | (R) CH(CH₃)—C(CH₃)₃ | H | H |
| A-54 | (R) CH(CH₃)—C(CH₃)₃ | CH₃ | H |
| A-55 | (R) CH(CH₃)—C(CH₃)₃ | CH₂CH₃ | H |
| A-56 | (±) CH(CH₃)—CF₃ | H | H |
| A-57 | (±) CH(CH₃)—CF₃ | CH₃ | H |
| A-58 | (±) CH(CH₃)—CF₃ | CH₂CH₃ | H |
| A-59 | (S) CH(CH₃)—CF₃ | H | H |
| A-60 | (S) CH(CH₃)—CF₃ | CH₃ | H |
| A-61 | (S) CH(CH₃)—CF₃ | CH₂CH₃ | H |
| A-62 | (R) CH(CH₃)—CF₃ | H | H |
| A-63 | (R) CH(CH₃)—CF₃ | CH₃ | H |
| A-64 | (R) CH(CH₃)—CF₃ | CH₂CH₃ | H |
| A-65 | (±) CH(CH₃)—CCl₃ | H | H |
| A-66 | (±) CH(CH₃)—CCl₃ | CH₃ | H |
| A-67 | (±) CH(CH₃)—CCl₃ | CH₂CH₃ | H |
| A-68 | (S) CH(CH₃)—CCl₃ | H | H |
| A-69 | (S) CH(CH₃)—CCl₃ | CH₃ | H |
| A-70 | (S) CH(CH₃)—CCl₃ | CH₂CH₃ | H |
| A-71 | (R) CH(CH₃)—CCl₃ | H | H |
| A-72 | (R) CH(CH₃)—CCl₃ | CH₃ | H |
| A-73 | (R) CH(CH₃)—CCl₃ | CH₂CH₃ | H |
| A-74 | CH₂CF₂CF₃ | H | H |
| A-75 | CH₂CF₂CF₃ | CH₃ | H |
| A-76 | CH₂CF₂CF₃ | CH₂CH₃ | H |
| A-77 | CH₂(CF₂)₂CF₃ | H | H |
| A-78 | CH₂(CF₂)₂CF₃ | CH₃ | H |
| A-79 | CH₂(CF₂)₂CF₃ | CH₂CH₃ | H |
| A-80 | CH₂C(CH₃)═CH₂ | H | H |
| A-81 | CH₂C(CH₃)═CH₂ | CH₃ | H |
| A-82 | CH₂C(CH₃)═CH₂ | CH₂CH₃ | H |
| A-83 | CH₂CH═CH₂ | H | H |
| A-84 | CH₂CH═CH₂ | CH₃ | H |
| A-85 | CH₂CH═CH₂ | CH₂CH₃ | H |
| A-86 | (±) CH(CH₃)CH═CH₂ | H | H |
| A-87 | (±) CH(CH₃)CH═CH₂ | CH₃ | H |
| A-88 | (±) CH(CH₃)CH═CH₂ | CH₂CH₃ | H |
| A-89 | (S) CH(CH₃)CH═CH₂ | H | H |
| A-90 | (S) CH(CH₃)CH═CH₂ | CH₃ | H |
| A-91 | (S) CH(CH₃)CH═CH₂ | CH₂CH₃ | H |
| A-92 | (R) CH(CH₃)CH═CH₂ | H | H |
| A-93 | (R) CH(CH₃)CH═CH₂ | CH₃ | H |
| A-94 | (R) CH(CH₃)CH═CH₂ | CH₂CH₃ | H |
| A-95 | (( ) CH(CH3)C(CH3)═CH2 | H | H |
| A-96 | (( ) CH(CH3)C(CH3)═CH2 | CH₃ | H |
| A-97 | (( ) CH(CH3)C(CH3)═CH2 | CH₂CH₃ | H |
| A-98 | (S) CH(CH3)C(CH3)═CH2 | H | H |
| A-99 | (S) CH(CH3)C(CH3)═CH2 | CH₃ | H |
| A-100 | (S) CH(CH3)C(CH3)═CH2 | CH₂CH₃ | H |
| A-101 | (R) CH(CH3)C(CH3)═CH2 | H | H |
| A-102 | (R) CH(CH3)C(CH3)═CH2 | CH₃ | H |
| A-103 | (R) CH(CH₃)C(CH₃)═CH₂ | CH₂CH₃ | H |
| A-104 | CH₂—C≡CH | H | H |
| A-105 | CH₂—C≡CH | CH₃ | H |
| A-106 | CH₂—C≡CH | CH₂CH₃ | H |
| A-107 | cyclopentyl | H | H |
| A-108 | cyclopentyl | CH₃ | H |
| A-109 | cyclopentyl | CH₂CH₃ | H |
| A-110 | cyclohexyl | H | H |
| A-111 | cyclohexyl | CH₃ | H |
| A-112 | cyclohexyl | CH₂CH₃ | H |
| A-113 | CH₂—C₆H₅ | H | H |
| A-114 | CH₂—C₆H₅ | CH₃ | H |
| A-115 | CH₂—C₆H₅ | CH₂CH₃ | H |
| A-116 | NH₂ | CH₂-c-C₆H₁₁ | H |
| A-117 | NH₂ | CH₂CH₃ | H |
| A-118 | NH₂ | CH₂CH₂CH₃ | H |
| A-119 | NH—CH₂—CH═CH₂ | H | H |
| A-120 | NH—CH₂—CH═CH₂ | CH₃ | H |
| A-121 | NH—CH₂—CH═CH₂ | CH₂CH₃ | H |
| A-122 | NH—C(CH₃)₃ | H | H |
| A-123 | N(CH₃)₂ | H | H |
| A-124 | NH(CH₃) | H | H |
| A-125 | —(CH₂)₂CH═CHCH₂— | | H |
| A-126 | —(CH₂)₂C(CH₃)═CHCH₂— | | H |
| A-127 | —CH(CH₃)CH₂—CH═CHCH₂— | | H |
| A-128 | —(CH₂)₂CH(CH₃)(CH₂)₂— | | H |
| A-129 | —(CH₂)₃CHFCH₂— | | H |
| A-130 | —(CH₂)₂CHF(CH₂)₂— | | H |
| A-131 | —CH₂CHF(CH₂)₃— | | H |
| A-132 | —(CH₂)₂CH(CF₃)(CH₂)₂— | | H |
| A-133 | —(CH₂)₂O(CH₂)₂— | | H |
| A-134 | —(CH₂)₂S(CH₂)₂— | | H |
| A-135 | —(CH₂)₅— | | H |
| A-136 | —(CH₂)₄— | | H |
| A-137 | —CH₂CH═CHCH₂— | | H |
| A-138 | —CH(CH₃)(CH₂)₃— | | H |
| A-139 | —CH₂CH(CH₃)(CH₂)₂— | | H |
| A-140 | —CH(CH₃)—(CH₂)₂—CH(CH₃)— | | H |
| A-141 | —CH(CH₃)—(CH₂)₄— | | H |
| A-142 | —CH₂—CH(CH₃)—(CH₂)₃— | | H |
| A-143 | —(CH₂)—CH(CH₃)—CH₂—CH(CH₃)—CH₂— | | H |
| A-144 | —CH(CH₂CH₃)—(CH₂)₄— | | H |
| A-145 | —(CH₂)₂—CHOH—(CH₂)₂— | | H |
| A-146 | —(CH₂)₆— | | H |
| A-147 | —CH(CH₃)—(CH₂)₅— | | H |
| A-148 | —(CH₂)₂—N(CH₃)—(CH₂)₂— | | H |
| A-149 | —N═CH—CH═CH— | | H |
| A-150 | —N═C(CH₃)—CH═C(CH₃)— | | H |
| A-151 | —N═C(CF₃)—CH═C(CF₃)— | | H |
| A-152 | H | H | F |
| A-153 | CH₃ | H | F |
| A-154 | CH₃ | CH₃ | F |
| A-155 | CH₂CH₃ | H | F |
| A-156 | CH₂CH₃ | CH₃ | F |
| A-157 | CH₂CH₃ | CH₂CH₃ | F |
| A-158 | CH₂CF₃ | H | F |
| A-159 | CH₂CF₃ | CH₃ | F |
| A-160 | CH₂CF₃ | CH₂CH₃ | F |
| A-161 | CH₂CCl₃ | H | F |
| A-162 | CH₂CCl₃ | CH₃ | F |
| A-163 | CH₂CCl₃ | CH₂CH₃ | F |
| A-164 | CH₂CH₂CH₃ | H | F |
| A-165 | CH₂CH₂CH₃ | CH₃ | F |
| A-166 | CH₂CH₂CH₃ | CH₂CH₃ | F |
| A-167 | CH₂CH₂CH₃ | CH₂CH₂CH₃ | F |
| A-168 | CH(CH₃)₂ | H | F |
| A-169 | CH(CH₃)₂ | CH₃ | F |
| A-170 | CH(CH₃)₂ | CH₂CH₃ | F |
| A-171 | CH₂CH(CH₃)₂ | H | F |
| A-172 | CH₂CH(CH₃)₂ | CH₃ | F |
| A-173 | CH₂CH(CH₃)₂ | CH₂CH₃ | F |
| A-174 | CH₂CH(CH₃)₂ | CH₂CH₂CH₃ | F |
| A-175 | CH₂CH₂CH₂CH₃ | H | F |
| A-176 | CH₂CH₂CH₂CH₃ | CH₃ | F |
| A-177 | CH₂CH₂CH₂CH₃ | CH₂CH₃ | F |
| A-178 | CH₂CH₂CH₂CH₃ | CH₂CH₂CH₃ | F |
| A-179 | CH₂CH₂CH₂CH₃ | CH₂CH₂CH₂CH₃ | F |
| A-180 | (±) CH(CH₃)—CH₂CH₃ | H | F |
| A-181 | (±) CH(CH₃)—CH₂CH₃ | CH₃ | F |
| A-182 | (±) CH(CH₃)—CH₂CH₃ | CH₂CH₃ | F |
| A-183 | (S) CH(CH₃)—CH₂CH₃ | H | F |
| A-184 | (S) CH(CH₃)—CH₂CH₃ | CH₃ | F |
| A-185 | (S) CH(CH₃)—CH₂CH₃ | CH₂CH₃ | F |
| A-186 | (R) CH(CH₃)—CH₂CH₃ | H | F |
| A-187 | (R) CH(CH₃)—CH₂CH₃ | CH₃ | F |
| A-188 | (R) CH(CH₃)—CH₂CH₃ | CH₂CH₃ | F |
| A-189 | (±) CH(CH₃)—CH(CH₃)₂ | H | F |
| A-190 | (±) CH(CH₃)—CH(CH₃)₂ | CH₃ | F |
| A-191 | (±) CH(CH₃)—CH(CH₃)₂ | CH₂CH₃ | F |
| A-192 | (S) CH(CH₃)—CH(CH₃)₂ | H | F |
| A-193 | (S) CH(CH₃)—CH(CH₃)₂ | CH₃ | F |
| A-194 | (S) CH(CH₃)—CH(CH₃)₂ | CH₂CH₃ | F |
| A-195 | (R) CH(CH₃)—CH(CH₃)₂ | H | F |
| A-196 | (R) CH(CH₃)—CH(CH₃)₂ | CH₃ | F |
| A-197 | (R) CH(CH₃)—CH(CH₃)₂ | CH₂CH₃ | F |
| A-198 | (±) CH(CH₃)—C(CH₃)₃ | H | F |
| A-199 | (±) CH(CH₃)—C(CH₃)₃ | CH₃ | F |
| A-200 | (±) CH(CH₃)—C(CH₃)₃ | CH₂CH₃ | F |
| A-201 | (S) CH(CH₃)—C(CH₃)₃ | H | F |
| A-202 | (S) CH(CH₃)—C(CH₃)₃ | CH₃ | F |
| A-203 | (S) CH(CH₃)—C(CH₃)₃ | CH₂CH₃ | F |
| A-204 | (R) CH(CH₃)—C(CH₃)₃ | H | F |
| A-205 | (R) CH(CH₃)—C(CH₃)₃ | CH₃ | F |
| A-206 | (R) CH(CH₃)—C(CH₃)₃ | CH₂CH₃ | F |
| A-207 | (±) CH(CH₃)—CF₃ | H | F |
| A-208 | (±) CH(CH₃)—CF₃ | CH₃ | F |
| A-209 | (±) CH(CH₃)—CF₃ | CH₂CH₃ | F |
| A-210 | (S) CH(CH₃)—CF₃ | H | F |
| A-211 | (S) CH(CH₃)—CF₃ | CH₃ | F |
| A-212 | (S) CH(CH₃)—CF₃ | CH₂CH₃ | F |
| A-213 | (R) CH(CH₃)—CF₃ | H | F |
| A-214 | (R) CH(CH₃)—CF₃ | CH₃ | F |
| A-215 | (R) CH(CH₃)—CF₃ | CH₂CH₃ | F |
| A-216 | (±) CH(CH₃)—CCl₃ | H | F |
| A-217 | (±) CH(CH₃)—CCl₃ | CH₃ | F |
| A-218 | (±) CH(CH₃)—CCl₃ | CH₂CH₃ | F |
| A-219 | (S) CH(CH₃)—CCl₃ | H | F |
| A-220 | (S) CH(CH₃)—CCl₃ | CH₃ | F |
| A-221 | (S) CH(CH₃)—CCl₃ | CH₂CH₃ | F |
| A-222 | (R) CH(CH₃)—CCl₃ | H | F |
| A-223 | (R) CH(CH₃)—CCl₃ | CH₃ | F |
| A-224 | (R) CH(CH₃)—CCl₃ | CH₂CH₃ | F |
| A-225 | CH₂CF₂CF₃ | H | F |
| A-226 | CH₂CF₂CF₃ | CH₃ | F |
| A-227 | CH₂CF₂CF₃ | CH₂CH₃ | F |
| A-228 | CH₂(CF₂)₂CF₃ | H | F |
| A-229 | CH₂(CF₂)₂CF₃ | CH₃ | F |
| A-230 | CH₂(CF₂)₂CF₃ | CH₂CH₃ | F |
| A-231 | CH₂C(CH₃)═CH₂ | H | F |
| A-232 | CH₂C(CH₃)═CH₂ | CH₃ | F |
| A-233 | CH₂C(CH₃)═CH₂ | CH₂CH₃ | F |
| A-234 | CH₂CH═CH₂ | H | F |
| A-235 | CH₂CH═CH₂ | CH₃ | F |
| A-236 | CH₂CH═CH₂ | CH₂CH₃ | F |
| A-237 | (±) CH(CH₃)CH═CH₂ | H | F |
| A-238 | (±) CH(CH₃)CH═CH₂ | CH₃ | F |
| A-239 | (±) CH(CH₃)CH═CH₂ | CH₂CH₃ | F |
| A-240 | (S) CH(CH₃)CH═CH₂ | H | F |
| A-241 | (S) CH(CH₃)CH═CH₂ | CH₃ | F |
| A-242 | (S) CH(CH₃)CH═CH₂ | CH₂CH₃ | F |
| A-243 | (R) CH(CH₃)CH═CH₂ | H | F |
| A-244 | (R) CH(CH₃)CH═CH₂ | CH₃ | F |
| A-245 | (R) CH(CH₃)CH═CH₂ | CH₂CH₃ | F |
| A-246 | (±)CH(CH₃)C(CH₃)═CH₂ | H | F |
| A-247 | (±) CH(CH₃)C(CH₃)═CH₂ | CH₃ | F |
| A-248 | (±)CH(CH₃)C(CH₃)═CH₂ | CH₂CH₃ | F |
| A-249 | (S) CH(CH₃)C(CH₃)═CH₂ | H | F |
| A-250 | (S) CH(CH₃)C(CH₃)═CH₂ | CH₃ | F |
| A-251 | (S) CH(CH₃)C(CH₃)═CH₂ | CH₂CH₃ | F |
| A-252 | (R) CH(CH₃)C(CH₃)═CH₂ | H | F |
| A-253 | (R) CH(CH₃)C(CH₃)═CH₂ | CH₃ | F |
| A-254 | (R) CH(CH₃)C(CH₃)═CH₂ | CH₂CH₃ | F |
| A-255 | CH₂—C≡CH | H | F |
| A-256 | CH₂—C≡CH | CH₃ | F |
| A-257 | CH₂—C≡CH | CH₂CH₃ | F |
| A-258 | cyclopentyl | H | F |
| A-259 | cyclopentyl | CH₃ | F |
| A-260 | cyclopentyl | CH₂CH₃ | F |
| A-261 | cyclohexyl | H | F |
| A-262 | cyclohexyl | CH₃ | F |
| A-263 | cyclohexyl | CH₂CH₃ | F |
| A-264 | CH₂—C₆H₅ | H | F |
| A-265 | CH₂—C₆H₅ | CH₃ | F |
| A-266 | CH₂—C₆H₅ | CH₂CH₃ | F |
| A-267 | NH₂ | CH₂-c-C₆H₁₁ | F |
| A-268 | NH₂ | CH₂CH₃ | F |
| A-269 | NH₂ | CH₂CH₂CH₃ | F |
| A-270 | NH—CH₂—CH═CH₂ | H | F |
| A-271 | NH—CH₂—CH═CH₂ | CH₃ | F |
| A-272 | NH—CH₂—CH═CH₂ | CH₂CH₃ | F |
| A-273 | NH—C(CH₃)₃ | H | F |
| A-274 | N(CH₃)₂ | H | F |
| A-275 | NH(CH₃) | H | F |
| A-276 | —(CH₂)₂CH═CHCH₂— | | F |
| A-277 | —(CH₂)₂C(CH₃)═CHCH₂— | | F |
| A-278 | —CH(CH₃)CH₂—CH═CHCH₂— | | F |
| A-279 | —(CH₂)₂CH(CH₃)(CH₂)₂— | | F |
| A-280 | —(CH₂)₃CHFCH₂— | | F |
| A-281 | —(CH₂)₂CHF(CH₂)₂— | | F |
| A-282 | —CH₂CHF(CH₂)₃— | | F |
| A-283 | —(CH₂)₂CH(CF₃)(CH₂)₂— | | F |
| A-284 | —(CH₂)₂O(CH₂)₂— | | F |
| A-285 | —(CH₂)₂S(CH₂)₂— | | F |
| A-286 | —(CH₂)₅— | | F |
| A-287 | —(CH₂)₄— | | F |
| A-288 | —CH₂CH═CHCH₂— | | F |
| A-289 | —CH(CH₃)(CH₂)₃— | | F |
| A-290 | —CH₂CH(CH₃)(CH₂)₂— | | F |
| A-291 | —CH(CH₃)—(CH₂)₂—CH(CH₃)— | | F |
| A-292 | —CH(CH₃)—(CH₂)₄— | | F |
| A-293 | —CH₂—CH(CH₃)—(CH₂)₃— | | F |
| A-294 | —(CH₂)—CH(CH₃)—CH₂—CH(CH₃)—CH₂— | | F |
| A-295 | —CH(CH₂CH₃)—(CH₂)₄— | | F |
| A-296 | —(CH₂)₂—CHOH—(CH₂)₂— | | F |
| A-297 | —(CH₂)₆— | | F |
| A-298 | —CH(CH₃)—(CH₂)₅— | | F |
| A-299 | —(CH₂)₂—N(CH₃)—(CH₂)₂— | | F |
| A-300 | —N═CH—CH═CH— | | F |
| A-301 | —N═C(CH₃)—CH═C(CH₃)— | | F |
| A-302 | —N═C(CF₃)—CH═C(CF₃)— | | F |
| A-303 | H | H | Cl |
| A-304 | CH₃ | H | Cl |
| A-305 | CH₃ | CH₃ | Cl |
| A-306 | CH₂CH₃ | H | Cl |
| A-307 | CH₂CH₃ | CH₃ | Cl |
| A-308 | CH₂CH₃ | CH₂CH₃ | Cl |
| A-309 | CH₂CF₃ | H | Cl |
| A-310 | CH₂CF₃ | CH₃ | Cl |
| A-311 | CH₂CF₃ | CH₂CH₃ | Cl |
| A-312 | CH₂CCl₃ | H | Cl |
| A-313 | CH₂CCl₃ | CH₃ | Cl |
| A-314 | CH₂CCl₃ | CH₂CH₃ | Cl |
| A-315 | CH₂CH₂CH₃ | H | Cl |
| A-316 | CH₂CH₂CH₃ | CH₃ | Cl |
| A-317 | CH₂CH₂CH₃ | CH₂CH₃ | Cl |
| A-318 | CH₂CH₂CH₃ | CH₂CH₂CH₃ | Cl |
| A-319 | CH(CH₃)₂ | H | Cl |
| A-320 | CH(CH₃)₂ | CH₃ | Cl |
| A-321 | CH(CH₃)₂ | CH₂CH₃ | Cl |
| A-322 | CH₂CH(CH₃)₂ | H | Cl |
| A-323 | CH₂CH(CH₃)₂ | CH₃ | Cl |
| A-324 | CH₂CH(CH₃)₂ | CH₂CH₃ | Cl |
| A-325 | CH₂CH(CH₃)₂ | CH₂CH₂CH₃ | Cl |
| A-326 | CH₂CH₂CH₂CH₃ | H | Cl |
| A-327 | CH₂CH₂CH₂CH₃ | CH₃ | Cl |
| A-328 | CH₂CH₂CH₂CH₃ | CH₂CH₃ | Cl |
| A-329 | CH₂CH₂CH₂CH₃ | CH₂CH₂CH₃ | Cl |
| A-330 | CH₂CH₂CH₂CH₃ | CH₂CH₂CH₂CH₃ | Cl |
| A-331 | (±) CH(CH₃)—CH₂CH₃ | H | Cl |
| A-332 | (±) CH(CH₃)—CH₂CH₃ | CH₃ | Cl |
| A-333 | (±) CH(CH₃)—CH₂CH₃ | CH₂CH₃ | Cl |
| A-334 | (S) CH(CH₃)—CH₂CH₃ | H | Cl |
| A-335 | (S) CH(CH₃)—CH₂CH₃ | CH₃ | Cl |
| A-336 | (S) CH(CH₃)—CH₂CH₃ | CH₂CH₃ | Cl |
| A-337 | (R) CH(CH₃)—CH₂CH₃ | H | Cl |
| A-338 | (R) CH(CH₃)—CH₂CH₃ | CH₃ | Cl |
| A-339 | (R) CH(CH₃)—CH₂CH₃ | CH₂CH₃ | Cl |
| A-340 | (±) CH(CH₃)—CH(CH₃)₂ | H | Cl |
| A-341 | (±) CH(CH₃)—CH(CH₃)₂ | CH₃ | Cl |
| A-342 | (±) CH(CH₃)—CH(CH₃)₂ | CH₂CH₃ | Cl |
| A-343 | (S) CH(CH₃)—CH(CH₃)₂ | H | Cl |
| A-344 | (S) CH(CH₃)—CH(CH₃)₂ | CH₃ | Cl |
| A-345 | (S) CH(CH₃)—CH(CH₃)₂ | CH₂CH₃ | Cl |
| A-346 | (R) CH(CH₃)—CH(CH₃)₂ | H | Cl |
| A-347 | (R) CH(CH₃)—CH(CH₃)₂ | CH₃ | Cl |
| A-348 | (R) CH(CH₃)—CH(CH₃)₂ | CH₂CH₃ | Cl |
| A-349 | (±) CH(CH₃)—C(CH₃)₃ | H | Cl |
| A-350 | (±) CH(CH₃)—C(CH₃)₃ | CH₃ | Cl |
| A-351 | (±) CH(CH₃)—C(CH₃)₃ | CH₂CH₃ | Cl |
| A-352 | (S) CH(CH₃)—C(CH₃)₃ | H | Cl |
| A-353 | (S) CH(CH₃)—C(CH₃)₃ | CH₃ | Cl |
| A-354 | (S) CH(CH₃)—C(CH₃)₃ | CH₂CH₃ | Cl |
| A-355 | (R) CH(CH₃)—C(CH₃)₃ | H | Cl |
| A-356 | (R) CH(CH₃)—C(CH₃)₃ | CH₃ | Cl |
| A-357 | (R) CH(CH₃)—C(CH₃)₃ | CH₂CH₃ | Cl |
| A-358 | (±) CH(CH₃)—CF₃ | H | Cl |
| A-359 | (±) CH(CH₃)—CF₃ | CH₃ | Cl |
| A-360 | (±) CH(CH₃)—CF₃ | CH₂CH₃ | Cl |
| A-361 | (S) CH(CH₃)—CF₃ | H | Cl |
| A-362 | (S) CH(CH₃)—CF₃ | CH₃ | Cl |
| A-363 | (S) CH(CH₃)—CF₃ | CH₂CH₃ | Cl |
| A-364 | (R) CH(CH₃)—CF₃ | H | Cl |
| A-365 | (R) CH(CH₃)—CF₃ | CH₃ | Cl |
| A-366 | (R) CH(CH₃)—CF₃ | CH₂CH₃ | Cl |
| A-367 | (±) CH(CH₃)—CCl₃ | H | Cl |
| A-368 | (±) CH(CH₃)—CCl₃ | CH₃ | Cl |
| A-369 | (±) CH(CH₃)—CCl₃ | CH₂CH₃ | Cl |
| A-370 | (S) CH(CH₃)—CCl₃ | H | Cl |
| A-371 | (S) CH(CH₃)—CCl₃ | CH₃ | Cl |
| A-372 | (S) CH(CH₃)—CCl₃ | CH₂CH₃ | Cl |
| A-373 | (R) CH(CH₃)—CCl₃ | H | Cl |
| A-374 | (R) CH(CH₃)—CCl₃ | CH₃ | Cl |
| A-375 | (R) CH(CH₃)—CCl₃ | CH₂CH₃ | Cl |
| A-376 | CH₂CF₂CF₃ | H | Cl |
| A-377 | CH₂CF₂CF₃ | CH₃ | Cl |
| A-378 | CH₂CF₂CF₃ | CH₂CH₃ | Cl |
| A-379 | CH₂(CF₂)₂CF₃ | H | Cl |
| A-380 | CH₂(CF₂)₂CF₃ | CH₃ | Cl |
| A-381 | CH₂(CF₂)₂CF₃ | CH₂CH₃ | Cl |
| A-382 | CH₂C(CH₃)═CH₂ | H | Cl |
| A-383 | CH₂C(CH₃)═CH₂ | CH₃ | Cl |
| A-384 | CH₂C(CH₃)═CH₂ | CH₂CH₃ | Cl |
| A-385 | CH₂CH═CH₂ | H | Cl |
| A-386 | CH₂CH═CH₂ | CH₃ | Cl |
| A-387 | CH₂CH═CH₂ | CH₂CH₃ | Cl |
| A-388 | (±) CH(CH₃)CH═CH₂ | H | Cl |
| A-389 | (±) CH(CH₃)CH═CH₂ | CH₃ | Cl |
| A-390 | (±) CH(CH₃)CH═CH₂ | CH₂CH₃ | Cl |
| A-391 | (S) CH(CH₃)CH═CH₂ | H | Cl |
| A-392 | (S) CH(CH₃)CH═CH₂ | CH₃ | Cl |
| A-393 | (S) CH(CH₃)CH═CH₂ | CH₂CH₃ | Cl |
| A-394 | (R) CH(CH₃)CH═CH₂ | H | Cl |
| A-395 | (R) CH(CH₃)CH═CH₂ | CH₃ | Cl |
| A-396 | (R) CH(CH₃)CH═CH₂ | CH₂CH₃ | Cl |
| A-397 | (±) CH(CH₃)C(CH₃)═CH₂ | H | Cl |
| A-398 | (±)CH(CH₃)C(CH₃)═CH₂ | CH₃ | Cl |
| A-399 | (±)CH(CH₃)C(CH₃)═CH₂ | CH₂CH₃ | Cl |
| A-400 | (S) CH(CH₃)C(CH₃)═CH₂ | H | Cl |
| A-401 | (S) CH(CH₃)C(CH₃)═CH₂ | CH₃ | Cl |
| A-402 | (S) CH(CH₃)C(CH₃)═CH₂ | CH₂CH₃ | Cl |
| A-403 | (R) CH(CH₃)C(CH₃)═CH₂ | H | Cl |
| A-404 | (R) CH(CH₃)C(CH₃)═CH₂ | CH₃ | Cl |
| A-405 | (R) CH(CH₃)C(CH₃)═CH₂ | CH₂CH₃ | Cl |
| A-406 | CH₂—C≡CH | H | Cl |
| A-407 | CH₂—C≡CH | CH₃ | Cl |
| A-408 | CH₂—C≡CH | CH₂CH₃ | Cl |
| A-409 | cyclopentyl | H | Cl |
| A-410 | cyclopentyl | CH₃ | Cl |
| A-411 | cyclopentyl | CH₂CH₃ | Cl |
| A-412 | cyclohexyl | H | Cl |
| A-413 | cyclohexyl | CH₃ | Cl |
| A-414 | cyclohexyl | CH₂CH₃ | Cl |
| A-415 | CH₂—C₆H₅ | H | Cl |
| A-416 | CH₂—C₆H₅ | CH₃ | Cl |
| A-417 | CH₂—C₆H₅ | CH₂CH₃ | Cl |
| A-418 | NH₂ | CH₂-c-C₆H₁₁ | Cl |
| A-419 | NH₂ | CH₂CH₃ | Cl |
| A-420 | NH₂ | CH₂CH₂CH₃ | Cl |
| A-421 | NH—CH₂—CH═CH₂ | H | Cl |
| A-422 | NH—CH₂—CH═CH₂ | CH₃ | Cl |
| A-423 | NH—CH₂—CH═CH₂ | CH₂CH₃ | Cl |
| A-424 | NH—C(CH₃)₃ | H | Cl |
| A-425 | N(CH₃)₂ | H | Cl |
| A-426 | NH(CH₃) | H | Cl |
| A-427 | —(CH₂)₂CH═CHCH₂— | | Cl |
| A-428 | —(CH₂)₂C(CH₃)═CHCH₂— | | Cl |
| A-429 | —CH(CH₃)CH₂—CH═CHCH₂— | | Cl |
| A-430 | —(CH₂)₂CH(CH₃)(CH₂)₂— | | Cl |
| A-431 | —(CH₂)₃CHFCH₂— | | Cl |
| A-432 | —(CH₂)₂CHF(CH₂)₂— | | Cl |
| A-433 | —CH₂CHF(CH₂)₃— | | Cl |
| A-434 | —(CH₂)₂CH(CF₃)(CH₂)₂— | | Cl |
| A-435 | —(CH₂)₂O(CH₂)₂— | | Cl |
| A-436 | —(CH₂)₂S(CH₂)₂— | | Cl |
| A-437 | —(CH₂)₅— | | Cl |
| A-438 | —(CH₂)₄— | | Cl |
| A-439 | —CH₂CH═CHCH₂— | | Cl |
| A-440 | —CH(CH₃)(CH₂)₃— | | Cl |
| A-441 | —CH₂CH(CH₃)(CH₂)₂— | | Cl |
| A-442 | —CH(CH₃)—(CH₂)₂—CH(CH₃)— | | Cl |
| A-443 | —CH(CH₃)—(CH₂)₄— | | Cl |
| A-444 | —CH₂—CH(CH₃)—(CH₂)₃— | | Cl |
| A-445 | —(CH₂)—CH(CH₃)—CH₂—CH(CH₃)—CH₂— | | Cl |
| A-446 | —CH(CH₂CH₃)—(CH₂)₄— | | Cl |
| A-447 | —(CH₂)₂—CHOH—(CH₂)₂— | | Cl |
| A-448 | —(CH₂)₆— | | Cl |
| A-449 | —CH(CH₃)—(CH₂)₅— | | Cl |
| A-450 | —(CH₂)₂—N(CH₃)—(CH₂)₂— | | Cl |
| A-451 | —N═CH—CH═CH— | | Cl |
| A-452 | —N═C(CH₃)—CH═C(CH₃)— | | Cl |
| A-453 | —N═C(CF₃)—CH═C(CF₃)— | | Cl |
| A-454 | H | H | Br |
| A-455 | CH₃ | H | Br |
| A-456 | CH₃ | CH₃ | Br |
| A-457 | CH₂CH₃ | H | Br |
| A-458 | CH₂CH₃ | CH₃ | Br |
| A-459 | CH₂CH₃ | CH₂CH₃ | Br |
| A-460 | CH₂CF₃ | H | Br |
| A-461 | CH₂CF₃ | CH₃ | Br |
| A-462 | CH₂CF₃ | CH₂CH₃ | Br |
| A-463 | CH₂CCl₃ | H | Br |
| A-464 | CH₂CCl₃ | CH₃ | Br |
| A-465 | CH₂CCl₃ | CH₂CH₃ | Br |
| A-466 | CH₂CH₂CH₃ | H | Br |
| A-467 | CH₂CH₂CH₃ | CH₃ | Br |
| A-468 | CH₂CH₂CH₃ | CH₂CH₃ | Br |
| A-469 | CH₂CH₂CH₃ | CH₂CH₂CH₃ | Br |
| A-470 | CH(CH₃)₂ | H | Br |
| A-471 | CH(CH₃)₂ | CH₃ | Br |
| A-472 | CH(CH₃)₂ | CH₂CH₃ | Br |
| A-473 | CH₂CH(CH₃)₂ | H | Br |
| A-474 | CH₂CH(CH₃)₂ | CH₃ | Br |
| A-475 | CH₂CH(CH₃)₂ | CH₂CH₃ | Br |
| A-476 | CH₂CH(CH₃)₂ | CH₂CH₂CH₃ | Br |
| A-477 | CH₂CH₂CH₂CH₃ | H | Br |
| A-478 | CH₂CH₂CH₂CH₃ | CH₃ | Br |
| A-479 | CH₂CH₂CH₂CH₃ | CH₂CH₃ | Br |
| A-480 | CH₂CH₂CH₂CH₃ | CH₂CH₂CH₃ | Br |
| A-481 | CH₂CH₂CH₂CH₃ | CH₂CH₂CH₂CH₃ | Br |
| A-482 | (±) CH(CH₃)—CH₂CH₃ | H | Br |
| A-483 | (±) CH(CH₃)—CH₂CH₃ | CH₃ | Br |
| A-484 | (±) CH(CH₃)—CH₂CH₃ | CH₂CH₃ | Br |
| A-485 | (S) CH(CH₃)—CH₂CH₃ | H | Br |
| A-486 | (S) CH(CH₃)—CH₂CH₃ | CH₃ | Br |
| A-487 | (S) CH(CH₃)—CH₂CH₃ | CH₂CH₃ | Br |
| A-488 | (R) CH(CH₃)—CH₂CH₃ | H | Br |
| A-489 | (R) CH(CH₃)—CH₂CH₃ | CH₃ | Br |
| A-490 | (R) CH(CH₃)—CH₂CH₃ | CH₂CH₃ | Br |
| A-491 | (±) CH(CH₃)—CH(CH₃)₂ | H | Br |
| A-492 | (±) CH(CH₃)—CH(CH₃)₂ | CH₃ | Br |
| A-493 | (±) CH(CH₃)—CH(CH₃)₂ | CH₂CH₃ | Br |
| A-494 | (S) CH(CH₃)—CH(CH₃)₂ | H | Br |
| A-495 | (S) CH(CH₃)—CH(CH₃)₂ | CH₃ | Br |
| A-496 | (S) CH(CH₃)—CH(CH₃)₂ | CH₂CH₃ | Br |
| A-497 | (R) CH(CH₃)—CH(CH₃)₂ | H | Br |
| A-498 | (R) CH(CH₃)—CH(CH₃)₂ | CH₃ | Br |
| A-499 | (R) CH(CH₃)—CH(CH₃)₂ | CH₂CH₃ | Br |
| A-500 | (±) CH(CH₃)—C(CH₃)₃ | H | Br |
| A-501 | (±) CH(CH₃)—C(CH₃)₃ | CH₃ | Br |
| A-502 | (±) CH(CH₃)—C(CH₃)₃ | CH₂CH₃ | Br |
| A-503 | (S) CH(CH₃)—C(CH₃)₃ | H | Br |
| A-504 | (S) CH(CH₃)—C(CH₃)₃ | CH₃ | Br |
| A-505 | (S) CH(CH₃)—C(CH₃)₃ | CH₂CH₃ | Br |
| A-506 | (R) CH(CH₃)—C(CH₃)₃ | H | Br |
| A-507 | (R) CH(CH₃)—C(CH₃)₃ | CH₃ | Br |
| A-508 | (R) CH(CH₃)—C(CH₃)₃ | CH₂CH₃ | Br |
| A-509 | (±) CH(CH₃)—CF₃ | H | Br |
| A-510 | (±) CH(CH₃)—CF₃ | CH₃ | Br |
| A-511 | (±) CH(CH₃)—CF₃ | CH₂CH₃ | Br |
| A-512 | (S) CH(CH₃)—CF₃ | H | Br |
| A-513 | (S) CH(CH₃)—CF₃ | CH₃ | Br |
| A-514 | (S) CH(CH₃)—CF₃ | CH₂CH₃ | Br |
| A-515 | (R) CH(CH₃)—CF₃ | H | Br |
| A-516 | (R) CH(CH₃)—CF₃ | CH₃ | Br |
| A-517 | (R) CH(CH₃)—CF₃ | CH₂CH₃ | Br |
| A-518 | (±) CH(CH₃)—CCl₃ | H | Br |
| A-519 | (±) CH(CH₃)—CCl₃ | CH₃ | Br |
| A-520 | (±) CH(CH₃)—CCl₃ | CH₂CH₃ | Br |
| A-521 | (S) CH(CH₃)—CCl₃ | H | Br |
| A-522 | (S) CH(CH₃)—CCl₃ | CH₃ | Br |
| A-523 | (S) CH(CH₃)—CCl₃ | CH₂CH₃ | Br |
| A-524 | (R) CH(CH₃)—CCl₃ | H | Br |
| A-525 | (R) CH(CH₃)—CCl₃ | CH₃ | Br |
| A-526 | (R) CH(CH₃)—CCl₃ | CH₂CH₃ | Br |
| A-527 | CH₂CF₂CF₃ | H | Br |
| A-528 | CH₂CF₂CF₃ | CH₃ | Br |
| A-529 | CH₂CF₂CF₃ | CH₂CH₃ | Br |
| A-530 | CH₂(CF₂)₂CF₃ | H | Br |
| A-531 | CH₂(CF₂)₂CF₃ | CH₃ | Br |
| A-532 | CH₂(CF₂)₂CF₃ | CH₂CH₃ | Br |
| A-533 | CH₂C(CH₃)═CH₂ | H | Br |
| A-534 | CH₂C(CH₃)═CH₂ | CH₃ | Br |
| A-535 | CH₂C(CH₃)═CH₂ | CH₂CH₃ | Br |
| A-536 | CH₂CH═CH₂ | H | Br |
| A-537 | CH₂CH═CH₂ | CH₃ | Br |
| A-538 | CH₂CH═CH₂ | CH₂CH₃ | Br |
| A-539 | (±) CH(CH₃)CH═CH₂ | H | Br |
| A-540 | (±) CH(CH₃)CH═CH₂ | CH₃ | Br |
| A-541 | (±) CH(CH₃)CH═CH₂ | CH₂CH₃ | Br |
| A-542 | (S) CH(CH₃)CH═CH₂ | H | Br |
| A-543 | (S) CH(CH₃)CH═CH₂ | CH₃ | Br |
| A-544 | (S) CH(CH₃)CH═CH₂ | CH₂CH₃ | Br |
| A-545 | (R) CH(CH₃)CH═CH₂ | H | Br |
| A-546 | (R) CH(CH₃)CH═CH₂ | CH₃ | Br |
| A-547 | (R) CH(CH₃)CH═CH₂ | CH₂CH₃ | Br |
| A-548 | (±)CH(CH₃)C(CH₃)═CH₂ | H | Br |
| A-549 | (±)CH(CH₃)C(CH₃)═CH₂ | CH₃ | Br |
| A-550 | (±)CH(CH₃)C(CH₃)═CH₂ | CH₂CH₃ | Br |
| A-551 | (S) CH(CH₃)C(CH₃)═CH₂ | H | Br |
| A-552 | (S) CH(CH₃)C(CH₃)═CH₂ | CH₃ | Br |
| A-553 | (S) CH(CH₃)C(CH₃)═CH₂ | CH₂CH₃ | Br |
| A-554 | (R) CH(CH₃)C(CH₃)═CH₂ | H | Br |
| A-555 | (R) CH(CH₃)C(CH₃)═CH₂ | CH₃ | Br |
| A-556 | (R) CH(CH₃)C(CH₃)═CH₂ | CH₂CH₃ | Br |
| A-557 | CH₂—C≡CH | H | Br |
| A-558 | CH₂—C≡CH | CH₃ | Br |
| A-559 | CH₂—C≡CH | CH₂CH₃ | Br |
| A-560 | cyclopentyl | H | Br |
| A-561 | cyclopentyl | CH₃ | Br |
| A-562 | cyclopentyl | CH₂CH₃ | Br |
| A-563 | cyclohexyl | H | Br |
| A-564 | cyclohexyl | CH₃ | Br |
| A-565 | cyclohexyl | CH₂CH₃ | Br |
| A-566 | CH₂—C₆H₅ | H | Br |
| A-567 | CH₂—C₆H₅ | CH₃ | Br |
| A-568 | CH₂—C₆H₅ | CH₂CH₃ | Br |
| A-569 | NH₂ | CH₂-c-C₆H₁₁ | Br |
| A-570 | NH₂ | CH₂CH₃ | Br |
| A-571 | NH₂ | CH₂CH₂CH₃ | Br |
| A-572 | NH—CH₂—CH═CH₂ | H | Br |
| A-573 | NH—CH₂—CH═CH₂ | CH₃ | Br |
| A-574 | NH—CH₂—CH═CH₂ | CH₂CH₃ | Br |
| A-575 | NH—C(CH₃)₃ | H | Br |
| A-576 | N(CH₃)₂ | H | Br |
| A-577 | NH(CH₃) | H | Br |
| A-578 | —(CH₂)₂CH═CHCH₂— | | Br |
| A-579 | —(CH₂)₂C(CH₃)═CHCH₂— | | Br |
| A-580 | —CH(CH₃)CH₂—CH═CHCH₂— | | Br |
| A-581 | —(CH₂)₂CH(CH₃)(CH₂)₂— | | Br |
| A-582 | —(CH₂)₃CHFCH₂— | | Br |
| A-583 | —(CH₂)₂CHF(CH₂)₂— | | Br |
| A-584 | —CH₂CHF(CH₂)₃— | | Br |
| A-585 | —(CH₂)₂CH(CF₃)(CH₂)₂— | | Br |
| A-586 | —(CH₂)₂O(CH₂)₂— | | Br |
| A-587 | —(CH₂)₂S(CH₂)₂— | | Br |
| A-588 | —(CH₂)₅— | | Br |
| A-589 | —(CH₂)₄— | | Br |
| A-590 | —CH₂CH═CHCH₂— | | Br |
| A-591 | —CH(CH₃)(CH₂)₃— | | Br |
| A-592 | —CH₂CH(CH₃)(CH₂)₂— | | Br |
| A-593 | —CH(CH₃)—(CH₂)₂—CH(CH₃)— | | Br |
| A-594 | —CH(CH₃)—(CH₂)₄— | | Br |
| A-595 | —CH₂—CH(CH₃)—(CH₂)₃— | | Br |
| A-596 | —(CH₂)—CH(CH₃)—CH₂—CH(CH₃)—CH₂— | | Br |
| A-597 | —CH(CH₂CH₃)—(CH₂)₄— | | Br |
| A-598 | —(CH₂)₂—CHOH—(CH₂)₂— | | Br |
| A-599 | —(CH₂)₆— | | Br |
| A-600 | —CH(CH₃)—(CH₂)₅— | | Br |
| A-601 | —(CH₂)₂—N(CH₃)—(CH₂)₂— | | Br |
| A-602 | —N═CH—CH═CH— | | Br |
| A-603 | —N═C(CH₃)—CH═C(CH₃)— | | Br |
| A-604 | —N═C(CF₃)—CH═C(CF₃)— | | Br |
| A-605 | H | H | methyl |
| A-606 | CH₃ | H | methyl |
| A-607 | CH₃ | CH₃ | methyl |
| A-608 | CH₂CH₃ | H | methyl |
| A-609 | CH₂CH₃ | CH₃ | methyl |
| A-610 | CH₂CH₃ | CH₂CH₃ | methyl |
| A-611 | CH₂CF₃ | H | methyl |
| A-612 | CH₂CF₃ | CH₃ | methyl |
| A-613 | CH₂CF₃ | CH₂CH₃ | methyl |
| A-614 | CH₂CCl₃ | H | methyl |
| A-615 | CH₂CCl₃ | CH₃ | methyl |
| A-616 | CH₂CCl₃ | CH₂CH₃ | methyl |
| A-617 | CH₂CH₂CH₃ | H | methyl |
| A-618 | CH₂CH₂CH₃ | CH₃ | methyl |
| A-619 | CH₂CH₂CH₃ | CH₂CH₃ | methyl |
| A-620 | CH₂CH₂CH₃ | CH₂CH₂CH₃ | methyl |
| A-621 | CH(CH₃)₂ | H | methyl |
| A-622 | CH(CH₃)₂ | CH₃ | methyl |
| A-623 | CH(CH₃)₂ | CH₂CH₃ | methyl |
| A-624 | CH₂CH(CH₃)₂ | H | methyl |
| A-625 | CH₂CH(CH₃)₂ | CH₃ | methyl |
| A-626 | CH₂CH(CH₃)₂ | CH₂CH₃ | methyl |
| A-627 | CH₂CH(CH₃)₂ | CH₂CH₂CH₃ | methyl |
| A-628 | CH₂CH₂CH₂CH₃ | H | methyl |
| A-629 | CH₂CH₂CH₂CH₃ | CH₃ | methyl |
| A-630 | CH₂CH₂CH₂CH₃ | CH₂CH₃ | methyl |
| A-631 | CH₂CH₂CH₂CH₃ | CH₂CH₂CH₃ | methyl |
| A-632 | CH₂CH₂CH₂CH₃ | CH₂CH₂CH₂CH₃ | methyl |
| A-633 | (±) CH(CH₃)—CH₂CH₃ | H | methyl |
| A-634 | (±) CH(CH₃)—CH₂CH₃ | CH₃ | methyl |
| A-635 | (±) CH(CH₃)—CH₂CH₃ | CH₂CH₃ | methyl |
| A-636 | (S) CH(CH₃)—CH₂CH₃ | H | methyl |
| A-637 | (S) CH(CH₃)—CH₂CH₃ | CH₃ | methyl |
| A-638 | (S) CH(CH₃)—CH₂CH₃ | CH₂CH₃ | methyl |
| A-639 | (R) CH(CH₃)—CH₂CH₃ | H | methyl |
| A-640 | (R) CH(CH₃)—CH₂CH₃ | CH₃ | methyl |
| A-641 | (R) CH(CH₃)—CH₂CH₃ | CH₂CH₃ | methyl |
| A-642 | (±) CH(CH₃)—CH(CH₃)₂ | H | methyl |
| A-643 | (±) CH(CH₃)—CH(CH₃)₂ | CH₃ | methyl |
| A-644 | (±) CH(CH₃)—CH(CH₃)₂ | CH₂CH₃ | methyl |
| A-645 | (S) CH(CH₃)—CH(CH₃)₂ | H | methyl |
| A-646 | (S) CH(CH₃)—CH(CH₃)₂ | CH₃ | methyl |
| A-647 | (S) CH(CH₃)—CH(CH₃)₂ | CH₂CH₃ | methyl |
| A-648 | (R) CH(CH₃)—CH(CH₃)₂ | H | methyl |
| A-649 | (R) CH(CH₃)—CH(CH₃)₂ | CH₃ | methyl |
| A-650 | (R) CH(CH₃)—CH(CH₃)₂ | CH₂CH₃ | methyl |
| A-651 | (±) CH(CH₃)—C(CH₃)₃ | H | methyl |
| A-652 | (±) CH(CH₃)—C(CH₃)₃ | CH₃ | methyl |
| A-653 | (±) CH(CH₃)—C(CH₃)₃ | CH₂CH₃ | methyl |
| A-654 | (S) CH(CH₃)—C(CH₃)₃ | H | methyl |
| A-655 | (S) CH(CH₃)—C(CH₃)₃ | CH₃ | methyl |
| A-656 | (S) CH(CH₃)—C(CH₃)₃ | CH₂CH₃ | methyl |
| A-657 | (R) CH(CH₃)—C(CH₃)₃ | H | methyl |
| A-658 | (R) CH(CH₃)—C(CH₃)₃ | CH₃ | methyl |
| A-659 | (R) CH(CH₃)—C(CH₃)₃ | CH₂CH₃ | methyl |
| A-660 | (±) CH(CH₃)—CF₃ | H | methyl |
| A-661 | (±) CH(CH₃)—CF₃ | CH₃ | methyl |
| A-662 | (±) CH(CH₃)—CF₃ | CH₂CH₃ | methyl |
| A-663 | (S) CH(CH₃)—CF₃ | H | methyl |
| A-664 | (S) CH(CH₃)—CF₃ | CH₃ | methyl |
| A-665 | (S) CH(CH₃)—CF₃ | CH₂CH₃ | methyl |
| A-666 | (R) CH(CH₃)—CF₃ | H | methyl |
| A-667 | (R) CH(CH₃)—CF₃ | CH₃ | methyl |
| A-668 | (R) CH(CH₃)—CF₃ | CH₂CH₃ | methyl |
| A-669 | (±) CH(CH₃)—CCl₃ | H | methyl |
| A-670 | (±) CH(CH₃)—CCl₃ | CH₃ | methyl |
| A-671 | (±) CH(CH₃)—CCl₃ | CH₂CH₃ | methyl |
| A-672 | (S) CH(CH₃)—CCl₃ | H | methyl |
| A-673 | (S) CH(CH₃)—CCl₃ | CH₃ | methyl |
| A-674 | (S) CH(CH₃)—CCl₃ | CH₂CH₃ | methyl |
| A-675 | (R) CH(CH₃)—CCl₃ | H | methyl |
| A-676 | (R) CH(CH₃)—CCl₃ | CH₃ | methyl |
| A-677 | (R) CH(CH₃)—CCl₃ | CH₂CH₃ | methyl |
| A-678 | CH₂CF₂CF₃ | H | methyl |
| A-679 | CH₂CF₂CF₃ | CH₃ | methyl |
| A-680 | CH₂CF₂CF₃ | CH₂CH₃ | methyl |
| A-681 | CH₂(CF₂)₂CF₃ | H | methyl |
| A-682 | CH₂(CF₂)₂CF₃ | CH₃ | methyl |
| A-683 | CH₂(CF₂)₂CF₃ | CH₂CH₃ | methyl |
| A-684 | CH₂C(CH₃)═CH₂ | H | methyl |
| A-685 | CH₂C(CH₃)═CH₂ | CH₃ | methyl |
| A-686 | CH₂C(CH₃)═CH₂ | CH₂CH₃ | methyl |
| A-687 | CH₂CH═CH₂ | H | methyl |
| A-688 | CH₂CH═CH₂ | CH₃ | methyl |
| A-689 | CH₂CH═CH₂ | CH₂CH₃ | methyl |
| A-690 | (±) CH(CH₃)CH═CH₂ | H | methyl |
| A-691 | (±) CH(CH₃)CH═CH₂ | CH₃ | methyl |
| A-692 | (±) CH(CH₃)CH═CH₂ | CH₂CH₃ | methyl |
| A-693 | (S) CH(CH₃)CH═CH₂ | H | methyl |
| A-694 | (S) CH(CH₃)CH═CH₂ | CH₃ | methyl |
| A-695 | (S) CH(CH₃)CH═CH₂ | CH₂CH₃ | methyl |
| A-696 | (R) CH(CH₃)CH═CH₂ | H | methyl |
| A-697 | (R) CH(CH₃)CH═CH₂ | CH₃ | methyl |
| A-698 | (R) CH(CH₃)CH═CH₂ | CH₂CH₃ | methyl |
| A-699 | (±)CH(CH₃)C(CH₃)═CH₂ | H | methyl |
| A-700 | (±)CH(CH₃)C(CH₃)═CH₂ | CH₃ | methyl |
| A-701 | (±)CH(CH₃)C(CH₃)═CH₂ | CH₂CH₃ | methyl |
| A-702 | (S) CH(CH₃)C(CH₃)═CH₂ | H | methyl |
| A-703 | (S) CH(CH₃)C(CH₃)═CH₂ | CH₃ | methyl |
| A-704 | (S) CH(CH₃)C(CH₃)═CH₂ | CH₂CH₃ | methyl |
| A-705 | (R) CH(CH₃)C(CH₃)═CH₂ | H | methyl |
| A-706 | (R) CH(CH₃)C(CH₃)═CH₂ | CH₃ | methyl |
| A-707 | (R) CH(CH₃)C(CH₃)═CH₂ | CH₂CH₃ | methyl |
| A-708 | CH₂—C≡CH | H | methyl |
| A-709 | CH₂—C≡CH | CH₃ | methyl |
| A-710 | CH₂—C≡CH | CH₂CH₃ | methyl |
| A-711 | cyclopentyl | H | methyl |
| A-712 | cyclopentyl | CH₃ | methyl |
| A-713 | cyclopentyl | CH₂CH₃ | methyl |
| A-714 | cyclohexyl | H | methyl |
| A-715 | cyclohexyl | CH₃ | methyl |
| A-716 | cyclohexyl | CH₂CH₃ | methyl |
| A-717 | CH₂—C₆H₅ | H | methyl |
| A-718 | CH₂—C₆H₅ | CH₃ | methyl |
| A-719 | CH₂—C₆H₅ | CH₂CH₃ | methyl |
| A-720 | NH₂ | CH₂-c-C₆H₁₁ | methyl |
| A-721 | NH₂ | CH₂CH₃ | methyl |
| A-722 | NH₂ | CH₂CH₂CH₃ | methyl |
| A-723 | NH—CH₂—CH═CH₂ | H | methyl |
| A-724 | NH—CH₂—CH═CH₂ | CH₃ | methyl |
| A-725 | NH—CH₂—CH═CH₂ | CH₂CH₃ | methyl |
| A-726 | NH—C(CH₃)₃ | H | methyl |
| A-727 | N(CH₃)₂ | H | methyl |
| A-728 | NH(CH₃) | H | methyl |
| A-729 | —(CH₂)₂CH═CHCH₂— | | methyl |
| A-730 | —(CH₂)₂C(CH₃)═CHCH₂— | | methyl |
| A-731 | —CH(CH₃)CH₂—CH═CHCH₂— | | methyl |
| A-732 | —(CH₂)₂CH(CH₃)(CH₂)₂— | | methyl |
| A-733 | —(CH₂)₃CHFCH₂— | | methyl |
| A-734 | —(CH₂)₂CHF(CH₂)₂— | | methyl |
| A-735 | —CH₂CHF(CH₂)₃— | | methyl |
| A-736 | —(CH₂)₂CH(CF₃)(CH₂)₂— | | methyl |
| A-737 | —(CH₂)₂O(CH₂)₂— | | methyl |
| A-738 | —(CH₂)₂S(CH₂)₂— | | methyl |
| A-739 | —(CH₂)₅— | | methyl |
| A-740 | —(CH₂)₄— | | methyl |
| A-741 | —CH₂CH═CHCH₂— | | methyl |
| A-742 | —CH(CH₃)(CH₂)₃— | | methyl |
| A-743 | —CH₂CH(CH₃)(CH₂)₂— | | methyl |
| A-744 | —CH(CH₃)—(CH₂)₂—CH(CH₃)— | | methyl |
| A-745 | —CH(CH₃)—(CH₂)₄— | | methyl |
| A-746 | —CH₂—CH(CH₃)—(CH₂)₃— | | methyl |
| A-747 | —(CH₂)—CH(CH₃)—CH₂—CH(CH₃)—CH₂— | | methyl |
| A-748 | —CH(CH₂CH₃)—(CH₂)₄— | | methyl |
| A-749 | —(CH₂)₂—CHOH—(CH₂)₂— | | methyl |
| A-750 | —(CH₂)₆— | | methyl |
| A-751 | —CH(CH₃)—(CH₂)₅— | | methyl |
| A-752 | —(CH₂)₂—N(CH₃)—(CH₂)₂— | | methyl |
| A-753 | —N═CH—CH═CH— | | methyl |
| A-754 | —N═C(CH₃)—CH═C(CH₃)— | | methyl |
| A-755 | —N═C(CF₃)—CH═C(CF₃)— | | methyl |
| A-756 | H | H | cyclopropyl |
| A-757 | CH₃ | H | cyclopropyl |
| A-758 | CH₃ | CH₃ | cyclopropyl |
| A-759 | CH₂CH₃ | H | cyclopropyl |
| A-760 | CH₂CH₃ | CH₃ | cyclopropyl |
| A-761 | CH₂CH₃ | CH₂CH₃ | cyclopropyl |
| A-762 | CH₂CF₃ | H | cyclopropyl |
| A-763 | CH₂CF₃ | CH₃ | cyclopropyl |
| A-764 | CH₂CF₃ | CH₂CH₃ | cyclopropyl |
| A-765 | CH₂CCl₃ | H | cyclopropyl |
| A-766 | CH₂CCl₃ | CH₃ | cyclopropyl |
| A-767 | CH₂CCl₃ | CH₂CH₃ | cyclopropyl |
| A-768 | CH₂CH₂CH₃ | H | cyclopropyl |
| A-769 | CH₂CH₂CH₃ | CH₃ | cyclopropyl |
| A-770 | CH₂CH₂CH₃ | CH₂CH₃ | cyclopropyl |
| A-771 | CH₂CH₂CH₃ | CH₂CH₂CH₃ | cyclopropyl |
| A-772 | CH(CH₃)₂ | H | cyclopropyl |
| A-773 | CH(CH₃)₂ | CH₃ | cyclopropyl |
| A-774 | CH(CH₃)₂ | CH₂CH₃ | cyclopropyl |
| A-775 | CH₂CH(CH₃)₂ | H | cyclopropyl |
| A-776 | CH₂CH(CH₃)₂ | CH₃ | cyclopropyl |
| A-777 | CH₂CH(CH₃)₂ | CH₂CH₃ | cyclopropyl |
| A-778 | CH₂CH(CH₃)₂ | CH₂CH₂CH₃ | cyclopropyl |
| A-779 | CH₂CH₂CH₂CH₃ | H | cyclopropyl |
| A-780 | CH₂CH₂CH₂CH₃ | CH₃ | cyclopropyl |
| A-781 | CH₂CH₂CH₂CH₃ | CH₂CH₃ | cyclopropyl |
| A-782 | CH₂CH₂CH₂CH₃ | CH₂CH₂CH₃ | cyclopropyl |
| A-783 | CH₂CH₂CH₂CH₃ | CH₂CH₂CH₂CH₃ | cyclopropyl |
| A-784 | (±) CH(CH₃)—CH₂CH₃ | H | cyclopropyl |
| A-785 | (±) CH(CH₃)—CH₂CH₃ | CH₃ | cyclopropyl |
| A-786 | (±) CH(CH₃)—CH₂CH₃ | CH₂CH₃ | cyclopropyl |
| A-787 | (S) CH(CH₃)—CH₂CH₃ | H | cyclopropyl |
| A-788 | (S) CH(CH₃)—CH₂CH₃ | CH₃ | cyclopropyl |
| A-789 | (S) CH(CH₃)—CH₂CH₃ | CH₂CH₃ | cyclopropyl |
| A-790 | (R) CH(CH₃)—CH₂CH₃ | H | cyclopropyl |
| A-791 | (R) CH(CH₃)—CH₂CH₃ | CH₃ | cyclopropyl |
| A-792 | (R) CH(CH₃)—CH₂CH₃ | CH₂CH₃ | cyclopropyl |
| A-793 | (±) CH(CH₃)—CH(CH₃)₂ | H | cyclopropyl |
| A-794 | (±) CH(CH₃)—CH(CH₃)₂ | CH₃ | cyclopropyl |
| A-795 | (±) CH(CH₃)—CH(CH₃)₂ | CH₂CH₃ | cyclopropyl |
| A-796 | (S) CH(CH₃)—CH(CH₃)₂ | H | cyclopropyl |
| A-797 | (S) CH(CH₃)—CH(CH₃)₂ | CH₃ | cyclopropyl |
| A-798 | (S) CH(CH₃)—CH(CH₃)₂ | CH₂CH₃ | cyclopropyl |
| A-799 | (R) CH(CH₃)—CH(CH₃)₂ | H | cyclopropyl |
| A-800 | (R) CH(CH₃)—CH(CH₃)₂ | CH₃ | cyclopropyl |
| A-801 | (R) CH(CH₃)—CH(CH₃)₂ | CH₂CH₃ | cyclopropyl |
| A-802 | (±) CH(CH₃)—C(CH₃)₃ | H | cyclopropyl |
| A-803 | (±) CH(CH₃)—C(CH₃)₃ | CH₃ | cyclopropyl |
| A-804 | (±) CH(CH₃)—C(CH₃)₃ | CH₂CH₃ | cyclopropyl |
| A-805 | (S) CH(CH₃)—C(CH₃)₃ | H | cyclopropyl |
| A-806 | (S) CH(CH₃)—C(CH₃)₃ | CH₃ | cyclopropyl |
| A-807 | (S) CH(CH₃)—C(CH₃)₃ | CH₂CH₃ | cyclopropyl |
| A-808 | (R) CH(CH₃)—C(CH₃)₃ | H | cyclopropyl |
| A-809 | (R) CH(CH₃)—C(CH₃)₃ | CH₃ | cyclopropyl |
| A-810 | (R) CH(CH₃)—C(CH₃)₃ | CH₂CH₃ | cyclopropyl |
| A-811 | (±) CH(CH₃)—CF₃ | H | cyclopropyl |
| A-812 | (±) CH(CH₃)—CF₃ | CH₃ | cyclopropyl |
| A-813 | (±) CH(CH₃)—CF₃ | CH₂CH₃ | cyclopropyl |
| A-814 | (S) CH(CH₃)—CF₃ | H | cyclopropyl |
| A-815 | (S) CH(CH₃)—CF₃ | CH₃ | cyclopropyl |
| A-816 | (S) CH(CH₃)—CF₃ | CH₂CH₃ | cyclopropyl |
| A-817 | (R) CH(CH₃)—CF₃ | H | cyclopropyl |
| A-818 | (R) CH(CH₃)—CF₃ | CH₃ | cyclopropyl |
| A-819 | (R) CH(CH₃)—CF₃ | CH₂CH₃ | cyclopropyl |
| A-820 | (±) CH(CH₃)—CCl₃ | H | cyclopropyl |
| A-821 | (±) CH(CH₃)—CCl₃ | CH₃ | cyclopropyl |
| A-822 | (±) CH(CH₃)—CCl₃ | CH₂CH₃ | cyclopropyl |
| A-823 | (S) CH(CH₃)—CCl₃ | H | cyclopropyl |
| A-824 | (S) CH(CH₃)—CCl₃ | CH₃ | cyclopropyl |
| A-825 | (S) CH(CH₃)—CCl₃ | CH₂CH₃ | cyclopropyl |
| A-826 | (R) CH(CH₃)—CCl₃ | H | cyclopropyl |
| A-827 | (R) CH(CH₃)—CCl₃ | CH₃ | cyclopropyl |
| A-828 | (R) CH(CH₃)—CCl₃ | CH₂CH₃ | cyclopropyl |
| A-829 | CH₂CF₂CF₃ | H | cyclopropyl |
| A-830 | CH₂CF₂CF₃ | CH₃ | cyclopropyl |
| A-831 | CH₂CF₂CF₃ | CH₂CH₃ | cyclopropyl |
| A-832 | CH₂(CF₂)₂CF₃ | H | cyclopropyl |
| A-833 | CH₂(CF₂)₂CF₃ | CH₃ | cyclopropyl |
| A-834 | CH₂(CF₂)₂CF₃ | CH₂CH₃ | cyclopropyl |
| A-835 | CH₂C(CH₃)═CH₂ | H | cyclopropyl |
| A-836 | CH₂C(CH₃)═CH₂ | CH₃ | cyclopropyl |
| A-837 | CH₂C(CH₃)═CH₂ | CH₂CH₃ | cyclopropyl |
| A-838 | CH₂CH═CH₂ | H | cyclopropyl |
| A-839 | CH₂CH═CH₂ | CH₃ | cyclopropyl |
| A-840 | CH₂CH═CH₂ | CH₂CH₃ | cyclopropyl |
| A-841 | (±) CH(CH₃)CH═CH₂ | H | cyclopropyl |
| A-842 | (±) CH(CH₃)CH═CH₂ | CH₃ | cyclopropyl |
| A-843 | (±) CH(CH₃)CH═CH₂ | CH₂CH₃ | cyclopropyl |
| A-844 | (S) CH(CH₃)CH═CH₂ | H | cyclopropyl |
| A-845 | (S) CH(CH₃)CH═CH₂ | CH₃ | cyclopropyl |
| A-846 | (S) CH(CH₃)CH═CH₂ | CH₂CH₃ | cyclopropyl |
| A-847 | (R) CH(CH₃)CH═CH₂ | H | cyclopropyl |
| A-848 | (R) CH(CH₃)CH═CH₂ | CH₃ | cyclopropyl |
| A-849 | (R) CH(CH₃)CH═CH₂ | CH₂CH₃ | cyclopropyl |
| A-850 | (±)CH(CH₃)C(CH₃)═CH₂ | H | cyclopropyl |
| A-851 | (±)CH(CH₃)C(CH₃)═CH₂ | CH₃ | cyclopropyl |
| A-852 | (±)CH(CH₃)C(CH₃)═CH₂ | CH₂CH₃ | cyclopropyl |
| A-853 | (S) CH(CH₃)C(CH₃)═CH₂ | H | cyclopropyl |
| A-854 | (S) CH(CH₃)C(CH₃)═CH₂ | CH₃ | cyclopropyl |
| A-855 | (S) CH(CH₃)C(CH₃)═CH₂ | CH₂CH₃ | cyclopropyl |
| A-856 | (R) CH(CH₃)C(CH₃)═CH₂ | H | cyclopropyl |
| A-857 | (R) CH(CH₃)C(CH₃)═CH₂ | CH₃ | cyclopropyl |
| A-858 | (R) CH(CH₃)C(CH₃)═CH₂ | CH₂CH₃ | cyclopropyl |
| A-859 | CH₂—C≡CH | H | cyclopropyl |
| A-860 | CH₂—C≡CH | CH₃ | cyclopropyl |
| A-861 | CH₂—C≡CH | CH₂CH₃ | cyclopropyl |
| A-862 | cyclopentyl | H | cyclopropyl |
| A-863 | cyclopentyl | CH₃ | cyclopropyl |
| A-864 | cyclopentyl | CH₂CH₃ | cyclopropyl |
| A-865 | cyclohexyl | H | cyclopropyl |
| A-866 | cyclohexyl | CH₃ | cyclopropyl |
| A-867 | cyclohexyl | CH₂CH₃ | cyclopropyl |
| A-868 | CH₂—C₆H₅ | H | cyclopropyl |
| A-869 | CH₂—C₆H₅ | CH₃ | cyclopropyl |
| A-870 | CH₂—C₆H₅ | CH₂CH₃ | cyclopropyl |
| A-871 | NH₂ | CH₂-c-C₆H₁₁ | cyclopropyl |
| A-872 | NH₂ | CH₂CH₃ | cyclopropyl |
| A-873 | NH₂ | CH₂CH₂CH₃ | cyclopropyl |
| A-874 | NH—CH₂—CH═CH₂ | H | cyclopropyl |
| A-875 | NH—CH₂—CH═CH₂ | CH₃ | cyclopropyl |
| A-876 | NH—CH₂—CH═CH₂ | CH₂CH₃ | cyclopropyl |
| A-877 | NH—C(CH₃)₃ | H | cyclopropyl |
| A-878 | N(CH₃)₂ | H | cyclopropyl |
| A-879 | NH(CH₃) | H | cyclopropyl |
| A-880 | —(CH₂)₂CH═CHCH₂— | | cyclopropyl |
| A-881 | —(CH₂)₂C(CH₃)═CHCH₂— | | cyclopropyl |
| A-882 | —CH(CH₃)CH₂—CH═CHCH₂— | | cyclopropyl |
| A-883 | —(CH₂)₂CH(CH₃)(CH₂)₂— | | cyclopropyl |
| A-884 | —(CH₂)₃CHFCH₂— | | cyclopropyl |
| A-885 | —(CH₂)₂CHF(CH₂)₂— | | cyclopropyl |
| A-886 | —CH₂CHF(CH₂)₃— | | cyclopropyl |
| A-887 | —(CH₂)₂CH(CF₃)(CH₂)₂— | | cyclopropyl |
| A-888 | —(CH₂)₂O(CH₂)₂— | | cyclopropyl |
| A-889 | —(CH₂)₂S(CH₂)₂— | | cyclopropyl |
| A-890 | —(CH₂)₅— | | cyclopropyl |
| A-891 | —(CH₂)₄— | | cyclopropyl |
| A-892 | —CH₂CH═CHCH₂— | | cyclopropyl |
| A-893 | —CH(CH₃)(CH₂)₃— | | cyclopropyl |
| A-894 | —CH₂CH(CH₃)(CH₂)₂— | | cyclopropyl |
| A-895 | —CH(CH₃)—(CH₂)₂—CH(CH₃)— | | cyclopropyl |
| A-896 | —CH(CH₃)—(CH₂)₄— | | cyclopropyl |
| A-897 | —CH₂—CH(CH₃)—(CH₂)₃— | | cyclopropyl |
| A-898 | —(CH₂)—CH(CH₃)—CH₂—CH(CH₃)—CH₂— | | cyclopropyl |
| A-899 | —CH(CH₂CH₃)—(CH₂)₄— | | cyclopropyl |
| A-900 | —(CH₂)₂—CHOH—(CH₂)₂— | | cyclopropyl |
| A-901 | —(CH₂)₆— | | cyclopropyl |
| A-902 | —CH(CH₃)—(CH₂)₅— | | cyclopropyl |
| A-903 | —(CH₂)₂—N(CH₃)—(CH₂)₂— | | cyclopropyl |
| A-904 | —N═CH—CH═CH— | | cyclopropyl |
| A-905 | —N═C(CH₃)—CH═C(CH₃)— | | cyclopropyl |
| A-906 | —N═C(CF₃)—CH═C(CF₃)— | | cyclopropyl |
| A-907 | H | H | NH₂ |
| A-908 | H | H | ethyl |

TABLE B

| Row | Het | Y |
|---|---|---|
| B-1 | 4-ethylpyridin-2-yl | H |
| B-2 | 5-ethylpyridin-2-yl | H |
| B-3 | 6-ethylpyridin-2-yl | H |
| B-4 | 5-ethylpyridin-3-yl | H |
| B-5 | 6-ethylpyridin-3-yl | H |
| B-6 | 2-ethylpyridin-4-yl | H |
| B-7 | 4-n-propylpyridin-2-yl | H |
| B-8 | 5-n-propylpyridin-2-yl | H |
| B-9 | 6-n-propylpyridin-2-yl | H |
| B-10 | 5-n-propylpyridin-3-yl | H |
| B-11 | 6-n-propylpyridin-3-yl | H |
| B-12 | 2-n-propylpyridin-4-yl | H |
| B-13 | 4-i-propylpyridin-2-yl | H |
| B-14 | 5-i-propylpyridin-2-yl | H |
| B-15 | 6-i-propylpyridin-2-yl | H |
| B-16 | 5-i-propylpyridin-3-yl | H |
| B-17 | 6-i-propylpyridin-3-yl | H |
| B-18 | 2-i-propylpyridin-4-yl | H |
| B-19 | 4-n-butylpyridin-2-yl | H |
| B-20 | 5-n-butylpyridin-2-yl | H |
| B-21 | 6-n-butylpyridin-2-yl | H |
| B-22 | 5-n-butylpyridin-3-yl | H |
| B-23 | 6-n-butylpyridin-3-yl | H |
| B-24 | 2-n-butylpyridin-4-yl | H |
| B-25 | 4-t-butylpyridin-2-yl | H |
| B-26 | 5-t-butylpyridin-2-yl | H |
| B-27 | 6-t-butylpyridin-2-yl | H |
| B-28 | 5-t-butylpyridin-3-yl | H |
| B-29 | 6-t-butylpyridin-3-yl | H |
| B-30 | 2-t-butylpyridin-4-yl | H |
| B-31 | 5-ethylpyrazin-2-yl | H |
| B-32 | 6-ethylpyrazin-2-yl | H |
| B-33 | 5-n-propylpyrazin-2-yl | H |
| B-34 | 6-n-propylpyrazin-2-yl | H |
| B-35 | 5-i-propylpyrazin-2-yl | H |
| B-36 | 6-i-propylpyrazin-2-yl | H |
| B-37 | 5-n-butylpyrazin-2-yl | H |
| B-38 | 6-n-butylpyrazin-2-yl | H |
| B-39 | 5-t-butylpyrazin-2-yl | H |
| B-40 | 6-t-butylpyrazin-2-yl | H |
| B-41 | 5-ethylpyridazin-3-yl | H |
| B-42 | 6-ethylpyridazin-3-yl | H |
| B-43 | 6-ethylpyridazin-4-yl | H |
| B-44 | 5-n-propylpyridazin-3-yl | H |
| B-45 | 6-n-propylpyridazin-3-yl | H |
| B-46 | 6-n-propylpyridazin-4-yl | H |
| B-47 | 5-i-propylpyridazin-3-yl | H |
| B-48 | 6-i-propylpyridazin-3-yl | H |
| B-49 | 6-i-propylpyridazin-4-yl | H |
| B-50 | 5-n-butylpyridazin-3-yl | H |
| B-51 | 6-n-butylpyridazin-3-yl | H |
| B-52 | 6-n-butylpyridazin-4-yl | H |
| B-53 | 5-t-butylpyridazin-3-yl | H |
| B-54 | 6-t-butylpyridazin-3-yl | H |
| B-55 | 6-t-butylpyridazin-4-yl | H |
| B-56 | 4-ethyl-[1,3,5]-triazin-2-yl | H |
| B-57 | 4-n-propyl-[1,3,5]-triazin-2-yl | H |
| B-58 | 4-i-propyl-[1,3,5]-triazin-2-yl | H |
| B-59 | 4-n-butyl-[1,3,5]-triazin-2-yl | H |
| B-60 | 4-t-butyl-[1,3,5]-triazin-2-yl | H |
| B-61 | 5-ethyl-[1,2,4]-triazin-3-yl | H |
| B-62 | 6-ethyl-[1,2,4]-triazin-3-yl | H |
| B-63 | 3-ethyl-[1,2,4]-triazin-5-yl | H |
| B-64 | 3-ethyl-[1,2,4]-triazin-6-yl | H |
| B-65 | 5-n-propyl-[1,2,4]-triazin-3-yl | H |
| B-66 | 6-n-propyl-[1,2,4]-triazin-3-yl | H |
| B-67 | 3-n-propyl-[1,2,4]-triazin-5-yl | H |
| B-68 | 3-n-propyl-[1,2,4]-triazin-6-yl | H |
| B-69 | 5-i-propyl-[1,2,4]-triazin-3-yl | H |
| B-70 | 6-i-propyl-[1,2,4]-triazin-3-yl | H |
| B-71 | 3-i-propyl-[1,2,4]-triazin-5-yl | H |
| B-72 | 3-i-propyl-[1,2,4]-triazin-6-yl | H |
| B-73 | 5-n-butyl-[1,2,4]-triazin-3-yl | H |
| B-74 | 6-n-butyl-[1,2,4]-triazin-3-yl | H |
| B-75 | 3-n-butyl-[1,2,4]-triazin-5-yl | H |
| B-76 | 3-n-butyl-[1,2,4]-triazin-6-yl | H |
| B-77 | 5-t-butyl-[1,2,4]-triazin-3-yl | H |
| B-78 | 6-t-butyl-[1,2,4]-triazin-3-yl | H |
| B-79 | 3-t-butyl-[1,2,4]-triazin-5-yl | H |
| B-80 | 3-t-butyl-[1,2,4]-triazin-6-yl | H |
| B-81 | 2-ethylpyrimidin-4-yl | H |
| B-82 | 6-ethylpyrimidin-4-yl | H |
| B-83 | 4-ethylpyrimidin-2-yl | H |
| B-84 | 5-ethylpyrimidin-2-yl | H |
| B-85 | 2-ethylpyrimidin-5-yl | H |
| B-86 | 2-n-propylpyrimidin-4-yl | H |
| B-87 | 6-n-propylpyrimidin-4-yl | H |
| B-88 | 4-n-propylpyrimidin-2-yl | H |
| B-89 | 5-n-propylpyrimidin-2-yl | H |
| B-90 | 2-n-propylpyrimidin-5-yl | H |
| B-91 | 2-i-propylpyrimidin-4-yl | H |
| B-92 | 6-i-propylpyrimidin-4-yl | H |
| B-93 | 4-i-propylpyrimidin-2-yl | H |
| B-94 | 5-i-propylpyrimidin-2-yl | H |
| B-95 | 2-i-propylpyrimidin-5-yl | H |
| B-96 | 2-n-butylpyrimidin-4-yl | H |
| B-97 | 6-n-butylpyrimidin-4-yl | H |
| B-98 | 4-n-butylpyrimidin-2-yl | H |
| B-99 | 5-n-butylpyrimidin-2-yl | H |
| B-100 | 2-n-butylpyrimidin-5-yl | H |
| B-101 | 2-t-butylpyrimidin-4-yl | H |
| B-102 | 6-t-butylpyrimidin-4-yl | H |
| B-103 | 4-t-butylpyrimidin-2-yl | H |
| B-104 | 5-t-butylpyrimidin-2-yl | H |
| B-105 | 2-t-butylpyrimidin-5-yl | H |
| B-106 | 4-ethylpyridin-2-yl | methyl |
| B-107 | 5-ethylpyridin-2-yl | methyl |
| B-108 | 6-ethylpyridin-2-yl | methyl |
| B-109 | 5-ethylpyridin-3-yl | methyl |
| B-110 | 6-ethylpyridin-3-yl | methyl |
| B-111 | 2-ethylpyridin-4-yl | methyl |
| B-112 | 4-n-propylpyridin-2-yl | methyl |
| B-113 | 5-n-propylpyridin-2-yl | methyl |
| B-114 | 6-n-propylpyridin-2-yl | methyl |
| B-115 | 5-n-propylpyridin-3-yl | methyl |
| B-116 | 6-n-propylpyridin-3-yl | methyl |
| B-117 | 2-n-propylpyridin-4-yl | methyl |
| B-118 | 4-i-propylpyridin-2-yl | methyl |
| B-119 | 5-i-propylpyridin-2-yl | methyl |
| B-120 | 6-i-propylpyridin-2-yl | methyl |
| B-121 | 5-i-propylpyridin-3-yl | methyl |
| B-122 | 6-i-propylpyridin-3-yl | methyl |
| B-123 | 2-i-propylpyridin-4-yl | methyl |
| B-124 | 4-n-butylpyridin-2-yl | methyl |
| B-125 | 5-n-butylpyridin-2-yl | methyl |
| B-126 | 6-n-butylpyridin-2-yl | methyl |
| B-127 | 5-n-butylpyridin-3-yl | methyl |
| B-128 | 6-n-butylpyridin-3-yl | methyl |
| B-129 | 2-n-butylpyridin-4-yl | methyl |
| B-130 | 4-t-butylpyridin-2-yl | methyl |
| B-131 | 5-t-butylpyridin-2-yl | methyl |
| B-132 | 6-t-butylpyridin-2-yl | methyl |
| B-133 | 5-t-butylpyridin-3-yl | methyl |
| B-134 | 6-t-butylpyridin-3-yl | methyl |
| B-135 | 2-t-butylpyridin-4-yl | methyl |
| B-136 | 5-ethylpyrazin-2-yl | methyl |
| B-137 | 6-ethylpyrazin-2-yl | methyl |
| B-138 | 5-n-propylpyrazin-2-yl | methyl |
| B-139 | 6-n-propylpyrazin-2-yl | methyl |
| B-140 | 5-i-propylpyrazin-2-yl | methyl |
| B-141 | 6-i-propylpyrazin-2-yl | methyl |
| B-142 | 5-n-butylpyrazin-2-yl | methyl |
| B-143 | 6-n-butylpyrazin-2-yl | methyl |
| B-144 | 5-t-butylpyrazin-2-yl | methyl |
| B-145 | 6-t-butylpyrazin-2-yl | methyl |
| B-146 | 5-ethylpyridazin-3-yl | methyl |
| B-147 | 6-ethylpyridazin-3-yl | methyl |
| B-148 | 6-ethylpyridazin-4-yl | methyl |
| B-149 | 5-n-propylpyridazin-3-yl | methyl |
| B-150 | 6-n-propylpyridazin-3-yl | methyl |
| B-151 | 6-n-propylpyridazin-4-yl | methyl |
| B-152 | 5-i-propylpyridazin-3-yl | methyl |
| B-153 | 6-i-propylpyridazin-3-yl | methyl |
| B-154 | 6-i-propylpyridazin-4-yl | methyl |
| B-155 | 5-n-butylpyridazin-3-yl | methyl |
| B-156 | 6-n-butylpyridazin-3-yl | methyl |
| B-157 | 6-n-butylpyridazin-4-yl | methyl |
| B-158 | 5-t-butylpyridazin-3-yl | methyl |
| B-159 | 6-t-butylpyridazin-3-yl | methyl |
| B-160 | 6-t-butylpyridazin-4-yl | methyl |
| B-161 | 4-ethyl-[1,3,5]-triazin-2-yl | methyl |
| B-162 | 4-n-propyl-[1,3,5]-triazin-2-yl | methyl |
| B-163 | 4-i-propyl-[1,3,5]-triazin-2-yl | methyl |
| B-164 | 4-n-butyl-[1,3,5]-triazin-2-yl | methyl |
| B-165 | 4-t-butyl-[1,3,5]-triazin-2-yl | methyl |
| B-166 | 5-ethyl-[1,2,4]-triazin-3-yl | methyl |
| B-167 | 6-ethyl-[1,2,4]-triazin-3-yl | methyl |
| B-168 | 3-ethyl-[1,2,4]-triazin-5-yl | methyl |
| B-169 | 3-ethyl-[1,2,4]-triazin-6-yl | methyl |
| B-170 | 5-n-propyl-[1,2,4]-triazin-3-yl | methyl |
| B-171 | 6-n-propyl-[1,2,4]-triazin-3-yl | methyl |
| B-172 | 3-n-propyl-[1,2,4]-triazin-5-yl | methyl |
| B-173 | 3-n-propyl-[1,2,4]-triazin-6-yl | methyl |
| B-174 | 5-i-propyl-[1,2,4]-triazin-3-yl | methyl |
| B-175 | 6-i-propyl-[1,2,4]-triazin-3-yl | methyl |
| B-176 | 3-i-propyl-[1,2,4]-triazin-5-yl | methyl |
| B-177 | 3-i-propyl-[1,2,4]-triazin-6-yl | methyl |
| B-178 | 5-n-butyl-[1,2,4]-triazin-3-yl | methyl |
| B-179 | 6-n-butyl-[1,2,4]-triazin-3-yl | methyl |
| B-180 | 3-n-butyl-[1,2,4]-triazin-5-yl | methyl |
| B-181 | 3-n-butyl-[1,2,4]-triazin-6-yl | methyl |
| B-182 | 5-t-butyl-[1,2,4]-triazin-3-yl | methyl |
| B-183 | 6-t-butyl-[1,2,4]-triazin-3-yl | methyl |
| B-184 | 3-t-butyl-[1,2,4]-triazin-5-yl | methyl |
| B-185 | 3-t-butyl-[1,2,4]-triazin-6-yl | methyl |
| B-186 | 2-ethylpyrimidin-4-yl | methyl |
| B-187 | 6-ethylpyrimidin-4-yl | methyl |
| B-188 | 4-ethylpyrimidin-2-yl | methyl |
| B-189 | 5-ethylpyrimidin-2-yl | methyl |
| B-190 | 2-ethylpyrimidin-5-yl | methyl |
| B-191 | 2-n-propylpyrimidin-4-yl | methyl |
| B-192 | 6-n-propylpyrimidin-4-yl | methyl |
| B-193 | 4-n-propylpyrimidin-2-yl | methyl |
| B-194 | 5-n-propylpyrimidin-2-yl | methyl |
| B-195 | 2-n-propylpyrimidin-5-yl | methyl |
| B-196 | 2-i-propylpyrimidin-4-yl | methyl |
| B-197 | 6-i-propylpyrimidin-4-yl | methyl |
| B-198 | 4-i-propylpyrimidin-2-yl | methyl |
| B-199 | 5-i-propylpyrimidin-2-yl | methyl |
| B-200 | 2-i-propylpyrimidin-5-yl | methyl |
| B-201 | 2-n-butylpyrimidin-4-yl | methyl |
| B-202 | 6-n-butylpyrimidin-4-yl | methyl |
| B-203 | 4-n-butylpyrimidin-2-yl | methyl |
| B-204 | 5-n-butylpyrimidin-2-yl | methyl |
| B-205 | 2-n-butylpyrimidin-5-yl | methyl |
| B-206 | 2-t-butylpyrimidin-4-yl | methyl |
| B-207 | 6-t-butylpyrimidin-4-yl | methyl |
| B-208 | 4-t-butylpyrimidin-2-yl | methyl |
| B-209 | 5-t-butylpyrimidin-2-yl | methyl |
| B-210 | 2-t-butylpyrimidin-5-yl | methyl |
| B-211 | 4-ethylpyridin-2-yl | ethyl |
| B-212 | 5-ethylpyridin-2-yl | ethyl |
| B-213 | 6-ethylpyridin-2-yl | ethyl |
| B-214 | 5-ethylpyridin-3-yl | ethyl |
| B-215 | 6-ethylpyridin-3-yl | ethyl |
| B-216 | 2-ethylpyridin-4-yl | ethyl |
| B-217 | 4-n-propylpyridin-2-yl | ethyl |
| B-218 | 5-n-propylpyridin-2-yl | ethyl |
| B-219 | 6-n-propylpyridin-2-yl | ethyl |
| B-220 | 5-n-propylpyridin-3-yl | ethyl |
| B-221 | 6-n-propylpyridin-3-yl | ethyl |
| B-222 | 2-n-propylpyridin-4-yl | ethyl |
| B-223 | 4-i-propylpyridin-2-yl | ethyl |
| B-224 | 5-i-propylpyridin-2-yl | ethyl |
| B-225 | 6-i-propylpyridin-2-yl | ethyl |
| B-226 | 5-i-propylpyridin-3-yl | ethyl |
| B-227 | 6-i-propylpyridin-3-yl | ethyl |
| B-228 | 2-i-propylpyridin-4-yl | ethyl |
| B-229 | 4-n-butylpyridin-2-yl | ethyl |
| B-230 | 5-n-butylpyridin-2-yl | ethyl |
| B-231 | 6-n-butylpyridin-2-yl | ethyl |
| B-232 | 5-n-butylpyridin-3-yl | ethyl |
| B-233 | 6-n-butylpyridin-3-yl | ethyl |
| B-234 | 2-n-butylpyridin-4-yl | ethyl |
| B-235 | 4-t-butylpyridin-2-yl | ethyl |
| B-236 | 5-t-butylpyridin-2-yl | ethyl |
| B-237 | 6-t-butylpyridin-2-yl | ethyl |
| B-238 | 5-t-butylpyridin-3-yl | ethyl |
| B-239 | 6-t-butylpyridin-3-yl | ethyl |
| B-240 | 2-t-butylpyridin-4-yl | ethyl |
| B-241 | 5-ethylpyrazin-2-yl | ethyl |
| B-242 | 6-ethylpyrazin-2-yl | ethyl |
| B-243 | 5-n-propylpyrazin-2-yl | ethyl |
| B-244 | 6-n-propylpyrazin-2-yl | ethyl |
| B-245 | 5-i-propylpyrazin-2-yl | ethyl |
| B-246 | 6-i-propylpyrazin-2-yl | ethyl |
| B-247 | 5-n-butylpyrazin-2-yl | ethyl |
| B-248 | 6-n-butylpyrazin-2-yl | ethyl |
| B-249 | 5-t-butylpyrazin-2-yl | ethyl |
| B-250 | 6-t-butylpyrazin-2-yl | ethyl |
| B-251 | 5-ethylpyridazin-3-yl | ethyl |
| B-252 | 6-ethylpyridazin-3-yl | ethyl |
| B-253 | 6-ethylpyridazin-4-yl | ethyl |
| B-254 | 5-n-propylpyridazin-3-yl | ethyl |
| B-255 | 6-n-propylpyridazin-3-yl | ethyl |
| B-256 | 6-n-propylpyridazin-4-yl | ethyl |
| B-257 | 5-i-propylpyridazin-3-yl | ethyl |
| B-258 | 6-i-propylpyridazin-3-yl | ethyl |
| B-259 | 6-i-propylpyridazin-4-yl | ethyl |
| B-260 | 5-n-butylpyridazin-3-yl | ethyl |
| B-261 | 6-n-butylpyridazin-3-yl | ethyl |
| B-262 | 6-n-butylpyridazin-4-yl | ethyl |
| B-263 | 5-t-butylpyridazin-3-yl | ethyl |
| B-264 | 6-t-butylpyridazin-3-yl | ethyl |
| B-265 | 6-t-butylpyridazin-4-yl | ethyl |
| B-266 | 4-ethyl-[1,3,5]-triazin-2-yl | ethyl |
| B-267 | 4-n-propyl-[1,3,5]-triazin-2-yl | ethyl |
| B-268 | 4-i-propyl-[1,3,5]-triazin-2-yl | ethyl |
| B-269 | 4-n-butyl-[1,3,5]-triazin-2-yl | ethyl |
| B-270 | 4-t-butyl-[1,3,5]-triazin-2-yl | ethyl |
| B-271 | 5-ethyl-[1,2,4]-triazin-3-yl | ethyl |
| B-272 | 6-ethyl-[1,2,4]-triazin-3-yl | ethyl |
| B-273 | 3-ethyl-[1,2,4]-triazin-5-yl | ethyl |
| B-274 | 3-ethyl-[1,2,4]-triazin-6-yl | ethyl |
| B-275 | 5-n-propyl-[1,2,4]-triazin-3-yl | ethyl |
| B-276 | 6-n-propyl-[1,2,4]-triazin-3-yl | ethyl |
| B-277 | 3-n-propyl-[1,2,4]-triazin-5-yl | ethyl |
| B-278 | 3-n-propyl-[1,2,4]-triazin-6-yl | ethyl |
| B-279 | 5-i-propyl-[1,2,4]-triazin-3-yl | ethyl |
| B-280 | 6-i-propyl-[1,2,4]-triazin-3-yl | ethyl |
| B-281 | 3-i-propyl-[1,2,4]-triazin-5-yl | ethyl |
| B-282 | 3-i-propyl-[1,2,4]-triazin-6-yl | ethyl |
| B-283 | 5-n-butyl-[1,2,4]-triazin-3-yl | ethyl |
| B-284 | 6-n-butyl-[1,2,4]-triazin-3-yl | ethyl |
| B-285 | 3-n-butyl-[1,2,4]-triazin-5-yl | ethyl |
| B-286 | 3-n-butyl-[1,2,4]-triazin-6-yl | ethyl |
| B-287 | 5-t-butyl-[1,2,4]-triazin-3-yl | ethyl |
| B-288 | 6-t-butyl-[1,2,4]-triazin-3-yl | ethyl |
| B-289 | 3-t-butyl-[1,2,4]-triazin-5-yl | ethyl |
| B-290 | 3-t-butyl-[1,2,4]-triazin-6-yl | ethyl |
| B-291 | 2-ethylpyrimidin-4-yl | ethyl |
| B-292 | 6-ethylpyrimidin-4-yl | ethyl |
| B-293 | 4-ethylpyrimidin-2-yl | ethyl |
| B-294 | 5-ethylpyrimidin-2-yl | ethyl |
| B-295 | 2-ethylpyrimidin-5-yl | ethyl |
| B-296 | 2-n-propylpyrimidin-4-yl | ethyl |
| B-297 | 6-n-propylpyrimidin-4-yl | ethyl |
| B-298 | 4-n-propylpyrimidin-2-yl | ethyl |
| B-299 | 5-n-propylpyrimidin-2-yl | ethyl |
| B-300 | 2-n-propylpyrimidin-5-yl | ethyl |
| B-301 | 2-i-propylpyrimidin-4-yl | ethyl |
| B-302 | 6-i-propylpyrimidin-4-yl | ethyl |
| B-303 | 4-i-propylpyrimidin-2-yl | ethyl |
| B-304 | 5-i-propylpyrimidin-2-yl | ethyl |
| B-305 | 2-i-propylpyrimidin-5-yl | ethyl |
| B-306 | 2-n-butylpyrimidin-4-yl | ethyl |
| B-307 | 6-n-butylpyrimidin-4-yl | ethyl |
| B-308 | 4-n-butylpyrimidin-2-yl | ethyl |
| B-309 | 5-n-butylpyrimidin-2-yl | ethyl |
| B-310 | 2-n-butylpyrimidin-5-yl | ethyl |
| B-311 | 2-t-butylpyrimidin-4-yl | ethyl |
| B-312 | 6-t-butylpyrimidin-4-yl | ethyl |
| B-313 | 4-t-butylpyrimidin-2-yl | ethyl |
| B-314 | 5-t-butylpyrimidin-2-yl | ethyl |
| B-315 | 2-t-butylpyrimidin-5-yl | ethyl |
| B-316 | 4-ethylpyridin-2-yl | NH₂ |
| B-317 | 5-ethylpyridin-2-yl | NH₂ |
| B-318 | 6-ethylpyridin-2-yl | NH₂ |
| B-319 | 5-ethylpyridin-3-yl | NH₂ |
| B-320 | 6-ethylpyridin-3-yl | NH₂ |
| B-321 | 2-ethylpyridin-4-yl | NH₂ |
| B-322 | 4-n-propylpyridin-2-yl | NH₂ |
| B-323 | 5-n-propylpyridin-2-yl | NH₂ |
| B-324 | 6-n-propylpyridin-2-yl | NH₂ |
| B-325 | 5-n-propylpyridin-3-yl | NH₂ |
| B-326 | 6-n-propylpyridin-3-yl | NH₂ |
| B-327 | 2-n-propylpyridin-4-yl | NH₂ |
| B-328 | 4-i-propylpyridin-2-yl | NH₂ |
| B-329 | 5-i-propylpyridin-2-yl | NH₂ |
| B-330 | 6-i-propylpyridin-2-yl | NH₂ |
| B-331 | 5-i-propylpyridin-3-yl | NH₂ |
| B-332 | 6-i-propylpyridin-3-yl | NH₂ |
| B-333 | 2-i-propylpyridin-4-yl | NH₂ |
| B-334 | 4-n-butylpyridin-2-yl | NH₂ |
| B-335 | 5-n-butylpyridin-2-yl | NH₂ |
| B-336 | 6-n-butylpyridin-2-yl | NH₂ |
| B-337 | 5-n-butylpyridin-3-yl | NH₂ |
| B-338 | 6-n-butylpyridin-3-yl | NH₂ |
| B-339 | 2-n-butylpyridin-4-yl | NH₂ |
| B-340 | 4-t-butylpyridin-2-yl | NH₂ |
| B-341 | 5-t-butylpyridin-2-yl | NH₂ |
| B-342 | 6-t-butylpyridin-2-yl | NH₂ |
| B-343 | 5-t-butylpyridin-3-yl | NH₂ |
| B-344 | 6-t-butylpyridin-3-yl | NH₂ |
| B-345 | 2-t-butylpyridin-4-yl | NH₂ |
| B-346 | 5-ethylpyrazin-2-yl | NH₂ |
| B-347 | 6-ethylpyrazin-2-yl | NH₂ |
| B-348 | 5-n-propylpyrazin-2-yl | NH₂ |
| B-349 | 6-n-propylpyrazin-2-yl | NH₂ |
| B-350 | 5-i-propylpyrazin-2-yl | NH₂ |
| B-351 | 6-i-propylpyrazin-2-yl | NH₂ |
| B-352 | 5-n-butylpyrazin-2-yl | NH₂ |
| B-353 | 6-n-butylpyrazin-2-yl | NH₂ |
| B-354 | 5-t-butylpyrazin-2-yl | NH₂ |
| B-355 | 6-t-butylpyrazin-2-yl | NH₂ |
| B-356 | 5-ethylpyridazin-3-yl | NH₂ |
| B-357 | 6-ethylpyridazin-3-yl | NH₂ |
| B-358 | 6-ethylpyridazin-4-yl | NH₂ |
| B-359 | 5-n-propylpyridazin-3-yl | NH₂ |
| B-360 | 6-n-propylpyridazin-3-yl | NH₂ |
| B-361 | 6-n-propylpyridazin-4-yl | NH₂ |
| B-362 | 5-i-propylpyridazin-3-yl | NH₂ |
| B-363 | 6-i-propylpyridazin-3-yl | NH₂ |
| B-364 | 6-i-propylpyridazin-4-yl | NH₂ |
| B-365 | 5-n-butylpyridazin-3-yl | NH₂ |
| B-366 | 6-n-butylpyridazin-3-yl | NH₂ |
| B-367 | 6-n-butylpyridazin-4-yl | NH₂ |
| B-368 | 5-t-butylpyridazin-3-yl | NH₂ |
| B-369 | 6-t-butylpyridazin-3-yl | NH₂ |
| B-370 | 6-t-butylpyridazin-4-yl | NH₂ |
| B-371 | 4-ethyl-[1,3,5]-triazin-2-yl | NH₂ |
| B-372 | 4-n-propyl-[1,3,5]-triazin-2-yl | NH₂ |
| B-373 | 4-i-propyl-[1,3,5]-triazin-2-yl | NH₂ |
| B-374 | 4-n-butyl-[1,3,5]-triazin-2-yl | NH₂ |
| B-375 | 4-t-butyl-[1,3,5]-triazin-2-yl | NH₂ |
| B-376 | 5-ethyl-[1,2,4]-triazin-3-yl | NH₂ |
| B-377 | 6-ethyl-[1,2,4]-triazin-3-yl | NH₂ |
| B-378 | 3-ethyl-[1,2,4]-triazin-5-yl | NH₂ |
| B-379 | 3-ethyl-[1,2,4]-triazin-6-yl | NH₂ |
| B-380 | 5-n-propyl-[1,2,4]-triazin-3-yl | NH₂ |
| B-381 | 6-n-propyl-[1,2,4]-triazin-3-yl | NH₂ |
| B-382 | 3-n-propyl-[1,2,4]-triazin-5-yl | NH₂ |
| B-383 | 3-n-propyl-[1,2,4]-triazin-6-yl | NH₂ |
| B-384 | 5-i-propyl-[1,2,4]-triazin-3-yl | NH₂ |
| B-385 | 6-i-propyl-[1,2,4]-triazin-3-yl | NH₂ |
| B-386 | 3-i-propyl-[1,2,4]-triazin-5-yl | NH₂ |
| B-387 | 3-i-propyl-[1,2,4]-triazin-6-yl | NH₂ |
| B-388 | 5-n-butyl-[1,2,4]-triazin-3-yl | NH₂ |
| B-389 | 6-n-butyl-[1,2,4]-triazin-3-yl | NH₂ |
| B-390 | 3-n-butyl-[1,2,4]-triazin-5-yl | NH₂ |
| B-391 | 3-n-butyl-[1,2,4]-triazin-6-yl | NH₂ |
| B-392 | 5-t-butyl-[1,2,4]-triazin-3-yl | NH₂ |
| B-393 | 6-t-butyl-[1,2,4]-triazin-3-yl | NH₂ |
| B-394 | 3-t-butyl-[1,2,4]-triazin-5-yl | NH₂ |
| B-395 | 3-t-butyl-[1,2,4]-triazin-6-yl | NH₂ |
| B-396 | 2-ethylpyrimidin-4-yl | NH₂ |
| B-397 | 6-ethylpyrimidin-4-yl | NH₂ |
| B-398 | 4-ethylpyrimidin-2-yl | NH₂ |
| B-399 | 5-ethylpyrimidin-2-yl | NH₂ |
| B-400 | 2-ethylpyrimidin-5-yl | NH₂ |
| B-401 | 2-n-propylpyrimidin-4-yl | NH₂ |
| B-402 | 6-n-propylpyrimidin-4-yl | NH₂ |
| B-403 | 4-n-propylpyrimidin-2-yl | NH₂ |
| B-404 | 5-n-propylpyrimidin-2-yl | NH₂ |
| B-405 | 2-n-propylpyrimidin-5-yl | NH₂ |
| B-406 | 2-i-propylpyrimidin-4-yl | NH₂ |
| B-407 | 6-i-propylpyrimidin-4-yl | NH₂ |
| B-408 | 4-i-propylpyrimidin-2-yl | NH₂ |
| B-409 | 5-i-propylpyrimidin-2-yl | NH₂ |
| B-410 | 2-i-propylpyrimidin-5-yl | NH₂ |
| B-411 | 2-n-butylpyrimidin-4-yl | NH₂ |
| B-412 | 6-n-butylpyrimidin-4-yl | NH₂ |
| B-413 | 4-n-butylpyrimidin-2-yl | NH₂ |
| B-414 | 5-n-butylpyrimidin-2-yl | NH₂ |
| B-415 | 2-n-butylpyrimidin-5-yl | NH₂ |
| B-416 | 2-t-butylpyrimidin-4-yl | NH₂ |
| B-417 | 6-t-butylpyrimidin-4-yl | NH₂ |
| B-418 | 4-t-butylpyrimidin-2-yl | NH₂ |
| B-419 | 5-t-butylpyrimidin-2-yl | NH₂ |
| B-420 | 2-t-butylpyrimidin-5-yl | NH₂ |

The compounds of the formula (I) and/or their agriculturally acceptable salts are suitable as active compounds, in particular as fungicides. They are distinguished by excellent activity against a broad spectrum of phytopathogenic fungi, in particular from the class of the Ascomycetes, Deuteromycetes, Oomycetes and Basidiomycetes. Some of them are systemically active and can be used in crop protection as foliar fungicides, as fungicides for seed dressing and as soil fungicides.

They are particularly important in the control of a large number of fungi on various crop plants, such as wheat, rye, barley, oats, rice, corn, grass, bananas, cotton, soybeans, coffee, sugar cane, grapevines, fruit and ornamental plants and vegetables, such as cucumbers, beans, tomatoes, potatoes and cucurbits, and also on the seeds of these plants.

They are especially suitable for controlling the following plant diseases:
Alternaria species on vegetables, rapeseed, sugar beet and fruit and rice,
Aphanomyces species on sugar beet and vegetables,
Bipolaris and Drechslera species on corn, cereals, rice and lawns,
Blumeria graminis (powdery mildew) on cereals,
Botrytis cinerea (gray mold) on strawberries, vegetables, flowers and grapevines,
Bremia lactucae on lettuce,
Cercospora species on corn, soybeans, rice and sugar beet,
Cochliobolus species on corn, cereals, rice (e.g., Cochliobolus sativus on cereals, Cochliobolus miyabeanus on rice),
Colletotricum species on soybeans and cotton,
Drechslera species on cereals and corn,
Exserohilum species on corn,
Erysiphe cichoracearum and Sphaerotheca fuliginea on cucurbits,
Fusarium and Verticillium species on various plants,
Gaeumanomyces graminis on cereals,
Gibberella species on cereals and rice (e.g., Gibberella fujikuroi on rice),
Grain staining complex on rice,
Helminthosporium species on corn and rice,
Michrodochium nivale on cereals,
Mycosphaerella species on cereals, bananas and peanuts,
Phakopsara pachyrhizi and Phakopsara meibomiae on soybeans,
Phomopsis species on soybeans and sunflowers,
Phytophthora infestans on potatoes and tomatoes,
Plasmopara viticola on grapevines,
Podosphaera leucotricha on apples,
Pseudocercosporella herpotrichoides on cereals,
Pseudoperonospora species on hops and cucurbits,
Puccinia species on cereals and corn,
Pyrenophora species on cereals,
Pyricularia oryzae, Corticium sasakii, Sarocladium oryzae, S. attenuatum, Entyloma oryzae on rice,
Pyricularia grisea on lawns and cereals,
Pythium spp. on lawns, rice, corn, cotton, rapeseed, sunflowers, sugar beet, vegetables and other plants,
Rhizoctonia species on cotton, rice, potatoes, lawns, corn, rapeseed, potatoes, sugar beet, vegetables and other plants,
Sclerotinia species on rapeseed and sunflowers,
Septoria tritici and Stagonospora nodorum on wheat,
Erysiphe (syn. Uncinula) necator on grapevines,
Setospaeria species on corn and lawns,
Sphacelotheca reilinia on corn,
Thievaliopsis species on soybeans and cotton,
Tilletia species on cereals,
Ustilago species on cereals, corn and sugar beet, and
Venturia species (scab) on apples and pears.

The compounds (I) are furthermore suitable for controlling harmful fungi in the protection of materials (for example wood, paper, paint dispersions, fibers or fabrics) and in the protection of stored products. In the protection of wood, particular attention is paid to the following harmful fungi: Ascomycetes, such as Ophiostoma spp., Ceratocystis spp., Aureobasidium pullulans, Sclerophoma spp., Chaetomium spp., Humicola spp., Petriella spp., Trichurus spp.; Basidiomycetes, such as Coniophora spp., Coriolus spp., Gloeophyllum spp., Lentinus spp., Pleurotus spp., Poria spp., Serpula spp. and Tyromyces spp., Deuteromycetes, such as Aspergillus spp., Cladosporium spp., Penicillium spp., Trichoderma spp., Alternaria spp., Paecilomyces spp. and Zygomycetes, such as Mucor spp., additionally in the protection of materials the following yeasts: Candida spp. and Saccharomyces cerevisae.

In addition, the compounds of the formula (I) can also be used in crops which, owing to breeding including genetical engineering, are tolerant to attack by insects or fungi.

Accordingly, the present invention furthermore provides the use of the 7-amino-6-heteroaryl-1,2-4-triazolo[1,5-a]pyrimidines of the formula (I) according to the invention and/or their agriculturally acceptable salts for controlling phytopathogenic fungi.

The compounds of the formula (I) and/or their agriculturally acceptable salts are employed by treating the fungi or the plants, seeds, materials or soil to be protected from fungal attack with a fungicidally effective amount of these compounds. The application can be carried out both before and after the infection of the materials, plants or seeds by the fungi.

Accordingly, the present invention furthermore provides a method for controlling phytopathogenic fungi, which method comprises treating the fungi or the materials, plants, the soil or seed to be protected against fungal attack with an effective amount of at least one compound of the formula (I) according to the invention and/or an agriculturally acceptable salt thereof.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

Seed can be treated by methods known to the person skilled in the art, such as, for example, seed dressing, seed coating, seed dusting, seed soaking and seed pelleting.

In the treatment of seed, the amounts of active compound employed are generally from 1 to 1000 g/100 kg of seed, preferably from 1 to 200 g/100 kg, in particular from 5 to 100 g/100 kg.

Accordingly, the present invention furthermore provides seed comprising a compound of the formula (I) according to the invention in an amount of from 1 to 1000 g per 100 kg.

When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound per cubic meter of treated material.

The present invention furthermore provides a composition for controlling phytopathogenic fungi, which composition comprises at least one compound of the formula (I) according to the invention and/or an agriculturally acceptable salt thereof and at least one solid or liquid carrier.

The compounds (I) and/or their agriculturally acceptable salts can be converted into the customary formulations, for example solutions, emulsions, suspensions, dusts, powders, pastes and granules. The use form depends on the particular intended purpose; in each case, it should ensure a fine and even distribution of the compound according to the invention.

The formulations are prepared in a known manner, for example by extending the active compound with solvents and/or carriers, if desired using emulsifiers and dispersants. Solvents/auxiliaries Which are suitable are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example finely divided silica, silicates); emulsifiers such as nonionic and anionic emulsifiers (for example polyoxyethylene fatty alcohol ethers, alkylsulfonates and arylsulfonates) and dispersants such as lignosulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid With phenol and formaldehyde, polyoxyethylene octylphenol ether, ethoxylated isooctyl phenol, octylphenol, nonylphenol, alkylphenol polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

Formulations for seed treatment may further comprise binders and/or gelants and if appropriate dyes.

Binders can be added to increase the adhesion of the active compounds to the seed after the treatment. Suitable binders are for example EO/PO block copolymer surfactants, but also polyvinyl alcohols, polyvinylpyrrolidones, polyacrylates, polymethacrylates, polybutenes, polyisobutylenes, polystyrenes, polyethyleneamines, polyethyleneamides, polyethyleneimines (Lupasol®, Polymin®), polyethers, polyurethanes, polyvinyl acetates, Tylose and copolymers of these polymers. A suitable gelant is for example carrageen (Satiagel®).

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

The concentrations of active compound in the ready-to-use preparations can be varied within relatively wide ranges. In general, they are between 0.0001 and 10%, preferably between 0.01 and 1%.

The active compounds can also be used with great success in the ultra-low-volume (ULV) process, it being possible to apply formulations with more than 95% by weight of active compound or even the active compound without additives.

For the treatment of seed, the formulations in question give, after two-to-tenfold dilution, active compound concentrations of from 0.01 to 60% by Weight, preferably from 0.1 to 40% by weight, in the ready-to-use preparations.

The following are examples of formulations according to the invention: 1. Products for dilution with water

### A Water-Soluble Concentrates (SL, LS)

10 parts by weight of a compound according to the invention are dissolved with 90 parts by weight of water or with a water-soluble solvent. As an alternative, wetters or other auxiliaries are added. The active compound dissolves upon dilution with water. This gives a formulation having an active compound content of 10% by weight.

### B Dispersible Concentrates (DC)

20 parts by weight of a compound according to the invention are dissolved in 70 parts by weight of cyclohexanone with addition of 10 parts by weight of a dispersant, for example polyvinylpyrrolidone. Dilution with Water gives a dispersion. The active compound content is 20% by weight.

### C Emulsifiable Concentrates (EC)

15 parts by weight of a compound according to the invention are dissolved in 75 parts by weight of xylene with addition of calcium dodecylbenzenesulfonate and castor oil ethoxylate (in each case 5 parts by weight). Dilution with water gives an emulsion. The formulation has an active compound content of 15% by weight.

### D Emulsions (EW, EO, ES)

25 parts by weight of a compound according to the invention are dissolved in 35 parts by weight of xylene with addition of calcium dodecylbenzenesulfonate and castor oil ethoxylate (in each case 5 parts by weight). This mixture is added to 30 parts by weight of water by means of an emulsifying machine (e.g. Ultraturrax) and made into a homogeneous emulsion. Dilution with water gives an emulsion. The formulation has an active compound content of 25% by weight.

### E Suspensions (SC, OD, FS)

In an agitated ball mill, 20 parts by weight of a compound according to the invention are comminuted with addition of 10 parts by weight of dispersants and wetters and 70 parts by weight of water or an organic solvent to give a fine active compound suspension. Dilution with water gives a stable suspension of the active compound. The active compound content in the formulation is 20% by weight.

### F Water-Dispersible Granules and Water-Soluble Granules (WG, SG)

50 parts by weight of a compound according to the invention are ground finely with addition of 50 parts by weight of dispersants and wetters and made into water-dispersible or water-soluble granules by means of technical appliances (for example extrusion, spray tower, fluidized bed). Dilution with water gives a stable dispersion or solution of the active compound. The formulation has an active compound content of 50% by weight.

### G Water-Dispersible Powders and Water-Soluble Powders (WP, SP, SS, WS)

75 parts by weight of a compound according to the invention are ground in a rotor-stator mill with addition of 25 parts by weight of dispersants, wetters and silica gel. Dilution with water gives a stable dispersion or solution of the active compound. The active compound content of the formulation is 75% by weight.

### 2. Products to be Applied Undiluted

### H Dustable Powders (DP, DS)

5 parts by weight of a compound according to the invention are ground finely and mixed intimately with 95 parts by weight of finely divided kaolin. This gives a dustable product with an active compound content of 5% by weight.

### I Granules (GR, FG, GG, MG)

0.5 part by weight of a compound according to the invention is ground finely and associated with 99.5 parts by weight of carriers. Current methods are extrusion, spray-drying or the fluidized bed. This gives granules with an active compound content of 0.5% by weight to be applied undiluted.

### J ULV Solutions (UL)

10 parts by weight of a compound according to the invention are dissolved in 90 parts by weight of an organic solvent, for example xylene. This gives a product with an active compound content of 10% by weight to be applied undiluted.

Seed treatment typically utilizes water-soluble concentrates (LS), suspensions (FS), dusts (DS), water-dispersible and water-soluble powders (WS, SS), emulsions (ES), emulsifiable concentrates (EC) and gel formulations (GF). These formulations can be applied neat or preferably diluted to the seed. The application can take place prior to sowing.

Preference is given to using FS formulations for seed treatment. Such formulations typically comprise from 1 to 800 g/l of active compound, from 1 to 200 g/l of surfactants, from 0 to 200 g/l of antifreeze, from 0 to 400 g/l of binder, from 0 to 200 g/l of dyes and solvent, preferably water.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; the intention is to ensure in each case the finest possible distribution of the active compounds according to the invention.

Aqueous use forms can be prepared from emulsion concentrates, pastes or Wettable powders (sprayable powders, oil dispersions) by adding water. To prepare emulsions, pastes or oil dispersions, the substances, as such or dissolved in an oil or solvent, can be homogenized in water by means of a wetting agent, tackifier, dispersant or emulsifier. Alternatively, it is also possible to prepare concentrates composed of active substance, wetter, tackifier, dispersant or emulsifier and, if appropriate, solvent or oil, and such concentrates are suitable for dilution With water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply formulations comprising over 95% by weight of active compound, or even to apply the active compound without additives.

Various types of oils, wetters, adjuvants, herbicides, fungicides, other pesticides, or bactericides may be added to the active compounds, if appropriate not until immediately prior to use (tank mix). These agents can be admixed with the agents according to the invention in a weight ratio of 1:100 to 100:1, preferably 1:10 to 10:1.

Suitable adjuvants in this sense are in particular: organically modified polysiloxanes, for example Break Thru S 240®; alcohol alkoxylates, for example Atplus 245®, Atplus MBA 1303®, Plurafac LF 300® and Lutensol ON 30®; EO/PO block polymers, for example Pluronic RPE 2035® and Genapol B®; alcohol ethoxylates, for example Lutensol XP 80®; and sodium dioctylsulfosuccinate, for example Leophen RA®.

The compositions according to the invention can, in the application form as fungicides, also be present together with other active compounds, for example with herbicides, insecticides, growth regulators, fungicides or also with fertilizers. When mixing the compounds (I) or the compositions comprising them with one or more further active compounds, in particular fungicides, it is in many cases possible to broaden the activity spectrum or to prevent the development of resistance. In many cases, synergistic effects are obtained.

The invention furthermore provides a combination of at least one compound of the formula (I) according to the invention and/or an agriculturally acceptable salt thereof and at least one further fungicidally, insecticidally, herbicidally and/or growth-regulating active compound.

The following list of fungicides, together with which the compounds according to the invention may be used, is meant to illustrate the combination possibilities, but not to limit them:

### 1. Strobilurins

azoxystrobin, dimoxystrobin, enestroburin, fluoxastrobin, kresoxim-methyl, metominostrobin, picoxystrobin, pyraclostrobin, trifloxystrobin, orysastrobin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate, methyl 2-(ortho-((2,5-dimethylphenyloxymethylene)phenyl)-3-methoxyacrylate;

### 2. Carboxamides

carboxanilides: benalaxyl, benodanil, boscalid, carboxin, mepronil, fenfuram, fenhexamid, flutolanil, furametpyr, metalaxyl, ofurace, oxadixyl, oxycarboxin, penthiopyrad, thifluzamide, tiadinil, N-(4′-bromobiphenyl-2-yl)-4-difluoromethyl-2-methylthiazole-5-carboxamide, N-(4′-trifluoromethylbiphenyl-2-yl)-4-difluoromethyl-2-methylthiazole-5-carboxamide, N-(4′-chloro-3′-fluorobiphenyl-2-yl)-4-difluoromethyl-2-methylthiazole-5-carboxamide, N-(3′,4′-dichloro-4-fluoro-biphenyl-2-yl)-3-difluoromethyl-1-methylpyrazole-4-carboxamide, N-(2-cyanophenyl)-3,4-dichloroisothiazole-5-carboxamide;
carboxylic acid morpholides: dimethomorph, flumorph;
benzamides: flumetover, fluopicolide (picobenzamid), zoxamide;
other carboxamides: carpropamid, diclocymet, mandipropamid, N-(2-(4-[3-(4-chlorophenyl)prop-2-ynyloxy]-3-methoxyphenyl)ethyl)-2-methanesulfonylamino-3-methylbutyramide, N-(2-(4-[3-(4-chlorophenyl)prop-2-ynyloxy]-3-methoxyphenyl)-ethyl)-2-ethanesulfonylamino-3-methylbutyramide;

### 3. Azoles

triazoles: bitertanol, bromuconazole, cyproconazole, difenoconazole, diniconazole, enilconazole, epoxiconazole, fenbuconazole, flusilazole, fluquinconazole, flutriafol, hexaconazole, imibenconazole, ipconazole, metconazole, myclobutanil, penconazole, propiconazole, prothioconazole, simeconazole, tebuconazole, tetraconazole, triadimenol, triadimefon, triticonazole;
imidazoles: cyazofamid, imazalil, pefurazoate, prochloraz, triflumizole;
benzimidazoles: benomyl, carbendazim, fuberidazole, thiabendazole;
others: ethaboxam, etridiazole, hymexazole;
   4. Nitrogen-containing heterocyclyl compounds:
pyridines: fluazinam, pyrifenox, 3-[5-(4-chlorophenyl)-2,3-dimethylisoxazolidin-3-yl]pyridine;
pyrimidines: bupirimate, cyprodinil, ferimzone, fenarimol, mepanipyrim, nuarimol, pyrimethanil;
piperazines: triforine;
pyrroles: fludioxonil, fenpiclonil;
morpholines: aldimorph, dodemorph, fenpropimorph, tridemorph;
dicarboximides: iprodione, procymidone, vinclozolin;
others: acibenzolar-5-methyl, anilazine, captan, captafol, dazomet, diclomezine, fenoxanil, folpet, fenpropidin, famoxadone, fenamidone, octhilinone, probenazole, proquinazid, pyroquilon, quinoxyfen, tricyclazole, 5-chloro-7-(4-methylpiperidin-1-yl)-6-(2,4,6-trifluorophenyl)-[1,2,4]triazolo[1,5-a]pyrimidine, 2-butoxy-6-iodo-3-propylchroman-4-one, N,N-dimethyl-3-(3-bromo-6-fluoro-2-methylindole-1-sulfonyl)-[1,2,4]triazole-1-sulfonamide;
   5. Carbamates and dithiocarbamates
dithiocarbamates: ferbam, mancozeb, maneb, metiram, metam, propineb, thiram, zineb, ziram;
carbamates: diethofencarb, flubenthiavalicarb, iprovalicarb, propamocarb, methyl 3-(4-chlorophenyl)-3-(2-isopropoxycarbonylamino-3-methylbutyrylamino)propionate, 4-fluorophenyl N-(1-(1-(4-cyanophenyl)ethanesulfonyl)but-2-yl)carbamate;
   6. Other fungicides
guanidines: dodine, iminoctadine, guazatine;
antibiotics: kasugamycin, polyoxins, streptomycin, validamycin A;
organometal compounds: fentin salts;
sulfur-containing heterocyclyl compounds: isoprothiolane, dithianon;
organophosphorous compounds: edifenphos, fosetyl, fosetyl-aluminum, iprobenfos, pyrazophos, tolclofos-methyl, phosphorous acid and its salts;
organochlorine compounds: thiophanate methyl, chlorothalonil, dichlorfluanid, tolylfluanid, flusulfamide, phthalide, hexachlorobenzene, pencycuron, quintozene;
nitrophenyl derivatives: binapacryl, dinocap, dinobuton;
inorganic active compounds: Bordeaux mixture, copper acetate, copper hydroxide, copper oxychloride, basic copper sulfate, sulfur;
other: spiroxamine, cyflufenamid, cymoxanil, metrafenone.

The present invention furthermore relates to the pharmaceutical use of the compounds of the formula (I) according to the invention and/or the pharmaceutically acceptable salts thereof, in particular their use for controlling tumors in mammals such as, for example, humans.

### SYNTHESIS EXAMPLES

With appropriate modification of the starting materials, the procedures given in the synthesis examples below were used to obtain further compounds (I). The compounds obtained in this manner are listed in Table C below, together with physical data.

### 1. Synthesis of 6-(5-chloropyrimidin-4-yl)-5-methyl-7-(4-methylpiperidin-1-yl)-[1,2,4]triazolo[1,5-a]pyrimidine

### a) Dimethyl 2-[6-(5-chloropyrimidin-4-yl)-7-(4-methylpiperidin-1-yl)-[1,2,4]triazolo[1,5-a]pyrimidin-5-yl]malonate

With ice cooling, 0.037 g of 60% pure sodium hydride in white mineral oil were added carefully to 3 ml of dimethyl malonate, and the mixture was then diluted with 5 ml of diethylene glycol. A solution of 0.31 g (0.85 mmol) of 5-chloro-6-(5-chloropyrimidin-4-yl)-7-(4-methylpiperidin-1-yl)-[1,2,4]triazolo[1,5-a]pyrimidine in 3 ml of diethylene glycol dimethyl ether was then added, and the mixture was stirred at 50° C. for 4 h. After cooling, the mixture was taken up in ice water and extracted four times with ethyl acetate, and the combined organic phases were dried and concentrated. The residue was chromatographed on silica gel (cyclohexane:ethyl acetate=1:1). This gave 250 mg of the title compound.

### b) 6-(5-Chloropyrimidin-4-yl)-5-methyl-7-(4-methylpiperidin-1-yl)-[1,2,4]triazolo[1,5-a]pyrimidine

0.2 g (0.44 mmol) of dimethyl 2-[6-(5-chloropyrimidin-4-yl)-7-(4-methylpiperidin-1-yl)-[1,2,4]triazolo[1,5-a]pyrimidin-5-yl]malonate in 4 ml of conc. aqueous hydrochloric acid was stirred at 50° C. for 4 h and then at room temperature overnight. Using 10% strength aqueous sodium hydroxide solution, the solution Was adjusted to pH 6. The aqueous phase was extracted three times with ethyl acetate and the combined organic phases were dried and concentrated. This gave 30 mg of the title compound. (M+: 344)

All products were characterized by HPLC, mass spectrometry, by combined HPLC/mass spectrometry (High Performance Liquid Chromatography Mass Spectrometry) or by their melting point. For HPLC, an analytical RP-18 column (Chromolith Speed ROD from Merck KgaA, Germany), operated at 40° C., was used. Acetonitrile with a 0.1% by volume mixture of trifluoroacetic acid/water and 0.1% by volume of trifluoroacetic acid served as mobile phase. (The ratio of trifluoroacetic acid/water was changed from 5:95 to 95:5 over a period of 5 minutes). Mass spectrometry was carried out using a quadrupole mass spectrometer with electrospray ionization at 80V in the positive mode.

TABLE C

| | |
|---|---|
| | (I) |
| 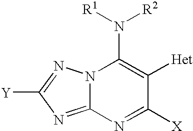 | |

| | | | | | physical |
|---|---|---|---|---|---|
| No. | NR¹R² | Het | X | Y | data |
| 1 | 4-methylpiperidin-1-yl | 5-chloropyrimidin-4-yl | CH₃ | H | M+: |
| | | | | | 344 |
| 2 | 4-methylpiperidin-1-yl | 3,5-dichloropyridin-2-yl | CH(COOCH₃)₂ | H | m.p. |
| | | | | | 95° C. |
| 3 | 4-methylpiperidin-1-yl | 3,5-dichloropyridin-2-yl | CH₃ | H | m.p. |
| | | | | | 162° C. |
| 4 | 4-methylpiperidin-1-yl | 3,5-dichloropyridin-4-yl | CH₃ | H | m.p. |
| | | | | | 149° C. |
| 5 | 4-methylpiperidin-1-yl | 3,5-dichloropyridin-4-yl | CH(COOCH₃)₂ | H | m.p. |
| | | | | | 216° C. |
| 6 | 4-methylpiperidin-1-yl | 3-bromopyridin-4-yl | CH(COOCH₃)₂ | H | m.p. |
| | | | | | 196° C. |
| 7 | 4-methylpiperidin-1-yl | 3-bromopyridin-4-yl | CH₃ | H | m.p. |
| | | | | | 208° C. |

### Examples of the Action of the Compounds According to the Invention Against Harmful Fungi

The respective active compound was prepared as a stock solution with 25 mg of active compound which was made up to 10 ml with a mixture of acetone and/or DMSO and the emulsifier Uniperol® EL (wetting agent having emulsifying and dispersing action based on ethoxylated alkylphenols) in a volume ratio solvent/emulsifier of 99 to 1. The solution was then made up to 100 ml with water. This stock solution was diluted to the active compound concentration stated below using the solvent/emulsifier/water mixture described.

### Use Example 1

### Activity Against Gray Mold on Bell Pepper Leaves Caused by Botrytis Cinerea, Protective Application

Bell pepper seedlings of the cultivar “Neusiedler ideal Elite” Were, after 2-3 leaves were well developed, sprayed to runoff point with an aqueous suspension having the concentration of active compound stated below. The next day, the treated plants were inoculated with a spore suspension of Botrytis cinerea which contained 1.7×10⁶ spores/ml in a 2% strength aqueous biomalt solution. The test plants were then placed in a dark climatized chamber at 22-24° C. and high atmospheric humidity. After 5 days, the extent of the fungal infection on the leaves could be determined visually in %.

The plants which had been treated with 250 ppm of the compound 3 or 4 showed an infection of less than 5%, whereas the untreated plants were 90% infected.

### Use Example 2

### Activity Against Net Blotch of Barley Caused by Pyrenophora teres, 1 Day Protective Application

Leaves of potted barley seedlings of the cultivar “Hanna” were sprayed to runoff point with an aqueous suspension having the concentration of active compound stated below. 24 hours after the spray coating had dried on, the test plants were inoculated with an aqueous spore suspension of Pyrenophora [syn. Drechslera] teres, the net blotch pathogen. The test plants were then placed in a greenhouse at temperatures of from 20 to 24° C. and 95-100% relative atmospheric humidity. After 6 days, the extent of the fungal infection on the leaves was determined visually in %.

### Use Example 3

### Activity Against Tomato Early Blight Caused by Alternaria Solani

Leaves of potted tomato plants were sprayed to runoff point with an aqueous suspension having the concentration of active compound stated below. The next day, the leaves were infected with an aqueous spore suspension of Alternaria solani in a 2% strength biomalt solution having a density of 0.17×10⁶ spores/ml. The plants were then placed in a water vapor-saturated chamber at temperatures between 20 and 22° C. After 5 days, the disease on the untreated but infected control plants had developed to such an extent that the infection could be determined visually in %.

The plants which had been treated with 250 ppm of the compound 3 or 4 showed an infection of less than 10%, whereas the untreated plants were 90% infected.

### Use Example 4

### Curative Activity Against Brown Rust of Wheat Caused by Puccinia Recondita

Leaves of potted wheat seedlings of the cultivar “Kanzler” were sprayed to runoff point with an aqueous suspension having the concentration of active compound stated below. The next day, the treated plants were inoculated with a spore suspension of brown rust of wheat (Puccinia recondite). The plants were then placed in a chamber with high atmospheric humidity (90 to 95%) at 20 to 22° C. for 24 hours. During this time, the spores germinated and the germ tubes penetrated into the leaf tissue. The next day, the test plants were returned to the greenhouse and cultivated at temperatures between 20 and 22° C. and at 65 to 70% relative atmospheric humidity for a further 7 days. The extent of the rust fungus development on the leaves was then determined visually.

### Use Example 5

### Protective Activity Against Puccinia Recondite on Wheat (Brown Rust of Wheat)

Leaves of potted wheat seedlings of the cultivar “Kanzler” were sprayed to runoff point with an aqueous suspension having the concentration of active compound stated below. The next day, the treated plants were inoculated with a spore suspension of brown rust of wheat (Puccinia recondita). The plants were then placed in a chamber with high atmospheric humidity (90 to 95%) at 20 to 22° C. for 24 hours. During this time, the spores germinated and the germ tubes penetrated into the leaf tissue. The next day, the test plants were returned to the greenhouse and cultivated at temperatures between 20 and 22° C. and at 65 to 70% relative atmospheric humidity for a further 7 days. The extent of the rust fungus development on the leaves was then determined visually.

### Use Example 6

### Activity Against the Speckled Leaf Blotch Pathogen Septoria Tritici in the Microtiter Test

The active compounds were formulated separately as a stock solution having a concentration of 10 000 ppm in DMSO.

The stock solution is pipetted into a microtiter plate (MTP) and diluted to the stated active compound concentration using an aqueous, malt-based nutrient medium for fungi. An aqueous spore suspension of Septoria tritici was then added. The plates were placed in a water vapor-saturated chamber at temperatures of 18° C. Using an absorption photometer, the MTPs were measured at 405 nm on day 7 after the inoculation. The parameters measured were compared to the growth of the active compound-free control variant and the fungus- and active compound-free blank value to determine the relative growth in % of the pathogens in the individual active compounds. The relative growth at 125 ppm of compound 1 was 0%.

1: A 7-aminomethyl-6-heteroaryl-1,2,4-triazolo[1,5-a]pyrimidine compound of the formula (I) 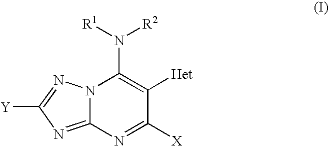
in which the substituents R¹, R², Het, X and Y are as defined below:
Het is a 6-membered heteroaromatic radical which contains one, two or three nitrogen atoms, where Het is unsubstituted or substituted by one, two, three or four identical or different substituents L,
R¹, R² independently of one another are hydrogen, (C₁-C₈)-alkyl, (C₁-C₈)-haloalkyl, (C₁-C₈)-alkoxy, (C₃-C₈)-cycloalkyl, (C₃-C₈)-cycloalkoxy, (C₅-C₁₀)-bicycloalkyl, (C₃-C₈)-halocycloalkyl, (C₂-C₈)-alkenyl, (C₂-C₈)-alkenyloxy, (C₄-C₁₀)-alkadienyl, (C₂-C₈)-haloalkenyl, (C₃-C₆)-cycloalkenyl, (C₃-C₆)-halocycloalkenyl, (C₂-C₈)-alkynyl, (C₂-C₈)-alkynyloxy, (C₂-C₈)-haloalkynyl, NH₂, (C₁-C₈)-alkylamino, di-(C₁-C₈)-alkylamino, phenyl, naphthyl or a five- or six-membered saturated, partially unsaturated or aromatic heterocycle which contains one, two, three or four heteroatoms from the group consisting of O, N and S,
R¹ and R² together with the nitrogen atom to which they are attached may also form a five- or six-membered heterocyclyl or heteroaryl which is attached via N and may contain one, two or three further heteroatoms from the group consisting of O, N and S as ring members and may carry one or more substituents from the group consisting of halogen, (C₁-C₆)-alkyl, (C₁-C₆)-haloalkyl, (C₂-C₆)-alkenyl, (C₂-C₆)-haloalkenyl, (C₁-C₆)-alkoxy, (C₁-C₆)-alkoxycarbonyl, (C₁-C₆)-haloalkoxy, (C₃-C₆)-alkenyloxy, (C₃-C₆)-haloalkenyloxy and in which two substituents attached to adjacent ring atoms may be (C₁-C₆)-alkylene, oxy-(C₂-C₄)-alkylene or oxy-(C₁-C₃)-alkyleneoxy;
R¹ and R² may carry one, two, three or four identical or different groups R^{a}:
R^{a} is halogen, cyano, nitro, hydroxyl, carboxyl, (C₁-C₆)-alkyl, (C₁-C₆)-haloalkyl, (C₁-C₆)-alkylcarbonyl, (C₃-C₆)-cycloalkyl, (C₁-C₆)-alkoxy, (C₁-C₆)-haloalkoxy, (C₁-C₆)-alkoxycarbonyl, (C₁-C₆)-alkylthio, (C₁-C₆)-alkylamino, di-(C₁-C₆)-alkyl-amino, (C₁-C₆)-alkylaminocarbonyl, di-(C₁-C₆)-alkylaminocarbonyl, (C₂-C₈)-alkenyl, (C₄-C₁₀)-alkadienyl, (C₂-C₈)-haloalkenyl, (C₃-C₈)-cycloalkenyl, (C₂-C₆)-alkenyloxy, (C₃-C₆)-haloalkenyloxy, (C₂-C₆)-alkynyl, (C₂-C₆)-haloalkynyl, (C₃-C₆)-alkynyloxy, (C₃-C₆)-haloalkynyloxy, (C₃-C₆)-cycloalkoxy, (C₃-C₆)-cyclo-alkenyloxy, oxy-(C₁-C₃)-alkyleneoxy, phenyl, naphthyl, a five-, six-, seven-, eight-, nine- or ten-membered saturated, partially unsaturated or aromatic heterocycle which contains one, two, three or four heteroatoms from the groups consisting of O, N and S,
where the aliphatic, alicyclic or aromatic groups in R^{a} for their part may be partially or fully halogenated and may carry one, two or three groups R^{b}:
R^{b} is halogen, cyano, nitro, hydroxyl, mercapto, amino, carboxyl, aminocarbonyl, aminothiocarbonyl, alkyl, haloalkyl, alkenyl, alkadienyl, alkenyloxy, alkynyloxy, alkoxy, haloalkoxy, alkylthio, alkylamino, dialkylamino, formyl, alkylcarbonyl, alkylsulfonyl, alkylsulfinyl, alkoxycarbonyl, alkylcarbonyloxy, alkylaminocarbonyl, dialkylaminocarbonyl, alkylaminothiocarbonyl, dialkylaminothiocarbonyl, where the alkyl groups in these radicals contain 1 to 6 carbon atoms and the alkenyl, alkadienyl or alkynyl groups mentioned in these radicals contain 2 to 8 carbon atoms;
or one, two or three of the following radicals:
cycloalkyl, bicycloalkyl, cycloalkoxy, heterocyclyl, heterocyclyloxy, where the cyclic systems contain 3 to 10 ring members; aryl, aryloxy, arylthio, aryl-(C₁-C₆)-alkoxy, aryl-(C₁-C₆)-alkyl, heteroaryl, heteroaryloxy, heteroarylthio, where the aryl radicals preferably contain 6, 7, 8, 9 or 10 ring members and the heteroaryl radicals 5 or 6 ring members, where the cyclic systems may be partially or fully halogenated or substituted by alkyl or haloalkyl groups;
X is (C₁-C₈)-alkyl, (C₂-C₈)-alkenyl, (C₂-C₈)-alkynyl; where these groups may be partially or fully halogenated and may carry one, two or three substituents selected from the group consisting of nitro, cyano, (C₁-C₂)-alkoxy, (C₁-C₄)-alkoxycarbonyl, amino, (C₁-C₄)-alkylamino and di-(C₁-C₄)-alkylamino;
Y is hydrogen, halogen, cyano, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₁-C₈)-alkoxy, (C₁-C₈)-haloalkoxy, (C₃-C₈)-cycloalkyl, (C₃-C₈)-halocycloalkyl, (C₁-C₈)-alkylthio, (C₁-C₈)-alkylsulfinyl, (C₁-C₈)-alkylsulfonyl, NH₂, (C₁-C₈)-alkylamino, di-(C₁-C₈)-alkylamino or C(═O)A2;
where
L is selected from the group consisting of halogen, cyano, hydroxyl, cyanato (OCN), nitro, (C₁-C₈)-alkyl, (C₂-C₁₀)-alkenyl, (C₂-C₁₀)-alkynyl, (C₁-C₆)-haloalkyl, (C₂-C₁₀)-haloalkenyl, (C₁-C₆)-alkoxy, (C₂-C₁₀)-alkenyloxy, (C₂-C₁₀)-alkynyloxy, (C₁-C₆)-haloalkoxy, (C₃-C₆)-cycloalkyl, (C₃-C₆)-cycloalkenyl, (C₃-C₆)-cycloalkoxy, (C₁-C₈)-alkoximinoalkyl, (C₂-C₁₀)-alkenyloximinoalkyl, (C₂-C₆)-alkynyloximinoalkyl, (C₂-C₁₀)-alkynylcarbonyl, (C₃-C₆)-cycloalkylcarbonyl, phenyl, a five-, six-, seven-, eight-, nine- or ten-membered saturated, partially unsaturated or aromatic heterocycle which contains one, two, three or four heteroatoms from the group consisting of O, N and S, where the phenyl and the heterocycle are unsubstituted or substituted by one, two, three or four substituents independently of one another selected from the group consisting of halogen, (C₁-C₆)-alkyl, (C₁-C₆)-haloalkyl, (C₂-C₆)-alkenyl, (C₂-C₆)-haloalkenyl, (C₁-C₆)-alkoxy, (C₁-C₆)-alkoxycarbonyl, (C₁-C₆)-haloalkoxy, (C₃-C₆)-alkenyloxy, (C₃-C₆)-haloalkenyloxy and in which two substituents attached to adjacent ring atoms may be (C₁-C₆)-alkylene, oxy-(C₂-C₄)-alkylene or oxy-(C₁-C₃)-alkyleneoxy; amino, NR⁵R⁶, NR⁵—(C═O)—R⁶, S(═O)ₙA¹, C(═O)A², C(═S)A², a group —C(═N—OR⁷)(R⁸R⁹) or a group —C(═N—NR¹⁰R¹¹)(NR¹²R¹³),
in which
R⁵, R⁶ independently of one another are selected from the group consisting of hydrogen, (C₁-C₆)-alkyl, (C₂-C₁₀)-alkenyl, (C₂-C₁₀)-alkynyl, (C₃-C₆)-cycloalkyl or (C₃-C₆)-cycloalkenyl, where the 5 last-mentioned radicals may be partially or fully halogenated and/or may carry one, two, three or four radicals selected from the group consisting of cyano, (C₁-C₄)-alkoximino, (C₂-C₄)-alkenyloximino, (C₂-C₄)-alkynyloximino and (C₁-C₄)-alkoxy;
A¹ is hydrogen, hydroxyl, (C₁-C₈)-alkyl, amino, (C₁-C₈)-alkylamino or di-(C₁-C₈)-alkylamino;
n is 0, 1 or 2;
A² is (C₂-C₈)-alkenyl, (C₁-C₈)-alkoxy, (C₁-C₆)-haloalkoxy, (C₂-C₁₀)-alkenyloxy, (C₂-C₁₀)-alkynyloxy or one of the groups mentioned under A¹;
R⁷, R⁸, R⁹, R¹⁰, R¹¹, R¹² and R¹³ independently of one another are selected from the group consisting of hydrogen, (C₁-C₆)-alkyl, (C₃-C₆)-cycloalkyl, (C₂-C₆)-alkenyl and (C₂-C₆)-alkynyl, where the four last mentioned radicals may have one, two, three, four, five or six radicals R^{a}; or
R⁸ and R⁹, R¹⁰ and R¹¹ and R¹² and R¹³ together with the nitrogen atom to which they are attached form a four-, five- or six-membered saturated or partially unsaturated ring which may carry one, two, three or four substituents independently of one another selected from the group R^{a};
or an agriculturally acceptable salt of a compound (I).

2: The compound according to claim 1 in which at least one of the radicals R¹ and R² is different from hydrogen.

3: The compound according to claim 1 in which R¹ and R² together with the nitrogen atom to which they are attached form a five- or six-membered heterocyclyl or heteroaryl which is attached via N and which may contain one, two or three further heteroatoms from the group consisting of O, N and S as ring members, where the heterocyclyl or heteroaryl may be unsubstituted or substituted by one or two identical or different substituents R^{a}.

4: The compound according to claim 1 in which R¹ and R² are hydrogen.

5: The compound according to claim 1 in which X is (C₁-C₄)-alkyl or (C₁-C₄)-haloalkyl.

6: The compound according to claim 1 in which Het contains at least one substituent located ortho to the point of attachment to the pyrimidine skeleton.

7: The compound according to claim 1 in which Het is pyrimidinyl which is unsubstituted or substituted by 1, 2 or 3 substituents L, as defined in claim 1.

8. (canceled)

9. A composition comprising at least one compound of the formula (I) according to claim 1 or an agriculturally acceptable salt thereof and at least one solid or liquid carrier.

10: A composition comprising at least one compound of the formula (I) according claim 1 or an agriculturally acceptable salt thereof and at least one further fungicidally, insecticidally or herbicidally active compound.

11: A method for controlling phytopathogenic fungi, which comprises treating the fungi or the materials, plants, the soil or seed to be protected against fungal attack with an effective amount of at least one compound of the formula (I) according to claim 1 or an agriculturally acceptable salt thereof.

12: Seed, comprising a compound of the formula (I) according to claim 1 in an amount of from 1 to 1000 g per 100 kg.

13: A process for preparing a compound of the formula (I) according to claim 1, comprising the step:
(a) contacting a 7-halo-1,2,4-triazolo[1,5-a]pyrimidine compound of the formula (II) 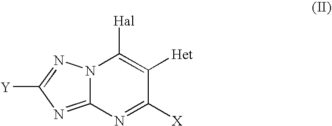
with an amine HNR¹R², where Hal is halogen wherein a compound of formula (I) is prepared.

14: A process for preparing a compound of the formula (II) according to claim 13, comprising the step:
(a1) contacting a compound of the formula (III) 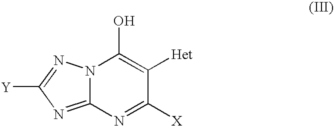
with a halogenating agent, wherein a compound of formula (II) is prepared.

15. A process for preparing a compound of the formula (III) according to claim 14, comprising the step:
(a2) of contacting a compound of the formula (IV) 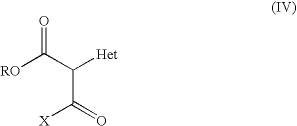
with a compound of the formula (V) 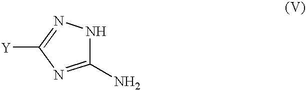
where R is (C₁-C₄)-alkyl wherein a compound of formula (III) is prepared.

16: A process for preparing a compound of the formula (I) according claim 1, comprising the step:
(a3) of contacting a compound of the formula (IIa) 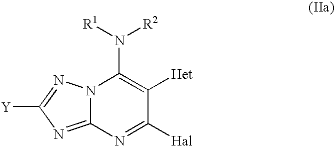
in which Hal is halogen with an organometallic compound X^{a}—Mt in which X^{a} is (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, cyano-(C₁-C₄)-alkyl or (C₁-C₄)-alkoxy-(C₁-C₄)-alkyl and Mt is lithium, magnesium or zinc.

17: A process for preparing a compound of the formula (I) according to any of claim 1 in which X is (C₁-C₈)-alkyl, comprising the step:
(a4) contacting a compound of the formula (IIa) 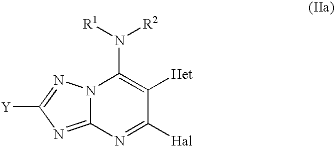
in which Hal is halogen with a compound of the formula (IVb) 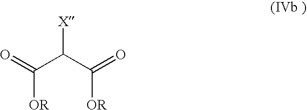
to give a compound of the formula (VI) 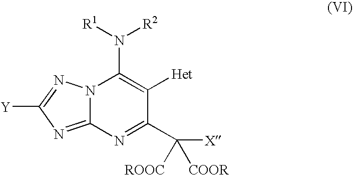
in which X″ is hydrogen or (C₁-C₇)-alkyl and R is (C₁-C₄)-alkyl;
(b4) hydrolysis of the compound (VI) obtained in step (c); and
(c4) decarboxylation of the hydrolysis product obtained in step (d).

18: A compound of the formula (II) according to claim 13.

19: A compound of the formula (III) according to claim 14.

20: A compound of the formula (VI) according to claim 17.
